(19) United States
(12) Patent Application Publication
(10) Pub. No.: US 2008/0044413 A1
(43) Pub. Date: Feb. 21, 2008

(54) EphA2 BiTE molecules and uses thereof

(76) Inventors: Scott A. Hammond, Olney, MD (US); Peter A. Kiener, Potomac, MD (US); Elizabeth Bruckheimer, Rockville, MD (US); Michael S. Kinch, Laytonsville, MD (US); Shannon Roff, Eldersburg, MD (US); Ralf Lutterbuese, Neuried (DE); Petra Lutterbuese, Neuried (DE); Bernd Schlereth, Germering (DE); Patrick Alexander Baeuerle, Gauting (DE); Peter Kufer, Moosburg (DE)

Correspondence Address: JONES DAY, 222 EAST 41ST ST, NEW YORK, NY, 10017, US

(21) Appl. No.: 11/645,290

(22) Filed: Dec. 21, 2006

Related U.S. Application Data
(60) Provisional application No. 60/753,368, filed on Dec. 21, 2005.

(51) Int. Cl. A61K 39/395; A61P 31/00; A61P 35/00; A61P 43/00; C07K 16/28

(52) U.S. Cl. 424/135.1; 530/387.3

(57) ABSTRACT

The present invention relates to bispecific single chain antibodies comprising a first binding domain that immunospecifically binds to the T-cell antigen CD3 and a second binding domain that immunospecifically binds to the EphA2 receptor. Such bispecific single chain antibodies are encompassed by the term “EphA2-BiTEs.” The present invention further relates to methods and compositions designed for the treatment, prevention and/or management of disorders associated with aberrant expression and/or activity of EphA2. Such disorders include, but are not limited to, cancer, non-cancer hyperproliferative cell disorders, and infections. The invention further relates to vectors comprising polynucleotides encoding the EphA2-BiTEs of the invention, host cells transformed therewith, and their use in the production of said EphA2-BiTEs. The invention also provides compositions, including pharmaceutical compositions, comprising any of the aforementioned EphA2-BiTEs, polynucleotides or vectors either alone or in combination with one or more prophylactic or therapeutic agents. Also disclosed are methods of screening for said EphA2-BiTEs and kits comprising any of the aforementioned compositions and diagnostic reagents.

This application claims benefit of U.S. Provisional Application No. 60/753,368, filed Dec. 21, 2005, which is incorporated by reference herein in its entirety.

### 1. FIELD OF THE INVENTION

The present invention relates to bispecific single chain antibodies comprising a first binding domain that immunospecifically binds to the T-cell antigen CD3 and a second binding domain that immunospecifically binds to the EphA2 receptor. Such bispecific single chain antibodies are encompassed by the term “EphA2-BiTEs.” The present invention further relates to methods and compositions designed for the treatment, prevention and/or management of disorders associated with aberrant expression and/or activity of EphA2. Such disorders include, but are not limited to, cancer, non-cancer hyperproliferative cell disorders, and infections. The invention further relates to vectors comprising polynucleotides encoding the EphA2-BiTEs of the invention, host cells transformed therewith, and their use in the production of said EphA2-BiTEs. The invention also provides compositions, including pharmaceutical compositions, comprising any of the aforementioned EphA2-BiTEs, polynucleotides or vectors either alone or in combination with one or more prophylactic or therapeutic agents. Also disclosed are methods of screening for said EphA2-BiTEs and kits comprising any of the aforementioned compositions and diagnostic reagents.

### 2. BACKGROUND OF THE INVENTION

2.1 EphA2

EphA2 is a 130 kDa receptor tyrosine kinase that is expressed in adult epithelia, where it is found at low levels and is enriched within sites of cell-cell adhesion (Zantek et al., 1999, Cell Growth & Differentiation 10(9):629-38; Lindberg et al., Mol. & Cell. Biol. 10:6316, 1990). This subcellular localization is thought to play a role in contact inhibition through the interaction of EphA2 with its ligands (known as EphrinsA1 to A5) that are anchored to the cell membrane on adjacent cells (Eph Nomenclature Committee, 1997, Cell 90:403-04; Cheng et al., 2002, Cytokine & Growth Factor Rev. 13:75-85). Engagement of EphA2 with its ligand results in autophosphorylation of EphA2 and its subsequent degradation (Walker-Daniels et al., 2002, Mol. Cancer. Res. 1(1):79-87; Carles-Kinch et al., 2002, Cancer Res. 62(10):2840-47). This signaling cascade also initiates downstream events that negatively regulate attachment to extracellular matrix adhesion molecules and thereby regulate cell growth and migration (Zantek et al., 1999, Cell Growth & Differentiation 10(9):629-38; Miao et al., 2000, Nat. Cell Biol. 2(2):62-69; Zelinski et al., 2001, Cancer Res. 61(5):2301-06).

EphA2 has been shown to be overexpressed in a number of different tumor types including melanoma, renal cell carcinoma, breast, prostate, colon, esophageal, cervical, lung, ovarian and bladder cancers (Carles-Kinch et al., 2002, Cancer Res. 62(10):2840-47). The highest levels of EphA2 expression are observed in the most aggressive cells, suggesting a role for EphA2 in disease progression. High levels of EphA2 have also been correlated with poor survival for non-small cell lung, esophageal, cervical and ovarian cancers (Kinch et al., 2003, Clin. Cancer Res. 9(2):613-18; Miyazaki et al., 2003, Int. J. Cancer 103(5) 657-63; Wu et al., 2004, Gynecol. Oncol. 94(2):312-19; Thaker et al., 2004, Clin. Cancer Res. 10(15):5145-50). Additionally, in pre-clinical models, it has been demonstrated that exogenous expression of EphA2 is sufficient to render a non-tumorigenic cell line tumorigenic in vitro and in vivo (Zelinski et al., 2001, Cancer Res. 61(5):2301-06).

2.2 BITE® Molecules

Bispecific T-cell engagers, or BiTEs®, are a form of bispecific antibodies that are based on tandemly arranged single-chain antibodies (reviewed in Wolf et al., 2005, Drug Discovery Today: in press). They form a single polypeptide chain of approximately 55 kDa and are secreted by Chinese hamster ovary (CHO) cells as a mixture of monomers and dimers. With one arm, BiTEs® bind to the epsilon (ε) subunit of human CD3, a protein component of the signal-transducing complex of the T-cell receptor on T-cells. With the other arm, BiTEs® recognize an antigen on target cells. T-cell activation is only seen when BiTEs® are presented to T-cells on the surface of target cells.

BiTEs® transiently tether T-cells and target cells. T-cell activation by BiTEs® involves upregulation of CD69, CD25 and various cell adhesion molecules, de novo expression and release of cytokines (e.g., IFN-γ, TNF-α, IL-6, IL-2, IL-4 and IL-10), upregulation of granzyme and perforin expression, and cell proliferation. Redirected target cell lysis by BiTEs® is independent of T-cell receptor specificity, presence of MHC class I and β2 microglobulin, and of any co-stimulatory stimuli. This independence from regular T-cell signals and recognition molecules may be explained by the induction through BiTEs® of regular cytolytic synapses and maximum membrane proximity. Displacement of negative regulatory proteins such as CD45 from BiTE®-induced synapses may alleviate the need for co-stimulation.

BiTEs® show redirected lysis in vitro with previously unstimulated peripheral polyclonal CD8- and CD4-positive T-cells. No activity is seen with naïve CD8- or CD4-positive T-cells. CD4 T-cells can upregulate granzyme B and perforin expression when stimulated with BiTEs® and thereby contribute to CD8-mediated target cell lysis. In vitro, redirected lysis is seen at low picomolar concentrations, suggesting that very low numbers of BiTE molecules need to be bound to target cells for triggering T-cells. In SCID mouse models, sub-μg doses of BiTEs® have been shown to completely prevent tumor outgrowth (Dreier et al., 2003, J Immunol. 170:4397-4402) and to eradicate solid tumors up to 200 mm³ (Schlereth et al., 2005, Cancer Res. 2005 65(7):2882-89).

BiTEs®, therefore, provide a unique opportunity to develop selective and efficacious antibody-based therapies against EphA2 for the treatment, prevention and/or management of disorders associated with aberrant expression and/or activity of EphA2 (e.g., cancer, non-cancer hyperproliferative cell disorders, and infections).

### 3. SUMMARY OF THE INVENTION

The present invention provides bispecific T-cell engagers (i.e., EphA2-BiTEs (in particular, EphA2-BiTEs, which are bispecific single chain antibodies)) that immunospecifically bind EphA2 and the T-cell antigen CD3, and methods of using the same to treat, prevent and/or manage disorders associated with aberrant expression and/or activity of EphA2. Such disorders include, for example, cancer, non-cancer hyperproliferative cell disorders, and infections. In one aspect, the EphA2-BiTEs are more efficient at eliminating cells that aberrantly express EphA2 than EphA2-specific antibodies known in the art. In a specific aspect, the EphA2-BiTEs are more efficient at eliminating EphA2-expressing cancer cells (in particular, EphA2-expressing malignant cancer cells) than EphA2 antibodies known in the art. In another aspect, the EphA2-BiTEs are more efficient at eliminating EphA2-expressing non-cancer hyperproliferative cells than EphA2 antibodies known in the art. In yet another aspect, the EphA2-BiTEs of the invention are more efficient at eliminating EphA2-expressing infected cells (in particular, cells infected with the Respiratory Syncytial Virus; “RSV”) than EphA2 antibodies known in the art. In another aspect, lower dosages of EphA2-BiTEs than EphA2-specific antibodies known in the art are needed to treat, prevent and/or manage disorders associated with aberrant expression and/or activity of EphA2.

The EphA2-specific bispecific T-cell engagers of the present invention comprise a first binding domain that immunospecifically binds to the T-cell antigen CD3 and a second binding domain that immunospecifically binds to EphA2 (hereinafter “EphA2-BiTEs,” “EphA2-BiTE molecules” or “EphA2 bispecific T-cell engagers). In one embodiment, the first binding domain immunospecifically binds to CD3. In a specific embodiment, the first binding domain immunospecifically binds to one or more of any subunit of CD3 (e.g., the gamma, delta, zeta, or eta subunit). In a preferred embodiment, the first binding domain immunospecifically binds to the epsilon (ε) subunit of CD3. In a specific embodiment, the first binding domain immunospecifically binds to the epsilon (ε) subunit of CD3 when said subunit is complexed with the delta subunit of CD3. In another embodiment, the binding domain that binds to CD3 is deimmunized. In another specific embodiment, the second binding domain immunospecifically binds to the extracellular domain of EphA2. In a preferred embodiment, the second binding domain of the EphA2-BiTEs, which are used in the treatment, prevention and/or management of cancer, immunospecifically binds to epitopes on EphA2 that are selectively exposed and/or increased on cancer cells but not non-cancer cells. In another preferred embodiment, the second binding domain of the EphA2-BiTEs of the invention immunospecifically binds to epitopes on EphA2 that are selectively exposed and/or increased on non-cancer hyperproliferative cells but not non-hyperproliferative cells. In another preferred embodiment, the second binding domain of the EphA2-BiTEs of the invention immunospecifically binds to epitopes on EphA2 that are selectively exposed and/or increased on infected cells but not non-infected cells.

In a specific embodiment, an EphA2-BiTE of the invention comprises: (1) a first binding domain comprises a variable heavy (VH) domain and a variable light (VL) domain of an antibody that immunospecifically binds to the T-cell antigen CD3; and (2) a second binding domain that comprises a VH domain and a VL domain of an antibody that immunospecifically binds to EphA2. In a specific embodiment, the VH domain and VL domains of the first binding domain are linked together by a linker of sufficient length to enable the domains to fold in such a way as to permit binding to the T-cell antigen CD3. Further to this embodiment, such a linker may comprise, for example, the sequence GEGTSTGS(G₂S)₂GGAD (SEQ ID NO.:57). In another specific embodiment, the VH domain and VL domains of the second binding domain are linked together by a linker of sufficient length to enable the domains to fold in such a way as to permit binding to EphA2. Further to this embodiment, such a linker may comprise, for example, the sequence (G₄S)₃ (SEQ ID NO:59). In another specific embodiment, the first and second binding domains are linked together by a linker of sufficient length to enable the domains to fold in such a way as to permit binding to the T-cell antigen CD3 and to EphA2. Further to this embodiment, such a linker may comprise, for example, the sequence G₄S (SEQ ID NO:58). In a specific embodiment, an EphA2-BiTE of the invention is Deimmunized anti-CD3×EA2 (VH/VL) (SEQ ID NO:65).

In accordance with the embodiment in the immediately preceding paragraph, the linkage is covalent. In a specific embodiment, the linkers of the invention comprise serine and glycine residues. The linkers of the EphA2-BiTEs, e.g., the linker between the VH and VL domains of the first binding domain that binds to CD3, the linker between the VH and VL domains of the second binding domain that binds to EphA2, and the linker between the first binding domain that binds to CD3 and the second binding domain that binds to EphA2 may be of any length sufficient to enable the domains to fold in such a way as to permit binding to the CD3 and EphA2 antigens, respectively. In certain embodiments, the linkers of the invention comprise a length of at least 5 residues, at least 10 residues, at least 15 residues, at least 20 residues, at least 25 residues, at least 30 residues or more. In other embodiments, the linkers of the invention comprises a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, or between 2-30 residues. In certain embodiments, the first binding domain is 5′ to the second binding domain. In other embodiments, the second binding domain is 5′ to the first binding domain. In certain embodiments, the first and second binding domains are single chain antibodies. In a specific embodiment, the first and second binding domains comprise single chain Fvs (scFvs).

In a specific embodiment, the invention provides a bispecific single chain antibody comprising (a) a first heavy chain variable domain and a first light chain variable domain each from an antibody that immunospecifically binds the ε chain of CD3, said first heavy chain variable domain covalently linked to said first light chain variable domain by a first linker of sufficient length (e.g., GEGTSTGS(G₂S)₂GGAD (SEQ ID NO.:57)) such that said first heavy chain variable domain and said first light chain variable domain fold to form a first binding domain that binds the ε subunit of CD3; and (b) a second heavy chain variable domain and a second light chain variable domain from an antibody that immunospecifically binds an epitope of EphA2 exposed on the cell surface, said second heavy chain variable domain covalently linked to said second light chain variable domain by a second linker of sufficient length (e.g., (G₄S)₃ (SEQ ID NO:59)) such that said second heavy chain variable domain and said second light chain variable domain fold to form a second binding domain that binds said epitope of EphA2, wherein said first binding domain and said second binding domain are covalently linked by a third linker of a length (e.g., G₄S (SEQ ID NO:58)) such that said first binding domain and said second binding domain fold independently of each other.

In specific embodiments, the EphA2-BiTEs of the invention comprise any of the following arrangements in the 5′ to 3′ direction: (1) VH_{CD3}-VL_{CD3}-VH_{EphA2}-VL-_{EphA2}; (2) VL_{CD3}-VH_{CD3}-VH_{EphA2}-VL_{EphA2}; (3) VL_{CD3}-VH_{CD3}-VL_{EphA2}-VH-_{EphA2}; (4) VH_{CD3}-VL_{CD3}-VL_{EphA2}-VH_{EphA2}; (5) VH_{EphA2}-VL_{EphA2}-VH_{CD3}-VL_{CD3}; (6) VL_{EphA2}-VH_{EphA2}-VH_{CD3}-VL_{CD3}; (7) VL_{EphA2}-VH_{EphA2}-VL_{CD3}-VH_{CD3}; or (8) VH_{EphA2}-VL_{EphA2}-VL_{CD3}-VH-_{CD3}. See, e.g., FIG. 14A for a generic depiction of the EphA2-BiTE constructs of the invention.

In a specific embodiment, the first binding domain of an EphA2-BiTE of the invention binds to the ε subunit of CD3 with a lower affinity than the second binding domain that binds EphA2. In one embodiment, the dissociation constant (K_{D}) of the first binding domain that binds to the ε subunit of CD3 is between 0.1×10⁻¹² M to 0.5×10⁻¹² M, 0.1×10⁻¹² M to 1×10⁻¹² M, 0.1×10⁻¹¹ M to 0.5×10⁻¹¹ M, 0.1×10⁻¹¹ M to 1×10⁻¹¹ M, 0.1×10⁻¹⁰ M to 0.5×10⁻¹⁰ M, 0.1×10⁻¹⁰ M to 1×10⁻¹⁰ M, 0.1×10⁻⁹ M to 0.5×10⁻⁹ M, 0.1×10⁻⁹M to 1×10⁻⁹ M, 0.1×10⁻⁸ M to 0.5×10⁻⁸ M, 0.1×10⁻⁸ M to 1×10⁻⁸ M, 0.1×10⁻⁷ M to 0.5×10⁻⁷ M, 0.1×10⁻⁷ M to 1×10⁻⁷ M, 1×10⁻⁷ M to 2×10⁻⁷ M, 1×10⁻⁷ M to 3×10⁻⁷ M, 1×10⁻⁷ M to 4×10⁻⁷ M, 1×10⁻⁷ M to 5×10⁻⁷ M, 1×10⁻⁷ M to 6×10⁻⁷M, 1×10⁻⁷ M to 7×10⁻⁷ M, 1×10⁻⁷ M to 8×10⁻⁷M, 1×10⁻⁷ M to 9×10⁻⁷ M, 1×10⁻⁷ M to 10×10⁻⁷ M, 0.1×10⁻⁶ M to 0.5×10⁻⁶ M, 0.1×10⁻⁶ M to 1×10⁻⁶ M, 1×10⁻⁶ M to 2×10⁻⁶M, 1×10⁻⁶M to 3×10⁻⁶M, 1×10⁻⁶ M to 4×10⁻⁶M, 1×10⁻⁶ M to 5×10⁻⁶ M, 1×10⁻⁶ M to 6×10⁻⁶ M, 1×10⁻⁶ M to 7×10⁻⁶M, 1×10⁻⁶ M to 8×10⁻⁶ M, 1×10⁻⁶ M to 9×10⁻⁶M, 1×10⁻⁶ M to 10×10⁻⁶ M, 0.1×10⁻⁵ M to 0.5×10⁻⁵ M, 0.1×10⁻⁵ M to 1×10⁻⁵ M, 1×10⁻⁵ M to 2×10⁻⁵ M, 1×10⁻⁵ M to 3×10⁻⁵ M, 1×10⁻⁵ M to 4×10⁻⁵ M, 1×10⁻⁵ M to 5×10⁻⁵ M, 1×10⁻⁵ M to 6×10⁻⁵ M, 1×10⁻⁵ M to 7×10⁻⁵ M, 1×10⁻⁵ M to 8×10⁻⁵ M, 1×10⁻⁵ M to 9×10⁻⁵ M, 1×10⁻⁵ M to 10×10⁻⁵ M. In a specific embodiment, the dissociation constant of the first domain that binds to the ε subunit of CD3 is 4×10⁻⁷ M. In another specific embodiment, the dissociation constant of the second domain that binds to EphA2 is between 0.1×10⁻¹² M to 0.5×10⁻¹¹ M, 0.1×10⁻¹² M to 1×10⁻¹² M, 0.1×10⁻¹¹ M to 0.5×10⁻¹¹ M, 0.1×10⁻¹¹ M to 1×10⁻¹¹ M, 0.1×10⁻¹⁰ M to 0.5×10⁻¹⁰ M, 0.1×10⁻¹⁰ M to 1×10⁻¹⁰ M, 0.1×10⁻⁹ M to 0.5×10⁻⁹ M, 0.1×10⁻⁹ M to 1×10⁻⁹ M, 0.1×10⁻⁸ M to 0.5×10⁻⁸M, 0.1×10⁻⁸ M to 1×10⁻⁸ M, 0.1×10⁻⁷M to 0.5×10⁻⁷ M, 0.1×10⁻⁷ M to 1×10⁻⁷ M, 1×10⁻⁷ M to 2×10⁻⁷ M, 1×10⁻⁷ M to 3×10⁻⁷ M, 1×10⁻⁷ M to 4×10⁻⁷ M, 1×10⁻⁷ M to 5×10⁻⁷M, 1×10⁻⁷ M to 6×10⁻⁷ M, 1×10⁻⁷ M to 7×10⁻⁷ M, 1×10⁻⁷ M to 8×10⁻⁷ M, 1×10⁻⁷ M to 9×10⁻⁷ M, 1×10⁻⁷ M to 10×10⁻⁷M, 0.1×10⁻⁶ M to 0.5×10⁻⁶ M, 0.1×10⁻⁶ M to 1×10⁻⁶M, 1×10⁻⁶ M to 2×10⁻⁶ M, 1×10⁻⁶ M to 3×10⁻⁶ M, 1×10⁻⁶ M to 4×10⁻⁶ M, 1'10⁻⁶ M to 5×10⁻⁶ M, 1×10⁻⁶ M to 6×10⁻⁶ M, 1×10⁻⁶ M to 7×10⁻⁶ M, 7×10⁻⁶ M to 8×10⁻⁶ M, 1×10⁻⁶ M to 9×10⁻⁶ M, 1×10⁻⁶M to 10×10⁻⁶ M, 0.1×10⁻⁵ M to 0.5×10⁻⁵ M, 0.1×10⁻⁵ M to 1×10⁻⁵M, 1×10⁻⁵ M to 2×10⁻⁵ M, 1×10⁻⁵ M to 3×10⁻⁵ M, 1×10⁻⁵ M to 4×10⁻⁵ M, 1×10⁻⁵ M to 5×10⁻⁵ M, 1×10⁻⁵ M to 6×10⁻⁵ M, 1×10⁻⁵ M to 7×10⁻⁵ M, 1×10⁻⁵ M to 8×10⁻⁵ M, 1×10⁻⁵ M to 9×10⁻⁵ M, 1×10⁻⁵ M to 10×10⁻⁵ M. In a specific embodiment, the dissociation constant of the second domain that binds to EphA2 is 1.13×10⁻⁷ M. In another specific embodiment, an EphA2-BiTE of the invention comprises a first binding domain that binds to the ε subunit of CD3 with a K_{D} of 4×10⁻⁷ M and a second binding domain that binds to EphA2 with a K_{D} of 1.13×10⁻⁷ M.

In specific embodiments, the EphA2-binding domain of an EphA2-BiTE of the invention has a high affinity constant and a low dissociation constant. In an alternative embodiment, the EphA2-binding domain of an EphA2-BiTE of the invention has a low affinity constant and a high dissociation constant. In another embodiment, the EphA2-binding domain of an EphA2-BiTE of the invention has a high affinity constant and a high dissociation constant. In another specific embodiment, the CD3-binding domain of an EphA2-BiTE of the invention binds to the ε subunit of CD3 with a lower affinity than the second binding domain that binds EphA2.

In a specific embodiment, the EphA2-BiTEs of the invention bind to EphA2 first and then bind to CD3. In another specific embodiment, the EphA2-BiTEs of the invention cause lysis of target cells that express EphA2 as measured by a standard cytotoxicity assay known in the art or as described below in Section 6.2.6 and 6.3. In a specific embodiment, the EphA2-BiTEs of the invention mediate lysis of target cells (e.g., cancer, non-cancer hyperproliferative or infected cells that express EphA2) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of EphA2 expression in the normal cells, cells of a normal, healthy subject and/or a population of normal, healthy cells that express EphA2 as measured by a flow-cytometry-based assay as described in Section 6.0 below. In yet another embodiment, the EphA2-BiTEs of the invention do not activate (e.g., phosphorylate) EphA2 when bound to EphA2 as measured by an immunoprecipitation/Western blot assay as described in Section 6.0 below. In a specific embodiment, less than 20%, less than 15%, or less than 5% of the population of EphA2 receptors are activated (e.g., phosphorylated) when bound to an EphA2-BiTE of the invention.

The present invention further provides compositions comprising the EphA2-BiTEs of the invention. In particular, the present invention provides pharmaceutical compositions comprising the EphA2-BiTEs of the invention and one or more pharmaceutical carriers or excipients. The present invention provides aqueous formulations, lyophilized formulations, gels, and surgical implants containing any of the EphA2-BiTEs of the invention. The present invention also provides kits comprising one or more EphA2-BiTEs of the invention, in one or more containers, and instructions for use of such EphA2-BiTEs.

The present invention provides compositions and methods for treating, preventing and/or managing cancer associated with aberrant EphA2 expression (e.g., overexpression) and/or activity, the methods comprising administering to a subject in need thereof an EphA2-BiTE of the invention. In a specific embodiment, the present invention provides methods for treating, preventing and/or managing cancer associated with aberrant EphA2 expression, the methods comprising administering to a subject in need thereof a prophylactically or therapeutically effective amount of an EphA2-BiTE of the invention.

In one embodiment, the cancer to be treated, prevented and/or managed is of an epithelial cell origin. In yet another embodiment, the cancer cells of the cancer of a subject to be to be treated, prevented and/or managed overexpress EphA2 relative to normal cells of said subject, cells of a normal, healthy subject and/or a population of normal, healthy cells. In a preferred embodiment, some EphA2 expressed by the cancer cells is not bound to ligand, either as a result of decreased cell-cell contacts, altered subcellular localization, or increases in amount of EphA2 relative to ligand. In a preferred embodiment, the cancer to be treated, prevented and/or managed is malignant.

The EphA2-BiTEs of the invention can be administered in combination with one or more other cancer therapies. In particular, the present invention provides methods of treating, preventing and/or managing cancer, the methods comprising administering to a subject in need thereof a therapeutically or prophylactically effective amount of one or more EphA2-BiTEs of the invention in combination with the administration of a therapeutically or prophylactically effective amount of one or more other therapies. Non-limiting of examples of other therapies include chemotherapies, hormonal therapies, biological therapies/immunotherapies, radiation and surgery.

Increased expression of EphA2 has been found to be associated with infections by certain intracellular pathogens, in particular, RSV (see, e.g., U.S. Appn. Ser. No. 11/259,266, filed Oct. 27, 2005, titled “Use of Modulators of EphA2 and EphrinA1 for the Treatment and Prevention of Infections,” which is incorporated by reference herein in its entirety). Accordingly, the invention also provides compositions and methods designed for the treatment, prevention and/or management of a pathogen infection, including, but not limited to, a viral infection, a bacterial infection, a fungal infection and a protozoan infection (examples of such pathogens are disclosed in, e.g., U.S. Appn. Ser. No. 11/259,266, filed Oct. 27, 2005, titled “Use of Modulators of EphA2 and EphrinA1 for the Treatment and Prevention of Infections,” (and in particular, paragraphs [006], [0046], [0047], and [0057]) which is incorporated by reference herein in its entirety). In particular, the present invention provides methods for treating, preventing and/or managing an infection where the expression of EphA2 is upregulated in infected cells (e.g., infected EphA2-expressing cells), said methods comprising administering to a subject in need thereof an effective amount of one or more EphA2-BiTEs of the invention, and optionally, an effective amount of a therapy other than an EphA2-BiTE. In a specific embodiment, the pathogen infections to be treated, prevented and/or managed in accordance with the methods of the invention are intracellular pathogen infections. In another specific embodiment, the pathogen infection to be treated, prevented and/or managed in accordance with the methods of the invention is a RSV infection.

In another aspect of the invention, increased expression of EphA2 has been found to be associated with certain non-cancer hyperproliferative cell disorders such as, for example, those disclosed in U.S. Pat. Pub. No. US 2005-0059592 A1, entitled “EphA2 and Hyperproliferative Cell Disorders,” which is incorporated by reference herein in its entirety. Accordingly, the invention also provides compositions and methods designed for the treatment, prevention and/or management of hyperproliferative cell disorders (non-limiting examples of such disorders are disclosed in, e.g., U.S. Pat. Pub. No. 2005-0059592, entitled “EphA2 and Hyperproliferative Cell Disorders,” (and in particular paragraph [0035]) which is incorporated by reference herein in its entirety. In particular, the present invention provides methods for treating, preventing and/or managing a hyperproliferative cell disorder where the expression of EphA2 is upregulated in cells affected by such a disorder, said methods comprising administering to a subject in need thereof an effective amount of one or more EphA2-BiTEs of the invention, and optionally, an effective amount of a therapy other than an EphA2-BiTE. In a specific embodiment, the hyperproliferative cell disorder to be treated, prevented and/or managed in accordance with the methods of the invention are asthma, COPD, lung fibrosis, asbestosis, IPF, DIP, UIP, kidney fibrosis, liver fibrosis, other fibroses, bronchial hyper-responsiveness, psoriasis, seborrheic dermatitis, cystic fibrosis, or a hyperproliferative endothelial cell disorder, such as restenosis, hyperproliferative vascular disease, Behcet's Syndrome, atherosclerosis, macular degeneration, or a hyperproliferative fibroblast disorder.

The methods and compositions of the invention are useful not only in untreated cancer patients but are also useful in the treatment of cancer patients partially or completely refractory to current standard and experimental cancer therapies, including but not limited to chemotherapies, hormonal therapies, biological therapies, immunotherapies, radiation therapies, and/or surgery as well as to improve the efficacy of such treatments. Accordingly, in a preferred embodiment, the invention provides therapeutic and prophylactic methods for the treatment, prevention and/or management of cancer that has been shown to be or may be refractory or non-responsive to therapies other than those comprising administration of EphA2-BiTEs of the invention. In a specific embodiment, one or more EphA2-BiTEs of the invention are administered to a patient refractory or non-responsive to a non-EphA2-BiTE-based therapy (i.e., a therapy other than an EphA2-BiTE) to render the patient non-refractory or responsive. In certain embodiments, the therapy to which the patient had previously been refractory or non-responsive can then be administered with therapeutic effect.

The methods and compositions of the invention are useful not only in untreated patients with a non-cancer hyperproliferative cell disorder, but are also useful in the treatment of such patients partially or completely refractory to current standard and experimental therapies for non-cancer hyperproliferative cell disorders, including but not limited to chemotherapies, hormonal therapies, biological therapies, immunotherapies, radiation therapies, and/or surgery as well as to improve the efficacy of such treatments. Accordingly, in a preferred embodiment, the invention provides therapeutic and prophylactic methods for the treatment, prevention and/or management of non-cancer hyperproliferative cell disorders that have been shown to be or may be refractory or non-responsive to therapies other than those comprising administration of EphA2-BiTEs of the invention. In a specific embodiment, one or more EphA2-BiTEs of the invention are administered to a patient refractory or non-responsive to a non-EphA2-BiTE-based therapy (i.e., a therapy other than an EphA2-BiTE) to render the patient non-refractory or responsive. In certain embodiments, the therapy to which the patient had previously been refractory or non-responsive can then be administered with therapeutic effect.

In another embodiment, methods and compositions of the invention are useful not only in untreated patients infected with a pathogen (e.g., a virus, bacteria, fungus or protozoa pathogen), but are also useful in the treatment of patients partially or completely refractory to current standard and experimental therapies for infections, including but not limited to antiviral, antibacterial, antifungal and/or other antimicrobial agents. Accordingly, in a preferred embodiment, the invention provides therapeutic and prophylactic methods for the treatment, prevention and/or management of infections that have been shown to be or may be refractory or non-responsive to therapies other than those comprising administration of EphA2-BiTEs of the invention. In a specific embodiment, one or more EphA2-BiTEs of the invention are administered to a patient refractory or non-responsive to a non-EphA2-BiTE-based therapy (i.e., a therapy other than an EphA2-BiTE) to render the patient non-refractory or responsive. In certain embodiments, the therapy to which the patient had previously been refractory or non-responsive can then be administered with therapeutic effect.

In addition, the present invention provides methods of screening for EphA2-BiTEs of the invention. In particular, EphA2-BiTEs may be screened for binding to EphA2, particularly the extracellular domain of EphA2, and the T-cell antigen CD3, using routine immunological techniques such as, for example, radioimmunoassays (RIAs), enzyme-linked immunosorbent assays (ELISA), flow cytometry assays and those described in Section 6 below. In one embodiment, to identify EphA2-BiTEs, candidate EphA2-BiTEs may be screened for the ability to initiate redirected lysis of EphA2-positive target cells (e.g., cancer cells, non-cancer hyperproliferative cells, or infected cells that express EphA2). In another embodiment, candidate EphA2-BiTEs may be screened for the ability to have anti-tumor activity in vivo (e.g., in a NOD/SCID mouse xenograft model).

The invention also provides for methods of screening for bispecific single chain antibody constructs that bind to other Eph receptors of the A and B type, i.e., other Eph receptor-BiTEs using the methods described herein to identify EphA2-specific BiTEs. See Eph Nomenclature Committee, 1997, Cell 90(3):403-4; and Cheng et al., 2002, Cytokine & Growth Factor Rev. 13:75-85, each of which is incorporated by reference herein in its entirety, for a list of the Eph receptor family members that may be used as targets to identify other Eph receptor-specific BiTE molecules.

In another embodiment, to identify EphA2-BiTEs that preferentially bind an EphA2 epitope exposed on cancer cells but not non-cancer cells, non-cancer hyperproliferative cells or on infected cells but not non-infected cells, EphA2-BiTEs may also be screened for the ability to preferentially bind EphA2 that is not bound to ligand, e.g., Ephrin A1, and that is not localized to cell-cell contacts. In a specific embodiment, the invention provides methods for identifying tissue affected by a disorder associated with aberrant EphA2 expression and/or activity, comprising using an EphA2-BiTE of the invention in an epitope exclusion assay (see, e.g., Sections 6.2.4, 6.3.8 and 6.7.2, infra). In accordance with this embodiment, EphA2-BiTEs of the invention bind to EphA2 epitopes accessible or expose only on cells of tissues affected by a disorder associated with aberrant EphA2 expression and/or activity (e.g., cancer, non-cancer cells, hyperproliferative cells or infected cells that express EphA2), and not cells of normal tissues of the same tissue type. Any method known in the art to determine antibody binding/localization on a cell can be used to screen candidate BiTEs for desirable binding properties. In a specific embodiment, standard assays known in the art such as nuclear magnetic resonance (NMR) microscopy, immunofluorescence microscopy, flow cytometry or surface plasmon resonance assays are used to determine the binding characteristics of a particular EphA2-BiTE. In this embodiment, EphA2-BiTEs that bind poorly to EphA2 when it is bound to its ligand and localized to cell-cell contacts but bind well to free EphA2 on a cell are encompassed by the invention. In another specific embodiment, EphA2-BiTEs are selected for their ability to compete with ligands (e.g., cell-anchored or purified ligands) for binding to EphA2 using cell-based or ELISA assays.

### 3.1 Definitions

As used herein, the term “aberrant” in the context of EphA2 expression, in certain embodiments, refers to the expression of EphA2 that is increased on a cell, e.g., a cancer cell, a non-cancer hyperproliferative cell, or an infected cell of a subject, relative to the level of EphA2 expression in the normal cells of said subject, cells of a normal, healthy subject and/or a population of normal, healthy cells. Increased EphA2 expression refers to an increase in the expression of EphA2 in the cells of a subject with a disorder associated with aberrant expression of EphA2, relative to the level of EphA2 expression in normal cells of said subject, cells of a normal, healthy subject and/or a population of normal, healthy cells. In a specific embodiment, the level of EphA2 expression in the cells of a subject with a disorder associated with aberrant expression of EphA2 is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of EphA2 expression in the normal cells of said subject, cells of a normal, healthy subject and/or a population of normal, healthy cells, as measured by a standard assay known in the art or as described in Section 6.0 below (e.g., a flow cytometry assay). In a specific embodiment, the expression of EphA2 is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold on a cancer cell, a non-cancer hyperproliferative cell, or an infected cell, relative to the level of EphA2 expression in the normal cells of said subject, cells of a normal, healthy subject and/or a population of normal, healthy cells, as measured by a standard assay known in the art or as described in Section 6.0 below (e.g., a flow cytometry assay). In another embodiment, the term “aberrant” in the context of EphA2 expression refers to the expression of EphA2 wherein certain epitopes of EphA2 are selectively exposed on a cancer cell, a non-cancer hyperproliferative cell, or an infected cell from a subject, and not on normal cells of said subject, cells of a normal, healthy subject and/or a population of normal, healthy cells. In another embodiment, the term “aberrant” in the context of EphA2 expression refers to the expression of EphA2 wherein the subcellular localization of EphA2 is altered in a cell (at, e.g., sites other than sites of cell-cell contact), as measured by a standard assay known in the art or by methods described in Section 6 below such as, for example, immunofluorescence staining.

As used herein, the term “agent” refers to a molecule that has a desired biological effect. Agents include, but are not limited to, proteinaceous molecules (e.g., peptides, polypeptides, proteins and antibodies (e.g., bispecific single chain antibodies)), vaccines, small molecules (less than 1000 daltons), inorganic or organic compounds, and nucleic acid molecules (including, but not limited to, double-stranded or single-stranded DNA, or double-stranded or single-stranded RNA (e.g., antisense, RNAi, etc.), aptamers, as well as triple helix nucleic acid molecules). Agents can be derived or obtained from any known organism (including, but not limited to, animals (e.g., mammals (human and non-human mammals)), plants, bacteria, fungi, and protista, or viruses) or from a library of synthetic molecules.

As used herein, the term “analog” in the context of a proteinaceous agent (e.g., a peptide, polypeptide, protein or antibody) refers to a proteinaceous agent that possesses a similar or identical function as a second proteinaceous but does not necessarily comprise a similar or identical amino acid sequence or structure of the second proteinaceous agent. A proteinaceous agent that has a similar amino acid sequence refers to a proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure of the second proteinaceous agent. The structure of a proteinaceous agent can be determined by methods known to those skilled in the art, including but not limited to, X-ray crystallography, nuclear magnetic resonance, and crystallographic electron microscopy. Preferably, a proteinaceous agent of the invention has EphA2-BiTE activity.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87: 2264-2268, modified as in Karlin and Altschul, 1993, Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4: 11-17. Such an algorithm is incorporated in the ALIGN program (version-2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the terms “antibody” or “antibodies” refer to molecules that contain an antigen binding site, e.g., immunoglobulin molecules and immunologically active fragments of immunoglobulin molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or a subclass of immunoglobulin molecule. Antibodies include, but are not limited to, synthetic antibodies, monoclonal antibodies, single domain antibodies, single chain antibodies, recombinantly produced antibodies, multispecific antibodies (including bispecific antibodies), human antibodies, humanized antibodies, chimeric antibodies, intrabodies, scFvs (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above.

In a specific embodiment, an EphA2-BiTE of the invention is a bispecific single chain antibody which comprises: (1) a first binding domain that comprises a variable heavy (VH) domain and a variable light (VL) domain of an antibody that immunospecifically binds to the T-cell antigen CD3; and (2) a second binding domain that immunospecifically binds to EphA2. In another specific embodiment, the first binding domain and second binding domain of an EphA2-BiTE of the invention each comprises a single chain Fv (scFv). The variable heavy domain and/or variable light domain of the first binding domain of an EphA2-BiTE may be obtained or derived from any type of antibody that immunospecifically binds to CD3. The variable heavy domain and/or variable light domain of the second binding domain of an EphA2-BiTE may be obtained or derived from any type of antibody that immunospecifically binds to EphA2. Non-limiting examples of the types of antibodies include synthetic antibodies, monoclonal antibodies, single domain antibodies, single chain antibodies, recombinantly produced antibodies, multispecific antibodies (including bispecific antibodies), human antibodies, humanized antibodies, chimeric antibodies, intrabodies, scFvs (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above.

As used herein, the term “binding domain” refers to a domain comprising a three-dimensional structure capable of immunospecifically binding to an epitope. Thus, in one embodiment, said domain can comprise the VH and/or VL domain of an antibody chain, preferably at least the VH domain. In another embodiment, the binding domain may comprise at least one complementarity determining region (CDR) of an antibody chain recognizing the EphA2 and CD3 antigens, respectively. In this respect, it is noted that the domains of the binding domains present in the EphA2-BiTE of the invention may not only be derived from antibodies but also from other EphA2 or CD3 binding proteins, such as naturally occurring surface receptors or ligands.

As used herein, the terms “deimmunized,” “deimmunization” or grammatically related variants thereof denote modification of the first and/or second binding domain vis-à-vis an original wild type construct by rendering said wild type construct non-immunogenic or less immunogenic in humans. Deimmunization approaches are well known in the art and are disclosed in, e.g., International Pub. Nos. WO 00/34317 (and in particular, pp. 1-14); WO 98/52976 (and in particular, Examples 1-6 on pp. 18-38); WO 02/079415 (and in particular, pp. 2-8 and Examples 1-10 at pp. 15-43); and WO 92/10755 (and in particular, pp. 6-9), each of which is incorporated by reference herein in its entirety. The term “deimmunized” also relates to constructs, which show reduced propensity to generate T-cell epitopes. In accordance with this invention, the term “reduced propensity to generate T-cell epitopes” relates to the removal of T-cell epitopes leading to specific T-cell activation. Moreover, “reduced propensity to generate T-cell epitopes” refers to the substitution of amino acids contributing to the formation of T-cell epitopes, i.e., substitution of amino acids which are essential for formation of a T-cell epitope. In other words, “reduced propensity to generate T-cell epitopes” relates to reduced immunogenicity or reduced capacity to induce antigen-dependent T-cell proliferation. The term T-cell epitope relates to short peptide sequences which can be released during the degradation of peptides, polypeptides or proteins within cells and subsequently presented by molecules of the major histocompatibility complex (MHC) in order to trigger activation of T-cell (see, e.g., International Pub. No. WO 02/066514, which is incorporated by reference herein in its entirety). For peptides presented by MHC class II such activation of T-cells can then give rise to an antibody response by direct stimulation of B cells to produce said antibodies. “Reduced propensity to generate T-cell epitopes” and/or “deimmunization” may be measured by techniques known in the art. Preferably, deimmunization of proteins may be tested in vitro by a T-cell proliferation assay. In this assay, peripheral blood mononuclear cells (PBMCs) from donors representing >80% of HLA-DR alleles in the world are screened for proliferation in response to either wild type or deimmunized peptides. Ideally cell proliferation is only detected upon loading of the antigen-presenting cells with wild type peptides. Alternatively, deimmunization may be tested by expressing HLA-DR tetramers representing all haplotypes. These tetramers may be tested for peptide binding or loaded with peptides substitute for antigen-presenting cells in proliferation assays. In order to determine whether deimmunized peptides are presented on HLA-DR haplotypes, binding of, e.g., fluorescence-labeled peptides on PBMCs can be measured. Furthermore, deimmunization can be confirmed by determining whether antibodies against the deimmunized molecules have been formed after administration in patients. Preferably, antibody derived molecules are deimmunized in the framework regions and most of the CDR regions are not modified in order to generate reduced propensity to induce T-cell epitopes so that the binding affinity of the CDR regions is not affected. Even elimination of one T-cell epitope results in reduced immunogenicity. In specific embodiments, the immunogenicity of the binding domain that binds to an antigen of interest (e.g., CD3) is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or at least 100% compared to a non-immunized control polypeptide or protein or fragment thereof, as measured by a standard assay described above (e.g., a T-cell proliferation assay) or known in the art. In summary, the approaches discussed above help reduce the immunogenicity of the therapeutic bispecific single chain antibodies (e.g., EphA2-BiTEs) described herein when they are administered to patients having a disorder associated with aberrant expression and/or activity of EphA2 (e.g., cancer, non-cancer hyperproliferative cell disorders and infections). For example, the first binding domain which binds to the epsilon subunit of CD3 is deimmunized. Preferably, the arrangement of the variable regions in this CD3-binding domain is VH-VL.

As used herein, the term “derivative” in the context of a proteinaceous agent (e.g., proteins, polypeptides, peptides, and antibodies) refers to a proteinaceous agent that comprises the amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, a derivative of a proteinaceous agent may be produced, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may also be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses an identical function(s) as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl, nitryl, or amine group. An organic molecule may also, for example, be esterified, alkylated and/or phosphorylated.

As used herein, the term “effective amount” refers to the amount of a therapy (e.g., a prophylactic or therapeutic agent) which is sufficient to reduce and/or ameliorate the severity and/or duration of a disorder associated with aberrant expression and/or activity of EphA2, or a symptom thereof, prevent the advancement of said disorder, cause regression of said disorder, prevent the recurrence, development, or onset of one or more symptoms associated with said a disorder associated with aberrant expression and/or activity of EphA2, or enhance or improve the prophylactic or therapeutic effect(s) of another therapy (e.g., another prophylactic or therapeutic agent).

As used herein, the terms “elderly human,” “elderly,” or variations thereof refer to a human 65 years old or older, preferably 70 years old or older.

As used herein, the term “endogenous ligand” or “natural ligand” refers to a molecule that normally binds a particular receptor in vivo. For example, EphrinA1 is an endogenous ligand of EphA2.

As used herein, the term “EphA2 polypeptide” refers to EphA2, an analog, derivative or a fragment thereof, or a fusion protein comprising EphA2, an analog, derivative or a fragment thereof. The EphA2 polypeptide may be from any species. In a specific embodiment, the EphA2 polypeptide is human. In certain embodiments, the term “EphA2 polypeptide” refers to the mature, processed form of EphA2. In other embodiments, the term “EphA2 polypeptide” refers to an immature form of EphA2. In accordance with this embodiment, the antibodies of the invention immunospecifically bind to the portion of the immature form of EphA2 that corresponds to the mature, processed form of EphA2. In a specific embodiment, the term “EphA2 polypeptide” refers to the extracellular domain of EphA2 or a fragment thereof. In certain embodiments, in the context of a cell, an EphA2-BiTE of the invention binds to the extracellular domain (i.e., an epitope of EphA2 that is exposed on the cell surface) of an EphA2.

The nucleotide and/or amino acid sequences of EphA2 polypeptides can be found in the literature or public databases, or the nucleotide and/or amino acid sequences can be determined using cloning and sequencing techniques known to one of skill in the art. For example, the nucleotide sequence of human EphA2 can be found in the GenBank database (see, e.g., Accession Nos. BC037166, M59371 and M36395). The amino acid sequence of human EphA2 can be found in the GenBank database (see, e.g., Accession Nos. AAH37166 and AAA53375). Additional non-limiting examples of amino acid sequences of EphA2 are listed in the following table.

| Species | GenBank Accession No. |
|---|---|
| Mouse | NP_034269, AAH06954 |
| Rat | XP_345597 |

As used herein, the term “epitope” refers to sites or fragments of a polypeptide or protein having antigenic or immunogenic activity in an animal, preferably in a mammal, and most preferably in a human. In specific embodiments, the term “epitope” refers to a fragment of an EphA2 polypeptide or a fragment of a CD3 polypeptide having antigenic or immunogenic activity in an animal, preferably in a mammal, and most preferably in a human. An epitope having immunogenic activity is a site or fragment of a polypeptide or protein that elicits an antibody response in an animal. In specific embodiments, an epitope having immunogenic activity is a fragment of an EphA2 polypeptide or a fragment of a CD3 polypeptide that elicits an antibody response in an animal. An epitope having antigenic activity is a site or fragment of a polypeptide or protein to which an antibody immunospecifically binds as determined by any method well-known to one of skill in the art, for example, by immunoassays such as RIAs or ELISA assays. In specific embodiments, an epitope having antigenic activity is a fragment of an EphA2 polypeptide or a fragment of a CD3 polypeptide to which an antibody immunospecifically binds as determined by any method well known in the art, for example, by immunoassays. Antigenic epitopes need not necessarily be immunogenic.

As used herein, the term “fragment” in the context of a proteinaceous agent refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of another polypeptide or protein. In a specific embodiment, the term “fragment” in the context of a proteinaceous agent refers to a peptide or polypeptide comprising an amino acid sequence ranging from 10 to 15, 10 to 20, 10 to 30, 10 to 40, 10 to 50, 10 to 60, 10 to 70, 10 to 80, 10 to 100, 10 to 125, 10 to 150, 10 to 175, 10 to 200, 10 to 250, or 10 to 300 contiguous amino acid residues of another polypeptide or protein.

In a specific embodiment, a fragment is a fragment of EphA2, or an antibody that immunospecifically binds to an EphA2. In another specific embodiment, a fragment is a fragment of CD3 or an antibody that immunospecifically binds to CD3. In an embodiment, a fragment of a protein or polypeptide retains at least one function of the protein or polypeptide. In another embodiment, a fragment of a polypeptide or protein retains at least two, three, four, or five functions of the polypeptide or protein. In a preferred embodiment, a fragment of an antibody that immunospecifically binds to an EphA2 polypeptide or fragment thereof retains the ability to immunospecifically bind to an EphA2 polypeptide or fragment thereof. In another preferred embodiment, a fragment of an antibody that immunospecifically binds to a CD3 polypeptide or fragment thereof retains the ability to immunospecifically bind to a CD3 polypeptide or fragment thereof. Preferably, antibody fragments are epitope-binding fragments.

As used herein, the term “fusion protein” refers to a polypeptide or protein that comprises the amino acid sequence of a first polypeptide or protein or fragment, analog or derivative thereof, and the amino acid sequence of a heterologous polypeptide or protein (i.e., a second polypeptide or protein or fragment, analog or derivative thereof different than the first polypeptide or protein or fragment, analog or derivative thereof, or not normally part of the first polypeptide or protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides, or peptides with immunomodulatory activity may be fused together to form a fusion protein. In a preferred embodiment, fusion proteins retain or have improved activity relative to the activity of the original polypeptide or protein prior to being fused to a heterologous protein, polypeptide, or peptide. In a specific embodiment, a fusion protein comprises a first binding domain that binds to the T-cell antigen CD3 and a second binding domain that binds to an antigen of interest (e.g., EphA2). In accordance with this embodiment, a fusion protein is an EphA2-BiTE.

As used herein, the term “humanized antibody” refers to forms of non-human (e.g., murine) antibodies, preferably chimeric antibodies, which contain minimal sequence derived from non-human immunoglobulin. For the most part, humanized antibodies are human immunoglobulins (recipient antibody) in which hypervariable region or complementarity determining (CDR) residues of the recipient are replaced by hypervariable region residues or CDR residues from an antibody from a non-human species (donor antibody) such as mouse, rat, rabbit or non-human primate having the desired specificity, affinity, and capacity. In some instances, one or more Framework Region (FR) residues of the human immunoglobulin are replaced by corresponding non-human residues or other residues based upon structural modeling, e.g., to improve affinity of the humanized antibody. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a fragment of an immunoglobulin constant region (Fc), typically that of a human immunoglobulin. For further details regarding humanization methods and framework shuffling, see Dall'Acqua et al., 2005, Methods 36:43-60, Jones et al., 1986, Nature 321:522-525; Reichmann et al., 1988, Nature 332:323-329; Presta, 1992, Curr. Op. Struct. Biol. 2:593-596; and Queen et al., U.S. Pat. No. 5,585,089, and US Pat. Pub. No. 2005-0048617 A1, each of which is incorporated by reference herein in its entirety.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6.

Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

In one non-limiting example, stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “hypervariable region” refers to the amino acid residues of an antibody which are responsible for antigen binding. The hypervariable region comprises amino acid residues from a “Complementarity Determining Region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

As used herein, the term “immunomodulatory agent” refers to an agent that modulates a subject's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response. In a preferred embodiment of the invention, an immunomodulatory agent is an agent that inhibits or reduces a subject's immune response (i.e., an immunosuppressant agent).

As used herein, the term “immunospecifically binds to EphA2” and analogous terms in the context of anti-EphA2 antibodies, EphA2-BiTEs, and binding domains of EphA2-BiTEs, refer proteinaceous agents, including antibodies (e.g., EphA2-BiTEs which are bispecific single chain antibodies) and the binding domains of EphA2-BiTEs (e.g., scFvs of EphA2-BiTEs which are bispecific single chain antibodies) that specifically bind to an EphA2 polypeptide, and do not specifically bind to non-EphA2 polypeptides. Preferably, antibodies and binding domains of EphA2-BiTEs that specifically bind to an EphA2 polypeptide do not cross-react with other non-related antigens. In specific embodiments, anti-EphA2 antibodies of the invention bind to an EphA2 polypeptide with little or no cross-reactivity with other non-related antigens, as measured by a standard assay known in the art, such as an ELISA assay. In certain embodiments, antibodies and binding domains of EphA2-BiTEs that immunospecifically bind to an EphA2 polypeptide may be cross-reactive with related antigens (e.g., other types Eph receptors from the A and/or B family of Eph receptors). A peptide, polypeptide, protein, antibody or binding domain that immunospecifically binds to an EphA2 polypeptide may bind to other peptides, polypeptides, or proteins with lower affinity as determined by, e.g., immunoassays or other assays known in the art to detect binding affinity. Antibodies or binding domains that immunospecifically bind to an EphA2 polypeptide may be cross-reactive with related antigens. Preferably, antibodies or fragments thereof that immunospecifically bind to an EphA2 polypeptide can be identified, for example, by immunoassays or other techniques known to those of skill in the art. An antibody or fragment thereof binds specifically to an EphA2 polypeptide when it binds to an EphA2 polypeptide with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2^{nd} ed., Raven Press, New York at pages 332-336 for a discussion regarding antibody specificity. In another embodiment, an antibody that binds to an EphA2 polypeptide that is a fusion protein immunospecifically binds to the fragment of the fusion protein that is a EphA2 polypeptide. Preferably, antibodies and binding domains of EphA2-BiTEs that immunospecifically bind to an EphA2 polypeptide only modulate an EphA2 activity(ies) and do not significantly affect other activities. In a specific embodiment, antibodies and binding domains of EphA2-BiTEs that immunospecifically bind to an EphA2 polypeptide are preferably single chain antibodies (e.g., scFvs comprising a VH domain and a VL domain), which may have a low K_{off} rate (e.g., K_{off} less than 3×10⁻² s⁻¹).

As used herein, the term “immunospecifically binds to CD3” and analogous terms refer to proteinaceous agents that specifically bind to CD3 or a subunit thereof, and do not specifically bind to other antigens. Preferably, antibodies and binding domains of EphA2-BiTEs that immunospecifically bind to CD3 do not cross-react with non-related antigens.

As used herein, the term “in combination” in the context of administration of a therapy refers to the use of more than one therapy. The use of the term “in combination” does not restrict the order in which therapies are administered to a subject with a disorder associated with aberrant expression and/or activity of EphA2. A first therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly or concurrently with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to a disorder associated with aberrant expression and/or activity of EphA2. Any additional therapy can be administered in any order with the other additional therapies. In certain embodiments, EphA2-BiTEs of the invention can be administered in combination with one or more therapies (e.g., non-EphA2-BiTEs currently administered to treat, prevent and/or manage the disorder associated with aberrant expression and/or activity of EphA2, such as, for example, analgesic agents, anesthetic agents, antibiotics, antiviral agents, anti-fungal agents, anti-protozoal agents, immunomodulatory agents).

As used herein, the terms “increased” or “overexpress” or “overexpression” with respect to EphA2 expression refers to an increase in the expression of EphA2 in the cells of a subject with a disorder associated with aberrant expression and/or activity of EphA2, relative to the level of EphA2 expression in normal cells of said subject, cells of a normal, healthy subject and/or a population of normal, healthy cells as measured by any method known in the art, including but not limited to RIAs, ELISA assays, Western Blot analysis, flow cytrometry, or immunohistochemistry using antibodies that immunospecifically bind to EphA2. In a specific embodiment, the level of EphA2 expression in the cells of a subject with a disorder associated with aberrant expression and/or activity of EphA2 is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of EphA2 expression in the normal cells of said subject, cells of a normal, healthy subject and/or a population of normal, healthy cells.

As used herein, the terms “human infant” or “infant” or variations thereof refer to a human less than 24 months of age, preferably less than 12 months, less than 6 months, less than 3 months, less than 2 months, or less than 1 month of age.

As used herein, the terms “human infant born prematurely,” “preterm infant,” or “premature infant,” or variations thereof refer to a human born at less than 40 weeks of gestational age, preferably less than 35 weeks gestational age, who is less than 6 months old, preferably less than 3 months old, more preferably less than 2 months old, and most preferably less than 1 month old.

As used herein, the term “isolated” in the context of an organic or inorganic molecule (whether it be a small or large molecule), other than a proteinaceous agent or a nucleic acid, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In a preferred embodiment, an organic and/or inorganic molecule is isolated.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated. In a preferred embodiment, an EphA2-BiTE molecule of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, is preferably substantially free of other cellular material, or culture medium when produced by recombinant techniques, or substantially free of chemical precursors or other chemicals when chemically synthesized. In a specific embodiment, nucleic acid molecules are isolated. In a preferred embodiment, a nucleic acid molecule encoding an EphA2-BiTE molecule of the invention is isolated.

As used herein, the term “low tolerance” refers to a state in which the patient suffers from side effects from a therapy(s) so that the patient does not benefit from and/or will not continue therapy because of the adverse effects and/or the harm from side effects outweighs the benefit of the treatment.

As used herein, the terms “manage”, “managing” and “management” refer to the beneficial effects that a subject derives from a therapy, which does not result in a cure of a disorder. In certain embodiments, a subject is administered one or more therapies to “manage” a disorder associated with aberrant expression (e.g., overexpression) and/or activity of EphA2 (e.g., cancer, non-cancer hyperproliferative cell disorder or an infection) so as to prevent the progression or worsening of the disorder (i.e., hold disease progress).

As used herein, the term “pathology-causing cell phenotype” refers to a function that a cell affected by a disorder associated with aberrant expression and/or activity of EphA2 performs that causes or contributes to the pathological state of said disorder. Pathology-causing cell phenotypes include, but are not limited to, decreased cell/cell interaction, increased extracellular matrix deposition, increased migration, increased cell survival and/or proliferation of a cancer cell, a hyperproliferative cell, or a cell infected (e.g., an epithelial cell) by an infectious pathogen/agent (e.g., bacteria, virus, fungus or protozoan). One or more of these pathology-causing cell phenotypes causes or contributes to symptoms in a patient suffering from a disorder associated with aberrant expression and/or activity of EphA2 (e.g., cancer, a non-cancer hyperproliferative cell disorder, or an infection).

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “potentiate” in the context of the administration of a therapy to a subject refers to an improvement in the efficacy of a therapy at its common or approved dose.

As used herein, the terms “prevent,” “preventing,” and “prevention” in the context of therapies administered to a subject refer to the reduction or inhibition of the development, onset, spread or recurrence of a disorder associated with aberrant expression and/or activity of EphA2 or a symptom thereof in a subject resulting from the administration of a therapy (e.g., a prophylactic or therapeutic agent), or the administration of a combination of therapies (e.g., a combination of prophylactic or therapeutic agents). In a specific embodiment, the terms “prevent,” “preventing,” and “prevention” in the context of therapies administered to a subject refer to the increase in the time to recurrence or a decrease in the spread or progression of a disorder associated with aberrant expression and/or activity of EphA2.

As used herein, the term “prophylactic agent” refers to any agent that can prevent the development, recurrence, spread or onset of a disorder associated with aberrant expression (e.g., overexpression) and/or activity of EphA2 (e.g., cancer, a non-cancer hyperproliferative cell disorder, or an infection), or a symptom thereof. In certain embodiments, the term “prophylactic agent” refers to an EphA2-BiTE. In certain other embodiments, the term “prophylactic agent” refers to an agent other than an EphA2-BiTE. Preferably, a prophylactic agent is an agent which is known to be useful to or has been or is currently being used to the prevent or impede the onset, development, recurrence, progression and/or spread of a disorder associated with aberrant expression (e.g., overexpression) and/or activity of EphA2 (e.g., cancer, a non-cancer hyperproliferative cell disorder, or an infection), or one or more symptoms thereof.

As used herein, a “prophylactically effective amount” refers to the amount of a therapy (e.g., a prophylactic agent) that is sufficient to result in the prevention of the development, recurrence, spread or onset of a disorder associated with aberrant expression and/or activity of EphA2 (e.g., cancer, a non-cancer hyperproliferative cell disorder or an infection), or a symptom thereof. A prophylactically effective amount may refer to the amount of a therapy (e.g., a prophylactic agent) sufficient to prevent the development, recurrence, spread or onset of a disorder associated with aberrant expression and/or activity of EphA2 (e.g., cancer, a non-cancer hyperproliferative cell disorder or an infection), or a symptom thereof in, for example, those having previously suffered from such a disorder, or those who are immunocompromised or immunosuppressed, or are genetically predisposed to such an a disorder. A prophylactically effective amount may also refer to the amount of a therapy (e.g., a prophylactic agent) that provides a prophylactic benefit in the prevention of a disorder associated with aberrant expression and/or activity of EphA2 (e.g., cancer, a non-cancer hyperproliferative cell disorder or an infection), or a symptom thereof. Further, a prophylactically effective amount with respect to a therapy (e.g., a prophylactic agent of the invention) means that amount of the therapy (e.g., prophylactic agent) alone, or in combination with one or more other therapies (e.g., non-EphA2-BiTE therapies currently administered to prevent the disorder associated with aberrant expression and/or activity of EphA2 (e.g., cancer, a non-cancer hyperproliferative cell disorder or an infection), analgesic agents, anesthetic agents, antibiotics, or immunomodulatory agents) that provides a prophylactic benefit in the prevention of disorder associated with aberrant expression and/or activity of EphA2. Used in connection with an amount of an EphA2-BiTE of the invention, the term can encompass an amount that improves overall prophylaxis or enhances the prophylactic efficacy of or synergies with another therapy, (e.g., a prophylactic agent).

As used herein, a “protocol” includes dosing schedules and dosing regimens.

As used herein, the term “refractory” refers to a disorder associated with aberrant expression (e.g., overexpression) and/or activity of EphA2 (e.g., cancer, a non-cancer hyperproliferative cell disorder or an infection), that is not responsive to one or more therapies (e.g., currently available therapies). In certain embodiments, that a disorder associated with aberrant expression (e.g., overexpression) and/or activity of EphA2 (e.g., cancer, a non-cancer hyperproliferative cell disorder or an infection) is refractory to a therapy means that at least some significant portion of the symptoms associated with said disorder are not eliminated or lessened by that therapy. The determination of whether such disorder is refractory can be made either in vivo and/or in vitro by any method known in the art for assaying the effectiveness of therapy for a disorder associated with aberrant expression and/or activity of EphA2 (e.g., cancer, a non-cancer hyperproliferative cell disorder or an infection).

As used herein, the phrase “side effects” encompasses unwanted and adverse effects of a therapy (e.g., a prophylactic or therapeutic agent). Adverse effects are always unwanted, but unwanted effects are not necessarily adverse. An adverse effect from a therapy (e.g., a prophylactic or therapeutic agent) might be harmful or uncomfortable or risky. Examples of side effects include, but are not limited to, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physicians' Desk Reference (61^{st} ed., 2007).

As used herein, the term “single-chain Fv” or “scFv” refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994). In a specific embodiment, the EphA2-BiTEs of the invention are comprised of scFvs.

As used herein, the terms “subject” and “patient” are used interchangeably. As used herein, a subject is an animal, preferably a mammal such as a non-primate (e.g., cows, pigs, horses, cats, dogs, rats, etc.) and a primate (e.g., monkey (e.g., a rhesus monkey, a cynomolgus monkey or chimpanzee) and human), and most preferably a human. In one embodiment, the subject is a mammal, preferably a human, with a disorder associated with aberrant expression and/or activity of EphA2. Such disorders include, but are not limited to, cancer, non-cancer hyperproliferative cell disorders, and infections. In another embodiment, the subject is a mammal, preferably a human, at risk of developing a disorder associated with aberrant expression and/or activity of EphA2 (e.g., an immunocompromised or immunosuppressed mammal, or a genetically predisposed mammal). In another embodiment, the subject is not an immunocompromised or immunosuppressed mammal, preferably a human. In another embodiment, the subject is a mammal, preferably a human, with a lymphocyte count that is not under approximately 500 cells/mm³.

As used herein, the term “synergistic” refers to a combination of therapies (e.g., prophylactic or therapeutic agents) which is more effective than the additive effects of any two or more single therapies (e.g., one or more prophylactic or therapeutic agents). In one aspect, a synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a disorder associated with aberrant expression (e.g., overexpression) and/or activity of EphA2 (e.g., cancer, a non-cancer hyperproliferative cell disorder or an infection). In some embodiments, the ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a disorder associated with aberrant expression and/or activity (e.g., overexpression) of EphA2 (e.g., cancer, a non-cancer hyperproliferative cell disorder or an infection). In another aspect, a synergistic effect can result in improved efficacy of therapies (e.g., prophylactic or therapeutic agents) in the treatment, prevention and/or management of a disorder associated with aberrant expression (e.g., overexpression) and/or activity of EphA2 (e.g., cancer, a non-cancer hyperproliferative cell disorder or an infection). In another aspect, synergistic effect of a combination of therapies (e.g., prophylactic or therapeutic agents) may avoid or reduce adverse or unwanted side effects associated with the use of any single therapy.

As used herein, the term “therapeutic agent” refers to any agent that can be used in the treatment and/or management of a disorder associated with aberrant expression (e.g., overexpression) and/or activity of EphA2 (e.g., cancer, a non-cancer hyperproliferative cell disorder or an infection) or one or more symptoms thereof. In certain embodiments, the term “therapeutic agent” refers to an EphA2-BiTE molecule of the invention. In certain other embodiments, the term “therapeutic agent” refers an agent other than an EphA2-BiTE molecule of the invention. Preferably, a therapeutic agent is an agent which is known to be useful for, or has been or is currently being used for the treatment, prevention and/or management of a disorder associated with aberrant expression (e.g., overexpression) and/or activity of EphA2 (e.g., cancer, a non-cancer hyperproliferative cell disorder or an infection) or one or more symptoms thereof.

As used herein, a “therapeutically effective amount” in the context of cancer refers to the amount of a therapy alone, or in combination with other therapies, that provides a therapeutic benefit in the treatment and/or management of cancers. In one aspect, a therapeutically effective amount refers to the amount of a therapy sufficient to destroy, modify, control or remove primary, regional or metastatic cancer tissue. In another aspect, a therapeutically effective amount refers to the amount of a therapy sufficient to reduce the symptoms of a cancer. In another aspect, a therapeutically effective amount refers to the amount of a therapy sufficient to delay or minimize the spread of cancer. In a specific embodiment, a therapeutically effective amount of a therapy is an amount of a therapy sufficient to inhibit growth or proliferation of cancer cells, kill existing cancer cells (e.g., cause regression of the cancer), and/or prevent the spread of cancer cells to other tissues or areas (e.g., prevent metastasis). In another specific embodiment, a therapeutically effective amount of a therapy is the amount of a therapy sufficient to inhibit the growth of a tumor by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as measured by a standard method known in the art or as described in Section 6.4.1 below. Used in connection with an amount of an EphA2-BiTE of the invention, the term can encompass an amount that improves overall therapy, reduces or avoids unwanted effects, or enhances the therapeutic efficacy of or synergies with another therapy. In one embodiment, a therapeutically effective amount of a therapy reduces or avoids unwanted effects, or enhances the therapeutic efficacy of or synergies with another therapy by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline) in an assay known in the art or described herein.

As used herein, a “therapeutically effective amount” in the context of a non-cancer hyperproliferative cell disorder refers to the amount of a therapy alone, or in combination with other therapies, that provides a therapeutic benefit in the treatment and/or management of said disorder. In one aspect, a therapeutically effective amount refers to the amount of a therapy sufficient to destroy, modify, control or remove cells affected by a non-cancer hyperproliferative cell disorder. In another aspect, a therapeutically effective amount refers to the amount of a therapy sufficient to reduce the symptoms of a non-cancer hyperproliferative cell disorder. In another aspect, a therapeutically effective amount refers to the amount of a therapy sufficient to delay or minimize the spread of the a non-cancer hyperproliferative cell disorder. In a specific embodiment, a therapeutically effective amount of a therapy is an amount of a therapy sufficient to inhibit growth or proliferation of the a non-cancer hyperproliferative cell disorder, kill existing non-cancer hyperproliferative cells (e.g., cause regression of the disorder). In another specific embodiment, a therapeutically effective amount of a therapy is the amount of a therapy sufficient to inhibit the growth of the non-cancer hyperproliferative cells by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as measured by a standard method known in the art. Used in connection with an amount of an EphA2-BiTE of the invention, the term can encompass an amount that improves overall therapy, reduces or avoids unwanted effects, or enhances the therapeutic efficacy of or synergies with another therapy. In one embodiment, a therapeutically effective amount of a therapy reduces or avoids unwanted effects, or enhances the therapeutic efficacy of or synergies with another therapy by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline) in an assay known in the art or described herein.

As used herein, a “therapeutically effective amount” in the context of an infection refers to that amount of a therapy (e.g., a therapeutic agent) sufficient to reduce the severity of an infection, reduce the duration of an infection, ameliorate one or more symptoms of an infection, prevent the advancement of the infection, cause regression of the infection, or to enhance or improve the therapeutic effect(s) of another therapy. In certain embodiments, a therapeutically effective amount refers to the amount of a therapeutic agent sufficient to reduce or inhibit the replication of a pathogen, inhibit or reduce the infection of a cell by a pathogen, inhibit or reduce the production of pathogen proteins, inhibit or reduce the release of a pathogen, inhibit or reduce the spread of a pathogen to other tissues or subjects, or ameliorate one or more symptoms associated with the infection. In one embodiment, a therapeutically effective amount of a therapeutic agent reduces the replication or spread of a pathogen by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline) in an assay known in the art or described herein. Assays that may be used to measure replication of a pathogen, e.g., a virus, include but are not limited to infectivity and transforming assays as described in, e.g., H. M. Temin and H. Rubin. 1958. Characteristics of an assay for Rous sarcoma virus and Rous sarcoma cells in tissue culture. Virology 17: 669-688; and J. W. Hartley and W. P. Rowe. 1966. Production of altered cell foci in tissue culture by defective Moloney sarcoma virus particles. Proc. Natl. Acad. Sci. 55: 780-786, each of which is incorporated by reference herein in its entirety.

As used herein, the term “therapy” refers to any protocol, method and/or agent that can be used in the treatment, prevention and/or management of a disorder associated with aberrant expression (e.g., overexpression) and/or activity of EphA2 (e.g., cancer, a non-cancer hyperproliferative cell disorder or an infection). In certain embodiments, the terms “therapies” and “therapy” refer to a biological therapy, supportive therapy, and/or other therapies useful in the treatment, prevention and/or management of a disorder associated with aberrant expression (e.g., overexpression) and/or activity of EphA2 (e.g., cancer, a non-cancer hyperproliferative cell disorder or an infection), or one or more symptoms thereof known to one of skill in the art such as medical personnel.

As used herein, the terms “treat”, “treatment” and “treating” in the context of administering a therapy(ies) to a subject refer to the reduction or amelioration of the progression, severity, and/or duration of a disorder associated with aberrant expression (e.g., overexpression) and/or activity of EphA2 (e.g., cancer, a non-cancer hyperproliferative cell disorder or an infection), and/or the amelioration of one or more symptoms thereof resulting from the administration of one or more therapies (including, but not limited to, the administration of one or more prophylactic or therapeutic agents). In specific embodiments, the terms “treat”, “treatment” and “treating” in the context of administering a therapy(ies) to a subject refer to the reduction or amelioration of the progression, severity, and/or duration of a disorder associated with aberrant expression (e.g., overexpression) and/or activity of EphA2 (e.g., cancer) refers to a reduction in cancer cells by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline). In other embodiments, the terms “treat”, “treatment” and “treating” in the context of administering a therapy(ies) to a subject refer to the reduction or amelioration of the progression, severity, and/or duration of a disorder associated with aberrant expression (e.g., overexpression) and/or activity of EphA2 (e.g., cancer) refers to no change in cancer cell number, a reduction in hospitalization time, a reduction in mortality, or an increase in survival time of the subject with cancer.

### 4. DESCRIPTION OF THE FIGURES

FIGS. 1A-1B: Sequences of the VL and VH domains of the anti-EphA2 antibody EA2. The nucleotide and amino acid sequences of the VL domain (SEQ ID NOS: 1 and 2, respectively) are shown in (A). The nucleotide and amino acid sequences of the VH domain (SEQ ID NOS: 9 and 10, respectively) are shown in (B). The nucleotide and amino acid sequences of the CDRs are indicated in bold and are underlined. The EA2 antibody was previously described in U.S. Pat. Pub. No. US 2005-0152899 A1, and is incorporated by reference herein in its entirety.

FIGS. 2A-2B: Sequences of the VL and VH domains of the anti-EphA2 antibody EA5. The nucleotide and amino acid sequences of the VL domain (SEQ ID NOS: 17 and 18, respectively) are shown in (A). The nucleotide and amino acid sequences of the VH domain (SEQ ID NOS: 25 and 26, respectively) are shown in (B). The nucleotide and amino acid sequences of the CDRs are indicated in bold and are underlined. The EA5 antibody was previously described in U.S. Pat. Pub. No. US 2005-0152899 A1, and is incorporated by reference herein in its entirety.

FIG. 3: Structure of the EphA2-BiTE cDNA. The inserts cloned into the expression vector pEF-DHFR each comprised a leader with a 5′-terminal Kozak site for increased translation efficiency and a secretory signal sequence (murine Ig heavy chain leader). The leader is followed by four variable Ig domains as listed above. The linker peptide at the VL-VH or VH-VL junction of anti-EphA2 has a length of 15 amino acids (three repeats of the motif G4S). The linker peptide connecting the EphA2 with the CD3 binding specificity comprises one repeat of the motif G4S. Directly adjacent to the fourth domain is a C-terminal hexa-histidine (H6) sequence for detection and purification purposes. The indicated restriction enzyme sites were used for cloning the EphA2-BiTE constructs into the expression vector.

FIG. 4: Structure of the pEF-DHFR vector used express the EphA2-BiTEs in CHO cells. The pEF-DHFR vector is a 5.8 kb vector with the murine dihydrofolate reductase as selection marker forming a bi-cistronic transcription unit together with the gene to be expressed under the control of the human EF1α promoter. The eukaryotic expression vector is a derivative of the expression vector pMT2PC.

FIG. 5: Elution profile from EphA2-BiTE deimmunized anti-CD3×EA2 (VH/VL) containing protein fractions from a gelfiltration column. Peak 1: aggregates, peak 2: dimer, peak 3: monomer.

FIGS. 6A-6B: SDS PAGE (A) and anti-His tag Western blot (B) of purified EphA2-BiTE deimmunized anti-CD3×EA2 (VH/VL) fractions. The monomer fraction (lane 7) contained essentially pure EphA2 BiTE. No EphA2-BiTE was detectable in the aggregate.

FIGS. 7A-7B: Flow cytometric binding analysis of cynomolgus-reactive anti-CD3 parental antibodies. PBMC from cynomolgus monkeys were used to detect CD3-binding of parental antibodies cCD3-1 (A) and cCD3-2 (B) both conjugated with FITC. Cells were analyzed by flow cytometry on a FACS-Calibur (Becton Dickinson, Heidelberg). As shown here, both antibodies show distinct binding to the T-cell fraction of cynomolgus PBMC.

FIG. 8: Evidence for crossreactivity of EA2 and EA5 with Rhesus EphA2. EA2 and EA5 antibodies activated phosphorylation of EphA2 in CMMT110/CL cells as shown in the Western blot.

FIG. 9: Flow cytometric binding analysis of various anti-EphA2 surrogate BiTE constructs reacting with macaque CD3. Only those surrogate BiTE constructs based on macaque CD3 antibody cCD3-1 showed strong binding to CD3 and EphA2. Constructs based on cCD3-2 only show strong CD3-binding; the EphA2-binding turned out to be almost completely suppressed. Thus, cCD3-1 based surrogate BiTE constructs will be proceeded to production, purification and analysis of cytotoxic activity with cynomolgus T-cells.

FIGS. 10A-10B: Flow cytometric binding analysis of anti-EphA2 parental antibodies. EphA2 expressing A549 cells (human lung carcinoma cell line) (A), and MDA-MB-231 cells (human breast cancer) (B) were used. Cells (200,000) were incubated with 10 μg/ml of the respective antibody for 30 min on ice. The cells were subsequently washed twice in PBS. The binding of the primary antibody was detected via an phycoerythrin-conjugated murine Fc-gamma specific antibody (Dianova, order no. 115-116-071) diluted 1:100 in 50 μl PBS with 2% FCS. As negative control, an irrelevant antibody with the same isotype was used (thin line). Cells were analyzed by flow cytometry on a FACS-Calibur (Becton Dickinson, Heidelberg). As shown in the figure, mAb B322 showed the strongest binding signal followed by EA2 and EA5. The binding capabilities of the three different antibodies are shown in a histogram overlay (4^{th} peak from left=B322 antibody; 3^{rd} peak from left=EA2 antibody; 2^{nd} peak from left=EA5 antibody; far left peak—control).

FIGS. 11A-11C: Immunohistochemical analysis of EA2 and EA5 binding to normal tissues. (A) No primary antibody. EA5 demonstrated staining of intercalated discs in heart tissue (B), vascular and stromal smooth muscle elements of multiple organs (cytoplasm), colonic epithelium (cytoplasm), and uterine myometrium. Human tissue sections (C) were not stained with EA2 as compared with an isotype control monoclonal antibody 1A7.

FIG. 12: Bispecific binding of EphA2-BiTEs on different cell lines as determined by flow cytometry. EphA2-positive A549 cells (human lung carcinoma cell line) and MDA-MB-231 cells (human breast cancer cell line), as well as CD3-positive HP-BALL (human T-cell line) were used.

FIGS. 13A-13B: Potency of redirected lysis by five EphA2-BiTEs in (A) MDA MB 231 (breast), and (B) A549 (lung) cells. The BiTE construct deimmunized anti-CD3×EA2(VH/VL) consistently showed the highest potency in redirected lysis of breast and lung cancer cell lines.

FIGS. 14A-14B: Sequences of the deimmunized anti-CD3(VH-VL)×EA2 (VH-VL) EphA2-BiTE. Shown in (A) is a depiction of an EphA2-BiTE construct comprising a first binding domain that immunospecifically binds CD3 and a second binding domain that immunospecifically binds EphA2. Also depicted are linker sequences between the VH_{CD3}-VL_{CD3}, VL_{CD3}-VH_{EphA2} and VH_{EphA2}-VL_{EphA2} domains, respectively, in the 5′ to 3′ direction (left to right). The amino acid and nucleotide sequences of each linker are represented by SEQ ID NOS:27 and 28, 29 and 30, and 31 and 32, respectively. Shown in (B) is the full length sequence nucleotide sequence (SEQ ID NO:60) and amino acid sequence (SEQ ID NO:65) of the deimmunized anti-CD3(VH-VL)×EA2 (VH-VL) EphA2-BiTE. Bold letters represent the enzyme restriction sites of EcoRI and SalI, respectively. Italic upper case letters indicate the leader sequence, and double-underlined letters represent the stop codon. The linker and Histidine-tag sequences are underlined.

FIGS. 15A-15D: The cytotoxic activity of two different batches of EphA2 BiTE deimmunized anti-CD3×EA2(VH/VL) was compared with PBMCs (after depletion of CD16 positive cells) and stimulated CD8+ T-cells as effector cells. The EphA2-positive cell lines A549 and MDA-MB231 served as target cells. The E:T ratio was 10:1; and the incubation time was 18 hours. (A) A549 cell lysis mediated by EphA2-BiTE with PBMC; (B) A549 cell lysis mediated by EphA2-BiTE with stimulated CD8+ T-cells; (C) MDA-MB231 cell lysis mediated by EphA2-BiTE with PBMC and (D) MDA-MB231 cell lysis mediated by stimulated CD8+ T-cells.

FIG. 16: Variation of EphA2-BiTE cytotoxic activity with effector cell donor. EphA2-BiTE redirected T-cells from different subjects to mediate EphA2+ tumor cell killing. The cytotoxicity assay utilized SW480 target cells and CD3+ T-cells isolated from 49 individual human donors. For the majority of human donors, EphA2-BiTE mediated tumor cell killing at very low concentrations. The EC50 for EphA2-BiTE was between 1 and 110 ng/ml for all of the T-cell donors tested with a median (horizontal bar) of 24 ng/ml. Thus, for the majority of human T-cell donors, EphA2-BiTE is a highly potent molecule with killing activity observed at concentrations in the low ng/ml range.

FIGS. 17A-17B: Specificity of target cell binding—soluble EphA2 fusion protein competes for binding of deimmunized anti-CD3×EA2(VH/VL). The target binding specificity of BiTE deimmunized anti-CD3×EA2(VH/VL) was tested in a competition assay (A). As shown in the figure, coincubation of BiTE deimmunized anti-CD3×EA2(VH/VL) with a 10-fold weight excess of soluble EphA2 Fc fusion protein completely blocked binding of the BiTE to A549 human lung cancer cells (B). This shows that BiTE binding to tumor cells is specifically mediated by recognition of EphA2 target.

FIG. 18: Target cell specificity of EphA2-BiTE deimmunized anti-CD3×EA2 (VH/VL): killing of antigen positive cells. The cytotoxic activity of the deimmunized anti-CD3×EA2 BiTE strictly depends on the expression of EphA2 on the transfected B16F10 target cells; EphA2-negative B16F10 cells are completely resistant to deimmunized anti-CD3×EA2 mediated lysis.

FIGS. 19A-19C: Specificity of deimmunized anti-CD3×EA2 (VH/VL) in vitro cytotoxic activity. (A) Specificity of target cell lysis. CD3+ T-cell enriched PBMC were incubated with EphA2 positive SW480 (closed symbols) or EphA2 negative SK− MEL-28 (open symbols) cells in the presence of serial dilutions of deimmunized anti-CD3×EA2 (VH/VL) (squares) or bscCD19×CD3 (triangles) for 42 hours at 37° C. (B and C) Parental antibodies to CD3 (DiL2K) and EphA2 (EA2) inhibit bscEphA2×CD3 lysis of EphA2 positive tumor cells. CD3+ T-cells were incubated in 200 μl of culture medium with SW480 cells in the presence of serial dilutions of deimmunized anti-CD3×EA2 (VH/VL) alone (open squares), or in the presence of 10 μg (triangle) or 100 μg (circle) of DiL2K in panel B or EA2 in panel C for 42 hours at 37° C.; results are presented as the percentage of lysed target cells. Error bars indicate the standard deviation (SD) of duplicate measurements. Note: the magnitude of lysis approaches 100% for each deimmunized anti-CD3×EA2 (VH/VL) curve.

FIGS. 20(A)-20(D): Renal cell carcinoma and prostate cancer cell killing mediated by EphA2-BiTE. Cytotoxicity assays demonstrated EphA2-BiTE mediated tumor cell killing of ACHN (A), Caki 2 (B), PC3 (C), and DU145 (D) cells with EC₅₀ values of 3, 30, 6, and 0.8 ng/ml, respectively. A negative control BiTE specific for CD19 did not redirect T-cells to lyse target cells.

FIGS. 21A-21B: Effects of EphA2-BiTE killing upon EphA2 levels on target cells. (A) Cytotoxicity assays demonstrated EphA2-BiTE mediated tumor cell killing of SW480 cells with EC50 value of 6 ng/ml. A negative control BiTE specific for CD19 did not redirect T-cells to lyse target cells. (B) After completion of an in vitro cytotoxicity assay, the remaining live SW480 target cells were measured for the presence of EphA2 or EphA2-BiTE. The geometric mean fluorescence intensity of live SW480 cells stained with an isotype control, EphA2 expression (B233 staining), or EphA2-BiTE (BiTE) was plotted versus the dose of input BiTE. EphA2-BiTE redirected T-cells to first kill target cells with high EphA2 expression levels. Interestingly, not kill all target cells were lysed even though EphA2-BiTE was bound to the cells. Thus, EphA2-BiTE mediated T-cell killing of most EphA2+ target cells although a subset of EphA2+ cells may be resistant.

FIGS. 22A-22D: Characterization of deimmunized anti-CD3×EA2 (VH/VL) in vitro cytotoxicity. (A) Kinetics of redirected lysis. CD3+ T-cells were incubated with SW480 cells in the presence of serial dilutions of deimmunized anti-CD3×EA2 (VH/VL) for 4 (square), 18 (triangle), 24 (inverted triangle), or 42 (diamond) hours at 37° C. Error bars indicate the SD. EC50 values were estimated from the curves and are listed. (B) Influence of effector to target ratio on redirected lysis. CD3+ T-cells were incubated with SW480 cells at a ratio of 20:1 (black square), 10:1 (black triangle), 5:1 (open inverted triangle), 1:1 (open diamond), 1:2 (grey circle), or 1:5 (grey square) in the presence of serial dilutions of deimmunized anti-CD3×EA2 (VH/VL) for 42 hours at 37° C. Error bars indicate the SD. EC50 values were estimated from the curves and are listed. (C) Effects of available EphA2 receptor binding sites on the potency of redirected lysis. The estimated number of EphA2 molecules per cell for each tumor line (HeyA8, SW480, A549 human carcinoma lines and M14 and A375 melanoma lines) was plotted against the respective EC50 values of the percentage of target cells lysed from four separate experiments. P value and r2 of the linear regression curve are listed on the graph. (D) Effects of EphA2 surface density on redirected lysis. CD3+ T-cells were incubated with the cell lines HeyA8 (black squares; 107,000 EphA2 receptors per cell), M14 (grey circle; 2,400 EphA2 receptors per cells), and SKMEL-28 (open square; EphA2 receptors were below limit of detection) in the presence of serial dilutions of deimmunized anti-CD3×EA2 (VH/VL) for 18 hours at 37° C. Error bars indicate the SD. EC50 values were estimated from the curves and are listed.

FIG. 23: Stability of EphA2-BiTE deimmunized anti-CD3×EA2 (VH/VL) in human plasma. The plasma stability of the EphA2-specific BiTE was tested under different incubation conditions followed by ED50 determination of cytotoxic activity in a standard 51-chromium release assay.

FIGS. 24A-E: Target epitope exclusion on non-transformed cells by deimmunized anti-CD3×EA2(VH/VL). Video microscopy was employed to visualize the attack of CD8+ T-cells against non-transformed MCF10A cells in the presence of BiTE deimmunized anti-CD3×EA2 (VH/VL) and a non-epitope excluding control BiTE. (A) Non-transformed MCF10A co-incubated with CD8⁺ T-cells (E:T ratio 1:1) and 100 ng/ml deimmunized anti-CD3×EA2 (VH/VL). (B) Non-transformed MCF10A co-incubated with CD8⁺ T-cells (E:T ratio 1:1) without BiTE. (C) Non-transformed MCF10A co-incubated with CD8⁺ T-cells (E:T ratio 1:1) and 100 ng/ml of a pan-carcinoma non epitope excluding control BiTE. (D) Lung carcinoma cell line A549 coincubated with CD8⁺ T-cells (E:T ratio 1:1) and 100 ng/ml deimmunized anti-CD3×EA2 (VH/VL). (E) Same setup as in (D). Overlay of transmitting light microscopy and fluorescence light microscopy picture in presence of propidium iodide to visualize lysed cells (light gray).

FIG. 25: Determination of target affinity of deimmunized anti-CD3×EA2(VH/VL) by surface plasmon resonance. The formation and dissociation of BiTE/EphA2 complexes was monitored by surface plasmon resonance using a Biacore 3000 system. The K_{D} is estimated at 45 nM.

FIG. 26: Negative control test groups for in vivo anti-tumor effect of deimmunized anti-CD3×EA2(VH/VL) in SW480 model. 5×10⁶ SW480 cells alone (“SW480 only” and “deimmunized anti-CD3×EA2”) or admixed with human 2.5×10⁶ CD3+ T-cells (“PBS”, “non-relevant BiTE”, and “add. T-cells i.v., PBS”) were subcutaneously injected into female NOD/SCID mice. One group of mice received 2.5×10⁶ CD3+ T-cells intravenously injected (“add. T-cells, i.v., PBS”). Mice were treated daily for five consecutive days with BiTE (“deimmunized anti-CD3×EA2 only”), PBS (“PBS”; “add. T-cells i.v., PBS”), or a non-relevant BiTE. Mean tumor volume of six mice/group are shown.

FIG. 27: Deimmunized anti-CD3×EA2(VH/VL) daily treatment for five consecutive days induced a dose-dependent inhibition of SW480 tumor outgrowth in the presence of CD3+ effector T-cells. **Highly significant (p<0.01); *significant (p<0.05) by Student's T test.

FIG. 28: No effect of peripheral T-cells on anti-tumor effect of deimmunized anti-CD3×EA2(VH/VL). Mean tumor volume of six mice/group is shown. **Highly significant (p<0.01).

FIGS. 29A-29B: Amino acid sequences of the VL and VH domains of the anti-EphA2 antibody 233. Shown in (A) is the amino acid sequence of the VL domain (SEQ ID NO:33). Shown in (B) is the amino acid sequence of the VH domain (SEQ ID NO:37). The sequences of the CDRs are boxed. The 233 antibody was previously described in U.S. Pat. Pub. No. US 2004-0028685 A1, and is incorporated by reference herein in its entirety.

FIGS. 30A-30B: Amino acid sequences of the VL and VH domains of the anti-EphA2 antibody 3F2. Shown in (A) is the amino acid sequence of the VL domain (SEQ ID NO:41). Shown in (B) is the amino acid sequence of the VH domain (SEQ ID NO:45). The sequences of the CDRs are boxed. The 3F2 antibody was previously described in U.S. patent application Ser. No. 11/203,251, filed Aug. 15, 2005, and is incorporated by reference herein in its entirety.

FIG. 31: Linear map of 4H5 scFv insertion site in the MD102 expression vector.

FIGS. 32A-32B: Sequences of the 4H5 VH-VL scFV humanized clone. Shown in (A) is the nucleotide sequence (SEQ ID NO:67). Shown in (B) is the amino acid sequence (SEQ ID NO:68). The CDRs are boxed. The 4H5 antibody was previously described in U.S. Pat. Appn. Ser. No. 2005-0048617 A1, and is incorporated by reference herein in its entirety.

FIG. 33A-33B: Nucleotide and amino acid sequence of the VH VL 2A4 scFv. Shown in (A) is the nucleotide sequence (SEQ ID NO:75). Shown in (B) is the amino acid sequence (SEQ ID NO:76). The CDRs are boxed.

FIGS. 34A-34B: Nucleotide and amino acid sequence of the VH VL 2E7 scFv. (A) depicts the nucleotide sequence (SEQ ID NO:83); and (B) depicts the amino acid sequence (SEQ ID NO:84).

FIGS. 35A-35B: Nucleotide and amino acid sequence of the VH VL 12E2 scFv. (A) depicts the nucleotide sequence (SEQ ID NO:91); and (B) depicts the amino acid sequence (SEQ ID NO:92).

FIG. 36: ELISA titration of scFv supernatants of combinatorial affinity optimized variants on immobilized human EphA2.

FIG. 37: Amino acid sequence alignment of the affinity optimized variants 2A4, 2E7 and 12E2 with the humanized 4H5 scFv.

FIG. 38: This figure depicts the sequence of the combinatorial primers (L1, L2, L3, L4, L5, H6, H7, H8, H9, H10, H11, H12, and H13) used in the affinity optimization of Fab 2G6 (SEQ ID NOS: 99-111, respectively).

FIG. 39: Amino acid sequence alignment of the variable regions of murine B233, humanized 2G6 and affinity optimized 3F2 mAbs are provided.

FIG. 40: Anti-human EphA2 antibodies bind to cynomolgus EphA2. Purified murine (EA2) and humanized (3F2 and 4H5) anti-human antibodies bound to human EphA2 on SW480 colon carcinoma cells and cynomolgus EphA2 expressed on transfected CHO cells. Bound antibodies were detected by flow cytometry using an anti-mouse or human IgG (H+L) Alexafluor 488 antibody. Antibodies were compared with a nonbinding negative control antibody (R347). EA2, 3F2, and 4H5 bound to human and cynomolgus EphA2.

FIG. 41: Selective tumor cell recognition by a subset of EphA2 antibodies. Monolayers of nontransformed (MCF-10A) and malignant (MDA-MB-231) mammary epithelial cells were fixed and labeled with the indicated antibodies (G5, EA5, EA2, 3F2, or 4H5), then visualized using immunofluorescence microscopy. Antibodies excluded (yes) or not excluded (no) from sites of cell-cell contact of MCF-10A cells are indicated. Thus, EA5 and EA2 epitopes are selectively excluded by the normal architecture that typifies nontransformed epithelial cells but becomes accessible after malignant transformation.

FIGS. 42A-42B: Amino acid sequences of the VL and VH domains of G5. Shown in (A) is the amino acid sequence of VL domain (SEQ ID NO:49). Shown in (B) is the amino acid sequence of the VH domain (SEQ ID NO:53). The G5 antibody was previously described in U.S. Appn. Ser. No. 11/165,023, filed Jun. 24, 2005, and is incorporated by reference herein in its entirety.

FIG. 43: Cytotoxic potency comparison of four EphA2-specific BiTEs made from two different production runs of the humanized 3F2 monoclonal antibody. In vitro cytotoxic assays compared the ability of four different 3F2-based anti-EphA2 BiTE constructs to mediate redirected T-cell lysis of EphA2+MDA-MB-231 cells. The table lists potency (i.e., EC50) values for each construct purified from two separate production runs. The BiTE construct deimmunized anti-CD3×3F2 (VH/VL) consistently showed the greatest potency.

FIG. 44: Independent cytotoxic potency comparison of four 3F2-based EphA2-specific BiTEs. In vitro cytotoxic assay compared the ability of four different 3F2-based anti-EphA2 BiTE constructs to mediate redirected T-cell lysis of EphA2+SW480 cells. The BiTE construct deimmunized anti-CD3×3F2 (VL/VH) showed the greatest potency.

FIG. 45: Specificity of target cell binding of 4H5-based anti-EphA2 BiTES to EphA2+ and CD3+ expressing cells. The target binding specificity of the various 4H5-based BiTE constructs was examined using the flow cytometry-based assay described above. EphA2+MDA MB 231 breast cancer cells and CD3+ HP Ball human T-cells were used as target cells. The deimmunized anti-CD3×EA2 (VH/VL) BiTE was used as a positive control. As shown in the figure, each of the 4H5-based EphA2-BiTE constructs bound to cells expressing both EphA2 and CD3.

FIG. 46: Specificity of target cell binding of the humanized affinity matured 4H5-based BiTE constructs 12E2, 2E7 and 2A4. The target binding specificity of the various 4H5-based BiTE constructs was examined using the flow cytometry-based assay described above. EphA2+MDA MB 231 breast cancer cells and CD3+ HP Ball human T-cells were used as target cells. As shown in the figure, each of the 4H5-based EphA2-BiTE constructs bound to cells expressing both EphA2 and CD3.

FIG. 47: Specificity of target cell binding of purified monomers. As shown in the figure, the purified monomers of the affinity matured 4H5-based EphA2-BiTE constructs bound to EphA2+MDA MB 231 breast cancer cells and CD3+ HP Ball human T-cells target cells at a concentration of 5 μg/ml using the flow cytometry-based assay as described above.

FIG. 48: Cytotoxic potency comparison of four EphA2-specific BiTEs of the humanized 4H5 monoclonal antibody. Cytotoxicity assays based on Chromium-51 release were performed to determine redirected target cell lysis by the various anti-EphA2 BiTE constructs in the presence of stimulated human CD8+ T-cells. The EphA2-positive tumor cell line MDA-MB-231 was loaded with Chromium-51 and served as target cells. The various anti-EphA2 BiTE constructs were titrated over a broad range of concentrations. The assay duration was 18 hours, and the effector-to-target ratio 10:1. EphA2-BiTE concentrations required for half-maximal lysis (i.e., EC50) with different production batches were estimated using a four-parameter non-linear fit model.

FIGS. 49A-49B: This figure provides a direct comparison of the potency of target cell lysis of MDA-MB-231 cells of the various 3F2 (A) and (B) 4H5-based EphA2 constructs as measured by a Chromium-51 based cytotoxicity assay.

FIG. 50: Cytotoxicity activity induced by affinity matured 4H5-based EphA2-BiTEs 12E4, 2E7 and 2A4. This figure demonstrate the potency of target cell lysis of the various 4H5-based EphA2-BiTE constructs (12E4, 2E7 and 2A4). Cells from the EphA2-positive tumor cell line A549 were loaded with Chromium-51 and served as target cells. Stimulated human CD8+ T-cells served as effector cells. The various anti-EphA2 BiTE constructs were titrated over a broad range of concentrations. The assay duration was 18 hours, and the effector-to-target ratio 10:1. EphA2-BiTE concentrations required for half-maximal lysis (i.e., EC50) with different production batches were estimated using a four-parameter non-linear fit model.

FIG. 51: Cytotoxicity activity induced by affinity matured 4H5-based EphA2-BiTEs 12E4, 2E7 and 2A4. This figure demonstrate the potency of target cell lysis of the various 4H5-based EphA2-BiTE constructs (12E4, 2E7 and 2A4). Cells from the EphA2-positive tumor cell lines A549 and SW480 were loaded with Chromium-51 and served as target cells. Unstimulated human CD3+ T-cells served as effector cells. The various anti-EphA2 BiTE constructs were titrated over a broad range of concentrations. The assay duration was 42 hours, and the effector-to-target ratio 5:1. EphA2-BiTE concentrations required for half-maximal lysis (i.e., EC50) with different production batches were estimated using a four-parameter non-linear fit model.

FIG. 52: No activation of EphA2 by 4H5-based EphA2-BiTEs as measured by EphA2 phosphorylation. This figure demonstrates that neither the 2A4 nor the 2E7 BiTE construct induced EphA2 phosphorylation in EphA2-expressing cells.

FIGS. 53A-53B: In vivo efficacy of affinity matured 4H5-based EphA2-BiTE constructs. The in vivo potency of the 2A4- and 2E7-BiTE constructs were evaluated in immunodeficient NOD/SCID mice with a human colon carcinoma xenograft model. The human colon carcinoma cell line SW480 was selected for the establishment of a human xenograft model since SW480 cells express EphA2. Results of the study are shown here.

### 5. DETAILED DESCRIPTION OF THE INVENTION

As detailed below, the present invention relates to bispecific single chain antibodies comprising a first binding domain that immunospecifically binds to the T-cell antigen CD3 and a second binding domain that immunospecifically binds to the EphA2 receptor. Such bispecific single chain antibodies are encompassed by the term “EphA2-BiTEs.” The present invention further relates to methods and compositions designed for the treatment, prevention and/or management of disorders associated with aberrant expression and/or activity of EphA2. Such disorders include, but are not limited to, cancer, non-cancer hyperproliferative cell disorders, and infections. The invention further relates to vectors comprising polynucleotides encoding the EphA2-BiTEs of the invention, host cells transformed therewith, and their use in the production of said EphA2-BiTEs. The invention also provides compositions, including pharmaceutical compositions, comprising any of the aforementioned EphA2-BiTEs, polynucleotides or vectors either alone or in combination with one or more prophylactic or therapeutic agents. Also disclosed are methods of screening for said EphA2-BiTEs and kits comprising any of the aforementioned compositions and diagnostic reagents.

5.1 EphA2-BiTEs

The present invention provides bispecific T-cell engagers (i.e., EphA2-BiTEs (in particular, EphA2-BiTEs which are bispecific single chain antibodies)) that immunospecifically bind EphA2 and the T-cell antigen CD3, and methods of using the same to treat, prevent and/or manage disorders associated with aberrant expression and/or activity of EphA2. Such disorders include, for example, cancer, non-cancer hyperproliferative cell disorders, and infections. In one aspect, the EphA2-BiTEs are more efficient at eliminating cells that aberrantly express EphA2 than EphA2-specific antibodies known in the art. In a specific aspect, the EphA2-BiTEs are more efficient at eliminating EphA2-expressing cancer cells (in particular, EphA2-expressing malignant cancer cells) than EphA2 antibodies known in the art. In another aspect, the EphA2-BiTEs are more efficient at eliminating EphA2-expressing non-cancer hyperproliferative cells than EphA2 antibodies known in the art. In yet another aspect, the EphA2-BiTEs of the invention are more efficient at eliminating EphA2-expressing infected cells (in particular, cells infected with the Respiratory Syncytial Virus; “RSV”) than EphA2 antibodies known in the art. In another aspect, lower dosages of EphA2-BiTEs than EphA2-specific antibodies known in the art are needed to treat, prevent and/or manage disorders associated with aberrant expression and/or activity of EphA2.

The EphA2-specific BiTEs of the present invention comprise a first binding domain that immunospecifically binds to the T-cell antigen CD3 and a second binding domain that immunospecifically binds to EphA2 (hereinafter “EphA2-BiTEs” or “EphA2-BiTE molecules”). In one embodiment, the first binding domain immunospecifically binds to CD3. In a specific embodiment, the first binding domain immunospecifically binds to one or more of any subunit of CD3 (e.g., the gamma, delta, zeta, or eta subunit). In a preferred embodiment, the first binding domain immunospecifically binds to the epsilon (ε) subunit of CD3. In a specific embodiment, the first binding domain immunospecifically binds to the epsilon (ε) subunit of CD3 when said subunit is complexed with the delta subunit of CD3. In another embodiment, the binding domain that binds to CD3 is deimmunized. In another specific embodiment, the second binding domain immunospecifically binds to the extracellular domain of EphA2. In a preferred embodiment, the second binding domain of the EphA2-BiTEs, which are used in the treatment, prevention and/or management of cancer, immunospecifically binds to epitopes on EphA2 that are selectively exposed and/or increased on cancer cells but not non-cancer cells. In another preferred embodiment, the second binding domain of the EphA2-BiTEs of the invention immunospecifically binds to epitopes on EphA2 that are selectively exposed and/or increased on non-cancer hyperproliferative cells but not normal cells. In another preferred embodiment, the second binding domain of EphA2-BiTEs immunospecifically binds to epitopes on EphA2 that are selectively exposed and/or increased on infected cells but not non-infected cells.

The present invention provides EphA2-BiTEs in which the first binding domain comprises a variable heavy (VH) domain and a variable light (VL) domain of an antibody that immunospecifically binds to the T-cell antigen CD3 and a second binding domain that comprises a VH domain and a VL domain of an antibody that immunospecifically binds to EphA2. In a specific embodiment, the VH domain and VL domains of the first binding domain are linked together by a linker of sufficient length to enable the domains to fold in such a way as to permit binding to the T-cell antigen CD3. Further to this embodiment, such a linker may comprise, for example, the sequence GEGTSTGS(G₂S)₂GGAD (SEQ ID NO.:57). In another specific embodiment, the VH domain and VL domains of the second binding domain are linked together by a linker of sufficient length to enable the domains to fold in such a way as to permit binding to EphA2. Further to this embodiment, such a linker may comprise, for example, the sequence (G₄S)₃ (SEQ ID NO:59). In another specific embodiment, the first and second binding domains are linked together by a linker of sufficient length to enable the domains to fold in such a way as to permit binding to the T-cell antigen CD3 and to EphA2. Further to this embodiment, such a linker may comprise, for example, the sequence G₄S (SEQ ID NO:58). In a specific embodiment, an EphA2-BiTE of the invention has the amino acid sequence of (SEQ ID NO:65). In a specific embodiment, an EphA2-BiTE of the invention comprises a single domain antibody.

In accordance with the embodiment in the preceding paragraph, the linkage is covalent. In a specific embodiment, the linkers of the invention comprise serine and glycine residues. The linkers of the EphA2-BiTEs, e.g., the linker between the VH and VL domains of the first binding domain that binds to CD3, the linker between the VH and VL domains of the second binding domain that binds to EphA2, and the linker between the first binding domain that binds to CD3 and the second binding domain that binds to EphA2 may be of any length sufficient to enable the domains to fold in such a way as to permit binding to the CD3 and EphA2 antigens, respectively. In certain embodiments, the linkers of the invention comprise a length of at least 5 residues, at least 10 residues, at least 15 residues, at least 20 residues, at least 25 residues, at least 30 residues or more. In other embodiments, the linkers of the invention comprises a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, or between 2-30 residues. In certain embodiments, the first binding domain is 5′ to the second binding domain. In other embodiments, the second binding domain is 5′ to the first binding domain. In certain embodiments, the first and second binding domains are single chain antibodies. In a specific embodiment, the first and second binding domains comprise single chain Fvs (scFvs).

In another specific embodiment, the invention provides a bispecific single chain antibody comprising (a) a first heavy chain variable domain and a first light chain variable domain each from an antibody that immunospecifically binds the ε chain of CD3, said first heavy chain variable domain covalently linked to said first light chain variable domain by a first linker of sufficient length (e.g., GEGTSTGS(G₂S)₂GGAD (SEQ ID NO.:57)) such that said first heavy chain variable domain and said first light chain variable domain fold to form a first binding domain that binds the ε subunit of CD3; and (b) a second heavy chain variable domain and a second light chain variable domain from an antibody that immunospecifically binds an epitope of EphA2 exposed on the cell surface, said second heavy chain variable domain covalently linked to said second light chain variable domain by a second linker of sufficient length (e.g., (G₄S)₃ (SEQ ID NO:59)) such that said second heavy chain variable domain and said second light chain variable domain fold to form a second binding domain that binds said epitope of EphA2, wherein said first binding domain and said second binding domain are covalently linked by a third linker of a length (e.g., G₄S (SEQ ID NO:58)) such that said first binding domain and said second binding domain fold independently of each other.

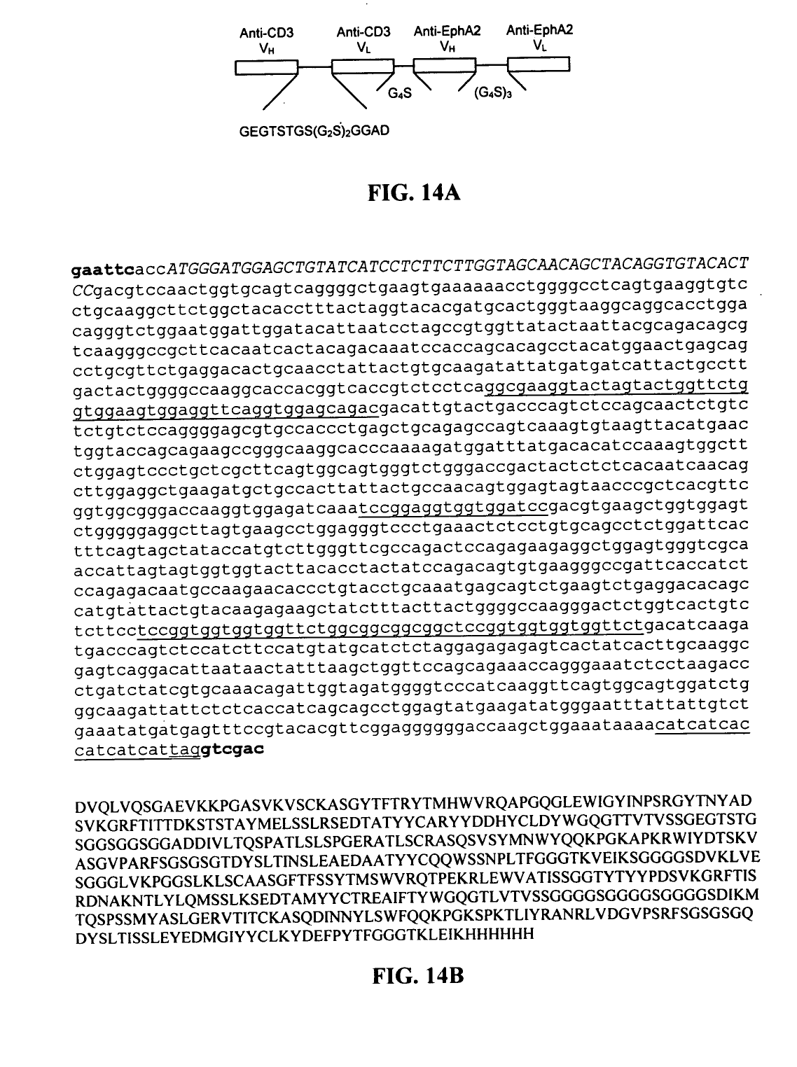

In specific embodiments, the EphA2-BiTEs of the invention comprise any of the following arrangements in the 5′ to 3′ direction: (1) VH_{CD3}-VL_{CD3}-VH_{EphA2}-VL-EphA2; (2) VL_{CD3}-VH_{CD3}-VH_{EphA2}-VL_{EphA2}; (3) VL_{CD3}-VH_{CD3}-VL_{EphA2}-VH-_{EphA2}; (4) VH_{CD3}-VL_{CD3}-VL_{EphA2}-VH_{EphA2}; (5) VH_{EphA2}-VL_{EphA2}-VH_{CD3}-VL_{CD3}; (6) VL_{EphA2}-VH_{EphA2}-VH_{CD3}-VL_{CD3}; (7) VL_{EphA2}-VH_{EphA2}-VL_{CD3}-VH_{CD3}; or (8) VH_{EphA2}-VL_{EphA2}-VL_{CD3}-VH-_{CD3}. See, e.g., FIG. 14A for a generic depiction of the EphA2-BiTE constructs of the invention.

As is well known, an antibody fragment which contains a complete antigen recognition and binding domain, in certain circumstances may consist of a dimer of one heavy and one light chain variable domain (VH and VL) in non-covalent association. In this configuration that corresponds to the one found in native antibodies, the three complementarity determining regions (CDRs) of each variable domain interact to define an antigen binding site on the surface of the VH-VL dimer. Collectively, the six CDRs confer antigen binding specificity to the antibody. Frameworks (FRs) flanking the CDRs have a tertiary structure which is essentially conserved in native immunoglobulins in species as diverse as humans and mouse. These FRs serve to hold the CDRs in their appropriate orientation. The constant domains are not required for binding function, but may aid in stabilizing the VH-VL interaction. Even a single variable domain (or half of an Fv comprising only three CDRs specific for an antigen) has the ability to recognize and bind antigen with high conformational stability (see, e.g., Dumoulin et al., 2002, Protein Science 11:500-515). Hence, said domain of the binding domain of an EphA2-BiTE of the invention may comprise a pair of VH-VL domains either of the same or of different immunoglobulins. The order of the VH and VL domains within the polypeptide chain is not decisive for the present invention; the invention encompasses all possible arrangements of the variable domains. It is important, however, that the VH and VL domains are arranged so that the antigen binding domain can properly fold to recognize and bind antigen. In a specific embodiment, the domains of the EphA2-binding domain can be from the same or from a different immunoglobulin.

Accordingly, the EphA2-BiTEs of the invention may comprise any arrangement of the variable heavy domain and variable light domain of each antibody (e.g., the VH and VL domains of an antibody which immunospecifically binds to CD3 and the VH and VL domains of an antibody which immunospecifically binds to another antigen of interest (e.g., EphA2)), and each domain may be located at the N-terminal or C-terminal portion of the BiTE molecule. For example, and not by way of limitation, an EphA2-BiTE molecule of the invention may comprise the following arrangements as outlined in the following table:

| N-terminal Position | C-terminal Position |
|---|---|
| EA2 (VL/VH) | deimmunized anti- |
| EA2 (VH/VL) | CD3 (VH/VL) |
| EA5 (VL/VH) | deimmunized anti- |
| EA5 (VH/VL) | CD3 (VH/VL) |
| deimmunized anti- | EA2 (VL/VH) |
| CD3 (VH/VL) | EA2 (VH/VL) |
| deimmunized anti- | EA5 (VL/VH) |
| CD3 (VH/VL) | EA5 (VH/VL) |

Said binding domains discussed above are preferably connected by a flexible linker, preferably by a polypeptide linker disposed between said domains, wherein said polypeptide linker comprises plural, hydrophilic, peptide-bonded amino acids of a length sufficient to span the distance between the C-terminal end of one of said domains comprising said binding domains and the N-terminal end of the other of said domains comprising said binding domains when the polypeptide of the invention assumes a conformation suitable for binding when disposed in aqueous solution. Preferably, said polypeptide linker comprises a plurality of glycine, alanine and/or serine residues. It is further preferred that said polypeptide linker comprises a plurality of consecutive copies of an amino acid sequence. Usually, the polypeptide linker comprises 1 to 15 amino acids although polypeptide linkers of more than 15 amino acids may work as well. In certain embodiments, the linkers of the invention comprise a length of at least 5 residues, at least 10 residues, at least 15 residues, at least 20 residues, at least 25 residues, at least 30 residues or more. In other embodiments, the linkers of the invention comprises a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, or between 2-30 residues. Examples of linkers that may be used in accordance with the methods of the invention are found in FIG. 3 (SEQ ID NOS.:57-59).

In an embodiment of the invention, an antibody or ligand that immunospecifically binds to EphA2 will comprise a portion of the BiTE molecule. For example, the VH and/or VL (preferably a scFV) of an antibody that immunospecifically binds to EphA2 can be fused to an anti-CD3 binding portion such as that of the molecule described above, thus creating a bispecific single chain antibody that targets EphA2. In addition to the VH and/or VL domains of an antibody EphA2, other molecules that bind EphA2 can comprise the EphA2-BiTE, for example receptors or ligands (e.g., an Ephrin).

In a specific embodiment, an EphA2-BiTE of the invention is a bispecific single chain antibody that comprises a first binding domain that immunospecifically binds CD3, and a second binding domain that immunospecifically binds EphA2. In a specific embodiment, the first binding domain comprises a deimmunized version of the VH and VL domains of an antibody that immunospecifically binds to CD3. Also in accordance with this embodiment, the second binding domain comprises the VH and VL domains of EA2. Preferably, the arrangement of the variable regions of an EphA2-BiTE of the invention is the following: VH-VL in the CD3 binding domain, and VH-VL in the EphA2 binding domain. Also in accordance with this embodiment, in a specific embodiment, the second binding domain comprises the VH and/or VL domain of EA2, EA3, EA4, EA5, 3F2, 4H5, 2A4, 2E7, 12E2, Eph099B-102.147, Eph099B-208.261, Eph099B-210.248, Eph099B-233.152, Eph101.530.241, 233 or G5.

Methods of producing the antibodies from which the EphA2-BiTES (in particular, the EphA2-BiTEs which are bispecific single chain antibodies) of the invention are derived are well known in the art and may be found, for example, US 2004-0091486 A1 (May 13, 2004), US 2004-0028685 A1 (Feb. 12, 2004) and WO 99/5440 (and references disclosed herein), each of which is incorporated by reference herein in its entirety. For information related to the affinity optimized EphA2 agonistic antibody variants 2A4, 2E7 and 12E2, see also U.S. Provisional Appn. No. 60/751,964, filed Dec. 21, 2005, entitled “Affinity Optimized EphA2 Agonistic Antibodies and Methods of Use Thereof,” which is incorporated by reference herein in its entirety.

5.1.1 EphA2 Antibodies

As discussed above, the invention encompasses bispecific T-cell engagers (i.e., EphA2-BiTEs (in particular, EphA2-BiTEs which are bispecific single chain antibodies) comprising least two binding domains specific for the EphA2 and CD3 antigens, respectively. The EphA2-BiTEs of the invention comprise a first binding domain that binds to the T-cell antigen CD3 and a second binding domain which binds to an EphA2 polypeptide or a fragment thereof.

In a specific embodiment, the binding domain that immunospecifically binds to EphA2 is a single chain Fv (scFv). As used herein, the term “single-chain Fv” or “scFv” refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994). In one embodiment, the binding domain of an EphA2-BiTE that immunospecifically binds to EphA2 is derived from a scFV produced from any antibody, including the EphA2 antibodies disclosed herein.

In specific embodiments, the EphA2 antibodies used to generate the EphA2-BiTEs include immunoglobulin molecules and immunologically active portions of immunoglobulin molecules, i.e., molecules that contain an antigen-binding domain that immunospecifically binds to an EphA2 antigen, e.g., one or more complementarity determining regions (CDRs) of an anti-EphA2 antibody. The EphA2 antibodies from which the EphA2 binding domain of the EphA2-BiTEs are derived can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

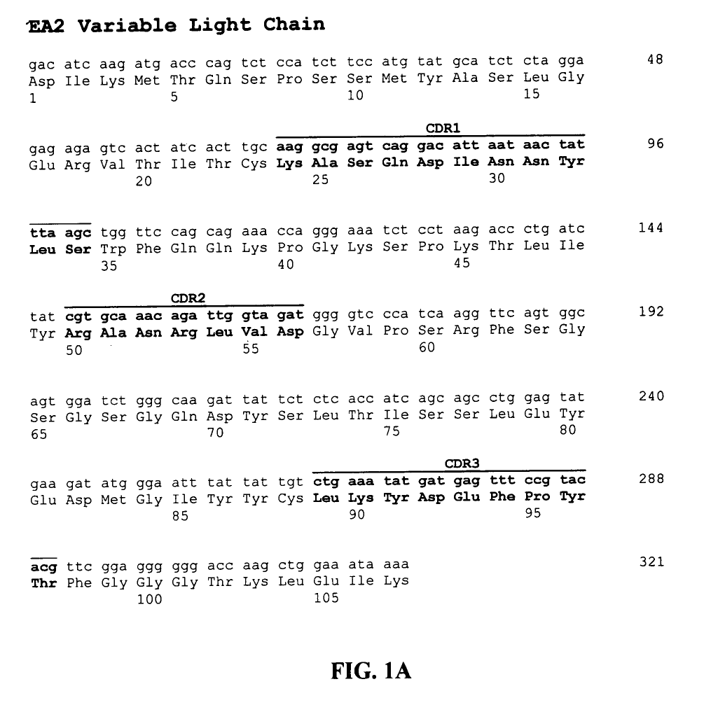

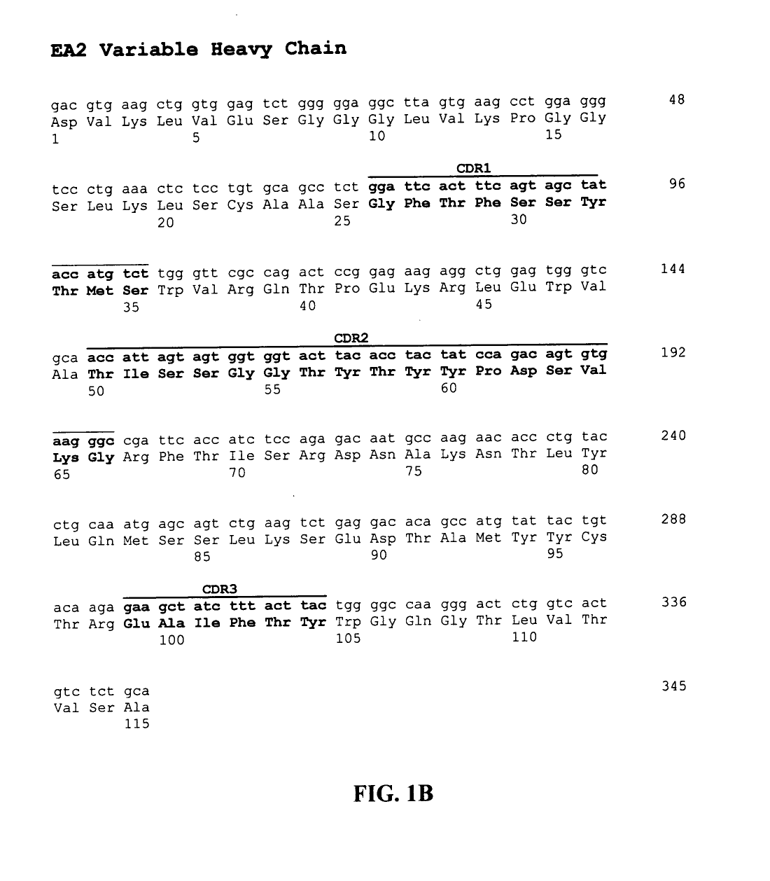

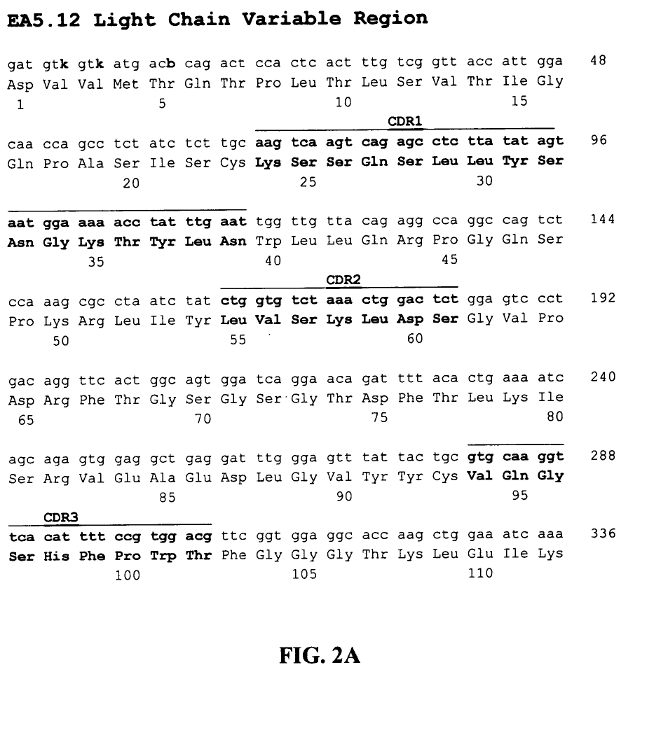

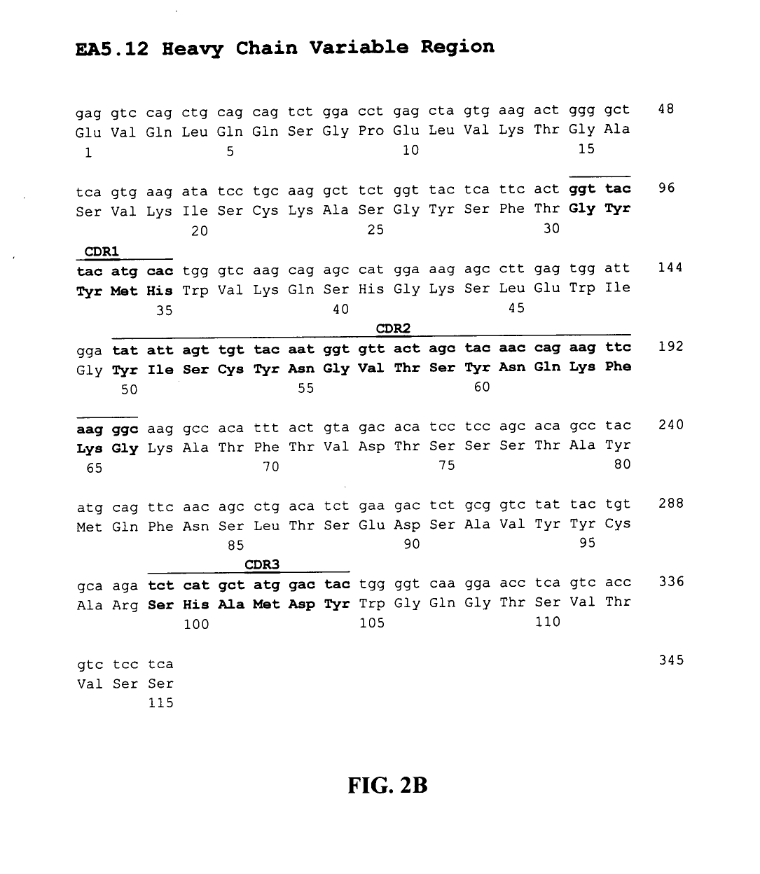

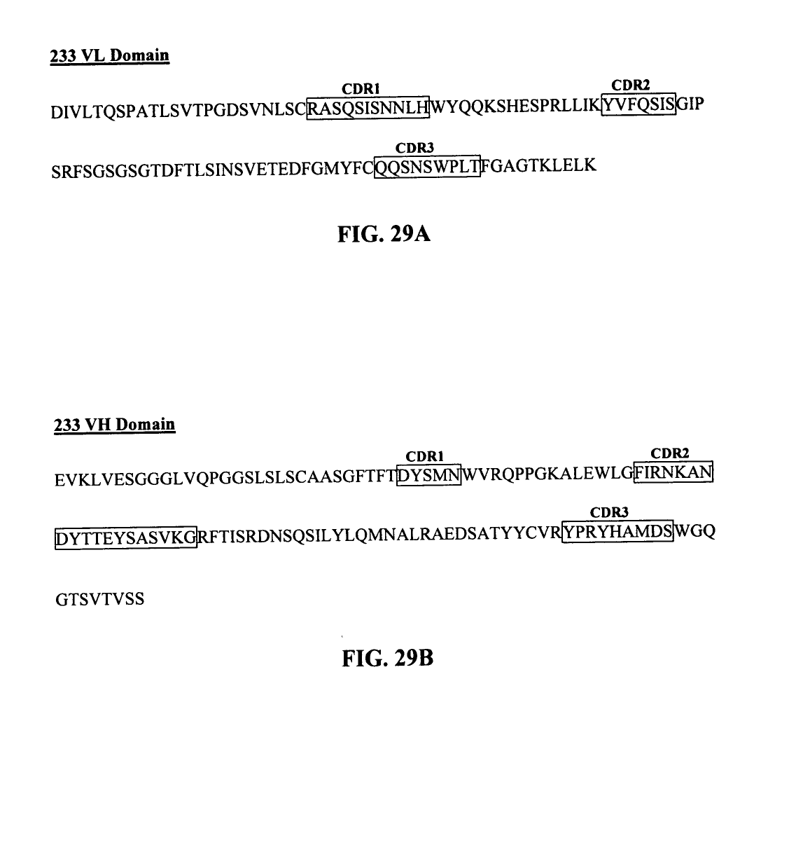

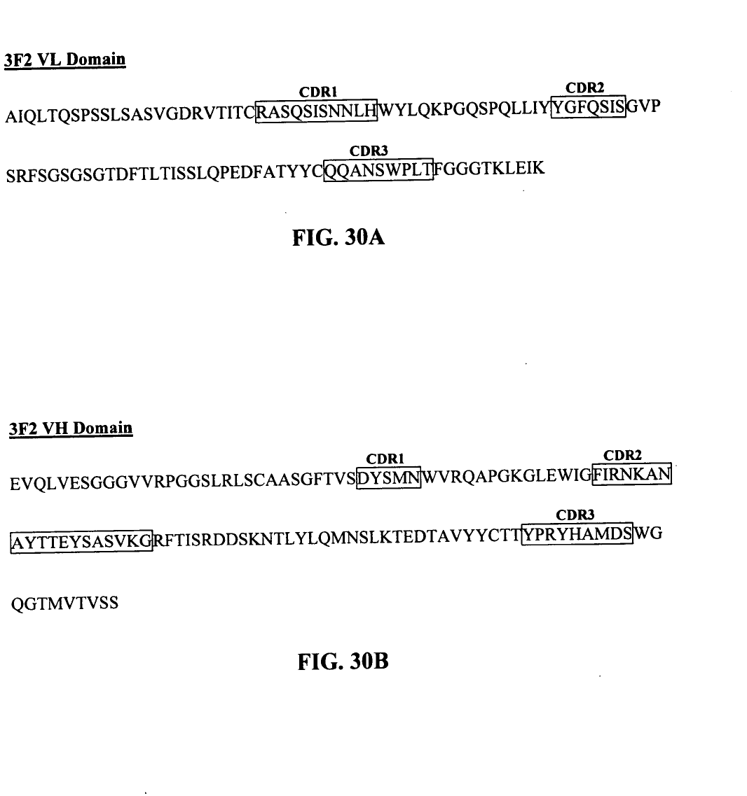

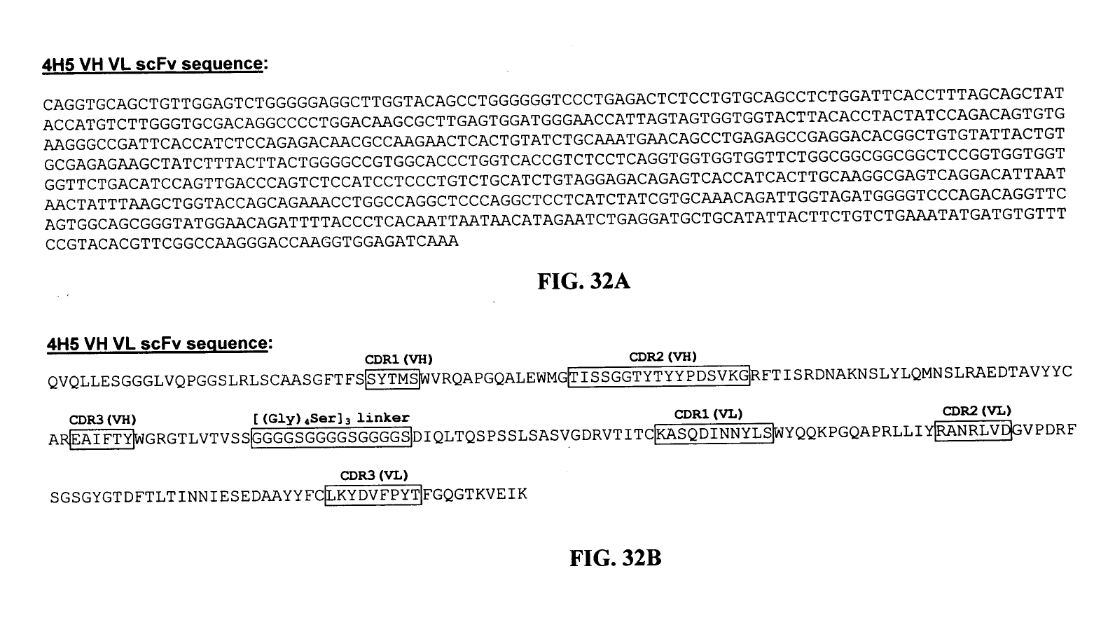

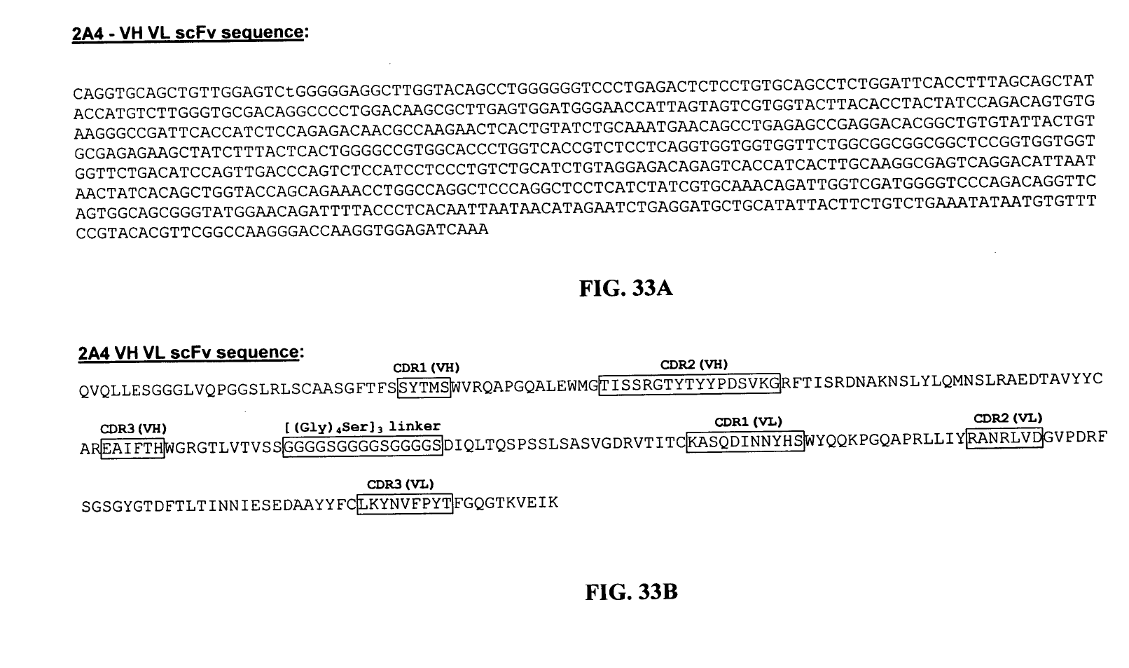

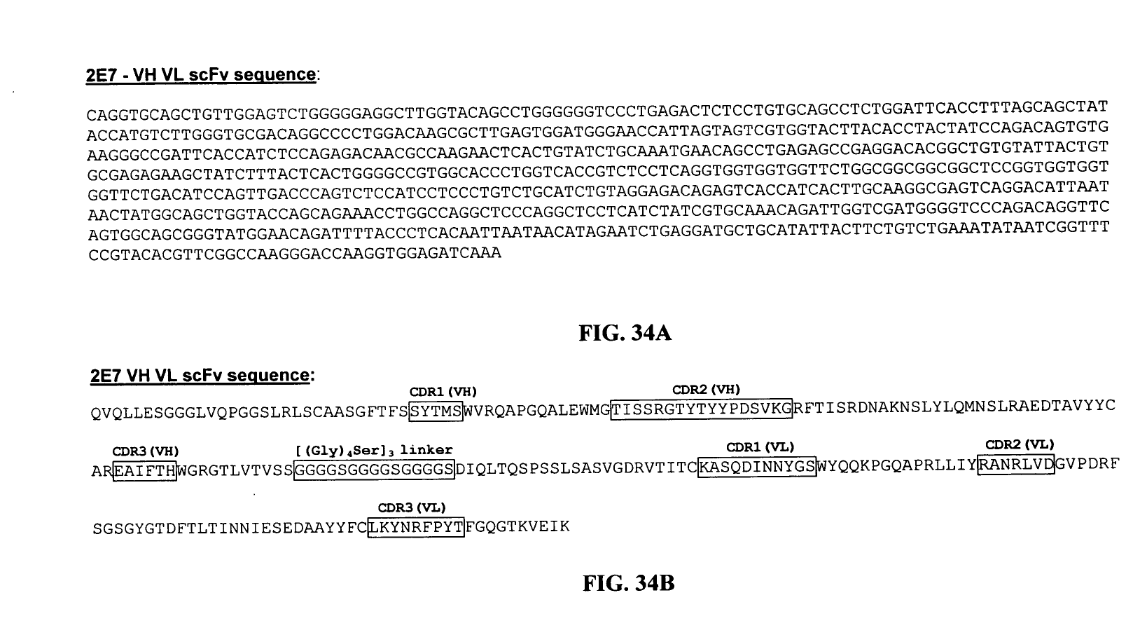

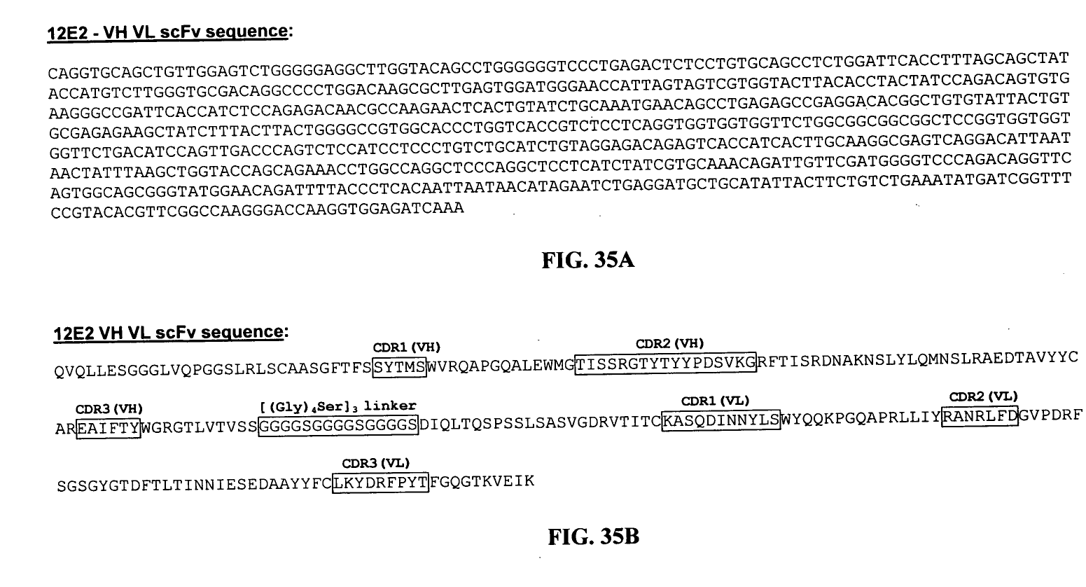

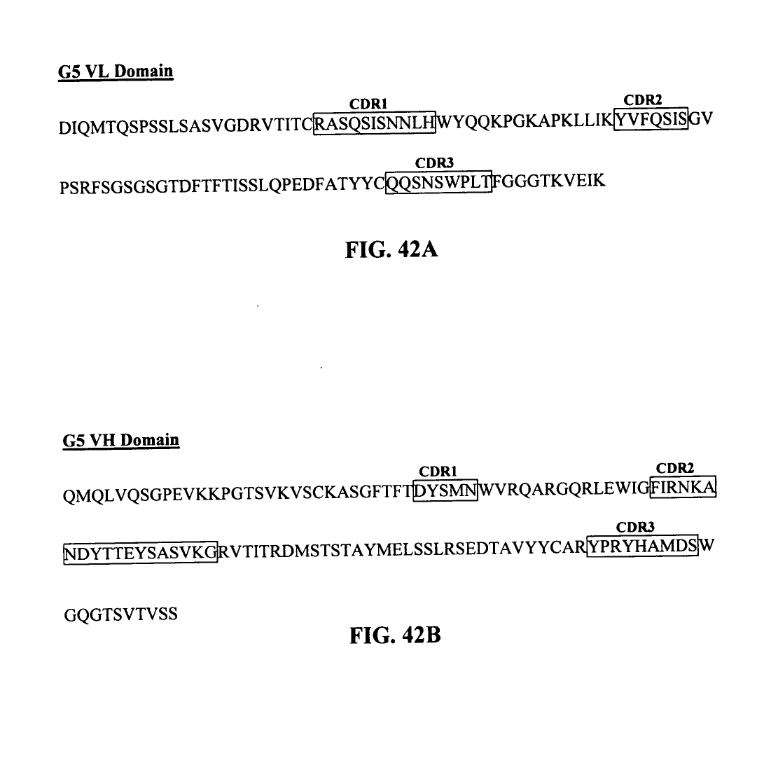

In other specific embodiments, the EphA2 antibodies used to generate the EphA2-BiTEs encompassed by the invention are EA2 (see FIG. 1), EA3, EA4, EA5 (see FIG. 2), 3F2 (see FIG. 30), 4H5 (see FIG. 32), 2A4 (FIG. 33), 2E7 (FIG. 34), 12E2 (FIG. 35), Eph099B-102.147, Eph099B-208.261, Eph099B-210.248, Eph099B-233.152, Eph1010.530.241, 233 (FIG. 29) and G5 (FIG. 42). See also, e.g., U.S. Patent Pub. Nos. US 2004-0091486 A1 (May 13, 2004), US 2004-0028685 A1 (Feb. 12, 2004), US 2005-0059592 A1 (Mar. 17, 2005), US 2005-0048617 A1, U.S. Appn. Ser. Nos. 11/165,023, filed Jun. 24, 2005 and 11/203,251, filed Aug. 15, 2005, each of which is incorporated by reference herein in its entirety. Hybridomas producing antibodies EA2 (strain EA2.31) and EA5 (strain EA5.12) of the invention have been deposited with the American Type Culture Collection (ATCC, P.O. Box 1549, Manassas, Va. 20108) on May 22, 2002, and assigned accession numbers PTA-4380 and PTA-4381, respectively and incorporated by reference. Eph099B-102.147, Eph099B-208.261, and Eph099B-210.248 were deposited with the ATCC on Aug. 7, 2002 and assigned accession nos. (PTA-4572, PTA-4573, and PTA-4574, respectively). Eph099B-233.152 was deposited with the ATCC on May 12, 2003 and assigned accession no. PTA-5194, and Eph101.530.241 was deposited with the ATCC on Sep. 26, 2002 and assigned accession no. PTA-4724. All of the aforementioned deposits were made under the provisions of the Budapest Treaty on the International Recognition of the Deposit of Microorganisms for the Purposes of Patent Procedures, and the accession numbers and dates corresponding to the respective antibodies are incorporated by reference herein in their entireties.

In a specific embodiment, the antibodies used to create the EphA2-BiTEs using the methods of the invention are human or humanized versions of the aforementioned EphA2 antibodies. Such humanized EphA2 antibodies and methods of production are disclosed in, e.g., U.S. Patent Appln. Publn. No. US 2005-0048617 A1, and Dall'Acqua et al., 2005, Methods 36:43-60, each of which is incorporated by reference herein in its entirety. In another specific embodiment, the EphA2 antibody used to generate an EphA2-BiTE of the invention is EA2. In yet another specific embodiment, the antibodies used to create an EphA2-BiTE using the methods of the invention are humanized affinity optimized versions of the aforementioned antibodies. In accordance with this embodiment, the binding domain that immunospecifically binds to EphA2 of an EphA2-BiTE of the invention is derived from 2A4, 2E7 and 12E2. For information related to the affinity optimized EphA2 agonistic antibody variants 2A4, 2E7 and 12E2, see also U.S. Provisional Appn. No. 60/751,964, filed Dec. 21, 2005, entitled “Affinity Optimized EphA2 Agonistic Antibodies and Methods of Use Thereof,” which is incorporated by reference herein in its entirety.

The sequences of the some EphA2 antibodies are disclosed in FIG. 1, 2, 29, 30, 32, 33, 34, 35, 37 or 39, or in the publications disclosing the sequences of the EphA2 antibodies cited above. Methods for preparing the EphA2 antibodies from which the EphA2-BiTEs of the invention are generated are disclosed, for example, in U.S. Patent Pub. Nos. US 2004-0091486 A1 (May 13, 2004), US 2004-0028685 A1 (Feb. 12, 2004), US 2005-0059592 A1 (Mar. 17, 2005), US 2005-0048617 A1, U.S. Appn. Ser. Nos. 11/165,023, filed Jun. 24, 2005 and 11/203,251, filed Aug. 15, 2005, each of which is incorporated by reference herein in its entirety.

The present invention provides EphA2-BiTEs derived from antibodies that immunospecifically bind to an EphA2 polypeptide, wherein said antibodies may comprise a VH CDR having an amino acid sequence of any one of the VH CDRs underlined in, for example, FIG. 1, 2, 29, 30, 32, 33, 34, 35, 37 or 39, or in the publications disclosing the EphA2 antibodies cited above. In particular, the invention provides EphA2-BiTEs comprising a binding domain that immunospecifically binds to EphA2, which binding domain comprises (or alternatively, consists of) one, two, three, four, five or more VH CDRs having an amino acid sequence of any of the VH CDRs listed in, for example, FIG. 1, 2, 29, 30, 32, 33, 34, 35, 37 or 39, or in the publications disclosing the sequences of the EphA2 antibodies cited above.

The present invention provides EphA2-BiTEs derived from antibodies that immunospecifically bind to an EphA2 polypeptide, wherein said antibodies may comprise a VL CDR having an amino acid sequence of any one of the VL CDRs underlined in, for example, FIG. 1, 2, 29, 30, 32, 33, 34, 35, 37 or 39, or in the publications disclosing the EphA2 antibodies cited above. In particular, the invention provides EphA2-BiTEs comprising a binding domain that immunospecifically binds to EphA2, which binding domain comprises (or alternatively, consists of) one, two, three, four, five or more VH CDRs having an amino acid sequence of any of the VL CDRs listed in, for example, FIG. 1, 2, 29, 30, 32, 33, 34, 35, 37 or 39, or in the publications disclosing the sequences of the EphA2 antibodies cited above.

The present invention provides EphA2-BiTEs comprising a binding domain that immunospecifically binds to EphA2, which binding domain comprises (or alternatively consists of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs disclosed in, for example, FIG. 1, 2, 29, 30, 32, 33, 34, 35, 37 or 39, or the publications disclosing the sequences of the EphA2 antibodies cited above.

In other embodiments, the invention provides EphA2-BiTEs comprising a binding domain that immunospecifically binds to EphA2, which binding domain comprises (or alternatively consists of) a VH and a VL domain of any of the aforementioned EphA2 antibodies mentioned supra, and for example as disclosed in FIG. 1, 2, 29, 30, 32, 33, 34, 35 or 37, or the publications disclosing the sequences of the EphA2 antibodies cited above. In a specific embodiment, the VH and VL domains are from 2A4, 2E7 and 12E2 antibodies. See, e.g., FIGS. 33, 34, 35 and 37.

In a specific embodiment, the invention provides EphA2-BiTEs comprising a binding domain that immunospecifically binds to EphA2, which binding domain is derived from antibodies that immunospecifically bind to an EphA2 polypeptide, wherein said antibodies have an association rate constant or kₒₙ rate (antibody (Ab)+antigen (Ag)→Ab-Ag) of at least 10⁴ M⁻¹s⁻¹, at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or in a range of about 10⁵ to 10⁸ M⁻¹s⁻¹, in a range of about 1.5×10⁵ M⁻¹s⁻¹ to 1×10⁷ M⁻¹s⁻¹, in a range of about 2×10⁵ to 1×10⁶ M⁻¹s⁻¹, or in a range of about 4.5×10⁵ to 10⁷ M⁻¹s⁻¹. In certain embodiments, an antibody that immunospecifically binds to an EphA2 polypeptide has a of at least 10⁴ M⁻¹s⁻¹, at least 2×10⁵ M⁻¹s⁻¹, at least 2.5×10⁵ M⁻¹s⁻¹, at least 5×10 M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹ as determined by a surface plasmon resonance assay.

In another specific embodiment, the invention provides EphA2-BiTEs comprising a binding domain that immunospecifically binds to EphA2, which binding domain is derived from antibodies that immunospecifically bind to an EphA2 polypeptide, wherein said antibodies have a k_{off} rate (antibody (Ab)+antigen (Ag)→Ab-Ag) of less than 10⁻³ s⁻¹, less than 5×10⁻³ s⁻¹, less than 10⁻⁴ s⁻¹, less than 2×10⁻⁴ s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or in a range of about 10⁻³ to 10⁻¹⁰ s⁻¹, in a range of about 10⁻⁴ to 10⁻⁸ s⁻¹, or in a range of about 10⁻⁵ to 10⁻⁸ s⁻¹. In certain embodiments, an antibody that immunospecifically binds to an EphA2 polypeptide has a k_{off} of 10⁻² s⁻¹, less than 5×10⁻³ s⁻¹, or less than 10⁻⁴ s⁻¹, as determined by a surface plasmon resonance assay.

In another specific embodiment, the invention provides EphA2-BiTEs comprising a binding domain that immunospecifically binds to EphA2, which binding domain is derived from antibodies that immunospecifically bind to an EphA2 polypeptide, wherein said antibodies have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or in a range of about 10² to 5×10⁵ M⁻¹, in a range of about 10⁴ to 1×10¹⁰ M⁻¹, or in a range of about 10⁵ to 1×10⁸ M⁻¹, as determined by a surface plasmon resonance assay.

In another specific embodiment, the invention provides EphA2-BiTEs comprising a binding domain that immunospecifically binds to EphA2, which binding domain is derived from antibodies that immunospecifically bind to an EphA2 polypeptide, wherein said antibodies have a dissociation constant or K_{d} (k_{off}/kₒₙ) of less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M or in a range of about 10⁻² M to 5×10⁻⁵ M, in a range of about 10⁻⁶ to 10⁻¹⁵ M, or in a range of about 10⁻⁸ to 10⁻¹⁴ M, as determined by a surface plasmon resonance assay.

In a specific embodiment, the invention provides EphA2-BiTEs that immunospecifically bind to an EphA2 polypeptide, wherein said EphA2-BiTEs have an association rate constant or kₒₙ rate (antibody (Ab)+antigen (Ag)→Ab-Ag) of at least 10⁴ M⁻¹s⁻¹, at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or in a range of about 10⁵ to 10⁸ M⁻¹s⁻¹, in a range of about 1.5×10⁵ M⁻¹s⁻¹ to 1×10⁷ M⁻¹s⁻¹, in a range of about 2×10⁵ to 1×10⁶ M⁻¹s⁻¹, or in a range of about 4.5×10⁵ to 10⁷ M⁻¹s⁻¹. In certain embodiments, an EphA2-BiTE that immunospecifically binds to an EphA2 polypeptide has a kₒₙ of at least 10⁴ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹ as determined by a surface plasmon resonance assay.

In another specific embodiment, the invention provides EphA2-BiTEs that immunospecifically bind to an EphA2 polypeptide, wherein said EphA2-BiTEs have a k_{off} rate (antibody (Ab)+antigen (Ag)→Ab-Ag) of less than 10⁻³ s⁻¹, less than 5×10⁻³ s⁻¹, less than 10⁻⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or in a range of about 10⁻³ to 10⁻¹⁰ s⁻¹, in a range of about 10⁻⁴ to 10⁻⁸ s⁻¹, or in a range of about 10⁻⁵ to 10⁻⁸ s⁻¹. In certain embodiments, an EphA2-BiTE that immunospecifically binds to an EphA2 polypeptide has a k_{off} of 10⁻² s⁻¹, less than 5×10⁻³ s⁻¹, or less than 10⁻⁴ s⁻¹, as determined by a surface plasmon resonance assay.

In another specific embodiment, the invention provides EphA2-BiTEs that immunospecifically bind to an EphA2 polypeptide, wherein said EphA2-BiTEs have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or in a range of about 10² to 5×10⁵ M⁻¹, in a range of about 10⁴ to 1×10¹⁰ M⁻¹, or in a range of about 10⁵ to 1×10⁸ M⁻¹, as determined by a surface plasmon resonance assay.

In another specific embodiment, the invention provides EphA2-BiTEs that immunospecifically bind to an EphA2 polypeptide, wherein said EphA2-BiTEs have a dissociation constant or K_{d} (k_{off}/kₒₙ) of less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M or in a range of about 10⁻² M to 5×10⁻⁵ M, in a range of about 10⁻⁶ to 10⁻¹⁵ M, or in a range of about 10⁻⁸ to 10⁻¹⁴ M, as determined by a surface plasmon resonance assay.

5.1.2 CD3 Antibodies

In specific embodiments of the invention, the EphA2-BiTEs comprise a binding domain that immunospecifically binds the CD3 T-cell antigen. Said CD3 T-cell antigen can be from any species (e.g., human). In a specific embodiment, the EphA2-BiTEs of the invention comprise a binding domain that immunospecifically binds to the one or more subunits of CD3 (e.g., the gamma, delta, zeta, or eta subunit). In a preferred embodiment, the first binding domain immunospecifically binds to the epsilon (ε) subunit of CD3. In a specific embodiment, the first binding domain immunospecifically binds to the epsilon (ε) subunit of CD3 when said subunit is complexed with the delta subunit of CD3. In a specific embodiment, the CD3 binding domain of an EphA2-BiTE of the invention is deimmunized. Deimmunization approaches are well known in the art and are disclosed in, e.g., International Pub. Nos. WO 00/34317 (and in particular, pp. 1-14); WO 98/52976 (and in particular, Examples 1-6 on pp. 18-38); WO 02/079415 (and in particular, pp. 2-8 and Examples 1-10 at pp. 15-43); and WO 92/10755 (and in particular, pp. 6-9), each of which is incorporated by reference herein in its entirety.

In a specific embodiment, the binding domain that immunospecifically binds to CD3 is a single chain Fv (scFv). As used herein, the term “single-chain Fv” or “scFv” refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994). In one embodiment, the EphA2-BiTEs of the invention are derived from scFvs produced from any of the EphA2 antibodies disclosed below.

In specific embodiments, the CD3 antibodies used to generate the EphA2-BiTEs include immunoglobulin molecules and immunologically active portions of immunoglobulin molecules, i.e., molecules that contain an antigen-binding domain that immunospecifically binds to a CD3 antigen, e.g., one or more complementarity determining regions (CDRs) of an anti-CD3 antibody. The CD3 antibodies from which the CD3 binding domain of the EphA2-BiTEs are derived can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Such CD3 antibodies may be from any species (e.g., rat, mouse, human, etc.).

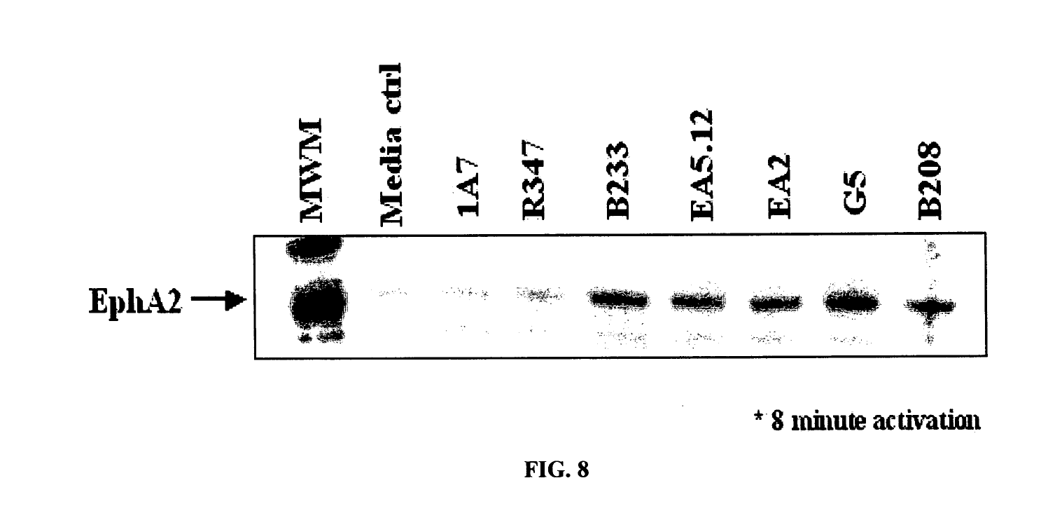

In a specific embodiment, the CD3 binding domain of an EphA2-BiTE of the invention may be produced as described in International Publication No. WO 99/54440 (p. 3 and FIG. 8), which is incorporated by reference herein in its entirety. Anti-CD3 antibodies from which said binding domain is derived are also described in, for example, Kipriyanov, 1998, Int. J. Cancer 77:763-772 (p. 763-765); Dreier et al., 2002, Int. J. Cancer 100:690-697 (p. 691), each of which is incorporated by reference herein in its entirety.

The present invention provides EphA2-BiTEs derived from antibodies that immunospecifically bind to a CD3 polypeptide, wherein said antibodies may comprise a VH CDR having an amino acid sequence of any one of the VH CDRs disclosed in, for example, the publications disclosing the CD3 antibodies or binding fragments cited above. In particular, the invention provides EphA2-BiTEs comprising a binding domain that immunospecifically binds to CD3, which binding domain comprises (or alternatively, consists of) one, two, three, four, five or more VH CDRs having an amino acid sequence of any of the VH CDRs disclosed in, for example, the publications disclosing the CD3 antibodies or binding fragments cited above.

The present invention provides EphA2-BiTEs derived from antibodies that immunospecifically bind to a CD3 polypeptide, wherein said antibodies may comprise a VL CDR having an amino acid sequence of any one of the VL CDRs disclosed in, for example, the publications disclosing the CD3 antibodies or binding fragments cited above. In particular, the invention provides EphA2-BiTEs comprising a binding domain that immunospecifically binds to CD3, which binding domain comprises (or alternatively, consists of) one, two, three, four, five or more VL CDRs having an amino acid sequence of any of the VL CDRs disclosed in, for example, the publications disclosing the CD3 antibodies or binding fragments cited above.

The present invention provides EphA2-BiTEs comprising a binding domain that immunospecifically binds to CD3 polypeptide, which binding domain comprises (or alternatively consists of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs of the CD3 antibodies described above.

In other embodiments, the invention provides EphA2-BiTEs derived from antibodies that immunospecifically bind to CD3, wherein said antibodies may comprise a VH and a VL domain of a CD3 antibody described above.

In a specific embodiment, the invention provides EphA2-BiTEs comprising a binding domain that immunospecifically binds to a CD3 polypeptide, which binding domain is derived from antibodies that immunospecifically bind to a CD3, wherein said antibodies have an association rate constant or kₒₙ rate (antibody (Ab)+antigen (Ag)→Ab-Ag) of at least 10⁴ M⁻¹s⁻¹, at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or in a range of about 10⁵ to 10⁸ M⁻¹s⁻¹, in a range of about 1.5×10⁵ M⁻¹s⁻¹ to 1×10⁷ M⁻¹s⁻¹, in a range of about 2×10⁵ to 1×10⁶ M⁻¹s⁻¹, or in a range of about 4.5×10⁵ to 10⁷ M⁻¹s⁻¹. In certain embodiments, an antibody that immunospecifically binds to an EphA2 polypeptide has a kₒₙ of at least 10⁴ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹ as determined by a surface plasmon resonance assay.

In another specific embodiment, the invention provides EphA2-BiTEs comprising a binding domain that immunospecifically binds to CD3, which binding domain is derived from antibodies that immunospecifically bind to a CD3 polypeptide, wherein said antibodies have a k_{off} rate (antibody (Ab)+antigen (Ag)→Ab-Ag) of less than 10⁻³ s⁻¹, less than 5×10⁻³ s⁻¹, less than 10⁻⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or in a range of about 10⁻³ to 10⁻¹⁰ s⁻¹, in a range of about 10⁻⁴ to 10⁻⁸ s⁻¹, or in a range of about 10⁻⁵ to 10⁻⁸ s⁻¹. In certain embodiments, an antibody that immunospecifically binds to an EphA2 polypeptide has a k_{off} of 10⁻² s⁻¹, less than 5×10⁻³ s⁻¹, or less than 10⁻⁴ s⁻¹, as determined by a surface plasmon resonance assay.

In another specific embodiment, the invention provides EphA2-BiTEs comprising a binding domain that immunospecifically binds to CD3, which binding domain is derived from antibodies that immunospecifically bind to a CD3 polypeptide, wherein said antibodies have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or in a range of about 10² to 5×10⁵ M⁻¹, in a range of about 10⁴ to 1×10¹⁰ M⁻¹, or in a range of about 10⁵ to 1×10⁸ M⁻¹, as determined by a surface plasmon resonance assay.

In another specific embodiment, the invention provides EphA2-BiTEs comprising a binding domain that immunospecifically binds to CD3, which binding domain is derived from antibodies that immunospecifically bind to a CD3 polypeptide, wherein said antibodies have a dissociation constant or K_{d} (k_{off}/kₒₙ) of less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M or in a range of about 10⁻² M to 5×10⁻⁵ M, in a range of about 10⁻⁶ to 10⁻¹⁵ M, or in a range of about 10⁻⁸ to 10⁻¹⁴ M, as determined by a surface plasmon resonance assay.

In a specific embodiment, the invention provides EphA2-BiTEs that immunospecifically binds to a CD3 polypeptide, wherein said EphA2-BiTEs have an association rate constant or kₒₙ rate (antibody (Ab)+antigen (Ag)→Ab-Ag) of at least 10⁴ M⁻¹s⁻¹, at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or in a range of about 10⁵ to 10⁸ M⁻¹s⁻¹, in a range of about 1.5×10⁵ M⁻¹s⁻¹ to 10×10⁷ M⁻¹s⁻¹, in a range of about 2×10⁵ to 1×10⁶ M⁻¹s⁻¹, or in a range of about 4.5×10⁵ to 10⁷ M⁻¹s⁻¹. In certain embodiments, an EphA2-BiTE that immunospecifically binds to an EphA2 polypeptide has a kₒₙ of at least 10⁴ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷M⁻¹s⁻¹, or at least 10⁸ M⁻¹s⁻¹ as determined by a surface plasmon resonance assay.

In another specific embodiment, the invention provides EphA2-BiTEs that immunospecifically binds to a CD3 polypeptide, wherein said EphA2-BiTEs have a k_{off} rate (antibody (Ab)+antigen (Ag)→Ab-Ag) of less than 10⁻³ s⁻¹, less than 5×10⁻³ s⁻¹, less than 10⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or in a range of about 10⁻³ to 10⁻¹⁰ s⁻¹, in a range of about 10⁻⁴ to 10⁻⁸ s⁻¹, or in a range of about 10⁻⁵ to 10⁻⁸ s⁻¹. In certain embodiments, an EphA2-BiTE that immunospecifically binds to an EphA2 polypeptide has a k_{off} of 10⁻² s⁻¹, less than 5×10⁻³ s⁻¹, or less than 10⁻⁴ s⁻¹, as determined by a surface plasmon resonance assay.

In another specific embodiment, the invention provides EphA2-BiTEs that immunospecifically binds to a CD3 polypeptide, wherein said EphA2-BiTEs have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or in a range of about 10² to 5×10⁵ M⁻¹, in a range of about 10⁴ to 1×10¹⁰ M⁻¹, or in a range of about 10⁵ to 1×10⁸ M⁻¹, as determined by a surface plasmon resonance assay.

In another specific embodiment, the invention provides EphA2-BiTEs that immunospecifically binds to a CD3 polypeptide, wherein said EphA2-BiTEs have a dissociation constant or K_{d} (k_{off}/kₒₙ) of less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M or in a range of about 10⁻² M to 5×10⁻⁵ M, in a range of about 10⁻⁶ to 10⁻¹⁵ M, or in a range of about 10⁻⁸ to 10⁻¹⁴ M, as determined by a surface plasmon resonance assay.

5.1.3 EphA2-BiTE Conjugates

The present invention further relates to bispecific T-cell engagers (i.e., EphA2-BiTEs (in particular, EphA2-BiTEs which are bispecific single chain antibodies) comprising at least one further domain, said domain being linked by covalent or non-covalent bonds. The additional domain may be of a predefined specificity or function. Accordingly, the present invention relates to the use of the EphA2-BiTEs of the invention recombinantly fused or chemically conjugated (including both covalent and non-covalent conjugations) to a heterologous polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes compositions comprising heterologous polypeptides fused or conjugated to EphA2-BiTE fragments. For example, the heterologous polypeptides may be fused or conjugated to a Fab fragment, Fd fragment, Fv fragment, F(ab)₂ fragment, or fragment thereof. Methods for fusing or conjugating polypeptides to antibody fragments are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341 (said references incorporated by reference in their entireties).

Additional fusion proteins, e.g., of any of the aforementioned EphA2-BiTEs, may be generated through the techniques of gene-shuffling, motif-shuffling, exon-shuffling, and/or codon-shuffling (collectively referred to as “DNA shuffling”). DNA shuffling may be employed to alter the activities of antibodies of the invention (e.g., antibodies with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). EphA2-BiTEs, or the encoded EphA2-BiTEs, may be altered by being subjected to random mutagenesis by error-prone PCR, random nucleotide insertion or other methods prior to recombination. One or more fragments of a polynucleotide encoding an antibody or antibody fragment, which fragments immunospecifically bind to EphA2 may be recombined with one or more components, motifs, sections, parts, domains, fragments, etc. of one or more heterologous molecules.

Moreover, the EphA2-BiTEs or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, EphA2-BiTEs of the present invention or fragments or variants thereof are conjugated to a diagnostic or detectable agent. Such EphA2-BiTEs can be useful for monitoring or prognosing the development or progression of a cancer as part of a clinical testing procedure, such as determining the efficacy of a particular therapy. Additionally, such antibodies can be useful for monitoring or prognosing the development or progression of a pre-cancerous condition associated with cells that overexpress EphA2 (e.g., high-grade prostatic intraepithelial neoplasia (PIN), fibroadenoma of the breast, fibrocystic disease, or compound nevi). In one embodiment, an exposed EphA2 epitope antibody is conjugated to a diagnostic or detectable agent. In a more specific embodiment, the antibody is an EphA2-BiTE.

Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In one embodiment, the localization of an EphA2-BiTE to a diseased tissue(s) (e.g., a tumor) can be determined by detecting a labeled form of the EphA2-BiTE in the tissue. In a specific embodiment, a labeled EphA2-BiTE is detected in vivo in a subject according to a method comprising the steps of: (a) administering to the subject an effective amount of a labeled EphA2-BiTE, and (b) detecting the labeled EphA2-BiTE in the subject following a time interval sufficient to allow the labeled EphA2-BiTE to concentrate at sites in the subject where EphA2 is expressed. In accordance with this embodiment, the labeled EphA2-BiTE may be administered to the subject according to any suitable method in the art, for example, parenterally or intraperitoneally. Further, in accordance with this embodiment, the effective amount of the labeled EphA2-BiTE is the amount which permits the detection of the EphA2-BiTE in the subject. This amount will vary according to the particular subject, the label used, and the detection method employed. For example, it is understood in the art that the size of the subject and the imaging system used will determine the amount of labeled EphA2-BiTE needed to detect the EphA2-BiTE in a subject using an imaging means. In the case of a radiolabeled EphA2-BiTE for a human subject, the amount of labeled EphA2-BiTE administered is measured in terms of radioactivity, for example from about 5 to 20 millicuries of ⁹⁹Tc. The time interval following the administration of the labeled EphA2-BiTE which is sufficient to allow the labeled EphA2-BiTE to concentrate at sites in the subject where the EphA2 is expressed will vary depending on several factors, for example, the type of label used, the mode of administration, and the part of the subject's body that is imaged. In a particular embodiment, the time interval that is sufficient is 6 to 48 hours, 6 to 24 hours, or 6 to 12 hours. In another embodiment the time interval is 5 to 20 days or 5 to 10 days.

The presence of a labeled EphA2-BiTE can be detected in the subject using imaging means known in the art. In general, the imaging means employed depends upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), and sonography. In a specific embodiment, the EphA2-BiTE is labeled with a radioisotope and is detected in the patient using a radiation responsive surgical instrument (Thurston et al., U.S. Pat. No. 5,441,050). In another embodiment, the EphA2-BiTE is labeled with a fluorescent compound and is detected in the patient using a fluorescence responsive scanning instrument. In another embodiment, the EphA2-BiTE is labeled with a positron emitting metal and is detected in the patient using positron emission-tomography. In yet another embodiment, the EphA2-BiTE is labeled with a paramagnetic label and is detected in a patient using magnetic resonance imaging (MRI).

The present invention further encompasses uses of EphA2-BiTEs or fragments thereof conjugated to a therapeutic agent.

An EphA2-BiTE of the invention may be conjugated to a non-macromolecular therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an EphA2-BiTE may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an EphA2-BiTE can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

In a specific embodiment, the conjugated EphA2-BiTE comprises one domain that preferably binds an EphA2 epitope exposed on cancer cells but not on non-cancer cells, or on infected cells but not on non-infected cells (i.e., exposed EphA2 epitope antibody), and a second domain that preferably binds CD3.

Techniques for conjugating therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to antibodies are well known, see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to a moiety does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216, each of which is incorporated herein by reference in its entirety.

EphA2-BiTEs may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.2 EphA2-BiTE Polynucleotides of the Invention

The present invention provides the sequences of the polynucleotides encoding the EphA2-BiTEs disclosed herein. In a specific embodiment, the polynucleotides of the invention that encode the EphA2-BiTEs comprise a first nucleotide sequence encoding a first binding domain and a second nucleotide sequence encoding a second binding domain, and nucleotide sequences encoding linker sequences that link the first and second binding domains. See, e.g., FIG. 3 for a general depiction of an EphA2-BiTE polynucleotide construct. The present invention also provides polynucleotide sequences encoding EphA2-BiTEs in which the nucleotide sequences encoding the first and/or second binding domains hybridize to the nucleotide sequences of one or more variable domains of an anti-EphA2 antibody known in the art or described herein (e.g., EA2, 4H5, 2A4, 2E7 or 12E2) and/or an anti-CD3 antibody known in the art or described herein.

In one embodiment, EphA2-BiTEs produced from polynucleotides that hybridize to polynucleotides encoding EphA2-BiTEs that modulate the expression and/EphA2 and/or induce redirected lysis of EphA2-overexpressing cells by T-cells in an assay well known to the art or described herein. In another embodiment, EphA2-BiTEs used in the methods of the invention include polypeptides produced from polynucleotides that hybridize to polynucleotides encoding a fragments of EphA2-BiTEs. Conditions for hybridization include, but are not limited to, stringent hybridization conditions such as hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

Once the nucleotide sequence of the EphA2-BiTE used in the methods of the invention is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate polypeptides having a different amino acid sequence, for example to create amino acid substitutions, deletions, and/or insertions.

In specific embodiments, such polypeptides have at least 1, at least 2, at least 3, at least 4, at least 5, or at least 6 amino acid substitutions, insertions and/or deletions. Included among possible substitutions are conservative substitutions, in which the amino acid sequence is modified by replacing one or more amino acids with different amino acids which have similar chemical or structural characteristics and/or do not significantly alter the biological function of the peptide.

Standard techniques known to those of skill in the art can be used to introduce mutations in the nucleotide sequence encoding an EphA2-BiTE of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded EphA2-BiTE can be inserted into an expression vector and expressed (e.g., in a heterologous host cell) and the activity of the protein can be determined as described below.

The present invention also encompasses the use of bispecific single chain antibodies comprising the amino acid sequence of any EphA2-BiTEs described herein with mutations (e.g., one or more amino acid substitutions) in the framework or variable regions of the first and/or second binding domains. Preferably, these mutations maintain or enhance the avidity and/or affinity of the EphA2-BiTEs for EphA2 and/or CD3 to which they immunospecifically bind. Standard techniques known to those skilled in the art (e.g., immunoassays or ELISA assays) can be used to assay the degree of binding between a polypeptide EphA2-BiTE and its binding partner.

5.3 Methods of Producing EphA2-BiTEs

5.3.1 Recombinant Expression of an EphA2-BiTE

The EphA2-BiTEs of the invention can be produced by any method known in the art or disclosed herein, in particular, by chemical synthesis or preferably, by recombinant expression techniques described supra (see, e.g., WO 99/54440, which is incorporated by reference herein in its entirety). See also Section 6, infra, for detailed examples of methods for producing EphA2-BiTEs of the invention.

The polynucleotides of the invention can be used alone or as part of a vector to express the polypeptides of the invention (e.g., EphA2-BiTEs) in cells, for example, in gene therapy or diagnosis of disorders associated with aberrant expression (e.g., overexpression) and/or activity of EphA2 (e.g., cancer, a non-cancer hyperproliferative cell disorder or an infection). The polynucleotides or vectors containing the DNA sequence(s) encoding any of the polypeptides of the invention is introduced into the cells which in turn produce the polypeptide of interest (e.g., an EphA2-BiTE). Gene therapy, which is based on introducing therapeutic genes into cells by ex-vivo or in-vivo techniques is one of the most important applications of gene transfer.

Accordingly, recombinant expression of an EphA2-BiTE of the invention requires construction of an expression vector containing a polynucleotide sequence that encodes the EphA2-BiTE. See, e.g., FIG. 3. The polynucleotides encoding the EphA2-BiTEs described herein may be obtained and sequenced by any method known in the art. For example, a polynucleotide encoding an EphA2-BiTE used in the methods of the invention may be assembled from chemically synthesized oligonucleotides (e.g., as described in Kutmeier et al., 1994, BioTechniques 17:242), which, briefly, involves the synthesis of overlapping oligonucleotides containing fragments of the sequence encoding the polypeptide, annealing and ligating of those oligonucleotides, and then amplification of the ligated oligonucleotides by PCR.

Once a polynucleotide encoding an EphA2-BiTE of the invention has been obtained, the vector for the production of the EphA2-BiTE molecule may be produced by recombinant DNA technology using techniques well known in the art. Thus, methods for preparing a protein by expressing a polynucleotide containing an EphA2-BiTE encoding nucleotide sequence are described herein. Methods which are well known to those skilled in the art can be used to construct expression vectors containing EphA2-BiTE coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an EphA2-BiTE of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an EphA2-BiTE of the invention. Thus, the invention includes host cells containing a polynucleotide encoding an EphA2-BiTE of the invention, fragments thereof (e.g., first and/or second binding domains of an EphA2-BiTE), operably linked to a heterologous promoter.

A variety of host-expression vector systems may be utilized to express the EphA2-BiTEs of the invention or fragments thereof (e.g., a first and/or second binding domain of an EphA2-BiTE) (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an EphA2-BiTE molecule of the invention in situ. These include but are not limited to microorganisms such as bacteria (e.g., E. coli and B. subtilis) transformed with recombinant bacteriophage DNA, plasmid DNA or cosmid DNA expression vectors containing EphA2-BiTE coding sequences; yeast (e.g., Saccharomyces Pichia) transformed with recombinant yeast expression vectors containing EphA2-BiTE coding sequences; insect cell systems infected with recombinant virus expression vectors (e.g., baculovirus) containing EphA2-BiTE coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing EphA2-BiTE coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, are used for the expression of a recombinant EphA2-BiTE molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, BioTechnology 8:2). In a specific embodiment, the expression of nucleotide sequences encoding EphA2-BiTE s is regulated by a constitutive promoter, inducible promoter or tissue specific promoter. Alternatively, the polynucleotides of the invention may be expressed in a transgenic plant expression system, such as, for instance, the LEX System™ disclosed in U.S. Pat. No. 6,040,498, international application published on Feb. 18, 1999 (WO 99/07210), and international application published on Feb. 7, 2002 (WO 02/10414). Another transgenic plant expression system that may be utilized for the polynucleotides of the invention is the Plantibodies™ technology described in U.S. Pat. Nos. 5,202,422; 5,639,947; 5,959,177; and 6,417,429.

In bacterial systems, a number of expression vectors may be advantageously selected depending upon the use intended for the EphA2-BiTE molecule or fragment thereof being expressed. For example, when a large quantity of such a protein is to be produced, for the generation of pharmaceutical compositions of an EphA2-BiTE molecule, vectors which direct the expression of high levels of fusion protein products that are readily purified may be desirable. Such vectors include, but are not limited to, the E. coli expression vector pUR278 (Ruther et al., 1983, EMBO 12:1791), in which the EphA2-BiTE coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety. Another microbial system that may be used to express the polynucleotides of the invention is the Pfenex Expression Technology is based on novel strains of Pseudomonas fluorescens, as described in Squires et al., BioProcess Int'l p. 54 Dec. 2004; and Squires et al., Specialty Chemicals July/August 2004.

In an insect system, Autographa californica nuclear polyhedrosis virus (AcNPV) is used as a vector to express foreign genes. The virus grows in Spodoptera frugiperda cells. The EphA2-BiTE coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the EphA2-BiTE coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the EphA2-BiTE molecule in infected hosts (e.g., see Logan & Shenk, 1984, PNAS 8 1:355-359). Specific initiation signals may also be required for efficient translation of inserted EphA2-BiTE coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, BT483, Hs578T, HTB2, BT20, NS1, and T47D, NS0 (a murine myeloma cell line that does not endogenously produce any immunoglobulin chains), CRL7030 and HsS78BsT-cells.

For long-term, high-yield production of recombinant proteins, stable expression is preferred. For example, cell lines which stably express the EphA2-BiTE molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the EphA2-BiTE molecule. Such engineered cell lines may be particularly useful in screening and evaluation of compositions that interact directly or indirectly with the EphA2-BiTE molecule.

A number of selection systems may be used, including but not limited to, the herpes simplex virus thymidine kinase (Wigler et al., 1977, Cell 11:223), glutamine synthase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk−, gs−, hgprt− or aprt− cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an EphA2-BiTE molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing EphA2-BiTE is amplifiable, increase in the level of inhibitor present in culture of host cell will increase the number of copies of the marker gene. Since the amplified region is associated with the EphA2-BiTE gene, production of the EphA2-BiTE will also increase (Crouse et al., 1983, Mol. Cell. Biol. 3:257).

The host cell may be co-transfected with two expression vectors of the invention, the first vector encoding a first binding domain of an EphA2-BiTE or a variable heavy domain of an antibody (e.g., an anti-EphA2 antibody or an anti-CD3 antibody) and the second vector encoding a second binding domain of an EphA2-BiTE or a variable light domain of an antibody (e.g., an anti-EphA2 antibody or an anti-CD3 antibody). The first and second binding domains of the EphA2-BiTE can be purified using techniques known in the art and the two domains can be chemically linked by methods known in the art.

The host cell may be transfected with an expression vector of the invention encoding an EphA2-BiTE of the invention. The vector may contain a single vector which encodes, and is capable of expressing, both variable and light domains of an antibody (e.g., anti-EphA2 antibody or anti-CD3 antibody), or both the first and second binding domains of an EphA2-BiTE (Proudfoot, 1986, Nature 322:52; and Kohler, 1980, PNAS 77:2197). The coding sequences for the variable domains or binding domains may comprise cDNA or genomic DNA.

Once an EphA2-BiTE of the invention or a binding domain thereof has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the EphA2-BiTEs of the present invention or fragments thereof may be fused to heterologous polypeptide sequences described herein or otherwise known in the art to facilitate purification.

In a specific embodiment, the recombinantly produced EphA2-BiTEs of the invention comprise a first binding domain and a second binding domain, wherein the first binding domain comprises a VH domain and a VL domain, linked together by a linker of sufficient length to enable the domains to fold in such a way as to permit binding to the CD3 T-cell antigen. In another specific embodiment, the second binding domain comprises a VH domain and a VL domain, and the VH and VL domains are linked together by a linker of sufficient length to enable the domains to fold in such a way as to permit binding to EphA2. In another specific embodiment, the first and second binding domains are linked together by a linker of sufficient length to enable the domains to fold in such a way as to permit binding to CD3 and EphA2. In certain embodiments, the first and second binding domains are scFvs. In other embodiments, the binding domain that binds to CD3 is deimmunized.

5.4 Prophylactic/Therapeutic Methods

The present invention relates to pharmaceutical compositions and prophylactic and therapeutic regimens designed to treat, prevent and/or manage disorders associated with the aberrant expression and/or activity of EphA2 (e.g., cancer, non-cancer hyperproliferative cell disorders and infections) in a subject, comprising administering one or more EphA2-BiTEs.

In a specific embodiment, the EphA2-BiTEs of the invention are administered to a subject to treat, prevent and/or manage disorders associated with the aberrant expression and/or activity of EphA2 (e.g., cancer, non-cancer hyperproliferative cell disorders and infections). In certain embodiments, the EphA2-BiTEs of the invention are administered in combination with one or more other therapies. In certain embodiments, one or more EphA2-BiTEs of the invention are administered to a mammal, preferably a human, concurrently with one or more other therapies. Preferably, such therapies are useful for the treatment of such disorders. The term “concurrently” is not limited to the administration of therapies at exactly the same time, but rather it is meant that the EphA2-BiTEs of the invention and the other therapy are administered to a subject in a sequence and within a time interval such that the antibodies of the invention can act together with the other therapy to provide an increased benefit than if they were administered otherwise. For example, each therapy may be administered at the same time or sequentially in any order at different points in time; however, if not administered at the same time, they should be administered sufficiently close in time so as to provide the desired therapeutic or prophylactic effect. Each therapy can be administered separately, in any appropriate form and by any suitable route. In other embodiments, the EphA2-BiTEs of the invention are administered before, concurrently or after surgery. Preferably the surgery completely removes localized tumors or reduces the size of large tumors. Surgery can also be done as a preventive measure or to relieve pain.

In various embodiments, the therapies are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapies administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physicians'Desk Reference (61^{st} ed., 2007).

5.4.1 Patient Population

5.4.1.1. Cancer Patients

The invention provides methods for treating, preventing and/or managing cancer by administrating to a subject a therapeutically or prophylactically effective amount of one or more EphA2-BiTEs of the invention. In another embodiment, the EphA2-BiTEs of the invention can be administered in combination with one or more other therapies. The subject is an animal, preferably a mammal such as a non-primate (e.g., cows, pigs, horses, cats, dogs, rats, etc.) and a primate (e.g., monkey, such as a cynomolgous monkey and a human). In a preferred embodiment, the subject is a human.

Specific examples of cancers that can be treated by the methods encompassed by the invention include, but are not limited to, cancers that over express EphA2. In a further embodiment, the cancer is of an epithelial origin. Examples of such cancers are cancer of the lung, colon, prostate, breast, and skin. Additional cancers are listed by example and not by limitation in Section 5.4.1.2, infra. In particular embodiments, methods of the invention can be used to treat, prevent and/or manage metastasis from primary tumors.

The methods and compositions of the invention comprise the administration of one or more EphA2-BiTEs of the invention to subjects/patients suffering from or expected to suffer from cancer, e.g., have a genetic predisposition for a particular type of cancer, have been exposed to a carcinogen, or are in remission from a particular cancer. As used herein, “cancer” refers to primary or metastatic cancers. Such patients may or may not have been previously treated for cancer. The methods and compositions of the invention may be used as any line of therapy, e.g., a first, second, third, etc. line of therapy. Included in the invention is also the treatment of patients undergoing other cancer therapies and the methods and compositions of the invention can be used before any adverse effects or intolerance of these other cancer therapies occurs. The invention also encompasses methods for administering one or more EphA2-BiTEs of the invention to treat, prevent and/or manage symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of the therapy(ies) on cancer cells, using the art-accepted meanings of “refractory” in such a context. In various embodiments, a cancer is refractory where the number of cancer cells has not been significantly reduced, or has increased. The invention also encompasses methods for administering one or more EphA2-BiTEs to prevent the onset or recurrence of cancer in patients predisposed to having cancer.

In particular embodiments, the EphA2-BiTEs of the invention, or other therapeutics that reduce EphA2 expression, are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis.

In alternate embodiments, the invention provides methods for treating patients' cancer by administering one or more EphA2-BiTEs of the invention in combination with any other therapy or to patients who have proven refractory to other treatments but are no longer on these treatments. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more EphA2-BiTEs of the invention are administered to prevent the recurrence of cancer.

In preferred embodiments, the existing treatment is chemotherapy. In particular embodiments, the existing treatment includes administration of chemotherapies including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc. Among these patients are patients treated with radiation therapy, hormonal therapy and/or biological therapy/immunotherapy. Also among these patients are those who have undergone surgery for the treatment of cancer.

Alternatively, the invention also encompasses methods for treating patients undergoing or having undergone radiation therapy. Among these are patients being treated or previously treated with chemotherapy, hormonal therapy and/or biological therapy/immunotherapy. Also among these patients are those who have undergone surgery for the treatment of cancer.

In other embodiments, the invention encompasses methods for treating patients undergoing or having undergone hormonal therapy and/or biological therapy/immunotherapy. Among these are patients being treated or having been treated with chemotherapy and/or radiation therapy. Also among these patients are those who have undergone surgery for the treatment of cancer.

Additionally, the invention also provides methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

In other embodiments, the invention provides administration of one or more EphA2-BiTEs of the invention without any other cancer therapies for the treatment of cancer, but who have proved refractory to such treatments. In specific embodiments, patients refractory to other cancer therapies are administered one or more EphA2-BiTEs of the invention in the absence of cancer therapies.

In other embodiments, patients with a pre-cancerous condition associated with cells that overexpress EphA2 can be administered EphA2-BiTEs of the invention to treat the disorder and decrease the likelihood that it will progress to malignant cancer. In specific embodiments, the pre-cancerous condition is high-grade prostatic intraepithelial neoplasia (PIN), fibroadenoma of the breast, fibrocystic disease, or compound nevi.

5.4.1.2. Cancers

Cancers and related disorders that can treated, prevented and/or managed by methods and compositions of the present invention include but are not limited to: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungaling (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment, prevention and/or management of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative cell disorders, are treated or prevented in the skin, lung, colon, breast, prostate, bladder, kidney, pancreas, ovary, or uterus. In other specific embodiments, sarcoma, melanoma, or leukemia is treated or prevented.

In specific embodiments, the cancers to be treated, prevented and/or managed by the methods and compositions of the invention are of an epithelial origin. In other embodiments, the cancer is malignant and overexpresses EphA2. In a specific embodiment, the cancer comprises cells that aberrantly express EphA2. In other embodiments, the disorder to be treated is a pre-cancerous condition associated with cells that overexpress EphA2. In a specific embodiments, the pre-cancerous condition is high-grade prostatic intraepithelial neoplasia (PIN), fibroadenoma of the breast, fibrocystic disease, or compound nevi.

In specific embodiments, the methods and compositions of the invention are used for the treatment, prevention and/or management of breast, colon, ovarian, lung, and prostate cancers and skin cancer, such as melanoma.

5.4.1.3. Treatment of Breast Cancer

In specific embodiments, patients with breast cancer are administered an effective amount of one or more EphA2-BiTEs of the invention. In another embodiment, the EphA2-BiTEs of the invention can be administered in combination with an effective amount of one or more other agents useful for breast cancer therapy including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), herceptin, tamoxifen, the combination of tamoxifen and cytotoxic chemotherapy, taxanes (such as docetaxel and paclitaxel). In a further embodiment, EphA2-BiTEs of the invention can be administered with taxanes plus standard doxorubicin and cyclophosphamide for adjuvant treatment of node-positive, localized breast cancer.

In a specific embodiment, patients with pre-cancerous fibroadenoma of the breast or fibrocystic disease are administered an EphA2-BiTE of the invention to treat the disorder and decrease the likelihood that it will progress to malignant breast cancer.

5.4.1.4. Treatment of Colon Cancer

In specific embodiments, patients with colon cancer are administered an effective amount of one or more EphA2-BiTEs of the invention. In another embodiment, the antibodies of the invention can be administered in combination with an effective amount of one or more other agents useful for colon cancer therapy including but not limited to: the combination of 5-FU and leucovorin, the combination of 5-FU and levamisole, irinotecan (CPT-11) or the combination of irinotecan, 5-FU and leucovorin (IFL).

5.4.1.5. Treatment of Prostate Cancer

In specific embodiments, patients with prostate cancer are administered an effective amount of one or more EphA2-BiTEs of the invention. In another embodiment, the EphA2-BiTEs of the invention can be administered in combination with an effective amount of one or more other agents useful for prostate cancer therapy including but not limited to: external-beam radiation therapy, interstitial implantation of radioisotopes (i.e., 125, palladium, iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel.

In a specific embodiment, patients with pre-cancerous high-grade prostatic intraepithelial neoplasia (PIN) are administered an EphA2-BiTE of the invention to treat the disorder and decrease the likelihood that it will progress to malignant prostate cancer.

5.4.1.6. Treatment of Melanoma

In specific embodiments, patients with melanoma are administered an effective amount of one or more EphA2-BiTEs of the invention. In another embodiment, the EphA2-BiTEs of the invention can be administered in combination with an effective amount of one or more other agents useful for melanoma cancer therapy including but not limited to: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), interferon alpha (IFN-α), and interleukin-2 (IL-2). In a specific embodiment, an effective amount of one or more EphA2-BiTEs of the invention can be administered in combination with isolated hyperthermic limb perfusion (ILP) with melphalan (L-PAM), with or without tumor necrosis factor-alpha (TNF-alpha) to patients with multiple brain metastases, bone metastases, and spinal cord compression to achieve symptom relief and some shrinkage of the tumor with radiation therapy.

In a specific embodiment, patients with pre-cancerous compound nevi are administered an EphA2-BiTE of the invention to treat the disorder and decrease the likelihood that it will progress to malignant melanoma.

5.4.1.7. Treatment of Ovarian Cancer

In specific embodiments, patients with ovarian cancer are administered an effective amount of one or more EphA2-BiTEs of the invention. In another embodiment, the EphA2-BiTEs of the invention can be administered in combination with an effective amount of one or more other agents useful for ovarian cancer therapy including but not limited to: intraperitoneal radiation therapy, such as P³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. It is contemplated that an effective amount of one or more EphA2-BiTEs of the invention is administered in combination with the administration Taxol for patients with platinum-refractory disease. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors.

5.4.1.8. Treatment of Lung Cancers

In specific embodiments, patients with small lung cell cancer are administered an effective amount of one or more EphA2-BiTEs of the invention. In another embodiment, the EphA2-BiTEs of the invention can be administered in combination with an effective amount of one or more other agents useful for lung cancer therapy including but not limited to: thoracic radiation therapy, cisplatin, vincristine, doxorubicin, and etoposide, alone or in combination, the combination of cyclophosphamide, doxorubicin, vincristine/etoposide, and cisplatin (CAV/EP), local palliation with endobronchial laser therapy, endobronchial stents, and/or brachytherapy.

In other specific embodiments, patients with non-small lung cell cancer are administered an effective amount of one or more EphA2-BiTEs of the invention in combination with an effective amount of one or more other agents useful for lung cancer therapy including but not limited to: palliative radiation therapy, the combination of cisplatin, vinblastine and mitomycin, the combination of cisplatin and vinorelbine, paclitaxel, docetaxel or gemcitabine, the combination of carboplatin and paclitaxel, interstitial radiation therapy for endobronchial lesions or stereotactic radiosurgery.

5.4.2 Other Prophylactic/Therapeutic Agents

In some embodiments, therapy by administration of one or more EphA2-BiTEs of the invention is combined with the administration of one or more therapies such as, but not limited to, chemotherapies, radiation therapies, hormonal therapies, and/or biological therapies/immunotherapies. Prophylactic or therapeutic agents include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules. Prophylactic or therapeutic agents can be derived from any known organism (including, but not limited to, animals, plants, bacteria, fungi, and protista, or viruses) or from a library of synthetic molecules. Such therapies can be administered prior to, concurrently, or after the administration of one or more EphA2-BiTEs of the invention.

In a specific embodiment, the methods of the invention encompass administration of an EphA2-BiTE of the invention in combination with the administration of one or more prophylactic/therapeutic agents that are inhibitors of kinases such as, but not limited to, ABL, ACK, AFK, AKT (e.g., AKT-1, AKT-2, and AKT-3), ALK, AMP-PK, ATM, Aurora1, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44 mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). In preferred embodiments, an EphA2-BiTE of the invention is administered in combination with the administration of one or more prophylactic/therapeutic agents that are inhibitors of Eph receptor kinases (e.g., EphA2). In a most preferred embodiment, an of the invention is administered in combination with the administration of one or more prophylactic/therapeutic agents that are inhibitors of EphA2.

In another specific embodiment, the methods of the invention encompass administration of an EphA2-BiTE of the invention in combination with the administration of one or more prophylactic/therapeutic agents that are angiogenesis inhibitors such as, but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-β); Vasculostatin; Vasostatin (calreticulin fragment); ZD6126; ZD6474; farnesyl transferase inhibitors (FTI); and bisphosphonates.

In another specific embodiment, the methods of the invention encompass administration of an EphA2-BiTE of the invention in combination with the administration of one or more prophylactic/therapeutic agents that are anti-cancer agents such as, but not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi I mimetics, semustine, senescence derived inhibitor 1, sense oligonucleotides, signal transduction inhibitors, signal transduction modulators, single chain antigen binding protein, sizofuran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, VITAXIN®, vorozole, zanoterone, zeniplatin, zilascorb, and zinostatin stimalamer. Preferred additional anti-cancer drugs are 5-fluorouracil and leucovorin.

In more particular embodiments, the present invention also comprises the administration of one or more EphA2-BiTEs of the invention in combination with the administration of one or more therapies such as, but not limited to anti-cancer agents such as those disclosed in Table 1, preferably for the treatment of breast, ovary, melanoma, prostate, colon and lung cancers as described above.

TABLE 1

| Therapeutic Agent | Administration | Dose | Intervals |
|---|---|---|---|
| doxorubicin | Intravenous | 60-75 mg/m² on Day 1 | 21 day intervals |
| hydrochloride | | | |
| (Adriamycin RDF ® | | | |
| and Adriamycin | | | |
| PFS ®) | | | |
| epirubicin | Intravenous | 100-120 mg/m² on Day 1 of | 3-4 week cycles |
| hydrochloride | | each cycle or divided equally | |
| (Ellence ™) | | and given on Days 1-8 of the | |
| | | cycle | |
| fluorousacil | Intravenous | How supplied: | |
| | | 5 ml and 10 ml vials | |
| | | (containing 250 and 500 mg | |
| | | flourouracil respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for 4 courses |
| (Taxol ®) | | | (administered sequentially to |
| | | | doxorubicin-containing |
| | | | combination chemotherapy) |
| tamoxifen citrate | Oral | 20-40 mg | Daily |
| (Nolvadex ®) | (tablet) | Dosages greater than 20 mg | |
| | | should be given in divided | |
| | | doses (morning and evening) | |
| leucovorin calcium | Intravenous or | How supplied: | Dosage is unclear from text. |
| for injection | intramuscular | 350 mg vial | PDR 3610 |
| | injection | | |
| luprolide acetate | Single | 1 mg (0.2 ml or 20 unit mark) | Once a day |
| (Lupron ®) | subcutaneous | | |
| | injection | | |
| flutamide | Oral (capsule) | 250 mg | 3 times a day at 8 hour |
| (Eulexin ®) | | (capsules contain 125 mg | intervals (total daily dosage |
| | | flutamide each) | 750 mg) |
| nilutamide | Oral | 300 mg or 150 mg | 300 mg once a day for 30 |
| (Nilandron ®) | (tablet) | (tablets contain 50 or 150 mg | days followed by 150 mg |
| | | nilutamide each) | once a day |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/ml | |
| ketoconazole | Cream | 2% cream applied once or | |
| (Nizoral ®) | | twice daily depending on | |
| | | symptoms | |
| prednisone | Oral | Initial dosage may vary from | |
| | (tablet) | 5 mg to 60 mg per day | |
| | | depending on the specific | |
| | | disease entity being treated. | |
| estramustine | Oral | 14 mg/kg of body weight | Daily given in 3 or 4 divided |
| phosphate sodium | (capsule) | (i.e. one 140 mg capsule for | doses |
| (Emcyt ®) | | each 10 kg or 22 lb of body | |
| | | weight) | |
| etoposide or VP-16 | Intravenous | 5 ml of 20 mg/ml solution | |
| | | (100 mg) | |
| dacarbazine | Intravenous | 2-4.5 mg/kg | Once a day for 10 days. |
| (DTIC-Dome ®) | | | May be repeated at 4 week |
| | | | intervals |
| polifeprosan 20 with | wafer placed in | 8 wafers, each containing 7.7 | |
| carmustine implant | resection cavity | mg of carmustine, for a total | |
| (BCNU) (nitrosourea) | | of 61.6 mg, if size and shape | |
| (Gliadel ®) | | of resection cavity allows | |
| cisplatin | Injection | [n/a in PDR 861] | |
| | | How supplied: | |
| | | solution of 1 mg/ml in multi- | |
| | | dose vials of 50 mL and | |
| | | 100 mL | |
| mitomycin | Injection | supplied in 5 mg and 20 mg | |
| | | vials (containing 5 mg and 20 | |
| | | mg mitomycin) | |
| gemcitabine HCl | Intravenous | For NSCLC-2 schedules | 4 week schedule- |
| (Gemzar ®) | | have been investigated and | Days 1, 8 and 15 of each 28- |
| | | the optimum schedule has not | day cycle. Cisplatin |
| | | been determined | intravenously at 100 mg/m² |
| | | 4 week schedule- | on day 1 after the infusion of |
| | | administration intravenously | Gemzar. |
| | | at 1000 mg/m² over 30 | 3 week schedule- |
| | | minutes on 3 week schedule- | Days 1 and 8 of each 21 day |
| | | Gemzar administered | cycle. Cisplatin at dosage of |
| | | intravenously at 1250 mg/m² | 100 mg/m² administered |
| | | over 30 minutes | intravenously after |
| | | | administration of Gemzar on |
| | | | day 1. |
| carboplatin | Intravenous | Single agent therapy: | Every 4 weeks |
| (Paraplatin ®) | | 360 mg/m² I.V. on day 1 | |
| | | (infusion lasting 15 minutes | |
| | | or longer) | |
| | | Other dosage calculations: | |
| | | Combination therapy with | |
| | | cyclophosphamide, Dose | |
| | | adjustment recommendations, | |
| | | Formula dosing, etc. | |
| ifosamide | Intravenous | 1.2 g/m² daily | 5 consecutive days |
| (Ifex ®) | | | Repeat every 3 weeks or |
| | | | after recovery from |
| | | | hematologic toxicity |
| topotecan | Intravenous | 1.5 mg/m² by intravenous | 5 consecutive days, starting |
| hydrochloride | | infusion over 30 minutes | on day 1 of 21 day course |
| (Hycamtin ®) | | daily | |

The invention also encompasses administration of the EphA2-BiTEs of the invention in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physicians' Desk Reference (61^{st} ed., 2007).

5.4.2.1. Patients with Hyperproliferative Cell Disorders

The present invention provides methods for treating, preventing and/or managing a hyperproliferative cell disorder or a symptom thereof, the methods comprising administering to a subject one or more EphA2-BiTE of the invention alone or in combination with therapies other than an EphA2-BiTE. The subject is an animal, preferably a mammal such as non-primate (e.g., cows, pigs, horses, cats, dogs, rats, etc.) and a primate (e.g., monkey, such as a cynomolgous monkey or human). In a preferred embodiment, the subject is a human.

In a specific embodiment, the hyperproliferative cell disorder is not cancer. Non-limiting examples of hyperproliferative cell disorders to be treated, prevented and/or managed by the methods of the invention are disclosed, e.g., in U.S. Pat. Pub. No. 2005-0059592, entitled “EphA2 and Hyperproliferative Cell Disorders,” which is incorporated by reference herein in its entirety. Accordingly, the invention also provides compositions and methods designed for the treatment, prevention and/or management of hyperproliferative cell disorders (non-limiting examples of such disorders are disclosed in, e.g., U.S. Pat. Pub. No. 2005-0059592, entitled “EphA2 and Hyperproliferative Cell Disorders,” which is incorporated by reference herein in its entirety).

In particular, the present invention provides methods for treating, preventing and/or managing a hyperproliferative cell disorder where the expression of EphA2 is upregulated in cells affected by such a disorder, said methods comprising administering to a subject in need thereof an effective amount of one or more EphA2-BiTEs of the invention, and optionally, an effective amount of a therapy other than an EphA2-BiTE. In a preferred embodiment, the hyperproliferative cell disorder to be treated, prevented and/or managed in accordance with the methods of the invention are asthma, COPD, lung fibrosis, asbestosis, IPF, DIP, UIP, kidney fibrosis, liver fibrosis, other fibroses, bronchial hyper-responsiveness, psoriasis, seborrheic dermatitis, cystic fibrosis, or a hyperproliferative endothelial cell disorder, such as restenosis, hyperproliferative vascular disease, Behcet's Syndrome, atherosclerosis, macular degeneration, or a hyperproliferative fibroblast disorder.

5.4.2.2. Patients with Inflammatory and/or Autoimmune Disorders

The present invention provides methods for treating, managing and/or preventing an inflammatory or autoimmune disorder or a symptom thereof, the methods comprising administering to a subject one or more EphA2-BiTE of the invention alone or in combination with therapies other than an EphA2-BiTE. The subject is preferably a mammal such as a primate (e.g., monkey (e.g., a rhesus monkey, cynomolgus monkey or chimpanzee), or human). In a preferred embodiment, the subject is a human.

In particular, the present invention provides methods for treating, preventing, and/or managing an inflammatory or autoimmune disorder where the expression of EphA2 is upregulated in cells affected by such a disorder, said methods comprising administering to a subject in need thereof an effective amount of one or more EphA2-BiTEs of the invention, and optionally, an effective amount of a therapy other than an EphA2-BiTE. In specific embodiments, the inflammatory or autoimmune disorder to be treated are disorders that are disclosed in, e.g., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety.

In accordance with these embodiments, the EphA2-BiTEs of the invention are administered in combination with an effective amount of VITAXIN® (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety). In another specific embodiment, the invention provides methods for treating, preventing and/or managing an inflammatory or autoimmune disorder or one or more symptoms thereof, said methods comprising administering to a subject in need thereof an effective amount of one or more EphA2-BiTEs of the invention in combination with an effective amount of siplizumab (MedImmune, Inc., International Pub. No. WO 02/069904, which is incorporated herein by reference in its entirety). In another specific embodiment, the invention provides methods for treating, preventing and/or managing an inflammatory or autoimmune disorder or one or more symptoms thereof, said methods comprising administering to a subject in need thereof an effective amount of one or more EphA2-BiTEs of the invention in combination with an effective amount an anti-inflammatory agent disclosed in Section 5.4.2.4, infra.

5.4.2.3. Patients with Infections

The present invention provides methods for treating, preventing and/or managing an infection (in particular, an intracellular infection), or a symptom thereof, the methods comprising administering one or more EphA2-BiTE of the invention alone or in combination with therapies other than an EphA2-BiTE. The subject is preferably a mammal such as non-primate (e.g., cows, pigs, horses, cats, dogs, rats, etc.) and a primate (e.g., monkey, such as a cynomolgous monkey or human). In a preferred embodiment, the subject is a human.

The methods of the invention comprise the administration of one or more EphA2-BiTEs of the invention to patients suffering from or expected to suffer from (e.g., patients with a genetic predisposition for or patients that have previously suffered from) an infection. Such patients may have been previously treated or are currently being treated for the infection, e.g., with a non-EphA2-BiTE therapy. In a further embodiment, the methods of the invention comprise the administration of one or more EphA2-BiTEs of the invention to patients that are immunocompromised or immunosuppressed. In a certain embodiment, an EphA2-BiTE is not administered to patients that are immunocompromised or immunosuppressed. In accordance with the invention, an EphA2-BiTE may be used as any line of therapy, including, but not limited to, a first, second, third and fourth line of therapy. Further, in accordance with the invention, an EphA2-BiTE can be used before any adverse effects or intolerance of the non-EphA2-BiTE therapies occurs. The invention encompasses methods for administering one or more EphA2-BiTEs of the invention to prevent the onset or recurrence of an infection.

In one embodiment, the invention also provides methods of treatment, prevention and/or management of an infection as alternatives to current therapies. In a specific embodiment, the current therapy has proven or may prove too toxic (i.e., results in unacceptable or unbearable side effects) for the patient. In another embodiment, an EphA2-BiTE decreases the side effects as compared to the current therapy. In another embodiment, the patient has proven refractory to a current therapy. In such embodiments, the invention provides for the administration of one or more EphA2-BiTEs of the invention without any other anti-infection therapies. In certain embodiments, one or more EphA2-BiTEs of the invention can be administered to a patient in need thereof instead of another therapy to treat an infection. In one embodiment, the invention provides methods of treating, preventing and/or managing of an active infection. In another embodiment, the invention provides methods of treating, preventing and/or managing a latent infection. In another embodiment, the invention provides methods of preventing the recurrence of an acute infection. In yet another embodiment, the invention provides methods of treating, preventing and/or managing a chronic infection.

The present invention also encompasses methods for administering one or more EphA2-BiTEs of the invention to treat, prevent and/or manage symptoms of infections in patients that are or have become refractory to non-EphA2-BiTE therapies. The determination of whether the infection is refractory can be made either in vivo or in vitro by any method known in the art for assaying the effectiveness of a therapy on affected cells in the infection, particularly epithelial cells, or in patients that are or have become refractory to non-EphA2-BiTE therapies.

5.4.2.3.1 Viral Infections

Increased expression of EphA2 has been found to be associated with infections by certain intracellular pathogens, in particular, RSV (see, e.g., U.S. Appn. Ser. No. 11/259,266, filed Oct. 27, 2005, titled “Use of Modulators of EphA2 and EphrinA1 for the Treatment and Prevention of Infections,” which is incorporated by reference herein in its entirety). Accordingly, the invention also provides compositions and methods designed for the treatment, prevention and/or management of a pathogen infection, including, but not limited to, a viral infection such as for example, a RSV infection. One or more EphA2-BiTEs of the invention and compositions comprising said EphA2-BiTEs can be administered to a subject to treat, prevent and/or manage a viral infection or one or more symptoms thereof. In a preferred embodiment, the viral infection to be treated, prevented and/or managed in accordance with the methods of the present invention are intracellular viral infections. One or more EphA2-BiTEs of the invention and compositions comprising said antibodies may be administered in combination with one or more other therapies (e.g., one or more prophylactic or therapeutic agents) other than EphA2-BiTEs of the invention to a subject predisposed to or with a viral infection useful for the treatment, prevention and/or management of a viral infection. Non-limiting examples of such therapies include the agents described in Section 5.4.2.5, infra.

In a specific embodiment, the invention provides methods of treating, preventing and/or managing a viral infection or one or more symptoms thereof, said method comprising administering to a subject in need thereof an effective amount of one or more EphA2-BiTEs of the invention. In another embodiment, the invention provides a method of treating, preventing and/or managing a viral infection or one or more symptoms thereof, said method comprising administering to a subject in need thereof an effective amount of one or more EphA2-BiTEs of the invention and an effective amount of one or more therapies (e.g., one or more prophylactic or therapeutic agents) other than EphA2-BiTEs of the invention.

In certain embodiments, an effective amount of one or more EphA2-BiTEs of the invention is administered in combination with an effective amount of one or more therapies (e.g., one or more prophylactic or therapeutic agents) currently being used, have been used, or are known to be useful in the treatment, prevention and/or management of a viral infection or one or more symptoms thereof to a subject in need thereof. Therapies for a viral infection, include, but are not limited to, anti-viral agents such as acyclovir, amantadine, oseltamivir, ribaviran, palivizumab, and anamivir. In certain embodiments, an effective amount of one or more EphA2-BiTEs of the invention is administered in combination with one or more supportive measures to a subject in need thereof to treat, prevent and/or manage a viral infection or one or more symptoms thereof. Non-limiting examples of supportive measures include humidification of the air by an ultrasonic nebulizer, aerolized racemic epinephrine, oral dexamethasone, intravenous fluids, intubation, fever reducers (e.g., ibuprofen, acetometaphin), and antibiotic and/or anti-fungal therapy (i.e., to prevent or treat secondary bacterial infections).

Any type of viral infection or condition resulting from or associated with a viral infection can be treated, prevented and/or managed in accordance with the methods of the invention, said methods comprising administering an effective amount of one or more EphA2-BiTEs of the invention alone or in combination with an effective amount of another therapy (e.g., a prophylactic or therapeutic agent other than EphA2-BiTEs of the invention). Examples of viruses which cause viral infections include, but are not limited to, retroviruses (e.g., human T-cell lymphotrophic virus (HTLV) types I and II and human immunodeficiency virus (HIV, e.g., HIV-1 and HIV-2)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, hMPV, and pneumovirus), adenoviruses, bunyaviruses (e.g., hantavirus), comaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C and PIV), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). Biological responses to a viral infection include, but not limited to, elevated levels of IgE antibodies, increased proliferation and/or infiltration of T-cells, increased proliferation and/or infiltration of B cells, epithelial hyperplasia, and mucin production. In a specific embodiment, the invention also provides methods of treating, preventing and/or managing viral infections that are associated with or cause the common cold, viral pharyngitis, viral laryngitis, viral croup, viral bronchitis, influenza, parainfluenza viral diseases (“PIV”) diseases (e.g., croup, bronchiolitis, bronchitis, pneumonia), respiratory syncytial virus (“RSV”) diseases, metapneumavirus diseases, and adenovirus diseases (e.g., febrile respiratory disease, croup, bronchitis, pneumonia), said method comprising administering an effective amount of one or more EphA2-BiTEs of the invention alone or in combination with an effective amount of another therapy.

In a specific embodiment, influenza virus infections, PIV infections, hMPV infections, adenovirus infections, and/or RSV infections, or one or more of symptoms thereof are treated, prevented and/or managed in accordance with the methods of the invention. In a specific embodiment, the invention provides methods for treating, preventing and/or managing a RSV infection or one or more symptoms thereof, said methods comprising administering to a subject in need thereof an effective amount of one or more EphA2-BiTEs of the invention alone or in combination with one or more anti-viral agents such as, but not limited to, amantadine, rimantadine, oseltamivir, znamivir, ribaviran, RSV-IVIG (i.e., intravenous immune globulin infusion) (RESPIGAMT™, and palivizumab and those antibodies disclosed in U.S. patent application Ser. Nos. 09/996,288 and 09/996,265, both entitled “Methods of Administering/Dosing Anti-RSV Antibodies For Prophylaxis and Treatment,” filed Nov. 28, 2001. In certain embodiments, the viral infection treated, prevented and/or managed in accordance with the methods of the invention is not a RSV infection.

In a specific embodiment, the invention provides methods for treating, preventing and/or managing a PIV infection or one or more symptoms thereof, said methods comprising administering to a subject in need thereof an effective amount of one or more EphA2-BiTEs of the invention alone or in combination with an effective amount of one or more anti-viral agents such as, but not limited to, amantadine, rimantadine, oseltamivir, znamivir, ribaviran, and palivizumab. In another specific embodiment, the invention provides methods for treating, preventing and/or managing a hMPV infection or one or more symptoms thereof, said methods comprising of administering an effective amount of one or more antibodies of the invention alone or in combination with an effective amount of one or more anti-viral agents, such as, but not limited to, amantadine, rimantadine, oseltamivir, znamivir, ribaviran, and palivizumab to a subject in need thereof. In another specific embodiment, the invention provides methods for treating, preventing and/or managing influenza, said methods comprising administering an effective amount of one or more EphA2-BiTEs of the invention alone or in combination with an effective amount of an anti-viral agent such as, but not limited to zanamivir (RELENZA®), oseltamivir (TAMIFLU®), rimantadine, and amantadine (SYMADINE®; SYMMETREL®) to a subject in need thereof.

The invention provides methods for preventing the development of asthma in a subject who suffers from or had suffered from a viral respiratory infection, said methods comprising administering an effective amount of one or more EphA2-BiTEs of the invention alone or in combination with an effective amount of another therapy. In a specific embodiment, the subject is an elderly person (i.e., a human subject who is 65 years or older), an infant born prematurely, an infant, or a child. In another specific embodiment, the subject suffered from or suffers from RSV infection. In a specific embodiment, the infection is not a viral respiratory infection. In a further embodiment, the infection is not an RSV infection.

In a specific embodiment, the invention provides methods for treating, preventing and/or managing one or more secondary responses to a primary viral infection, said methods comprising of administering an effective amount of one or more EphA2-BiTEs of the invention alone or in combination with an effective amount of other therapies (e.g., other prophylactic or therapeutic agents). Examples of secondary responses to a primary viral infection, particularly a primary viral respiratory infection, include, but are not limited to, asthma-like responsiveness to mucosal stimula, elevated total respiratory resistance, increased susceptibility to secondary viral, bacterial, fungal and protozoan infections, and development of such conditions such as, but not limited to, pneumonia, croup, and febrile bronchitis. In a specific embodiment, the invention provides methods for treating, preventing and/or managing an acute viral infection. In a further embodiment, the invention provides methods for treating, preventing and/or managing a latent viral infection. In yet further embodiments, the invention provides methods for treating, preventing and/or managing an HIV infection or an HBV infection.

In a specific embodiment, the invention provides methods of treating, preventing and/or managing a viral infection or one or more symptoms thereof, said methods comprising administering to a subject in need thereof an effective amount of one or more EphA2-BiTEs of the invention in combination with an effective amount of VITAXIN® (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety). In another specific embodiment, the invention provides methods for treating, preventing and/or managing a viral infection or one or more symptoms thereof, said methods comprising administering to a subject in need thereof an effective amount of one or more EphA2-BiTEs of the invention in combination with an effective amount of siplizumab (MedImmune, Inc., International Pub. No. WO 02/069904, which is incorporated herein by reference in its entirety). In another embodiment, the invention provides methods of treating, preventing and/or managing a viral infection or one or more symptoms thereof, said methods comprising administering to a subject in need thereof an effective amount of one or more EphA2-BiTEs in combination with an effective amount of one or more anti-IL-9 antibodies such as those disclosed in U.S. Pat. Pub. No. 20050002934 (Jan. 6, 2005), which is incorporated herein by reference in its entirety. In yet another embodiment, the invention provides methods for treating, preventing and/or managing a viral infection or one or more symptoms thereof, said methods comprising administering to a subject in need thereof an effective amount of one or more EphA2-BiTEs of the invention in combination with an effective amount of two or more of the following: VITAXIN®, an anti-IL-9 antibody and/or siplizumab.

In one embodiment, an effective amount of one or more EphA2-BiTEs of the invention is administered in combination with an effective amount of one or more anti-IgE antibodies to a subject to treat, prevent and/or manage a viral infection or one or more symptoms thereof. In a specific embodiment, an effective amount of one or more antibodies of the invention is administered in combination with an effective amount of anti-IgE antibody TNX901 to a subject to treat, prevent and/or manage a viral infection or one or more symptoms thereof. In a specific embodiment, an effective amount of one or more antibodies of the invention is administered in combination with an effective amount of anti-IgE antibody rhuMAb-E25 omalizumab to a subject to treat, prevent and/or manage a viral infection or one or more symptoms thereof. In another embodiment, an effective amount of one or more EphA2-BiTEs of the invention is administered in combination with an effective amount of anti-IgE antibody HMK-12 to a subject to treat, prevent and/or manage a viral infection or one or more symptoms thereof. In a specific embodiment, an effective amount of one or more EphA2-BiTEs of the invention is administered in combination with an effective amount of anti-IgE antibody 6HD5 to a subject to treat, prevent and/or manage a viral infection or one or more symptoms thereof. In another embodiment, an effective amount of one or more antibodies of the invention is administered in combination with an effective amount of anti-IgE antibody MAb Hu-901 to a subject to treat, prevent and/or manage a viral infection or one or more symptoms thereof.

The invention encompasses methods for preventing the development of viral infections, in a patient expected to suffer from a viral infection or at increased risk of such an infection, e.g., patients with suppressed immune systems (e.g., organ-transplant recipients, AIDS patients, patients undergoing chemotherapy, the elderly, infants born prematurely, infants, children, patients with carcinoma of the esophagus with obstruction, patients with tracheobronchial fistula, patients with neurological diseases (e.g., caused by stroke, amyotrophic lateral sclerosis, multiple sclerosis, and myopathies), and patients already suffering from a viral infection). The patients may or may not have been previously treated for a viral infection.

The EphA2-BiTEs of the invention, compositions, or combination therapies of the invention may be used as any line of therapy, including but not limited to, the first, second, third, fourth, or fifth line of therapy, to treat, prevent and/or manage a viral infection or one or more symptom thereof. The invention also includes methods of treating, preventing and/or managing a viral infection, or one or more symptoms thereof in a patient undergoing therapies for other diseases or disorders associated increased in EphA2 expression. The invention encompasses methods of treating, preventing and/or managing a viral infection, or one or more symptoms thereof in a patient before any adverse effects or intolerance to therapies other than EphA2-BiTEs of the invention develops. The invention also encompasses methods of treating, preventing and/or managing a viral infection or a symptom thereof in refractory patients. In certain embodiments, a patient with a viral infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a viral infection is refractory when viral replication has not decreased or has increased. The invention also encompasses methods of preventing the onset or reoccurrence of viral infections in patients at risk of developing such infections. The invention also encompasses methods of treating, preventing and/or managing a viral infection or a symptom thereof in patients who are susceptible to adverse reactions to conventional therapies. The invention further encompasses methods for treating, preventing and/or managing a viral infection for which no anti-viral therapy is available.

The invention encompasses methods for treating, preventing and/or managing a viral infection or a symptom thereof in a patient who has proven refractory to therapies other than EphA2-BiTEs of the invention but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring viral infections despite management or treatment with existing therapies.

The present invention encompasses methods for treating, preventing and/or managing a viral infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to treat, prevent and/or manage a viral infection or one or more symptoms thereof.

Viral infection therapies and their dosages, routes of administration and recommended usage are known in the art and have been described in such literature as the Physicians' Desk Reference (61^{st} ed., 2007).

5.4.2.3.2 Bacterial Infections

The invention provides a method of treating, preventing and/or managing a bacterial infection, in particular an intracellular bacterial infection, or one or more symptoms thereof, said method comprising administering to a subject in need thereof an effective amount of one or more EphA2-BiTEs of the invention. Preferably, cells infected with the intracellular bacteria have increased EphA2 expression. In another embodiment, the invention provides a method of treating, preventing and/or managing a bacterial infection or one or more symptoms thereof, said method comprising administering to a subject in need thereof an effective amount of a one or more EphA2-BiTEs of the invention and an effective amount of one or more therapies (e.g., one or more prophylactic or therapeutic agents), other than EphA2-BiTEs of the invention. In a preferred embodiment, the bacterial infections to be treated, prevented and/or managed in accordance with the methods of the present invention are intracellular bacterial infections.

Any type of intracellular bacterial infection or condition resulting from or associated with a bacterial infection (e.g., a respiratory infection) can be treated, prevented and/or managed in accordance with the methods of invention. Examples of intracellular bacteria which cause infections include, but not limited to, Mycobacterium tuberculosis, Mycobacterium leprae, Salmonella enterica serovar Typhi, Brucella sp, Legionella sp, Listeria monocytogenes, Francisella tularensis, Rickettsia rickettsii; Rickettsia prowazekii; Rickettsia typhi; Rickettsia tsutsugamushi; Chlamydia trachomatis; Chlamydia psittaci; and Chlamydia pneumoniae. In certain embodiments, an intracellular bacterial infection treated, prevented and/or managed in accordance with the methods of the invention is not a respiratory bacterial infection. In other embodiments, an intracellular bacterial infection treated, prevented and/or managed in accordance with the methods of the invention is not a Salmonella species infection. In yet other embodiments, an intracellular bacterial infection treated, prevented and/or managed in accordance with the methods of the invention is not Salmonella dublin infection.

In a specific embodiment, the invention provides methods for treating, preventing and/or managing an intracellular bacterial infection or one or more symptoms thereof, said method comprising administering to a subject in need thereof an effective amount of one or more EphA2-BiTEs of the invention. In another embodiment, the invention provides a method of treating, preventing and/or managing an intracellular bacterial infection or one or more symptoms thereof, said method comprising administering to a subject in need thereof an effective amount of a one or more EphA2-BiTEs of the invention and an effective amount of one or more therapies (e.g., prophylactic or therapeutic agents), other than EphA2-BiTEs of the invention.

In certain embodiments, the invention provides methods to treat, prevent and/or manage a bacterial infection or one or more of the symptoms, said methods comprising administering to a subject in need thereof one or more EphA2-BiTEs of the invention in combination with and effective amount of one or more therapies (e.g., one or more prophylactic or therapeutic agents), other than EphA2-BiTEs of the invention, used to treat, prevent and/or manage bacterial infections. Therapies for bacterial infections, particularly, bacterial infections include, but are not limited to, anti-bacterial agents (e.g., aminoglycosides (e.g., gentamicin, tobramycin, amikacin, netilimicin) aztreonam, celphalosporins (e.g., cefaclor, cefadroxil, cephalexin, cephazolin), clindamycin, erythromycin, penicillin (e.g., penicillin V, crystalline penicillin G, procaine penicillin G), spectinomycin, and tetracycline (e.g., chlortetracycline, doxycycline, oxytetracycine)) and supportive therapy, such as supplemental and mechanical ventilation. In certain embodiments, one or more EphA2-BiTEs of the invention are administered in combination with one or more supportive measures to a subject in need thereof to treat, prevent and/or manage a bacterial infection or one or more symptoms thereof. Non-limiting examples of supportive measures include humidification of air by ultrasonic nebulizer, aerolized racemic epinephrine, oral dexamethasone, intravenous fluids, intubation, fever reducers (e.g., ibuprofen, acetometaphin), and more preferably, antibiotic or anti-viral therapy (i.e., to prevent or treat secondary infections).

The invention provides methods for treating, preventing and/or managing a biological response to a bacterial infection, such as, but not limited to, elevated levels of IgE antibodies, mast cell proliferation, degranulation, and/or infiltration, increased proliferation and/or infiltration of B cells, and increased proliferation and/or infiltration of T-cells, said methods comprising administering to a subject in need thereof an effective amount of one or more EphA2-BiTEs of the invention alone or in combination with an effective amount one or more therapies (e.g. a prophylactic or therapeutic agent) other than EphA2-BiTEs of the invention. The invention also provides methods of treating, preventing and/or managing respiratory conditions caused by or associated with bacterial infections, such as, but not limited to, pneumonia, recurrent aspiration pneumonia, legionellosis, whooping cough, meningitis, or tuberculosis, said methods comprising administering to a subject in need thereof an effective amount of one or more EphA2-BiTEs of the invention alone or in combination with an effective amount of another therapy.

In a specific embodiment, the methods of the invention are utilized to treat, prevent and/or manage a bacterial infection caused by Mycobacteria or one or more symptoms thereof, said method comprising administering to a subject in need thereof of an effective amount of one or more EphA2-BiTEs of the invention alone or in combination with an effective amount of one or more other therapies (e.g., one or more prophylactic or therapeutic agents) other than EphA2-BiTEs of the invention.

In a specific embodiment, the invention provides methods for treating, preventing and/or managing one or more secondary conditions or responses to a primary bacterial infection, preferably a primary bacterial infection, said method comprising administering to a subject in need thereof an effective amount of one or more EphA2-BiTEs of the invention alone or in combination with an effective amount of other therapies (e.g., other prophylactic or therapeutic agents). Examples of secondary conditions or responses to a primary bacterial infection, particularly a bacterial infection, include, but are not limited to, asthma-like responsiveness to mucosal stimula, elevated total resistance, increased susceptibility to secondary viral, bacterial, fungal and protozoan infections, and development of such conditions such as, but not limited to, pneumonia, croup, and febrile bronchitits.

In a specific embodiment, the methods of the invention are used to treat, prevent and/or manage a bacterial infection, or one or more symptoms thereof, said methods comprising administering to a subject in need thereof an effective amount of one or more EphA2-BiTEs of the invention in combination with an effective amount of VITAXIN® (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ₃ Antagonist in Combination With an HMG-CoA Reductase Inhibitor or a Bisphosphonate,” each of which is incorporated herewith by reference in its entirety).

In another specific embodiment, the methods of the invention are used to treat, prevent and/or manage a bacterial infection, or one or more symptoms thereof, said methods comprising administering to a subject in need thereof an effective amount of one or more EphA2-BiTEs of the invention in combination with an effective amount of siplizumab (MedImmune, Inc., International Pub. No. WO 02/069904). In another embodiment, the methods of the invention are used to treat, prevent and/or manage a bacterial infection or one or more symptoms thereof, said methods comprising administering to a subject in need thereof an effective amount of one or more EphA2-BiTEs in combination with an effective amount of one or more anti-II-9 antibodies (e.g., one of the anti-IL-9 antibodies described in U.S. Pat. Pub. No. 20050002934 (Jan. 6, 2005)), which is incorporated herein by reference in its entirety). In yet another embodiment, the invention provides methods of treating, preventing and/or managing a bacterial infection, or one or more symptoms thereof, said methods comprising administering an effective amount of one or more EphA2-BiTEs of the invention in combination with an effective amount of two or more of the following: VITAXIN®, siplizumab, and/or anti-II-9 antibodies.

The invention encompasses methods for preventing the development of bacterial infections, in a patient expected to suffer from a bacterial infection or at increased risk of such an infection, e.g., patients with suppressed immune systems (e.g., organ-transplant recipients, AIDS patients, patients undergoing chemotherapy, the elderly, infants born prematurely, infants, children, patients with carcinoma of the esophagus with obstruction, patients with tracheobronchial fistula, patients with neurological diseases (e.g., caused by stroke, amyotrophic lateral sclerosis, multiple sclerosis, and myopathies), and patients already suffering from an infection). The patients may or may not have been previously treated for an infection.

The EphA2-BiTEs of the invention or combination therapies of the invention may be used as any line of therapy, including but not limited to the first, second, third, fourth, or fifth line of therapy, to treat, prevent and/or manage a bacterial infection, or one or more symptom thereof. The invention also includes methods of treating, preventing and/or managing a bacterial infection, or one or more symptoms thereof in a patient undergoing therapies for other diseases or disorders. The invention encompasses methods of treating, preventing and/or managing a bacterial infection, or one or more symptoms thereof in a patient before any adverse effects or intolerance to therapies other than EphA2-BiTEs of the invention develops. The invention also encompasses methods of treating, preventing and/or managing a bacterial infection, or a symptom thereof in refractory patients. In certain embodiments, a patient with a bacterial infection is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a bacterial infection is refractory when bacterial replication has not decreased or has increased. The invention also encompasses methods of preventing the onset or reoccurrence of a bacterial infection, in patients at risk of developing such infection. The invention also encompasses methods of treating, managing and/or preventing a bacterial infection, or a symptom thereof in patients who are susceptible to adverse reactions to conventional therapies. The invention further encompasses methods for treating, preventing and/or managing bacterial infections, for which no anti-bacterial therapy is available.

The invention encompasses methods for treating, preventing and/or managing a bacterial infection, or a symptom thereof in a patient who has proven refractory to therapies other than EphA2-BiTEs of the invention, but are no longer on these therapies. In certain embodiments, the patients being managed or treated in accordance with the methods of this invention are patients already being treated with anti-inflammatory agents, antibiotics, anti-virals, anti-fungals, anti-protozoan agents, or other biological therapy/immunotherapy. Among these patients are refractory patients, patients who are too young for conventional therapies, and patients with reoccurring bacterial infections despite management or treatment with existing therapies.

The present invention encompasses methods treating, preventing and/or managing a bacterial infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to treat, prevent and/or manage a bacterial infection, or one or more symptoms thereof.

Bacterial infection therapies and their dosages, routes of administration and recommended usage are known in the art and have been described in such literature as the Physicians' Desk Reference (61^{st} ed., 2007).

5.4.2.3.3 Fungal Infections

One or more EphA2-BiTEs of the invention can be administered according to methods of the invention to a subject to treat, prevent and/or manage a fungal infection or one or more symptoms thereof. In a preferred embodiment, cells infected by fungi have increased EphA2 expression. One or more EphA2-BiTEs of the invention may be also administered to a subject to treat, manage, and/or ameliorate a fungal infection and/or one or more symptoms thereof in combination with one or more other therapies (e.g., one or more prophylactic or therapeutic agents) other than EphA2-BiTEs of the invention which are useful for the treatment, prevention and/or management of a fungal infection or one or more symptoms thereof. In a preferred embodiment, the fungal infections to be treated, prevented and/or managed in accordance with the methods of the present invention are intracellular fungal infections.

Any type of fungal infection or condition resulting from or associated with a fungal infection can be treated, prevented and/or managed in accordance with the methods of invention. Examples of fungus which cause fungal infections include, but not limited to, Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immilis, Conidiobolus species, Cryptococcus neoforms, Cunninghamella species, dermatophytes, Histoplasma capsulalum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes. In a specific embodiment, a fungal infection is not a respiratory fungal infection.

In a specific embodiment, the invention provides a method of treating, preventing and/or managing a fungal infection or one or more symptoms thereof, said method comprising administering to a subject in need thereof an effective amount of one or more EphA2-BiTEs of the invention. In another embodiment, the invention provides a method of treating, preventing and/or managing a fungal infection or one or more symptoms thereof, said method comprising administering to a subject in need thereof an effective amount of one or more EphA2-BiTEs of the invention and an effective amount of one or more therapies (e.g., one or more prophylactic or therapeutic agents) other than EphA2-BiTEs of the invention.

In certain embodiments, an effective amount of one or more antibodies is administered in combination with an effective amount of one or more therapies (e.g., one or more prophylactic or therapeutic agents), other than EphA2-BiTEs of the invention, which are currently being used, have been used, or are known to be useful in the treatment, prevention and/or management of a fungal infection, preferably a fungal infection, to a subject in need thereof. Therapies for fungal infections include, but are not limited to, anti-fungal agents such as azole drugs e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B colloidal dispersion (“ABCD”) (AMPHOTEC®), liposomal amphotericin B (AMBISONE®)), postassium iodide (KI), pyrimidine (e.g., flucytosine (ANCOBON®)), and voriconazole (VFEND®). In certain embodiments, an effective amount of one or more EphA2-BiTEs of the invention are administered in combination with one or more supportive measures to a subject in need thereof to treat, prevent and/or manage a fungal infection or one or more symptoms thereof. Non-limiting examples of supportive measures include humidification of the air by an ultrasonic nebulizer, aerolized racemic epinephrine, oral desamethasone, intravenous fluids, intubation, fever reducers (e.g., ibuprofen and acetometaphin), and anti-viral or anti-bacterial therapy (i.e., to prevent or treat secondary viral or bacterial infections).

The invention also provides methods for treating, preventing and/or managing a biological response to a fungal infection such as, but not limited to, elevated levels of IgE antibodies, elevated nerve growth factor (NGF) levels, mast cell proliferation, degranulation, and/or infiltration, increased proliferation and/or infiltration of B cells, and increased proliferation and/or infiltration of T-cells, said methods comprising administration of an effective amount of one or more EphA2-BiTEs alone or in combination with one or more other therapies.

In a specific embodiment, the invention provides methods for treating, preventing and/or managing one or more secondary conditions or responses to a primary fungal infection, preferably a primary fungal infection, said method comprising of administering to a subject in need thereof an effective amount of one or more EphA2-BiTEs of the invention alone or in combination with an effective amount of other therapies (e.g., other prophylactic or therapeutic agents) other than EphA2-BiTEs of the invention. Examples of secondary conditions or responses to a primary fungal infections, particularly primary fungal infection include, but are not limited to, asthma-like responsiveness to mucosal stimula, elevated total resistance, increased susceptibility to secondary viral, fungal, and fungal infections, and development of such conditions such as, but not limited to, pneumonia, croup, and febrile bronchitits.

In a specific embodiment, the invention provides methods to treat, prevent and/or manage a fungal infection or one or more symptoms thereof, said methods comprising administering to a subject in need thereof an effective amount of one or more EphA2-BiTEs of the invention in combination with an effective amount of VITAXIN® (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ₃ Antagonist in Combination With an HMG-CoA Reductase Inhibitor or a Bisphosphonate,” each of which is incorporated herewith by reference in its entirety) to a subject in need thereof.

In another embodiment, the invention provides methods of treating, preventing and/or managing a fungal infection or one or more symptoms thereof, said methods comprising administering to a subject in need thereof an effective amount of one or more EphA2-BiTEs of the invention in combination with an effective amount of siplizumab (MedImmune, Inc., International Pub. No. WO 02/069904) to a subject in need thereof. In another embodiment, the invention provides methods of treating, preventing and/or managing a fungal infection or one or more symptoms thereof, said methods comprising administering to a subject in need thereof an effective amount of one or more EphA2-BiTEs in combination with an effective amount of one or more anti-IL-9 antibodies (e.g., one or more of the anti-IL-9 antibodies described in U.S. Pat. Pub. No. 20050002934 (Jan. 6, 2005)), which is incorporated herein by reference in its entirety). In another embodiment, the invention provides methods of treating, preventing and/or managing a fungal infection or one or more symptoms thereof, said methods comprising administering to a subject in need thereof an effective amount of one or more EphA2-BiTEs in combination with an effective amount of two or more of the following: VITAXIN®, Siplizumab and/or anti-IL-9 antibodies.

The invention encompasses methods for preventing the development of fungal infections in a patient expected to suffer from a fungal infection, or at increased risk of such an infection. Such subjects include, but are not limited to, patients with suppressed immune systems (e.g., patients organ-transplant recipients, AIDS patients, patients undergoing chemotherapy, patients with carcinoma of the esophagus with obstruction, patients with tracheobronchial fistula, patients with neurological diseases (e.g., caused by stroke, amyotorphic lateral sclerosis, multiple sclerosis, and myopathies), and patients already suffering from a condition, particularly a infection). In a specific embodiment, the patient suffers from bronchopulmonary dysplasia, congenital heart disease, cystic fibrosis, and/or acquired or congenital immunodeficiency. In another specific embodiment, the patient is an infant born prematurely, an infant, a child, an elderly human, or a human in a group home, nursing home, or some other type of institution. The invention also encompasses methods of treating, preventing and/or managing a fungal infection or one or more symptoms thereof in patients who are susceptible to adverse reactions to conventional anti-fungal therapies for conditions for which no therapies are available.

The EphA2-BiTEs of the invention or combination therapies of the invention may be used as any line of therapy, including but not limited to the first, second, third, fourth, or fifth line of therapy, to treat, prevent and/or manage a fungal infection or one or more symptom thereof. The invention also includes methods of treating, preventing and/or managing a fungal infection or one or more symptoms thereof in a patient undergoing therapies for other disease or disorders. The invention encompasses methods of treating, preventing and/or managing a fungal infection or one or more symptoms thereof in a patient before any adverse effects or intolerance to therapies other EphA2-BiTEs of the invention develops. The invention also encompasses methods of treating, preventing and/or managing a fungal infection or a symptom thereof in refractory patients. In certain embodiments, a patient with a fungal infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a fungal infection, is refractory when fungal replication has not decreased or has increased. The invention also encompasses methods of preventing the onset or reoccurrence of fungal infections, in patients at risk of developing such infections. The invention also encompasses methods of treating, preventing and/or managing a fungal infection or a symptom thereof in patients who are susceptible to adverse reactions to conventional therapies. The invention further encompasses methods for treating, preventing and/or managing fungal infections, for which no anti-fungal therapy is available.

The invention encompasses methods for treating, preventing and/or managing a fungal infection, or a symptom thereof in a patient who has proven refractory to therapies other than EphA2-BiTEs of the invention but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring fungal infections despite management or treatment with existing therapies.

The present invention provides methods for treating, preventing and/or managing a fungal infection or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to treat, prevent and/or manage a fungal infection, or one or more symptoms thereof.

Fungal infection therapies and their dosages, routes of administration and recommended usage are known in the art and have been described in such literature as the Physicians' Desk Reference (61^{st} ed., 2007).

5.4.2.3.4 Protozoan Infections

One or more EphA2-BiTEs of the invention can be administered according to methods of the invention to a subject to treat, prevent and/or manage a protozoan infection or one or more symptoms thereof. In a preferred embodiment, cells infected by protozoa have increased EphA2 expression. One or more EphA2-BiTEs of the invention may be also administered to a subject to treat, manage, and/or ameliorate a protozoa infection or one or more symptoms thereof in combination with one or more other therapies (e.g., one or more prophylactic or therapeutic agents) other than EphA2-BiTEs of the invention which are useful for the treatment, prevention and/or management of a fungal infection or one or more symptoms thereof. In a preferred embodiment, the protozoan infections to be treated, prevented and/or managed in accordance with the methods of the present invention are intracellular protozoan infections.

Any type of protozoa infection or condition resulting from or associated with a protozoa infection can be treated, prevented and/or managed in accordance with the methods of invention. Examples of protozoa which cause infections include, but not limited to, Leishmania; Trypanosoma; Giardia; Trichomonas; Entamoeba; Dientamoeba; Naegleria and Acanthamoeba; Babesia; Plasmodium; Isospora; Sarcocystis; Toxoplasma; Enterocytozoon; Balantidium; and Pneumocystis.

In a specific embodiment, the invention provides a method of treating, preventing and/or managing a protozoa infection or one or more symptoms thereof, said method comprising administering to a subject in need thereof an effective amount of one or more EphA2-BiTEs of the invention. In another embodiment, the invention provides a method of treating, preventing and/or managing a protozoa infection or one or more symptoms thereof, said method comprising administering to a subject in need thereof an effective amount of one or more EphA2-BiTEs of the invention and an effective amount of one or more therapies (e.g., one or more prophylactic or therapeutic agents) other than EphA2-BiTEs of the invention.

In certain embodiments, an effective amount of one or more EphA2-BiTEs is administered in combination with an effective amount of one or more therapies (e.g., one or more prophylactic or therapeutic agents), other than EphA2-BiTEs of the invention, which are currently being used, have been used, or are known to be useful in the treatment, prevention and/or management of a protozoa infection, to a subject in need thereof. In certain embodiments, an effective amount of one or more EphA2-BiTEs of the invention are administered in combination with one or more supportive measures to a subject in need thereof to treat, prevent and/or manage a protozoa infection or one or more symptoms thereof. Non-limiting examples of supportive measures include humidification of the air by an ultrasonic nebulizer, aerolized racemic epinephrine, oral desamethasone, intravenous fluids, intubation, fever reducers (e.g., ibuprofen and acetometaphin), and anti-viral or anti-bacterial therapy (i.e., to prevent or treat secondary viral or bacterial infections).

The invention also provides methods for treating, preventing and/or managing a biological response to a protozoa infection such as, but not limited to, elevated levels of IgE antibodies, elevated nerve growth factor (NGF) levels, mast cell proliferation, degranulation, and/or infiltration, increased proliferation and/or infiltration of B cells, and increased proliferation and/or infiltration of T-cells, said methods comprising administration of an effective amount of one or more EphA2-BiTEs alone or in combination with one or more other therapies.

In a specific embodiment, the invention provides methods for treating, preventing and/or managing one or more secondary conditions or responses to a primary infection, preferably a primary protozoa infection, said method comprising of administering to a subject in need thereof an effective amount of one or more EphA2-BiTEs of the invention alone or in combination with an effective amount of other therapies (e.g., other prophylactic or therapeutic agents) other than EphA2-BiTEs of the invention.

In a specific embodiment, the invention provides methods to treat, prevent and/or manage a protozoa infection or one or more symptoms thereof, said methods comprising administering to a subject in need thereof an effective amount of one or more EphA2-BiTEs of the invention in combination with an effective amount of VITAXIN® (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ₃ Antagonist in Combination With an HMG-CoA Reductase Inhibitor or a Bisphosphonate,” each of which is incorporated herewith by reference in its entirety) to a subject in need thereof.

In another embodiment, the invention provides methods of treating, preventing and/or managing a protozoa infection or one or more symptoms thereof, said methods comprising administering to a subject in need thereof an effective amount of one or more EphA2-BiTEs of the invention in combination with an effective amount of siplizumab (MedImmune, Inc., International Pub. No. WO 02/069904) to a subject in need thereof. In another embodiment, the invention provides methods of treating, preventing and/or managing a protozoa infection or one or more symptoms thereof, said methods comprising administering to a subject in need thereof an effective amount of one or more EphA2-BiTEs in combination with an effective amount of one or more anti-IL-9 antibodies (e.g., the anti-IL-9 antibodies described in U.S. Pat. Pub. No. 20050002934 (Jan. 6, 2005)), which is incorporated herein by reference in its entirety). In another embodiment, the invention provides methods of treating, preventing and/or managing a protozoa infection or one or more symptoms thereof, said methods comprising administering to a subject in need thereof an effective amount of one or more EphA2-BiTEs in combination with an effective amount of two or more of the following: VITAXIN®, siplizumab and/or anti-IL-9 antibodies.

The invention encompasses methods for preventing the development of protozoa infections in a patient expected to suffer from a protozoa infection, or at increased risk of such an infection. Such subjects include, but are not limited to, patients with suppressed immune systems (e.g., patients organ-transplant recipients, AIDS patients, patients undergoing chemotherapy, patients with cancer, patients with tracheobronchial fistula, patients with neurological diseases (e.g., caused by stroke, amyotorphic lateral sclerosis, multiple sclerosis, and myopathies), and patients already suffering from a condition, particularly a infection). In a specific embodiment, the patient suffers from bronchopulmonary dysplasia, congenital heart disease, cystic fibrosis, and/or acquired or congenital immunodeficiency. In another specific embodiment, the patient is an infant born prematurely, an infant, a child, an elderly human, or a human in a group home, nursing home, or some other type of institution. The invention also encompasses methods of treating, preventing and/or managing a protozoa infection or one or more symptoms thereof in patients who are susceptible to adverse reactions to conventional anti-protozoa therapies for conditions for which no therapies are available.

The EphA2-BiTEs of the invention or combination therapies of the invention may be used as any line of therapy, including but not limited to the first, second, third, fourth, or fifth line of therapy, to treat, prevent and/or manage a protozoa infection or one or more symptom thereof. The invention also includes methods of treating, preventing and/or managing a protozoa infection or one or more symptoms thereof in a patient undergoing therapies for other disease or disorders. The invention encompasses methods of treating, preventing and/or managing a protozoa infection, or one or more symptoms thereof in a patient before any adverse effects or intolerance to therapies other EphA2-BiTEs of the invention develops. The invention also encompasses methods of treating, preventing and/or managing a protozoa infection, or a symptom thereof in refractory patients. In certain embodiments, a patient with a protozoa infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a protozoa infection is refractory when protozoa replication has not decreased or has increased. The invention also encompasses methods of preventing the onset or reoccurrence of protozoa infections, in patients at risk of developing such infections. The invention also encompasses methods of treating, preventing and/or managing a protozoa infection or a symptom thereof in patients who are susceptible to adverse reactions to conventional therapies. The invention further encompasses methods for treating, preventing and/or managing protozoa infections, for which no anti-protozoa therapy is available.

The invention encompasses methods for treating, preventing and/or managing a protozoa infection or a symptom thereof in a patient who has proven refractory to therapies other than EphA2-BiTEs of the invention but are no longer on these therapies. In certain embodiments, the patients being managed or treated in accordance with the methods of this invention are patients already being treated with antibiotics, anti-virals, anti-protozoa, or other biological therapy/immunotherapy. Among these patients are refractory patients, patients who are too young for conventional therapies, and patients with reoccurring protozoa infections despite management or treatment with existing therapies.

The present invention provides methods for treating, preventing and/or managing a protozoa infection or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to treat, prevent and/or manage a fungal infection or one or more symptoms thereof.

Protozoa infection therapies and their dosages, routes of administration and recommended usage are known in the art and have been described in such literature as the Physicians' Desk Reference (61^{st} ed., 2007).

5.4.2.4. Anti-Inflammatory Therapies

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes (e.g., montelukast (SINGULAIR™), zafirlukast (ACCOLATE™), pranlukast (ONON™), or zileuton (ZYFLO™)).

Anti-inflammatory therapies and their dosages, routes of administration, and recommended usage are known in the art and have been described in such literature as the Physicians' Desk Reference (61^{st} ed., 2007).

5.4.2.5. Anti-Viral Therapies

Any anti-viral agent well-known to one of skill in the art can be used in the compositions and the methods of the invention. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscamet, amantadine, rimantadine, saquinavir, indinavir, ritonavir, alpha-interferons and other interferons, and AZT.

In specific embodiments, the anti-viral agent is an immunomodulatory agent that is immunospecific for a viral antigen. As used herein, the term “viral antigen” includes, but is not limited to, any viral peptide, polypeptide and protein (e.g., HIV gp120, HIV nef, RSV F glycoprotein, RSV G glycoprotein, influenza virus neuraminidase, influenza virus hemagglutinin, HTLV tax, herpes simplex virus glycoprotein (e.g., gB, gC, gD, and gE) and hepatitis B surface antigen) that is capable of eliciting an immune response. Antibodies useful in this invention for treatment of a viral infection include, but are not limited to, antibodies against antigens of pathogenic viruses, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporiipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory synctial virus), and metapneumovirus (e.g., avian pneumovirus and human metapneumovirus)), picornaviridae (e.g., enterovirus, rhinovirus, hepatovirus (e.g., human hepatits A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Specific examples of antibodies available useful for the treatment of a viral infection include, but are not limited to, PRO542 (Progenics) which is a CD4 fusion antibody useful for the treatment of HIV infection; Ostavir (Protein Design Labs, Inc., CA) which is a human antibody useful for the treatment of hepatitis B virus; and Protovir (Protein Design Labs, Inc., CA) which is a humanized IgG1 antibody useful for the treatment of cytomegalovirus (CMV); and palivizumab (SYNAGIS®; MedImmune, Inc.; International Publication No. WO 02/43660) which is a humanized antibody useful for treatment of RSV.

In a specific embodiment, the anti-viral agents used in the compositions and methods of the invention inhibit or reduce a virus infection, inhibit or reduce the replication of a virus that causes an infection, or inhibit or reduce the spread of a virus that causes an infection to other cells or subjects. In another specific embodiment, the anti-viral agents used in the compositions and methods of the invention inhibit or reduce infection by RSV, hMPV, or PIV, inhibit or reduce the replication of RSV, hMPV, or PIV, or inhibit or reduce the spread of RSV, hMPV, or PIV to other cells or subjects. Examples of such agents and methods of treatment of RSV, hMPV, and/or PIV infections include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons. See U.S. Prov. Patent App. No. 60/398,475 filed Jul. 25, 2002, entitled “Methods of Treating and Preventing RSV, HMPV, and PIV Using Anti-RSV, Anti-HMPV, and Anti-PIV Antibodies,” and U.S. patent application Ser. No. 10/371,122 filed Feb. 21, 2003, which are incorporated herein by reference in its entirety.

In specific embodiments, the viral infection is RSV and the anti-viral antigen is an antibody that immunospecifically binds to an antigen of RSV. In certain embodiments, the anti-RSV-antigen antibody binds immunospecifically to an RSV antigen of the Group A of RSV. In other embodiments, the anti-RSV-antigen antibody binds immunospecifically to an RSV antigen of the Group B of RSV. In other embodiments, an antibody binds to an antigen of RSV of one Group and cross reacts with the analogous antigen of the other Group. In particular embodiments, the anti-RSV-antigen antibody binds immunospecifically to a RSV nucleoprotein, RSV phosphoprotein, RSV matrix protein, RSV small hydrophobic protein, RSV RNA-dependent RNA polymerase, RSV F protein, and/or RSV G protein. In additional specific embodiments, the anti-RSV-antigen antibody binds to allelic variants of a RSV nucleoprotein, a RSV nucleocapsid protein, a RSV phosphoprotein, a RSV matrix protein, a RSV attachment glycoprotein, a RSV fusion glycoprotein, a RSV nucleocapsid protein, a RSV matrix protein, a RSV small hydrophobic protein, a RSV RNA-dependent RNA polymerase, a RSV F protein, a RSV L protein, a RSV P protein, and/or a RSV G protein.

It should be recognized that antibodies that immunospecifically bind to a RSV antigen are known in the art. For example, palivizumab (SYNAGIS®) is a humanized monoclonal antibody presently used for the prevention of RSV infection in pediatric patients. In a specific embodiment, an antibody to be used with the methods of the present invention is palivizumab or an antibody-binding fragment thereof (e.g., a fragment containing one or more complementarity determining regions (CDRs) and preferably, the variable domain of palivizumab). The amino acid sequence of palivizumab is disclosed, e.g., in Johnson et al., 1997, J. Infection 176:1215-1224, and U.S. Pat. No. 5,824,307 and International Application Publication No.: WO 02/43660, entitled “Methods of Administering/Dosing Anti-RSV Antibodies for Prophylaxis and Treatment”, by Young et al., which are incorporated herein by reference in their entireties.

One or more antibodies or antigen-binding fragments thereof that bind immunospecifically to a RSV antigen comprise a Fc domain with a higher affinity for the FcRn receptor than the Fc domain of palivizumab can also be used in accordance with the invention. Such antibodies are described in U.S. patent application Ser. No. 10/020,354, filed Dec. 12, 2001, which is incorporated herein by reference in its entireties. Further, one or more of the anti-RSV-antigen antibodies A4B4; P12f2 P12f4; P11d4; A1e9; A12a6; A13c4; A17d4; A4B4; 1X-493L1; FR H3-3F4; M3H9; Y10H6; DG; AFFF; AFFF(1); 6H8; L1-7E5; L2-15B10; A13a11; A1h5; A4B4(1); A4B4-F52S; or A4B4L1FR-S28R can be used in accordance with the invention. These antibodies are disclosed in International Application Publication No.: WO 02/43660, entitled “Methods of Administering/Dosing Anti-RSV Antibodies for Prophylaxis and Treatment”, by Young et al., and U.S. Provisional Patent Application 60/398,475 filed Jul. 25, 2002, entitled “Methods of Treating and Preventing RSV, HMPV, and PIV Using Anti-RSV, Anti-HMPV, and Anti-PIV Antibodies” which are incorporated herein by reference in their entireties.

In certain embodiments, the anti-RSV-antigen antibodies are the anti-RSV-antigen antibodies of or are prepared by the methods of U.S. application Ser. No. 09/724,531, filed Nov. 28, 2000; Ser. No. 09/996,288, filed Nov. 28, 2001; and U.S. Pat. Publication No. US2003/0091584 A1, published May 15, 2003, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application Nos. 60/388,921, filed Jun. 14, 2002, and 60/388,920, filed Jun. 14, 2002, which are incorporated by reference herein in their entireties.

Anti-viral therapies and their dosages, routes of administration and recommended usage are known in the art and have been described in such literature as the Physicians' Desk Reference (61^{st} ed., 2007). Additional information on respiratory viral infections is available in Cecil Textbook of Medicine (18th ed., 1988).

5.4.2.6. Anti-Bacterial Therapies

Anti-bacterial agents and therapies well known to one of skill in the art for the treatment, prevention and/or management of bacterial infections can be used in the compositions and methods of the invention. Non-limiting examples of anti-bacterial agents include proteins, polypeptides, peptides, fusion proteins, antibodies, nucleic acid molecules, organic molecules, inorganic molecules, and small molecules that inhibit or reduce a bacterial infection, inhibit or reduce the replication of bacteria, or inhibit or reduce the spread of bacteria to other subjects. In particular, examples of anti-bacterial agents include, but are not limited to, penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In a preferred embodiment, the anti-bacterial agent is an agent that inhibits or reduces a bacterial infection, inhibits or reduces the replication of a bacteria that causes an infection, or inhibits or reduces the spread of a bacteria that causes an infection to other subjects. In cases in which the bacterial infection is a mycoplasma infection (e.g., pharyngitis, tracheobronchitis, and pneumonia), the anti-bacterial agent is preferably a tetracycline, erythromycin, or spectinomycin. In cases in which the bacterial infection is tuberculosis, the anti-bacterial agent is preferably, rifampcin, isonaizid, pyranzinamide, ethambutol, and streptomycin.

Anti-bacterial therapies and their dosages, routes of administration and recommended usage are known in the art and have been described in such literature as the Physicians' Desk Reference (61^{st} ed., 2007). Additional information on respiratory infections and anti-bacterial therapies is available in Cecil Textbook of Medicine (18th ed., 1988).

5.4.2.7. Anti-Fungal Therapies

Anti-fungal agents and therapies well known to one of skill in the art for treatment, prevention and/or management of a fungal infection or one or more symptoms thereof (e.g., a fungal respiratory infection) can be used in the compositions and methods of the invention. Non-limiting examples of anti-fungal agents include proteins, polypeptides, peptides, fusion proteins, antibodies, nucleic acid molecules, organic molecules, inorganic molecules, and small molecules that inhibit and/or reduce fungal infection, inhibit and/or reduce the replication of fungi, or inhibit and/or reduce the spread of fungi to other subjects. Specific examples of anti-fungal agents include, but are not limited to, azole drugs (e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B (FUNGIZONE®), amphotericin B lipid complex (“ABLC”)(ABELCET®), amphotericin B colloidal dispersion (“ABCD”)(AMPHOTEC®), liposomal amphotericin B (AMBISONE®)), potassium iodide (KI), pyrimidine (e.g., flucytosine (ANCOBON®)), and voriconazole (VFEND®). See, e.g., Table 3, infra for a list of specific anti-fungal agents and their recommended dosages.

TABLE 3
Anti-fungal Agents

| Anti-fungal Agent | Dosage |
|---|---|
| Amphotericin B | |
| ABELCET ® (lipid complex injection) | 5 mg/kg/day |
| AMBISOME ® (liposome for injection) | 3-5 mg/kg/day |
| AMPHOTEC ® (complex for injection) | 3-4 mg/kg/day |
| Caspofungin acetate (CANCIDAS ®) | 70 mg on day one followed by 50 |
| | mg/day |
| Fluconazole (DIFLUCAN ®) | up to 400 mg/day (adults) |
| | up to 12 mg/kg/day (children) |
| Itraconazole (SPORANOX ®) | 200-400 mg/day |
| Flucytosine (ANCOBON ®) | 50-150 mg/kg/day in divided dose |
| | every 6 hours |
| Liposomal nystatin | 1-4 mg/kg |
| Ketoconazole (NIZORAL ®) | 200 mg single daily dose up to |
| | 400 mg/day in two divided doses |
| | (adults) |
| | 3.3-6.6 mg/kg/day for children 2 |
| | years old and older |
| Voriconazole (VFEND ®) | 6 mg/kg i.v. loading dose every 12 |
| | hours for two doses, followed by |
| | maintenance dose of 4 mg/kg i.v. |
| | every 12 hours, then oral maintenance |
| | dose of 200-100 mg tablet |

In certain embodiments, the anti-fungal agent is an agent that inhibits or reduces a fungal infection, inhibits or reduces the replication of a fungus that causes an infection, or inhibits or reduces the spread of a fungus that causes an infection to other subjects. In cases in which the fungal infection is Blastomyces dermatitidis, the anti-fungal agent is preferably itraconazole, amphotericin B, fluconazole, or ketoconazole. In cases in which the fungal infection is pulmonary aspergilloma, the anti-fungal agent is preferably amphotericin B, liposomal amphotericin B, itraconazole, or fluconazole. In cases in which the fungal infection is histoplasmosis, the anti-fungal agent is preferably amphotericin B, itraconazole, fluconazole, or ketoconazole. In cases in which the fungal infection is coccidioidomycosis, the anti-fungal agent is preferably fluconazole or amphotericin B. In cases in which the fungal infection is cryptococcosis, the anti-fungal agent is preferably amphotericin B, fluconazole, or combination of the two agents. In cases in which the infection is chromomycosis, the anti-fungal agent is preferably itraconazole, fluconazole, or flucytosine. In cases in which the fungal infection is mucormycosis, the anti-fungal agent is preferably amphotericin B or liposomal amphotericin B. In cases in which the pulmonary or respiratory fungal infection is pseudoallescheriasis, the anti-fungal agent is preferably itraconazole ore miconazole.

Anti-fungal therapies and their dosages, routes of administration, and recommended usage are known in the art and have been described in such literature as Dodds et al., 2000 Pharmacotherapy 20(11) 1335-1355, the Physicians' Desk Reference (61^{st} ed., 2007) and the Merk Manual of Diagnosis and Therapy (17th ed., 1999).

5.4.2.8. Anti-Protozoan Therapies

Anti-protozoan agents and therapies well known to one of skill in the art for treatment, prevention and/or management of a protozoa infection or one or more symptoms thereof (e.g., a respiratory infection associated with a protozoa infection) can be used in the compositions and methods of the invention. Non-limiting examples of anti-protozoan agents include proteins, polypeptides, peptides, fusion proteins, antibodies, nucleic acid molecules, organic molecules, inorganic molecules, and small molecules that inhibit and/or reduce a protozoa infection, inhibit and/or reduce the replication of protozoa, or inhibit and/or reduce the spread of protozoa to other subjects. Specific examples of anti-protozoan agents include, but are not limited to, chloroquine phosphate (Aralen™); quinine sulfate plus one of the following: doxycycline, tetracycline, or clindamycin; atovaquone-proguanil (Malarone™); Mefloquine (Lariam™); metronidazole (Flagyl); tinidazole (Tindamax); 5-nitroimidazole (ornidazole), and agents described in U.S. Pat. No. 6,440,936.

In certain embodiments, the anti-protozoan agent is an agent that inhibits or reduces a protozoa infection, inhibits or reduces the replication of a protozoa that causes an infection, or inhibits or reduces the spread of a protozoa that causes an infection to other subjects. In cases in which the protozoan infection is Trichomoniasis, the anti-protozoan agent is preferably metronidazole (Flagyl), tinidazole (Tindamax), or 5-nitroimidazole (ornidazole). In cases in which the protozoan infection is malaria, the anti-protozan agent is preferably chloroquine phosphate (Aralen™); quinine sulfate plus one of the following: doxycycline, tetracycline, or clindamycin; quinidine gluconate plus one of the following: docycycline, tetracycline, or clindamycin; Fansidar™; Malarone™ (atovaquone 250 mg plus proguanil 100 mg); or Mefloquine (Larium™).

Anti-protozoan therapies and their dosages, routes of administration, and recommended usage are known in the art and have been described in such literature as Dodds et al., 2000 Pharmacotherapy 20(11) 1335-1355, the Physicians' Desk Reference (61^{st} ed., 2007); the Merk Manual of Diagnosis and Therapy (17th ed., 1999); and publications provided by the Centers for Disease Control and Prevention (CDC; http://www.cdc.gov) (Atlanta, Ga.).

5.5 Biological Activity of EphA2-BiTEs

Various in vitro and in vivo assays known in the art and described in detail below in Section 6 may be used to determine the biological activity of the EphA2-BiTEs produced by the methods of the invention. Such in vitro and in vivo assays allows for screening and identification of EphA2-BiTEs with desired biological activity such as, e.g., target cell specificity and efficient lysis of said cells. In particular, the invention provides methods to determine target cell specificity (e.g., the ability of the EphA2-BiTEs to immunospecifically bind to cells that express EphA2) using various immunoassays. The binding characteristics of the EphA2-BiTEs of the invention may also be determined, using for example, surface plasmon resonance to identify the affinity constants (K_{D}, Kₒₙ and K_{off}) of the EphA2-BiTEs produced by the methods of the invention. The present invention further provides assays that may be used to screen for EphA2-BiTEs with specific biological activities, such as for example, the ability to bind target cells (e.g., tumor cells that overexpress EphA2) to mediate lysis in vitro using various cytotoxicity assays. Such assays may be used to determine the toxicity and efficacy of the EphA2-BiTEs of the invention (e.g., to determine the dose lethal to 50% of the population of cells treated with the EphA2-BiTEs (LD₅₀)). Also provided are assays that may be used to determine the ability of the EphA2-BiTEs of the invention to mediate tumor cell killing in vivo using animal models.

5.5.1 Assays to Determine Target Cell Specificity

Various assays known to one of skill in the art can be used to determine the ability of the EphA2-BiTEs of the invention to immunospecifically bind to EphA2- and CD3-expressing cells (e.g., cells that overexpress EphA2 or cells wherein certain epitopes of EphA2 are selectively exposed). Such assays include, for example, RIAs, ELISA, Western Blot, immunohistochemistry, immunofluorescence and flow cytometry assays. For example, as described in Section 6.2.3 below, flow cytometry based assays can be used to determine the bispecific cell binding of the various EphA2-BiTE constructs. EphA2-positive cell lines, such as A549 cells (human lung carcinoma cells) and MDA-MB-231 cells (human breast cancer cells) can be used as target cells, and CD3-positive HP-Ball cells (human T-cell line) can be used as effector cells. The cells may be mixed together and incubated with an EphA2-BiTE of interest. Flow cytometry may be used to determine the binding intensities to each EphA2-positive target cell.

Epitope exclusion analyses may be conducted using immunofluorescence staining to determine whether the EphA2-BiTEs produced by the methods of the invention retain epitope exclusion as the parental antibodies from which they were derived. See Section 6.2.4 below.

5.5.2 Surface Plasmon Resonance Assays

Surface plasmon resonance assays may be performed to determine the binding characteristics of the EphA2-BiTEs of the invention. The affinity and dissociation constants as well as the association and dissociation rates (kₐ, k_{D}, kₒₙ and k_{off}) of the EphA2-BiTEs produced by the methods of the invention may be identified using this assay. See, e.g., 6.8.3 below. For example, surface plasmon resonance assays may be performed using the Sensor Chip CM5 (Biacore AB, Uppsala, Sweden) which contains a carboxymethyl (CM) dextran matrix and a Biacore® 3000 surface plasmon resonance (SPR) biosensor (Biacore AB, Uppsala, Sweden). CD3εγ is covalently attached to the CM dextran matrix using amine coupling chemistry. A reference surface is created by omission of the CD3εγ coupling step. An EphA2-Fc is captured via a high-affinity interaction between the Fc portion of EphA2Fc and a goat anti-human IgG (Fc) (KPL, Inc., Gaithersburg, Md.). Goat anti-human IgG (Fc) is covalently attached to the CM dextran matrix using amine coupling chemistry. Two anti-human IgG (Fc)-specific surfaces are then created. One of these surfaces is used as a reference surface while the other surface is used to create an EphA2-Fc-specific surface. Different concentrations of bscEphA2×CD3 are then prepared by serial dilution in HBS-EP (0.01 M HEPES pH 7.4, 0.15 M NaCl, 3 mM EDTA, 0.005% surfactant P20). EphA2-BiTEs are injected in a serial-flow manner across the CD3εγ-specific or EphA2-specific surface and its corresponding reference surface. Dissociation of bound EphA2-BiTEs is monitored in the presence of HBS-EP. Remaining bound material was removed with 10 mM disodium tetraborate pH 8.5, 1 M NaCl (for CD3εγ-specific surface) or 10 mM glycine pH 1.7 (for EphA2-Fc-specific surface).

5.5.3 Cytotoxicity Assays

As illustrated in Section 6.3.1 below, flow cytometry-based redirected cell cytotoxcity assays may be performed to determine the EphA2-BiTE cytotoxic activity with effector cell donors. For example, CD3+ T-cell enriched human peripheral blood mononuclear cells (“PBMCs”) may be isolated from healthy donors. Target cells, e.g., cells expressing EphA2, are labeled with a fluorescent membrane dye such as (DiOC18(3) or “DiO”). The cells are coincubated with an EphA2-BiTE of interest and are analyzed by flow cytometry after the addition of propidium iodide (PI). Target cell lysis may then be calculated as the percentage of DiO positive cells staining positive by PI.

In a specific embodiment, an EphA2-BiTE of the invention mediates lysis of cells associated with aberrant EphA2 expression and/or activity (e.g., cancer cells, non-cancer hyperproliferative cells or infected cells that express EphA2). In accordance with this embodiment, such cells are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of normal cells from the same cell line or subject, cells of a normal, healthy subject and/or a population of normal, healthy cells. In another specific embodiment, the EphA2-BiTEs do not mediate lysis of cells from normal tissues or normal cells from tissues of a healthy subject or control.

The biological activity (e.g., anti-cancer, anti-hyperproliferation, or anti-infective activities) of the therapies used in accordance with the present invention also can be determined by using various experimental animal models for the study of cancer such as the SCID mouse model or transgenic mice where a mouse EphA2 is replaced with the human EphA2, nude mice with human xenografts, animal models described in Section 6 infra, or any animal model (including hamsters, rabbits, etc.) known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

In specific embodiments, the cytotoxic effects of the EphA2-BiTEs of the invention may also be tested in vivo with a mouse model such as the non-diabetic/severe combined immunodeficiency (NOD/SCID) mouse model using a tumor xenograft such as the SW480 human colon carcinoma cell line. The SW480 cell line may be selected for establishment of a human xenograft model because SW480 cells express EphA2. Briefly, animals may be injected with a mixture of target (e.g., SW480 cells) and effector (human CD3+ T cells from healthy donors) or a target cells alone without effector cells. Tumor growth kinetics may be measured between the two treatment groups. See, e.g., Section 6.4.1 below.

Accordingly, the toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects. Specific examples of methods of determining the biological activity of the EphA2-BiTEs of the invention are also provided in Section 6 of the Examples, infra.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of a disorder associated with aberrant expression and/or activity of EphA2 (e.g., cancer, a non-hyperproliferative cell disorder, or an infection).

5.6 Compositions

The present invention provides compositions comprising one or more of the EphA2-BiTEs of the invention. The compositions of the invention also include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more EphA2-BiTEs of the invention and a pharmaceutically acceptable carrier. In a further embodiment, the composition of the invention further comprises an additional therapy that is not an EphA2-BiTE.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, synthetic or vegetable origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer an EphA2-BiTE of the invention or the combination of an EphA2-BiTE of the invention and a prophylactic agent or therapeutic agent useful for treating, preventing and/or managing a disorder associated with aberrant expression (e.g., overexpression) and/or activity of EphA2 (e.g., cancer, a non-cancer hyperproliferative cell disorder, or an infection), e.g., microparticles, microcapsules, recombinant cells capable of expressing the antibody or antibody fragment (see, e.g., Wu and Wu, 1987, J. Biol. Chem. 262:4429-4432), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, prophylactic or therapeutic agents of the invention are administered intramuscularly, intravenously, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the EphA2-BiTEs of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

5.6.1 Formulations

Pharmaceutical compositions for use in accordance with the present invention may be formulated in conventional manner using one or more physiologically acceptable carriers or excipients.

Thus, the EphA2-BiTEs of the invention and their physiologically acceptable salts and solvates may be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachette indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various chemotherapeutic, biological/immunotherapeutic and hormonal therapeutic agents are known in the art and often described in the Physicians' Desk Reference (61^{st} ed., 2007). For instance, in certain specific embodiments of the invention, the therapeutic agents of the invention can be formulated and supplied as provided in Table 2.

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the EphA2-BiTEs of the invention, are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

5.6.2 Dosage and Frequency of Administration

The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, and/or management of a disorder associated with aberrant expression (e.g., overexpression) and/or activity of EphA2 (e.g., cancer, a non-cancer hyperproliferative cell disorder, or an infection), or one or more symptoms thereof, can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physicians' Desk Reference (61^{st} ed., 2007).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

Exemplary dosages of the EphA2-BiTEs of the invention administered to a patient is typically 0.01 μg to 100 mg. A particularly preferred dosage is 0.1 μg to 10 mg, more preferably, 1 μg to 100 μg, and most preferably, 3 μg to 10 μg.

In a specific embodiment, the dosage of EphA2-BiTEs (e.g., antibodies, compositions, or combination therapies of the invention) administered to treat, prevent and/or manage a disorder associated with aberrant expression (e.g., overexpression) and/or activity of EphA2 (e.g., cancer, a non-cancer hyperproliferative cell disorder, or an infection), or one or more symptoms thereof, in a patient is 100 mg/kg or less, 50 mg/kg or less, 25 mg/kg or less, 10 mg/kg or less, 1 mg/kg or less, 500 μg/kg or less, 400 μg/kg or less, 300 μg/kg or less, 200 μg/kg or less, 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the EphA2-BiTEs or combination therapies of the invention administered to treat, prevent and/or manage a disorder associated with aberrant expression (e.g., overexpression) and/or activity of EphA2 (e.g., cancer, a non-cancer hyperproliferative cell disorder, or an infection), or one or more symptoms thereof, in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of an effective amount of one or EphA2-BiTEs of the invention, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the EphA2-BiTEs of the invention. In yet other embodiments, a subject is administered a dose of an effective amount of one or more EphA2-BiTEs of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies and a subsequent dose of an effective amount of one or more EphA2-BiTEs of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of treating, preventing and/or managing a disorder associated with aberrant expression (e.g., overexpression) and/or activity of EphA2 (e.g., cancer, a non-cancer hyperproliferative cell disorder, or an infection), or one or more symptoms thereof, said method comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, at least 120 μg, at least 150 μg, at least 300 μg, at least 400 μg, at least 500 μg, at least at least 1 mg, at least 5 mg, at least 10 mg, at least 25 mg, at least 50 mg, or at least 100 mg of one or more EphA2-BiTEs, combination therapies, or compositions of the invention. In another embodiment, the invention provides a method of treating, preventing and/or managing a disorder associated with aberrant expression (e.g., overexpression) and/or activity of EphA2 (e.g., cancer, a non-cancer hyperproliferative cell disorder, or an infection), or one or more symptoms thereof, said methods comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more EphA2-BiTEs, combination therapies, or compositions of the invention as a continuous infusion, once every 3 days, preferably, once every 4 days, once every 5 days, once every 6 days, once every 7 days, once every 8 days, once every 10 days, once every two weeks, once every three weeks, or once a month.

The present invention provides methods of treating, preventing and/or managing a disorder associated with aberrant expression (e.g., overexpression) and/or activity of EphA2 (e.g., cancer, a non-cancer hyperproliferative cell disorder, or an infection), or one or more symptoms thereof, said method comprising: (a) administering to a subject in need thereof one or more doses of a prophylactically or therapeutically effective amount of one or more EphA2-BiTEs, combination therapies, or compositions of the invention; and (b) monitoring the plasma level/concentration of the said administered EphA2-BiTEs in said subject after administration of a certain number of doses of the said EphA2-BiTEs. Moreover, said certain number of doses is 1, 2, 3, 4, 5, 6, 7, 8, 9, 10, 11, or 12 doses of a prophylactically or therapeutically effective amount one or more EphA2-BiTEs, compositions, or combination therapies of the invention. In another embodiment, the dose is administered as a continuous i.v. infusion.

In a specific embodiment, the invention provides a method of treating, preventing and/or managing a disorder associated with aberrant expression (e.g., overexpression) and/or activity of EphA2 (e.g., cancer, a non-cancer hyperproliferative cell disorder, or an infection), or one or more symptoms thereof, said method comprising: (a) administering to a subject in need thereof a dose of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more EphA2-BiTEs of the invention; and (b) administering one or more subsequent doses to said subject when the plasma level of the EphA2-BiTE administered in said subject is less than 0.1 μg/ml, preferably less than 0.25 μg/ml, less than 0.5 μg/ml, less than 0.75 μg/ml, or less than 1 μg/ml. In another embodiment, the invention provides a method of treating, preventing and/or managing a disorder associated with aberrant expression (e.g., overexpression) and/or activity of EphA2 (e.g., cancer, a non-cancer hyperproliferative cell disorder, or an infection), or one or more symptoms thereof, said method comprising: (a) administering to a subject in need thereof one or more doses of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more antibodies of the invention; (b) monitoring the plasma level of the administered EphA2-BiTEs of the invention in said subject after the administration of a certain number of doses; and (c) administering a subsequent dose of EphA2-BiTEs of the invention when the plasma level of the administered EphA2-BiTE in said subject is less than 0.1 μg/ml, preferably less than 0.25 μg/ml, less than 0.5 μg/ml, less than 0.75 μg/ml, or less than 1 μg/ml. In one embodiment, said certain number of doses is 1, 2, 3, 4, 5, 6, 7, 8, 9, 10, 11, or 12 doses of an effective amount of one or more EphA2-BiTEs of the invention or can be administered as a continuous i.v. infusion.

Therapies (e.g., prophylactic or therapeutic agents), other than the EphA2-BiTEs of the invention, which have been or are currently being used treat, prevent and/or manage a disorder associated with aberrant expression (e.g., overexpression) and/or activity of EphA2 (e.g., cancer, a non-cancer hyperproliferative cell disorder, or an infection) or one or more symptoms thereof can be administered in combination with one or more EphA2-BiTEs according to the methods of the invention to treat, prevent and/or manage a disorder associated with aberrant expression (e.g., overexpression) and/or activity of EphA2 (e.g., cancer, a non-cancer hyperproliferative cell disorder, or an infection) or one or more symptoms thereof. Preferably, the dosages of prophylactic or therapeutic agents used in combination therapies of the invention are lower than those which have been or are currently being used to treat, prevent and/or manage a disorder associated with aberrant expression (e.g., overexpression) and/or activity of EphA2 (e.g., cancer, a non-cancer hyperproliferative cell disorder, or an infection) or one or more symptoms thereof. The recommended dosages of agents currently used for the treatment, prevention and/or management of a disorder associated with aberrant expression (e.g., overexpression) and/or activity of EphA2 (e.g., cancer, a non-cancer hyperproliferative cell disorder, or an infection), or one or more symptoms thereof, can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physicians' Desk Reference (61^{st} ed., 2007), Medical Economics Co., Inc., Montvale, N.J., which are incorporated herein by reference in its entirety.

In various embodiments, the therapies (e.g., prophylactic or therapeutic agents) are administered as a continuous infusion, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same EphA2-BiTEs of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of a therapy (e.g., a prophylactic or therapeutic agent) other than an EphA2-BiTE of the invention may be repeated and the administration may be separated by at least at least 1 day, 2 days, 3 days, 5 days, 10 days, 15 days, 30 days, 45 days, 2 months, 75 days, 3 months, or at least 6 months.

5.7 Kits

The invention provides a pharmaceutical pack or kit comprising one or more containers filled with one or more EphA2-BiTEs of the invention, polynucleotides encoding said EphA2-BiTEs, and vectors expressing said polynucleotides. Additionally, one or more other prophylactic or therapeutic agents useful for the treatment of a disorder associated with aberrant expression (i.e., overexpression) and/or activity of EphA2 (e.g., cancer, a non-cancer hyperproliferative cell disorder, or an infection) can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises, in one or more containers, one or more EphA2-BiTEs of the invention and instructions for use. In another embodiment, a kit further comprises one or more other prophylactic or therapeutic agents useful for the treatment of a disorder associated with aberrant expression (i.e., overexpression) and/or activity of EphA2 (e.g., cancer, a non-cancer hyperproliferative cell disorder, or an infection), in one or more containers. In a specific embodiment, the EphA2-BiTE is deimmunized anti-CD3×EA2 (VH/VL). In certain embodiments, the other prophylactic or therapeutic agent is a chemotherapeutic. In other embodiments, the prophylactic or therapeutic agent is a biological or hormonal therapeutic. In yet other embodiments, the prophylactic or therapeutic agent is an anti-viral, anti-fungal, anti-protozoan, anti-bacterial, or an anti-autoimmune agent.

The present invention further provides kits comprising any of the diagnostic compositions discussed supra.

### 6. EXAMPLES

The following non-limiting examples demonstrate the production of EphA2-BiTEs and their characterization.

6.1 EphA2-BiTE Generation

6.1.1 Construction of Eight EphA2-Specific BiTEs from Two Parental Monoclonal Antibodies

Plasmids with coding sequences for variable domains of two murine anti-EphA2 antibodies called EA2 and EA5 as described in U.S. Pub. No. US 2004-0091486 A1 and application Ser. No. 11/544,322, filed Oct. 6, 2006, were utilized for EphA2-BiTE construction. See also FIGS. 1 and 2, respectively. An anti-CD3 single-chain antibody, a deimmunized derivative of the human CD3ε-specific murine mAb L2K called diL2K was used for fusion with the EphA2 single-chain antibodies (see Dreier et al., 2002, Int. J. Cancer 100:690-697, which is incorporated by reference herein in its entirety). cDNAs for eight different BiTE constructs were generated by PCR-based fusion. BiTE constructs had the following arrangements of variable (VH and VL) domains and positioning of anti-EphA2 and anti-CD3 single-chain antibodies:

BiTE Constructs Made

| N-terminal Position | C-terminal Position |
|---|---|
| EA2 (VL/VH) | deimmunized anti-CD3 |
| EA2 (VH/VL) | (VH/VL) |
| EA5 (VL/VH) | deimmunized anti-CD3 |
| EA5 (VH/VL) | (VH/VL) |
| deimmunized anti-CD3 | EA2 (VL/VH) |
| (VH/VL) | EA2 (VH/VL) |
| deimmunized anti-CD3 | EA5 (VL/VH) |
| (VH/VL) | EA5 (VH/VL) |

The structure of the BiTE cDNA is shown in FIG. 3. The inserts cloned into the expression vector pEF-DHFR each comprised a leader with a 5′-terminal Kozak site for increased translation efficiency and a secretory signal sequence (murine Ig heavy chain leader). The leader is followed by four variable Ig domains as listed above. The linker peptide at the VL-VH or VH-VL junction of anti-EphA2 has a length of 15 amino acids (three repeats of the motif G₄S; SEQ ID NO:59). The linker peptide connecting the EphA2 with the CD3 binding specificity comprises one repeat of the motif G₄S (SEQ ID NO:58). Directly adjacent to the fourth domain is a C-terminal hexa-histidine (H6) sequence (SEQ ID NO:66) for detection and purification purposes. The indicated restriction enzyme sites were used for cloning the BiTE constructs into the expression vector.

6.1.1.1. Vectors for BiTE Expression in CHO Cells

The pEF-DHFR vector is a 5.8 kb vector with the murine dihydrofolate reductase as selection marker forming a bi-cistronic transcription unit together with the gene to be expressed under the control of the human EF1α promoter. The eukaryotic expression vector is a derivative of the expression vector pMT2PC.

The EF1α promoter is followed by a multiple cloning site, an internal ribosomal entry site (IRES), the DHFR gene and a polyadenylation signal. Additional control sequences present in the vector are the bacterial origin of replication (ORI) and the gene for Ampicillin resistance (bla). In the following table, the various elements of the cDNA expression vector are summarized.

| Structure | Origin | | Function |
|---|---|---|---|
| Promoter of elongation factor 1α | EF1α-P | human | Control of the construct inserted |
| (Mizushima and Nagata 1990) | | | into the MCS and the DHFR |
| | | | selection marker |
| Multiple cloning site | MCS | synthetic | Polylinker with restriction sites for |
| | | | EcoRI, SmaI, XbaL and SalI |
| Internal ribosomal entry site | IRES | viral (polio) | Bi-cistronic expression of the |
| derived from human poliovirus | | | construct inserted into the MCS |
| RNA | | | and DHFR |
| (Pelletier and Sonenberg 1988) | | | |
| Dihydrofolate reductase | DHFR | murine | Marker for selection in mammalian |
| | | | cells suited for subsequent gene |
| | | | amplification |
| Polyadenylation signal derived | SV40 | viral | Transcription termination and |
| from early SV40 mRNA | poly A | | mRNA processing |
| Non-translated adenoviral tran- | VA 1 | viral | No effect in pEF-DHFR due to |
| script acting on tripartite leader | | | deletion of the adenovirus major |
| (TPL) of adenovirus major late | | | late promoter (MLP) |
| promoter (MLP) | | | |
| Bacterial origin of replication | ori | bacterial | Replication in E. coli |
| from pUC18 | | | |
| Ampicillin resistance gene | bla | bacterial | Marker for selection in E. coli |
| (Amp^{R} gene) | | | |

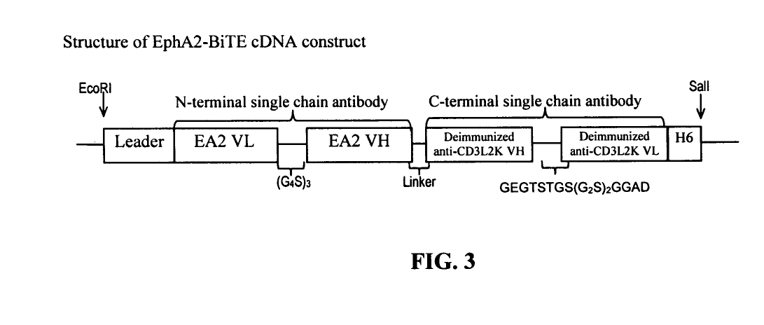

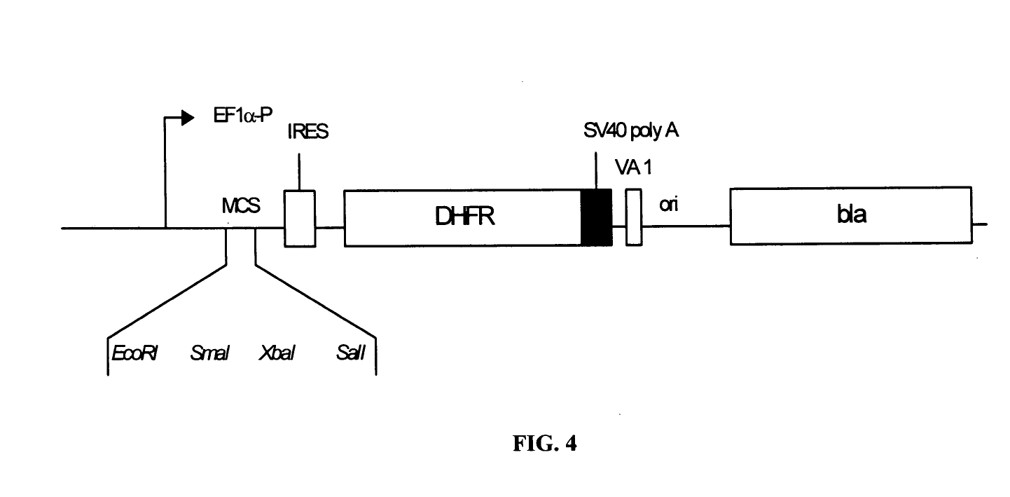

A depiction of the vector is shown in FIG. 4.

6.1.1.2. Host CHO Cell Line

The Chinese Hamster Ovary (CHO) cell line CHO/dhfr⁻, CRL 9096, was purchased from the American Type Culture Collection (“ATCC”; Manassas, Va.), and was characterized by Q-One, UK. Hypoxanthine and thymidine have to be added as supplements to the growth medium because the CHO/dhFr-cell line is deficient in dihydrofolate reductase.

6.1.1.3. BiTE Expression in CHO Cells

After transfection of the CHO/dhfr− cells with the eight expression vectors encoding EphA2 based BiTE constructs, each cell pool was cultivated in nucleoside-free HyQ PF CHO liquid soy medium (with 4.0 mM L-Glutamine with 0.1% Pluronic F-68; Cat.# SH30359.02; HyClone) for selection of DHFR-positive transfectants. Subsequently, the pool of transfected cells was subjected to a single gene amplification step using DHFR inhibitor methotrexate (MTX) at a concentration of 20 nM as a supplement to nucleoside-free medium.

6.1.1.4. Purification of EphA2-BiTEs from Culture Supernatants

Akta® FPLC System (Amersham) and Unicorn Software were used for chromatography. Immobilized metal affinity chromatography (“IMAC”) was performed using a Fractogel® column (Merck) which was loaded with ZnCl₂ according to the protocol provided by the manufacturer. The column was equilibrated with buffer A (20 mM sodium phosphate buffer pH 7.5, 0.4 M NaCl) and the cell culture supernatant (500 ml) was applied to the column (10 ml) at a flow rate of 3 ml/min. The column was washed with buffer A to remove unbound sample. Bound protein was eluted using a 2 step gradient of buffer B (20 mM sodium phosphate buffer pH 7.5, 0.4 M NaCl, 0.5 M Imidazol) according to the following:

Step 1: 10% buffer B in 6 column volumes;

Step 2: 100% buffer B in 6 column volumes.

Eluted protein fractions from step 2 were pooled for further purification.

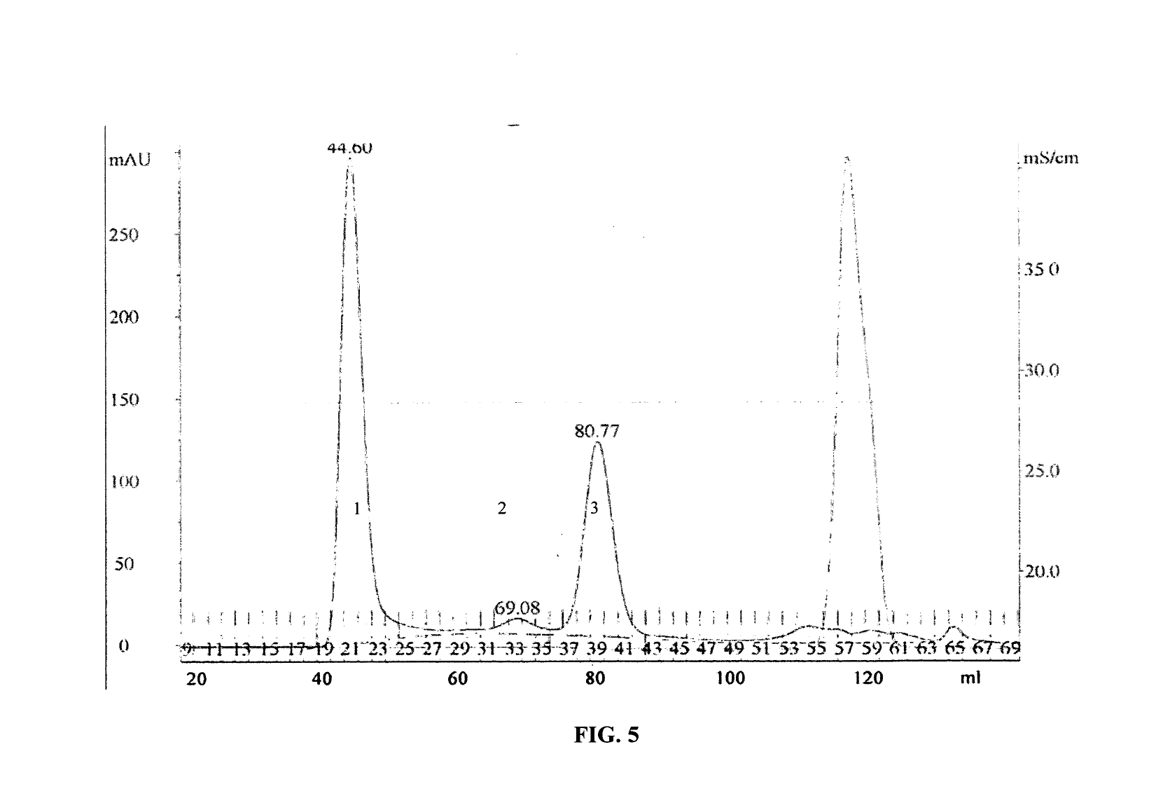

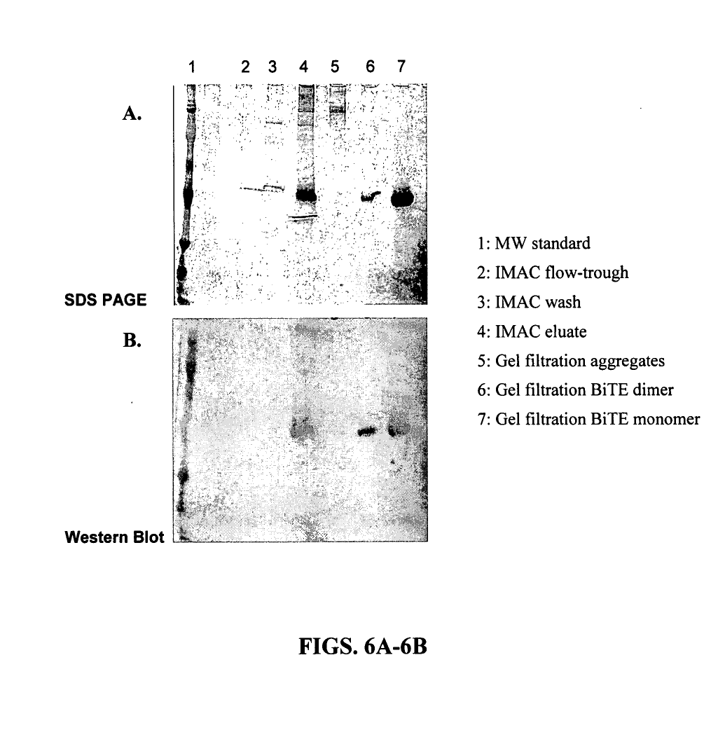

Gel filtration chromatography was performed on a Sephadex 200 HiPrep column (Amersham) equilibrated with PBS (Gibco) (FIG. 5). Eluted protein samples (at flow rate of 1 ml/min) were subjected to standard SDS-PAGE and Western Blot for detection (FIG. 6). Prior to purification, the column was calibrated for molecular weight determination (molecular weight marker kit, Sigma MW GF-200). Protein concentrations were determined using protein assay dye (MicroBCA, Pierce) and IgG (Biorad) as standard protein.

6.1.2 Surrogate EphA2 BiTEs Reactive with Macaque CD3 for Toxicity Studies

6.1.2.1. Flow Cytometric Binding Analysis of Cynomolgus-Reactive Anti-CD3 Parental Antibodies

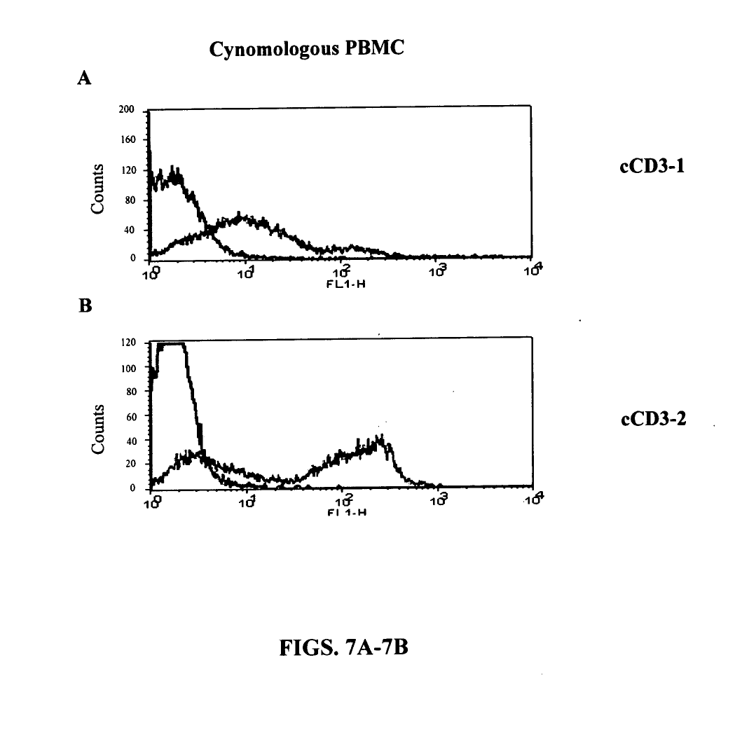

As a starting point for the EphA2-BiTE constructs to be tested for toxicology in cynomolgus monkeys, peripheral blood mononuclear cells (“PBMCs”) from cynomolgus monkeys were used to detect CD3 binding of two cynomolgus-reactive anti-CD3 parental antibodies, cCD3-1 and cCD3-2, both conjugated with FITC. For each sample 200,000 cells were incubated with the respective antibodies for 30 min on ice. Subsequently the cells were washed twice with PBS. As negative control an irrelevant antibody was used (black line). CD3-binding is represented by gray lines in the histogram overlays. Cells were analyzed by flow cytometry on a FACS-Calibur (Becton Dickinson, Heidelberg). Both antibodies show distinct binding to the T-cell fraction of cynomolgus PBMC. See FIG. 7.

6.1.2.2. Surrogate Molecules with a Substitute Target Specificity

The variable domains of the anti-CD3 parental antibodies cCD3-1 and cCD3-2 were used to construct BiTE molecules with substitute target specificity. Single-chain antibodies in different domain arrangements were created by PCR-based fusion. The following BiTE constructs were generated by combining these specificities with a substitute target specificity:

| N-terminal Position | C-terminal Position |
|---|---|
| Substitute Single Chain | cCD3-1 (VL/VH) |
| (VL/VH of target SCA) | cCD3-1 (VH/VL) |
| | cCD3-2 (VL/VH) |
| | cCD3-2 (VH/VL) |

Cloning and transfection of the surrogate constructs were carried out as described above.

6.1.2.3. Evidence For Cross-Reactivity of mAbs EA2 and EA5 with Rhesus EphA2

Sequencing of rhesus EphA2 from the rhesus CMMT110/CL line (97.7% homology to human EphA2) and partial sequencing of cynomolgus spleen cells (>99% homology to human EphA2) suggested that the human EphA2-specific monoclonal antibodies EA2 and EA5 may be able to cross-react with the non-human primate EphA2 molecules. Indeed, EA2 and EA5 antibodies activated phosphorylation of EphA2 in CMMT110/CL cells (FIG. 8), and weakly bound surface rhesus EphA2 on CMMT110/CL cells as measured by flow cytometry (1.2-1.8 fold change in MFI versus isotype control).

6.1.2.4. Construction of Macaque CD3-Reactive EA2 and EA5 BiTEs

Plasmids with coding sequences for variable chains of the anti-EphA2 antibodies EA2 and EA5 and macaque CD3-specific single-chain antibodies cCD3-1 and cCD3-2 were constructed by PCR-based fusion. The following twelve BiTE molecules were made:

| N-terminal Position | C-terminal Position |
|---|---|
| EA5 (VH/VL, EA5HL) | cCD3-1LH |
| | cCD3-1HL |
| | cCD3-2LH |
| | cCD3-2HL |
| EA2 (VH/VL, EA2HL) | cCD3-1LH |
| | cCD3-1HL |
| | cCD3-2LH |
| | cCD3-2HL |
| cCD3-1LH | EA2 (VH/VL, EA2HL) |
| cCD3-1HL | |
| cCD3-2LH | |
| cCD3-2HL | |

Cloning and transfection of the surrogate constructs were carried out as described above.

6.1.2.5. Flow Cytometric Binding Analysis of Various Anti-EphA2 Surrogate BiTE Constructs Reacting with Macaque CD3

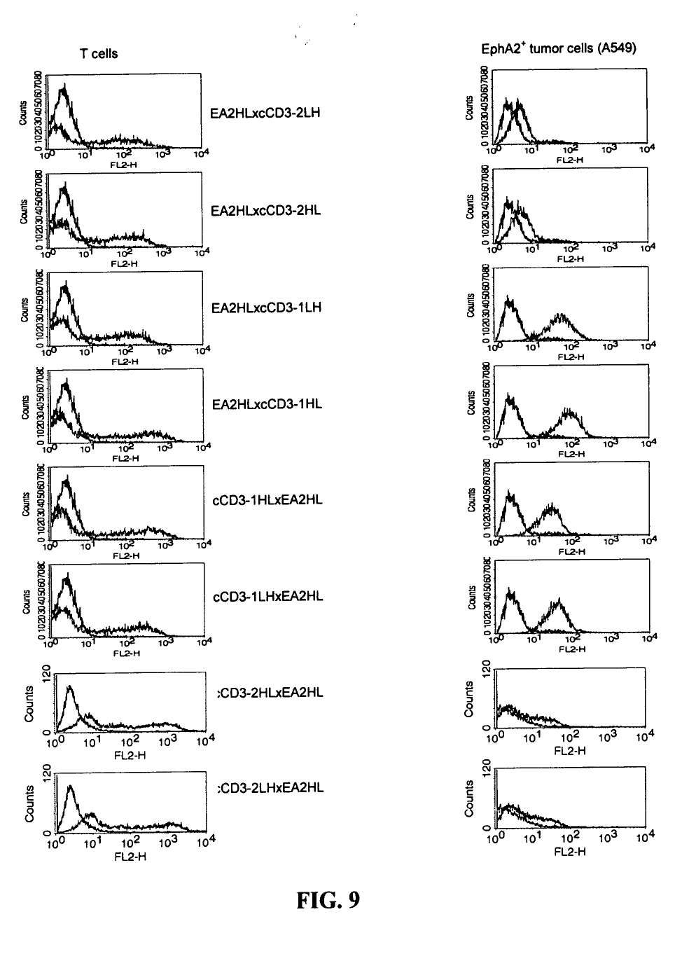

For each sample 200,000 cells (T-cells or EphA2+ tumor cells) were incubated with 50 μl pure cell culture supernatant of CHO cells transfected with the various Anti-EphA2 surrogate BiTE constructs for 30 min on ice. The cells were washed subsequently twice with PBS and bound construct was detected via its C-terminal Histidine Tag with a murine Penta His antibody (diluted to 5 μg/ml in 50 μl PBS with 2% FCS; Qiagen; Order No. 34660) followed by a washing step and a Phycoerythrin conjugated murine Fc gamma specific antibody (Dianova, order no. 115-116-071), diluted 1:100 in 50 μl PBS with 2% FCS (gray line). As a negative control, cell culture supernatants of untransfected cells was used (black line). Cells were analyzed by flow cytometry on a FACS-Calibur (Becton Dickinson, Heidelberg). As shown in FIG. 9, only those surrogate BiTE constructs based on macaque CD3 antibody cCD3-1 showed strong binding to CD3 and EphA2. Constructs based on cCD3-2 only show strong CD3-binding; the EphA2-binding turned out to be almost completely suppressed.

6.2 EphA2-BiTE Characterization

6.2.1 Flow Cytometric Binding Analysis of Anti-EphA2 Parental Antibodies

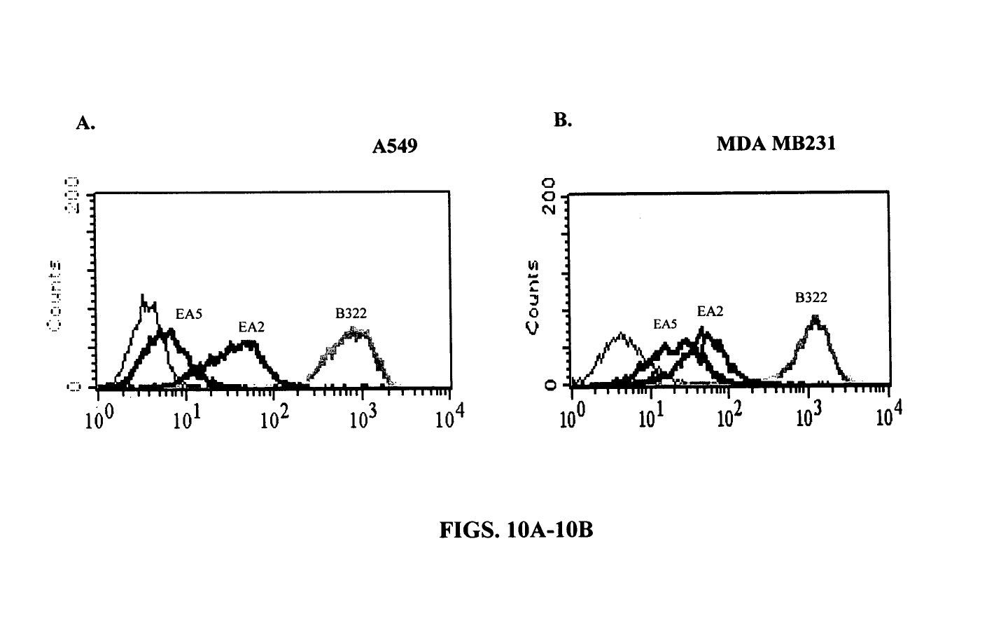

A flow cytometric analysis was performed to estimate the binding strength of the anti-EphA2 parental monoclonal antibodies B233, EA2 and EA5 (FIG. 10). See FIG. 29 for VL and VH domain sequences of the B233 monoclonal antibody. EphA2-expressing A549 cells (human lung carcinoma cell line) and MDA-MB-231 cells (human breast cancer) were used. 200,000 cells were incubated with 10 μg/ml of the respective antibody for 30 min on ice. The cells were subsequently washed twice in PBS. The binding of the primary antibody was detected via an phycoerythrin conjugated murine Fc-gamma specific antibody (Dianova, order no. 115-116-071) diluted 1:100 in 50111 PBS with 2% FCS. As negative control, an irrelevant antibody with the same isotype was used (thin line). Cells were analyzed by flow cytometry on a FACS-Calibur (Becton Dickinson, Heidelberg).

mAb B233 showed the strongest binding signal followed by EA2 and EA5. The binding capabilities of the three different antibodies are shown in a histogram overlay.

6.2.2 Tissue Cross-Reactivity (TCR) of Anti-EphA2 Parental Antibodies EA2 and EA5

Frozen human tissue sections were stained with the human EphA2-reactive monoclonal antibodies EA5 and EA2 using immunohistochemical methods to determine if the antibodies specifically bind to normal tissue. Briefly, a pre-complex of primary and secondary antibodies was produced with the unbound secondary binding sites blocked with appropriate species gamma globulins. Frozen sections were adhered to slides in 10% formalin and rinsed with 1× Tris-buffered saline (TBS) with 0.01% Tween 20. Endogenous peroxidases were blocked with a solution containing glucose oxidase (Sigma), β-D(+)-glucose (Sigma), and sodium azide (Sigma). An avidin/biotin vector kit (Vector Labs) blocked avidin/biotin reactive sites. The slides were then incubated with a protein blocking solution consisting of bovine serum albumin, casein, and normal goat serum. Tissue sections were incubated with precomplexed antibodies and then Vectastain ABC Elite Kit (Vector Labs), rinsed with 1×TBS, treated with DAB (Sigma), and counterstained with hematoxylin. Tissue sections were dehydrated, coverslipped, and imaged.

As shown in FIG. 11, EA5 demonstrated staining of intercalated discs in heart tissue (FIG. 11B), vascular and stromal smooth muscle elements of multiple organs (cytoplasm), colonic epithelium (cytoplasm), and uterine myometrium. Human tissue sections (FIG. 11C) were not stained with EA2 as compared with an isotype control monoclonal antibody 1A7, and as summarized by the table below.

| | 2 μg/ml | 2 μg/ml | Assay |
|---|---|---|---|
| Tissue | EA2 | 1A7 | cont. |
| MDA 231 cells | 1+ | Neg | Neg |
| Ovary | Neg | Neg | Neg |
| Stromal fibroblasts (cytoplasm) | | | |
| Pancreas | Neg | Neg | Neg |
| Spleen | Neg | Neg | Neg |
| Vascular and trabecular smooth muscle | | | |
| (cytoplasm) | | | |
| Adrenal | Neg | Neg | Neg |
| Testis | Neg | Neg | Neg |
| Interstitial and vascular smooth muscle | | | |
| (cytoplasm) | | | |
| Lymph Node | Neg | Neg | Neg |
| Stromal fibroblasts and vascular smooth | | | |
| muscle (cytoplasm) | | | |
| Urinary Bladder | Neg | Neg | Neg |
| Smooth muscle (cytoplasm) | | | |
| Kidney | Neg | Neg | Neg |
| Liver | Neg | Neg | Neg |
| Vascular smooth muscle (cytoplasm) | | | |
| Skin | Neg | Neg | Neg |
| Vascular smooth muscle (cyto) | | | |
| Lung | Neg | Neg | Neg |
| Vascular smooth muscle (cytoplasm) | | | |
| Prostate | Neg | Neg | Neg |
| Stromal and vascular smooth muscle | | | |
| (cytoplasm) | | | |
| Uterus | Neg | Neg | Neg |
| Myometrium | | | |
| (cytoplasm) | | | |
| Colon | Neg | Neg | Neg |
| Epithelium | | | |
| (cytoplasm) | | | |

Neg = Negative; 1+—weak; 2+—moderate; 3+ = strong; 4+ = intense
MDA 231 cells were used as a positive control for this assay.

6.2.3 Bispecific Cell Binding of EphA2-BiTE Constructs

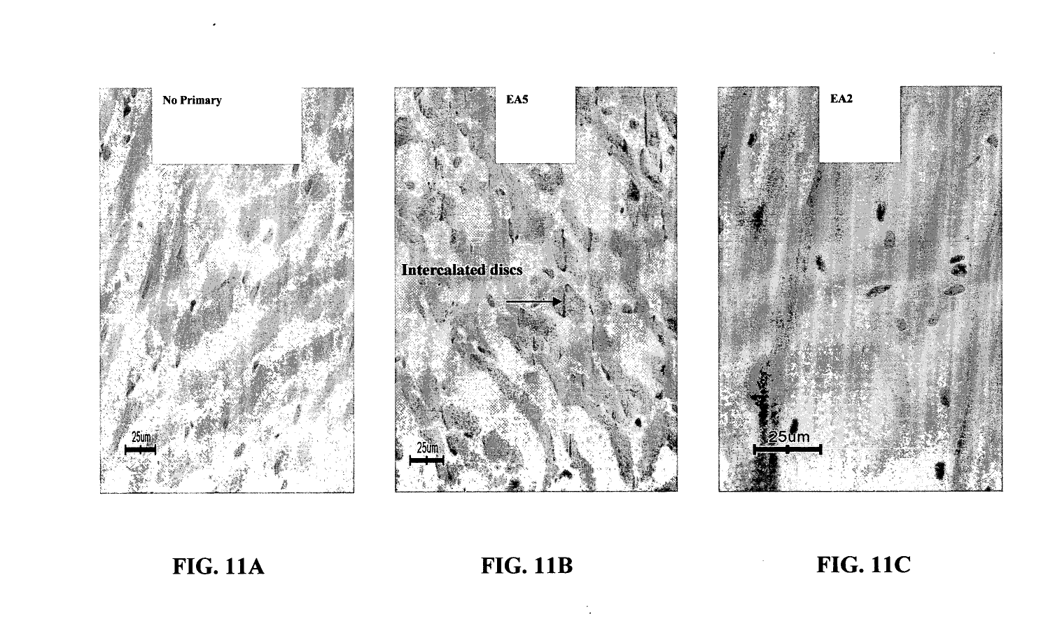

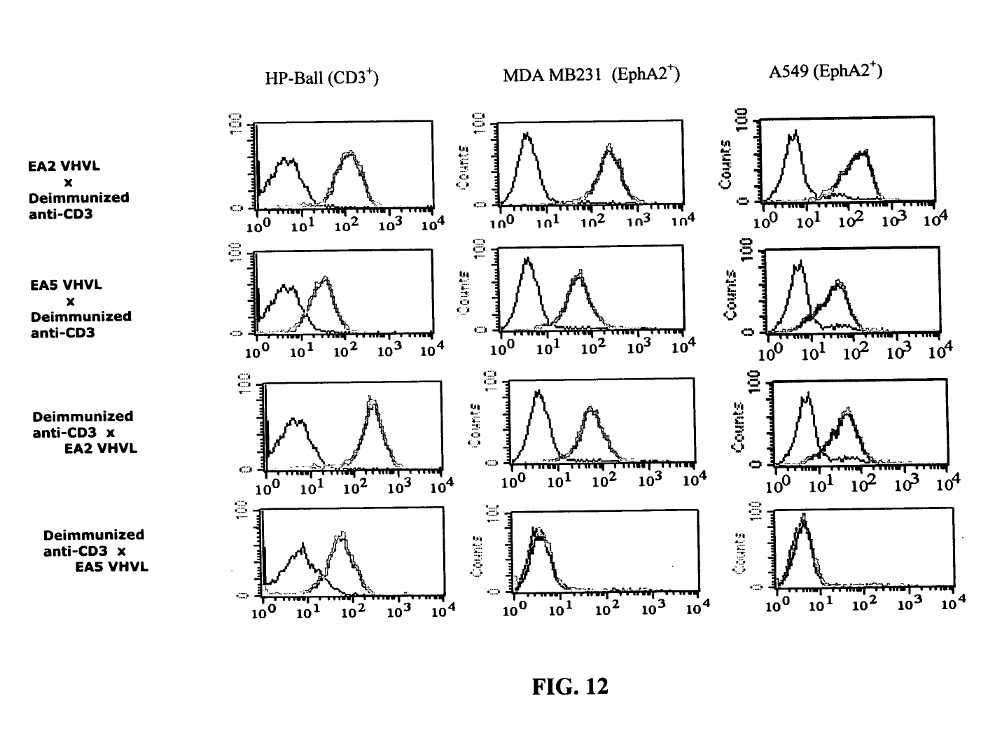

EphA2-positive A549 cells (human lung carcinoma cell line) and MDA-MB-231 cells (human breast cancer), as well as CD3-positive HP-BALL (human T-cell line) were used. 200,000 cells were incubated with 10 μg/ml of the purified BiTE monomer of four different EphA2 BiTE constructs for 30 min on ice. Cells were subsequently washed twice in PBS and bound construct was detected via its C-terminal hexahistidine tag with a murine penta-His antibody (diluted 1:20 in 50 μl PBS with 2% FCS; Qiagen; order no. 34660) followed by a washing step and a phycoerythrin conjugated murine Fc-gamma specific antibody (Dianova, order no. 115-116-071) diluted 1:100 in 50 μl PBS with 2% FCS (gray line). As a negative control, fresh cell culture medium from un-transfected CHO cells instead of culture supernatant from the transfectants was used (black line). Cells were analyzed by flow cytometry on a FACS-Calibur (Becton Dickinson, Heidelberg). See FIG. 12.

The following table summarizes the relative binding intensities of all eight generated EphA2-BiTE constructs as determined by flow cytometry.

| | | FACS Staining | | |
|---|---|---|---|---|
| | | MDA | | HP |
| EphA2 BiTE constructs | | MB231 | A549 | Ball |
| EA2 (VL/VH) | deimmunized | ++ | ++ | ++ |
| EA2 (VH/VL) | anti-CD3 | +++ | +++ | +++ |
| | (VH/VL) | | | |
| EA5 (VL/VH) | deimmunized | − | − | ++ |
| EA5 (VH/VL) | anti-CD3 | ++ | ++ | ++ |
| | (VH/VL) | | | |
| deimmunized | EA2 | ++ | ++ | +++ |
| anti-CD3 | (VL/VH) | | | |
| (VH/VL) | EA2 | ++ | ++ | +++ |
| | (VH/VL) | | | |
| deimmunized | EA5 | − | − | ++ |
| anti-CD3 | (VL/VH) | | | |
| (VH/VL) | EA5 | − | − | ++ |
| | (VH/VL) | | | |

All eight EA2 BiTE constructs showed more or less strong binding with their anti-CD3 SCA portion to CD3 expressed on HP Ball cells, indicating proper expression and folding of the anti-CD3 portion. All four BiTEs derived from mAb EA2 showed binding to EphA2-positive breast and lung cancer cell lines, whereas only one BiTE derived from mAb EA5 retained EphA2-specific binding activity. Monoclonal antibody EA2 thus appeared to be more suitable for BiTE construction than mAb EA5.

6.2.4 Epitope Exclusion by EphA2 BiTEs

The EphA2-positive non-transformed breast epithelial line MCF10A and the breast adenocarcinoma MDA-MB-231 were used in an immunofluorescence staining experiment to determine if EphA2 BiTE constructs (used at 10 μg/ml) retained epitope exclusion as their parental monoclonal antibodies EA2 and EA5. The results of the analysis are summarized in the table below. BiTE EA2 (VH/VL)-deimmunized anti-CD3 showed the strongest epitope exclusion.

| | | MDA-MB-231 | |
|---|---|---|---|
| Reagent | MCF10A Staining | Staining | Epitope Exclusion |
| mAb B233 | + | + | − |
| (+Control) | AT-cell-cell contacts | At membrane ruffles | |
| BiTE | +/− | +/− | +++ |
| EA2(VH/VL) | Weak & Diffuse | Diffuse Staining | Strong epitope |
| | | | exclusion |
| BiTE | +/− | +/− | + |
| EA2(VL/VH) | Weak & Diffuse | Diffuse Staining | Suggests epitope |
| | | | exclusion |
| BiTE | +/− | +/− | ++ |
| EA5(VH/VL) | Weak & Diffuse | Diffuse Staining | Epitope exclusion |
| BiTE | +/− | +/− | + |
| deimmunized | Weak & Diffuse | Diffuse Staining | Suggests epitope |
| anti-CD3- | | | exclusion |
| EA2(VH/VL) | | | |

6.2.5 Productivity of Monomeric EphA2-BiTEs

The productivity of five active EphA2 BiTEs was calculated from the monomer yield of a small scale production in roller bottles. Productivities of up to 1.1 mg/l purified monomer were obtained (see table below).

The productivity of a medium-scale production (2×10 L reactor) of a pool of amplified CHO cells (20 nM MTX) transfected with BiTE construct deimmunized anti-CD3×EA2(VH/VL) was 1.1 mg purified BiTE monomer/L cell culture supernatant.

| N-terminal Position | C-terminal Position | Production Level | Monomer:Dimer |
|---|---|---|---|
| EA2 (VL/VH) | deimmunized anti- | 1100 μg/l monomer | 1:0.13 |
| EA2 (VH/VL) | CD3 (VH/VL) | 664 μg/l monomer | 1:0.1 |
| EA5 (VL/VH) | deimmunized anti- | (no EphA2 binding) | |
| EA5 (VH/VL) | CD3 (VH/VL) | 306 μg/l monomer | 1:1.2 |
| deimmunized anti- | EA2 (VL/VH) | 153 μg/l monomer | 1:0.9 |
| CD3 (VH/VL) | EA2 (VH/VL) | 841 μg/l monomer | 1:0.1 |

| | | |
|---|---|---|
| deimmunized anti- | EA5 (VL/VH) | (no EphA2 binding) |
| CD3 (VH/VL) | EA5 (VH/VL) | (no EphA2 binding) |

6.2.6 Potency of Redirected Lysis of Five EphA2-BiTE Constructs

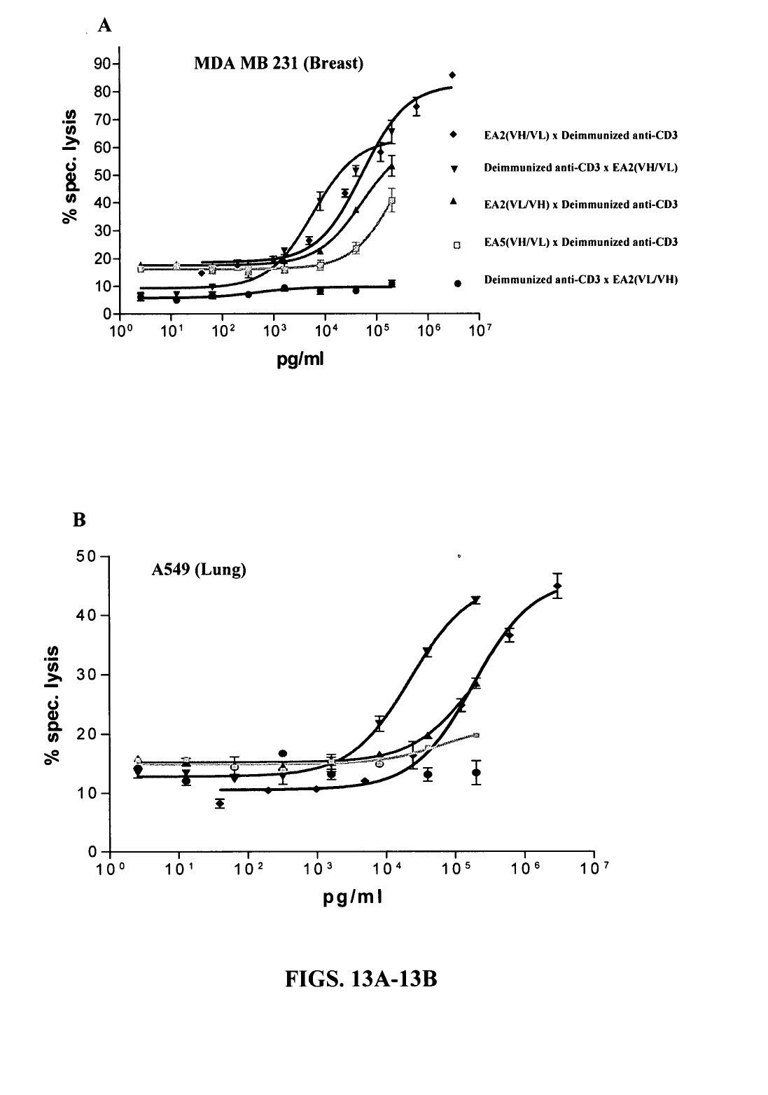

Cytotoxicity assays based on Chromium-51 release were performed to determine redirected target cell lysis by the various EphA2-BiTE constructs in the presence of human T-cells. In brief, PBMCs as a source of effector T-cells were obtained by Ficoll density gradient centrifugation. NK cells were depleted from the PBMC by CD16-directed magnetic beads to avoid T-cell independent cell lysis. EphA2-positive tumor cells lines MDA-MB231 and A549 were loaded with Chromium-51 and served as target cells. The various EphA2-BiTE constructs were titrated over a broad range of concentrations. The assay duration was 18 hours, and the effector-to-target ratio (E:T) 10:1. See FIG. 13.

The following table summarizes the BiTE concentrations required for half-maximal redirected cell lysis (i.e., EC50) of EphA2-positive target cell lines MDA MB231 and A549, respectively, as determined in two independent experiments. The following table summarizes the BiTE concentrations required for half-maximal redirected cell lysis (i.e., EC50) of EphA2-positive target cell lines MDA MB231 and A549, respectively, as determined in two independent experiments.

| | | EC₅₀ [ng/ml] | |
|---|---|---|---|
| EphA2-BiTE Constructs | | MDA MB231 | A549 |
| EA2 (VL/VH) | deimmunized | 53.8 | 170.1 |
| | anti-CD3 | 86.0 | not detectable |
| EA2 (VH/VL) | (VH/VL) | 55.9 | 183.9 |
| | | 72.1 | 163.4 |
| EA5 (VL/VH) | deimmunized | (no EphA2 binding) | |
| EA5 (VH/VL) | anti-CD3 | Very weak cytotoxicity | |
| | (VH/VL) | | |
| deimmunized | EA2 | Very weak cytotoxicity | |
| anti-CD3 | (VL/VH) | | |
| (VH/VL) | EA2 | 5.8 | 122.6 |
| | (VH/VL) | 8.6 | 24.8 |

| | | |
|---|---|---|
| deimmunized | EA5 | (no EphA2 binding) |
| anti-CD3 | (VL/VH) | |
| (VH/VL) | EA5 | (no EphA2 binding) |
| | (VH/VL) | |

The BiTE construct deimmunized anti-CD3×EA2(VH/VL) consistently showed the highest potency in redirected lysis of breast and lung cancer cell lines.

6.2.7 Selection of BiTE Deimmunized Anti-CD3×EA2(VH/VL) For Further Characterization

Since deimmunized anti-CD3×EA2(VH/VL) was found to be the most potent EphA2-BiTE construct in redirected lysis (ED50 values between 6 and 25 ng/ml with two target cell lines), and had the second best productivity of monomer (0.8-1.1 mg/l) of all five BiTE constructs that showed bispecific binding activity, it was selected for further characterization. See FIG. 14B for the nucleotide and amino acid sequence of the deimmunized anti-CD3×EA2 BiTE construct.

6.3 Further Characterization of the EphA2-BiTE “Deimmunized Anti-CD3×EA2 (VH/VL)”

6.3.1 Variation of Cytotoxic Activity with Production Batch and Effector Cell Donor

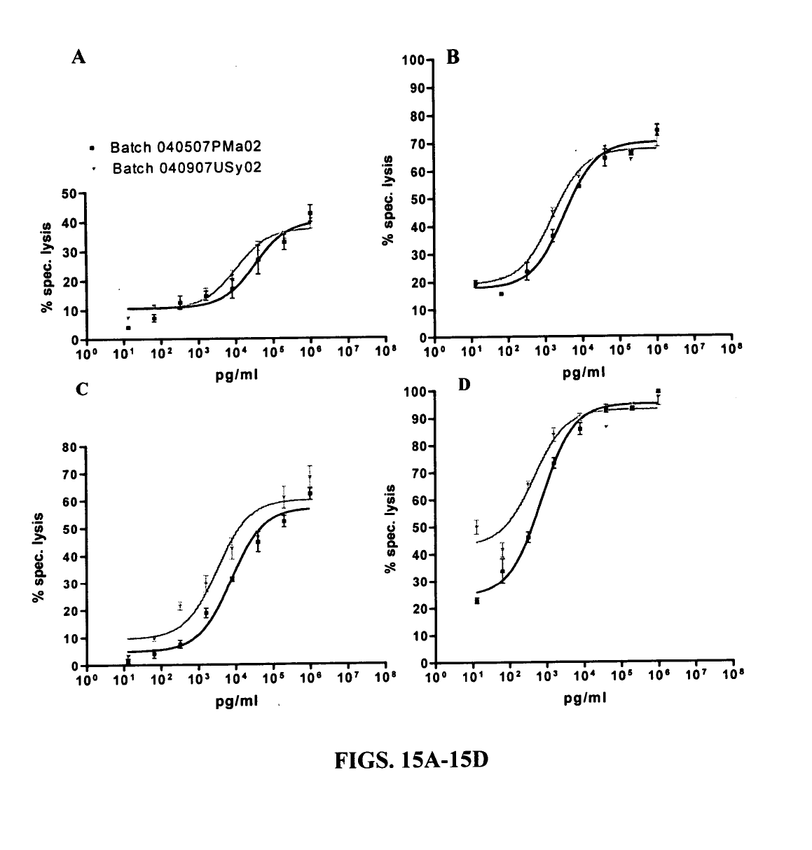

The cytotoxic activity of two different production lots of the EphA2-BiTE deimmunized anti-CD3×EA2(VH/VL) was compared using PBMCs (after depletion of CD16 positive cells) and stimulated CD8+ T-cells as effector cells. The EphA2-positive cell lines A549 and MDA-MB231 served as target cells. The E:T ratio was 10:1, and the incubation time was 18 hours. See FIG. 15.

The following table summarizes the half-maximal lysis (i.e., EC50) values for redirected target cell lysis obtained from the above dose-response analyses with the EphA2 BiTE from different production batches using either PBMC or stimulated CD8+ T-cells as effector cells.

| | |
|---|---|
| Effector | EC₅₀ [ng/m] |

| | Cells | A549 | MDA MB 231 |
|---|---|---|---|
| Batch | PBMC | 32.8 | 7.6 |
| 040507PMa02 | CD8 T-cells | 3.3 | 0.7 |
| Batch | PBMC | 10.2 | 3.2 |
| 040907USy02 | CD8 T-cells | 1.6 | 0.5 |

The variation of ED50 values for redirected target cell lysis was tested for the EphA2-BiTE deimmunized anti-CD3×EA2(VH/VL) with seven human PBMC donors

| | |
|---|---|
| Source of Effector | |
| Cells | EC₅₀ [ng/ml] |

| NK cell-depleted | | MDA-MB | |
|---|---|---|---|
| PBMC | A549 | 231 | Batch |
| Donor #1 | 20.9 | 15.4 | 040305PMa04 |
| Donor #2 | 22.6 | 5.8 | 040305PMa04 |
| Donor #3 | 24.8 | 8.6 | 040305PMa04 |
| Donor #4 | 28.5 | 23.9 | 040507PMa02 |
| Donor #5 | not done | 1.9 | 0405O7PMa02 |
| Donor #6 | 32.8 | 7.6 | 040507PMa02 |
| Donor #7 | 6.6 | 3.2 | 040907USy02 |

The EphA2 BiTE deimmunized anti-CD3×EA2(VH/VL) showed considerably higher cytotoxicity with stimulated CD8+ T-cells than with NK cell-depleted PBMC. Variation of biological activity between two different batches was in the range of 1.4 to 3.2-fold. Variation of potency was however more pronounced with human PBMC donors, where ED50 values between 1.9 and 23.9 ng/ml were observed. Donor-specific efficacy of T-cells may thus be a major source of BiTE activity variation.

Flow cytometry-based redirected cell cytotoxicity assays were also performed to determine variation of EphA2-BiTE cytotoxic activity with effector cell donors. CD3+ T-cell enriched PBMCs were used as effector cells and EphA2+SW480 colon carcinoma cells were used as target cells. Briefly, CD3+ T-cell enriched human PBMC (RosetteSep; StemCell Technologies) were isolated from healthy donors by ficoll density gradient centrifugation. Target cells were labeled with 3,3′-dioctadecyloxacarbocyanine (DiOC 18(3) or “DiO”; (Molecular Probes)) green fluorescent membrane dye for 5 minutes at 37° C. Effector and target cell mixtures were combined at a 5:1 ratio and transferred to a 96-well round bottom plate. Medium alone or serial dilutions of each BiTE construct was added to appropriate wells, incubated for 18 or 42 hours at 37° C., and analyzed by flow cytometry after addition of propidium iodide (PI) to a final concentration of 1 μg/ml. Target cell lysis was calculated as the percentage of DiO positive cells staining positive by PI. All incubations were conducted in duplicate. Calculation of EC50 values was conducted using a four parameter nonlinear fit model.

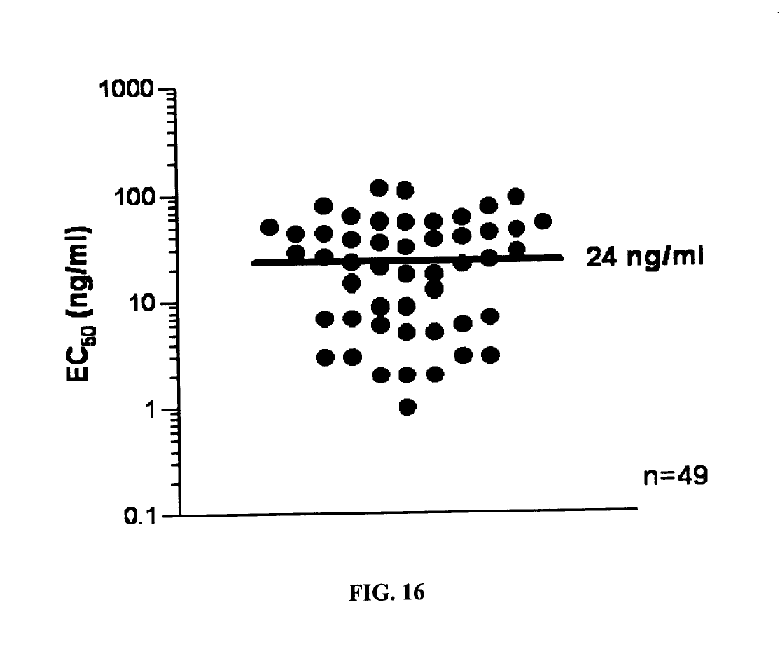

As shown in FIG. 16, EphA2-BiTE redirected T-cells from different subjects to mediate EphA2+ tumor cell killing. The flow cytometry-based cytotoxicity assay utilized SW480 target cells and CD3+ T-cells isolated from 49 individual human donors. For the majority of human donors, EphA2-BiTE mediated tumor cell killing at very low concentrations. The EC50 for EphA2-BiTE was between 1 and 110 ng/ml for all of the T-cell donors tested with a median (horizontal bar) of 24 ng/ml. Thus, for the majority of human T-cell donors, the EphA2-BiTE is a highly potent molecule with killing activity observed at concentrations in the low ng/ml range.

6.3.2 Specificity of Target cell Binding: Soluble EphA2 Fusion Protein Competes for Binding with Deimmunized Anti-CD3×EA2(VH/VL)

The target binding specificity of BiTE deimmunized anti-CD3×EA2(VH/VL) was tested in a competition assay. To this end, 200,000 EphA2-positive A549 cells were incubated with 5 μg/ml of the EA2 based BiTE construct either in the presence or absence of 50 μg/ml soluble EphA2 Fc fusion protein for 30 min on ice. The cells were subsequently washed twice in PBS. Binding of the construct was then detected via its C-terminal hexahistidine tag using a murine penta-His antibody (diluted 1:20 in 50 μl PBS with 2% FCS; Qiagen; order no. 34660) followed by a washing step and a phycoerythrin-conjugated murine Fc-gamma specific antibody (Dianova, order no. 115-116-071) diluted 1:100 in 50 μl PBS with 2% FCS (gray line). As negative control, only the secondary antibody was used (black line). Cells were analyzed by flow cytometry on a FACS Calibur instrument (Becton Dickinson, Heidelberg).

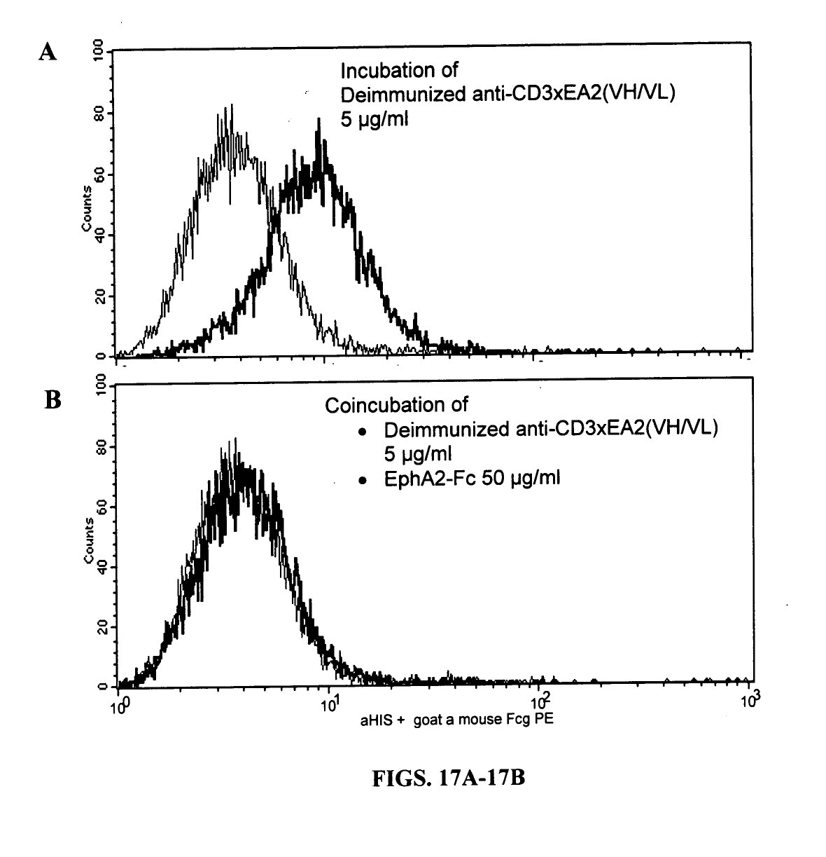

As shown in FIG. 17, coincubation of the EphA2-BiTE deimmunized anti-CD3×EA2 (VH/VL) with a 10-fold weight excess of soluble EphA2 Fc fusion protein completely blocked binding of the EphA2-BiTE to A549 human lung cancer cells. This shows that EphA2-BiTE binding to tumor cells is specifically mediated by recognition of the EphA2 target.

6.3.3 Target Cell Specificity of EphA2-BiTE Deimmunized Anti-CD3×EA2 (VH/VL): Killing of Antigen-Positive Cells

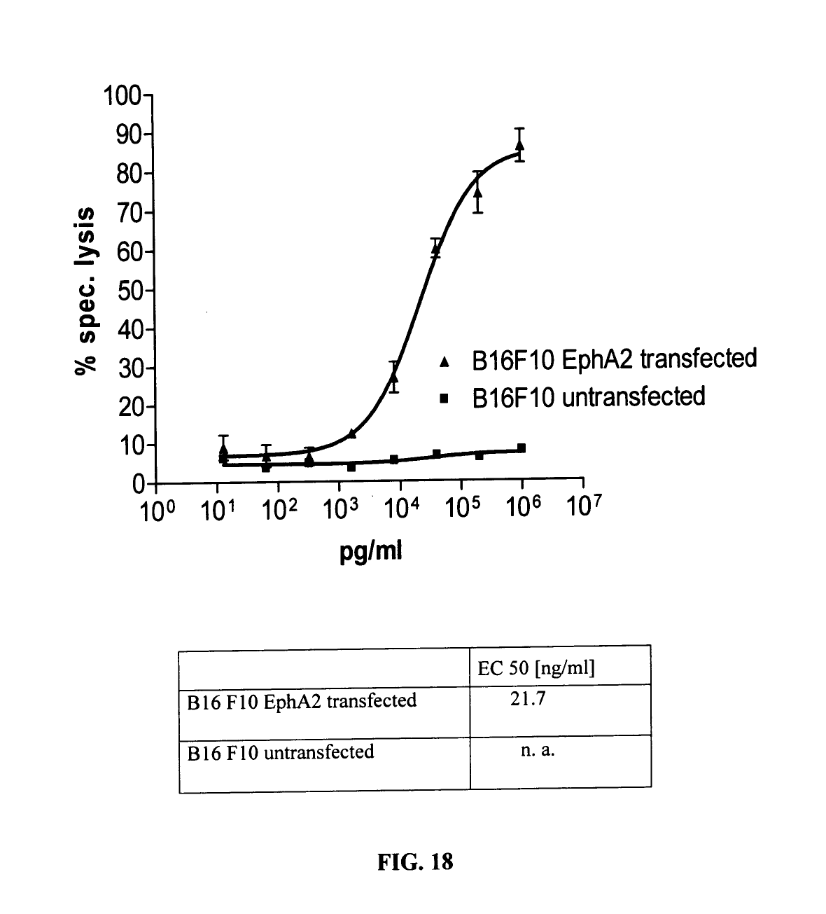

To ensure the retention of the target specificity after conversion from the original IgG into the BiTE format, a cytotoxicity assay with antigen positive and negative target cells was performed. For this purpose, EphA2-transfected B16F10 cells, a mouse melanoma cell line, were compared with untransfected cells. A standard chromium release assay using stimulated human CD8 T-cells as effectors at an E:T ratio of 10:1 and an assay duration of 18 hours was carried out with various concentrations of the EphA2-BiTE construct. See FIG. 18.

| | EC 50 [ng/ml] |
|---|---|
| B16 F10 EphA2 transfected | 21.7 |
| B16 F10 untransfected | n. a. |

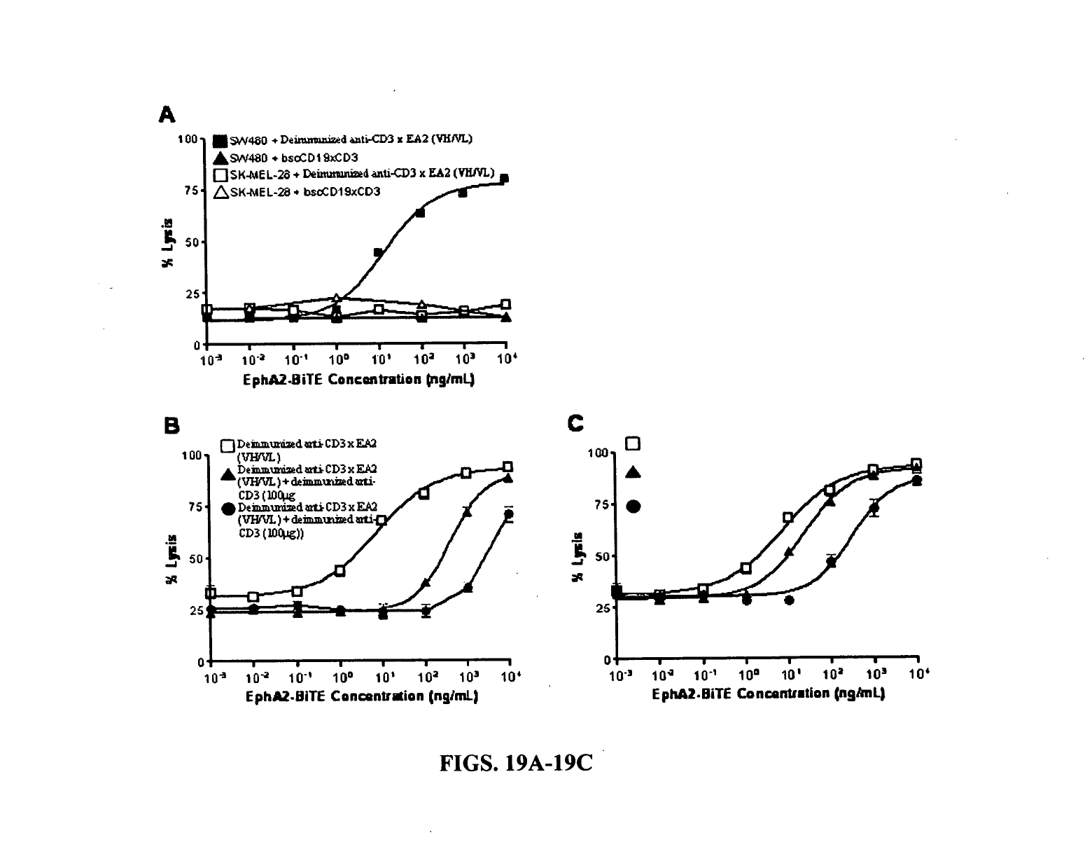

Specificity of target cell lysis was further substantiated by deimmunized anti-CD3×EA2 BiTE mediating lysis of EphA2+SW480 human colon carcinoma cells and not EphA2 negative SK-MEL28 melanoma cells. See FIG. 19. Parental antibodies specific for both EphA2 and CD3 blocked deimmunized anti-CD3×EA2 BiTE mediated lysis. Thus, cytotoxic activity of the deimmunized anti-CD3×EA2 BiTE strictly depends on the expression of EphA2 on the target cells; EphA2-negative cells are completely resistant to deimmunized anti-CD3×EA2 BiTE mediated lysis.

6.3.4 Renal Cell Carcinoma and Prostate Cancer Cell Killing Mediated by EphA2-BiTE

Flow cytometry-based redirected cellular cytotoxicity assays were conducted using CD3+ T-cell enriched human peripheral blood mononuclear cells (PBMC) as effector cells and EphA2+ACHN and Caki 2 renal cell carcinoma lines and PC3 and DU145 prostate cell lines as target cells. Briefly, CD3+ T-cell enriched human PBMC (RosetteSep; StemCell Technologies) were isolated from healthy donors by ficoll density gradient centrifugation. Target cells were labeled with 3,3′-dioctadecyloxacarbocyanine (DiOC18(3) or “DiO”; (Molecular Probes)) green fluorescent membrane dye for 5 minutes at 37° C. Effector and target cell mixtures were combined at a 5:1 ratio and transferred to a 96-well round bottom plate. Medium alone or serial dilutions of each BiTE construct was added to appropriate wells, incubated for 42 hours at 37° C., and analyzed by flow cytometry after addition of propidium iodide (PI) to a final concentration of 1 μg/ml. Target cell lysis was calculated as the percentage of DiO positive cells staining positive by PI. All incubations were conducted in duplicate. Calculation of EC50 values was conducted using a four parameter nonlinear fit model.

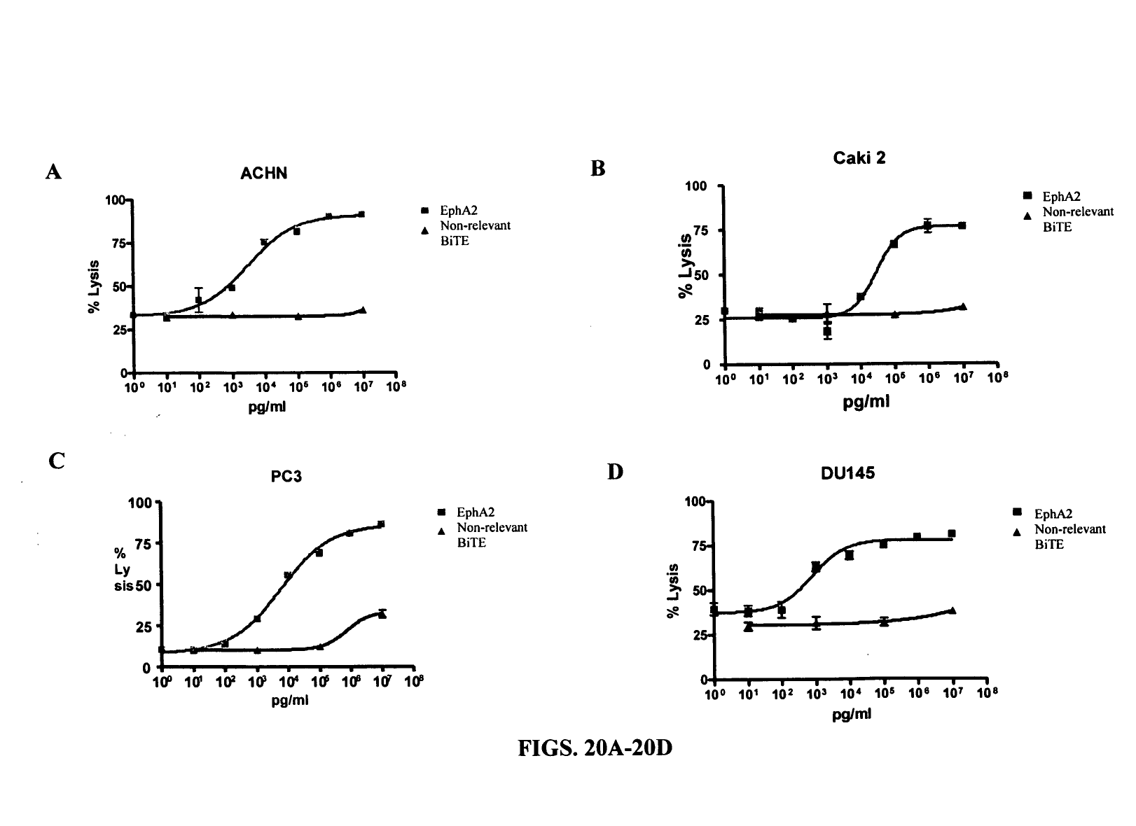

As shown in FIG. 20, EphA2-BiTE mediated redirected T-cell lysis of both renal cell carcinoma and prostate cancer cells.

6.3.5 Effects of EphA2-Bite Killing Upon EphA2 Levels on Target Cells

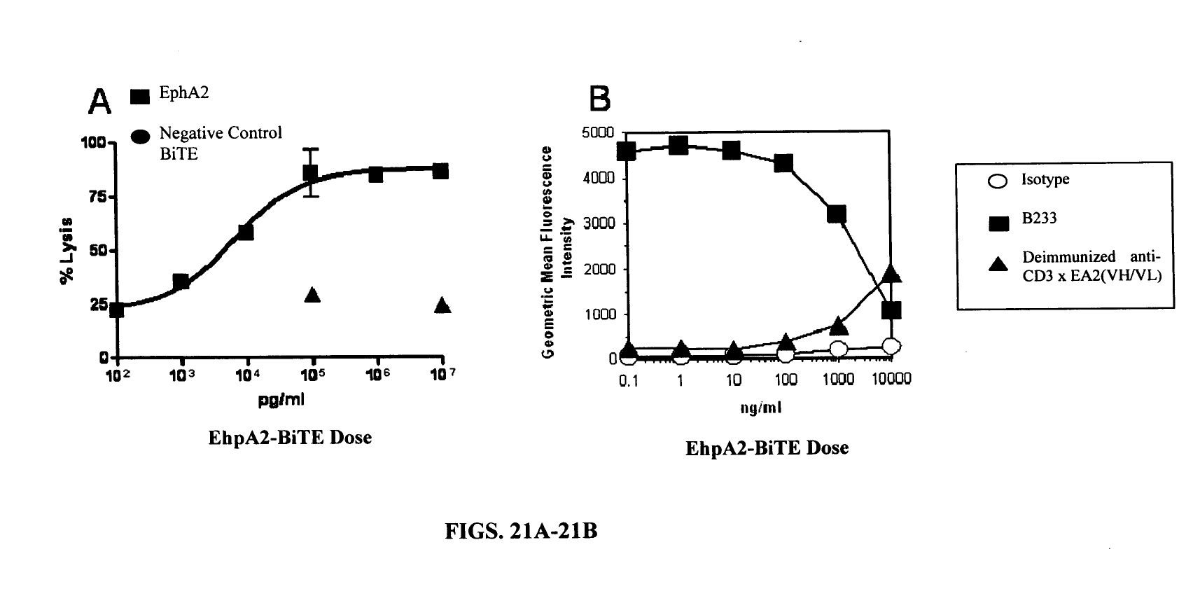

The cytotoxicity assay was performed as described above. Upon completion of the cytotoxicity assay, cultures were placed on ice and left untreated, or treated with 10 μg/ml of a non-binding isotype control mouse monoclonal antibody 1A7 or an anti-human EphA2 mouse monoclonal antibody B233 (see Dall'Acqua et al., 2005, Methods 36:43-60, which is incorporated by reference herein in its entirety). An APC-conjugated anti-pentahistidine antibody bound EphA2-BiTE that remained on the untreated cells. An APC-conjugated anti-mouse IgG (H+L) antibody resolved the amount of isotype or B233 bound to target cells. The geometric mean fluorescence intensity of isotype control, EphA2 and EphA2-BiTE versus input dose of EphA2-BiTE was graphed. See FIG. 21B.

6.3.6 Cytotoxicity Dependence on Time, Effector to Target Ratio, and Receptor Binding Sites

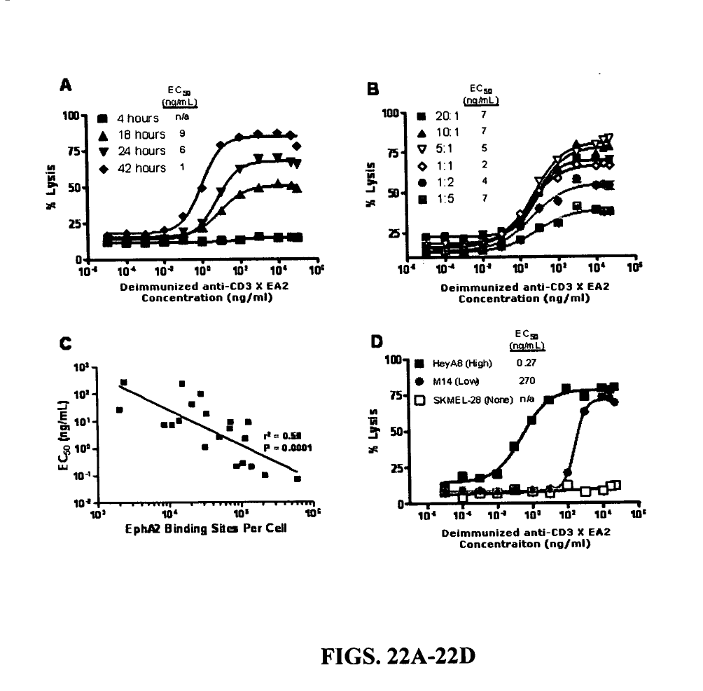

To explore the kinetics and potency of deimmunized anti-CD3×EA2 (VH/VL), several parameters of redirected tumor cell lysis were evaluated. An analysis of the time course of target cell killing revealed that in the presence of anti-CD3×EA2 (VH/VL), there was limited lysis by unstimulated CD3+ T-cells after 18 hours, and that maximal killing (>80% lysis) occurred by 42 hours (FIG. 22A). In most experiments, the magnitude of redirected T-cell lysis exceeded 80% of target cells. As an additional measure of potency, the ratio of effector to target cells was investigated. E:T ratios of 1:1 to 20:1 gave similarly high lytic activity, while E:T ratios of 1:2 and 1:5 still led to redirected lysis of SW480 tumor cells, albeit at a reduced percentage (FIG. 22B). Notably, the estimated EC50 values remained largely constant despite variations in incubation time (FIG. 22A; 1 to 9 ng/mL) or E:T ratio (FIG. 22B; 2 to 7 ng/mL).

Tumor cell targets, which expressed different levels of EphA2 on the surface, were evaluated to determine whether there was a threshold of surface target density required for the activity of anti-CD3×EA2 (VH/VL). Efficient redirected T-cell lysis was observed for all EphA2-expressing cell lines (FIG. 22C), including M14 cells, which expressed as few as 2,400 molecules of EphA2 per cell (FIG. 22D). The magnitude of lysis mediated by anti-CD3×EA2 (VH/VL) was similar for target cells irrespective of their surface target density (FIG. 22D). However, the surface density of EphA2 on target cells did have an impact on the efficiency of redirected lysis. A trend was observed in which the potency of anti-CD3×EA2 (VH/VL) increased as the number of EphA2 binding sites on the tumor cells increased (FIG. 22C). Together, these findings suggested that anti-CD3×EA2 (VH/VL) can potently and specifically redirect unstimulated human T-cells to lyse EphA2-expressing tumor cells, even when there are low levels of available binding sites on the tumor.

6.3.7 Stability of BiTE Deimmunized Anti-CD3×EA2 (VH/VL) in Human Plasma

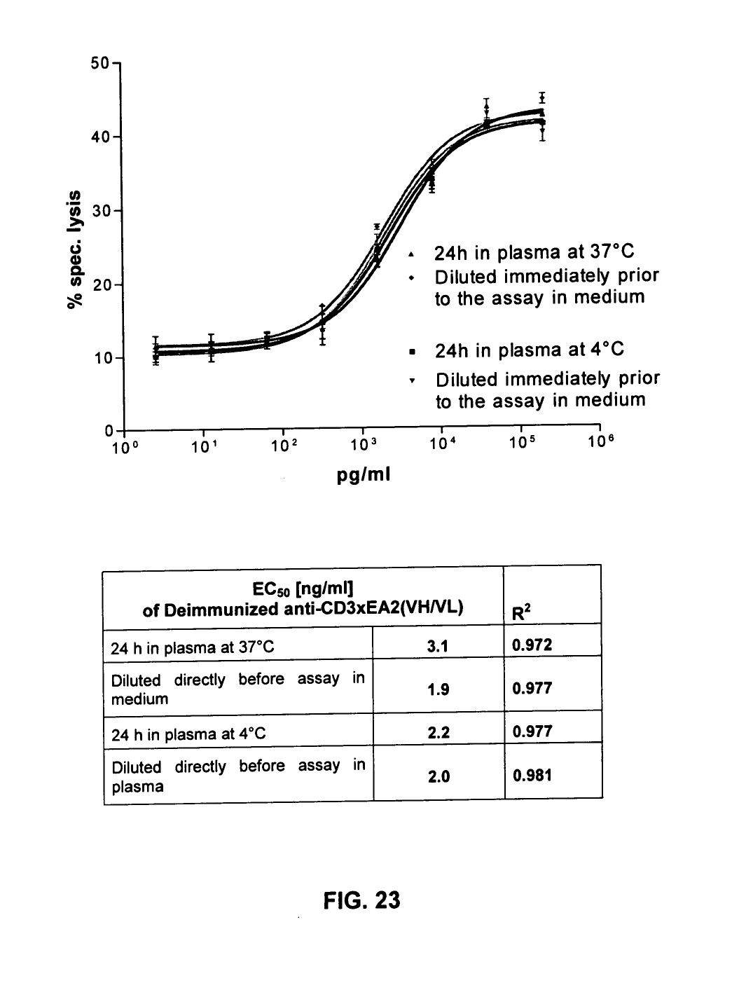

The plasma stability of the EphA2-specific BiTE was tested under different incubation conditions followed by ED50 determination of cytotoxic activity in a standard 51-chromium release assay. A human plasma pool was derived from the blood of five healthy donors collected by EDTA-coated syringes. Cellular components were removed by centrifugation and the upper plasma phase was collected and subsequently pooled. deimmunized anti-CD3×EA2 (VH/VL) BiTE was either incubated at 37° C. or 4° C. in the presence or absence of plasma. As controls, BiTE was diluted immediately prior to the cytotoxicity assay in plasma or RPMI-1640 medium, respectively. MDA-MB231 served as target cells; NK cell-depleted PBMC were used as effector cells. The effector:target (E:T) ratio was 10:1. The assay duration was 18 hours. See FIG. 23.

| EC₅₀ [ng/ml] | | |
|---|---|---|
| of deimmunized anti | | |
| CD3xEA2(VH/VL) | | R² |
| 24 h in plasma at 37° C. | 3.1 | 0.972 |
| Diluted directly before | 1.9 | 0.977 |
| assay in medium | | |
| 24 h in plasma at 4° C. | 2.2 | 0.977 |
| Diluted directly before | 2.0 | 0.981 |
| assay in plasma | | |

Deimmunized anti-CD3×EA2(VH/VL) proved to be stable as no major loss of cytotoxic activity could be detected after incubation in human plasma for 24 hours at 37° C.

6.3.8 Target Epitope Exclusion on Non-Transformed Cells by Deimmunized Anti-CD3×EA2(VH/VL)

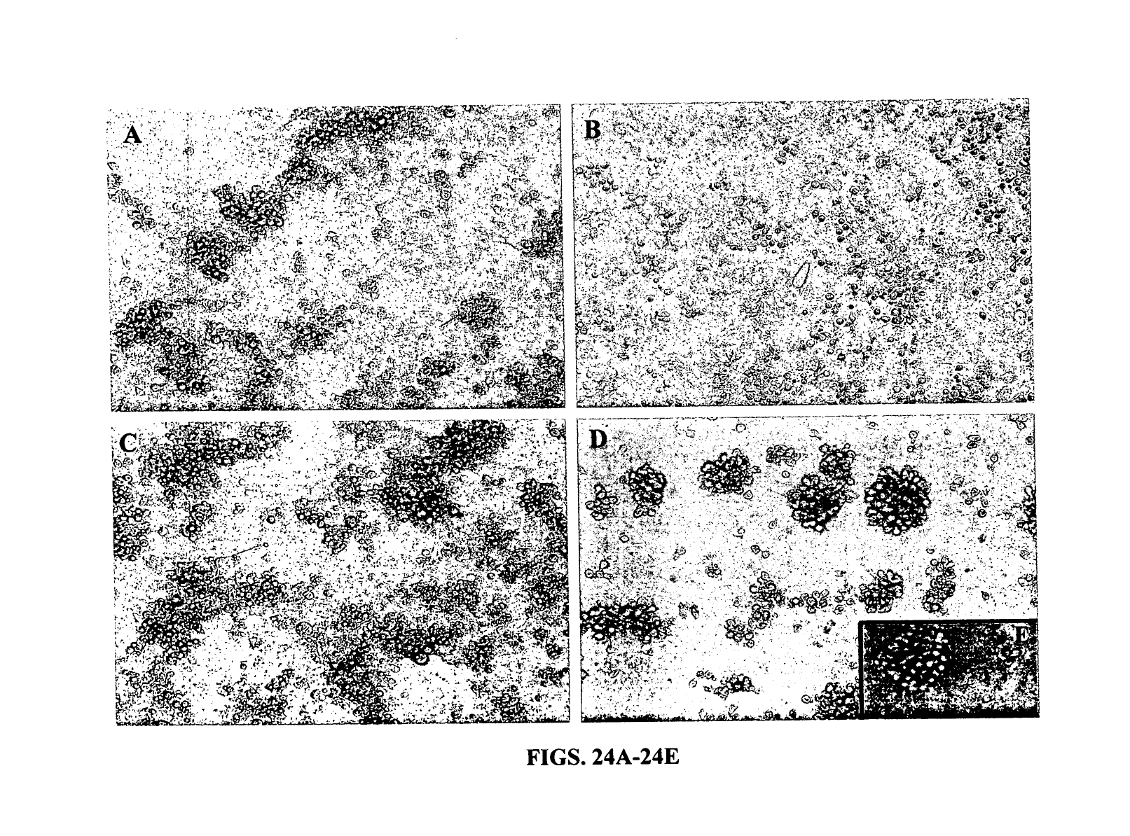

Video microscopy was employed to visualize the attack of CD8+ T-cells against non-transformed MCF10A cells in the presence of BiTE deimmunized anti-CD3×EA2 (VH/VL) and a non-epitope excluding control BiTE (see FIG. 24). Target cells were seeded for adherent cell growth 24 h before the beginning of the video recording into a 48-well plate. Directly before recording, a mixture of CD8+ T-cells and BiTEs were added to culture wells. Video microscopy was recorded for 20 hours with approximately one picture per minute. Propidium iodide (1 μg/ml) was added to wells after 18 hours. Single pictures were-converted to an AVI video movie, and transmitting light picture and fluorescence light pictures taken.

The videomicroscopic analysis during the assay duration of 18 hours exemplified the epitope exclusion by the EphA2-specific BiTE. While the pan-carcinoma positive control BiTE attacked the non-transformed MCF10A cells all across the intact cell monolayer (FIG. 24C), the EphA2-specific BiTE only showed T-cell activity at the border of the confluent cell layer where epitope exclusion through neighbouring cells is not possible (FIG. 24A). In the area of an intact monolayer, very little T-cell activity was noted, as seen in the absence of BiTE (FIG. 24B) where T-cells are just equally distributed on top of the monolayer. A monolayer of A549 carcinoma cells, which do not support epitope exclusion of EphA2, was completely destroyed by T-cells in the presence of deimmunized anti-CD3×EA2 (VH/VL). Addition of propidium iodide at the end of the assay allows the visualize dead carcinoma cells in cluster with T-cells. Such intensely stained cell clusters were present in at the end of the assay in each well except in control wells without BiTE.

6.3.9 Binding Constants of Deimmunized Anti-CD3×EA2 (VH/VL) for EphA2 Target

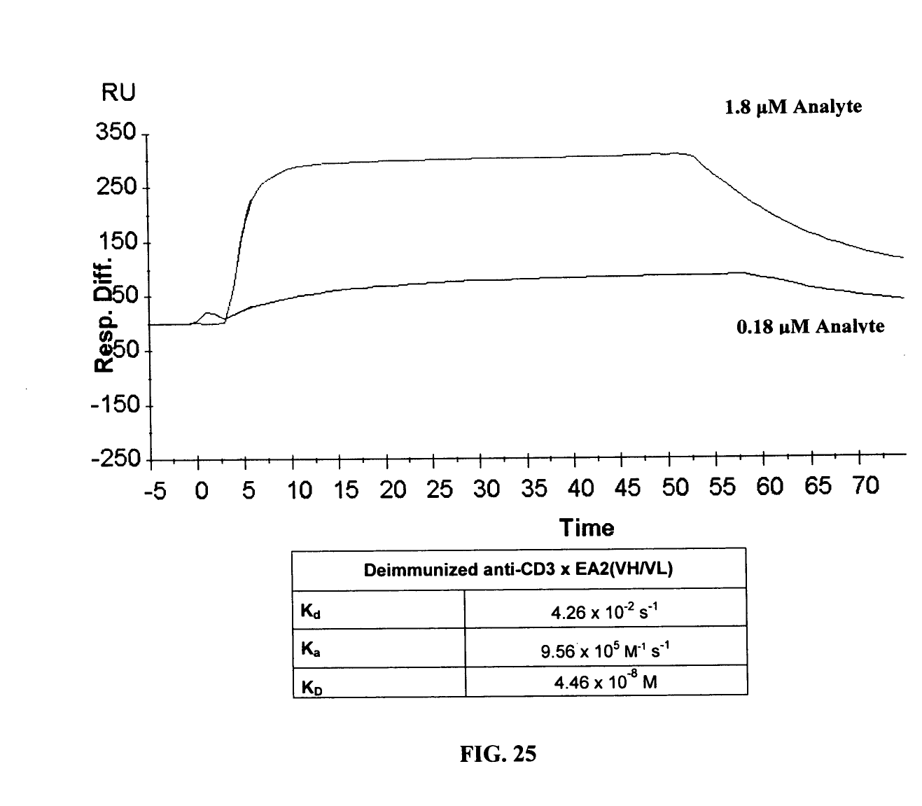

The formation and dissociation of BiTE/EphA2 complexes was monitored by surface plasmon resonance using a Biacore 3000 system. For this purpose, EphA2 Fc fusion protein was immobilized on a sensor chip with a CM5 carboxymethylated dextran matrix. Optimal immobilization conditions were identified through pH scouting (sodium acetate pH 4.0 to pH 5.5; disodium tetraborate pH 8.5). The optimal pH value was pH 4.0. After immobilization of 1000 RU to flow cell 2, 5,000 RU to flow cell 3 and 400 RU to flow cell 4, excess reactive groups were deactivated by ethanolamine. The affinity of the anti-EphA2 BiTE to immobilized EphA2 was determined by injecting different concentrations of the analyte deimmunized anti-CD3×EA2 VHVL diluted in HBS-EP buffer. To regenerate the surface, REGEN buffer (50 mM NaOH; 20 mM MES; 1 mM NaCl pH 6.4) was used. See FIG. 25.

Deimmunized anti-CD3x
EA2(VH/VL)

| | |
|---|---|
| K_{d} | 4.26 × 10⁻² s⁻¹ |
| Kₐ | 9.56 × 10⁵ M⁻¹ s⁻¹ |
| K_{D} | 4.46 × 10⁻⁸ M |

6.4 In Vivo Efficacy of EphA2-Specific BiTE Deimmunized Anti-CD3×EA2(VH/VL)

Deimmunized anti-CD3×EA2(VH/VL) is specific for human CD3 and primate EphA2 and therefore does not bind to mouse CD3 or EphA2. Anti-turn of the EphA2 BiTE deimmunized anti-CD3×EA2(VH/VL) could thus only be studied in immunodeficient NOD/SCID mice with a human colon carcinoma xenograft model.

6.4.1 NOD/SCID SW480 Xenograft Model

The human colon carcinoma cell line SW480 was selected for the establishment of a human xenograft model since SW480 cells express EphA2 and deimmunized anti-CD3×EA2(VH/VL) demonstrated redirected lysis of SW480 cells in vitro.

5×10⁶ SW480 cells were mixed with 2.5×10⁶ human CD3+ T-cells from healthy donors in a final volume of 0.2 ml PBS resulting in an E:T an ratio of 1:2. The T-cell effector/SW480 cell mixtures were subcutaneously injected into the right flank of each NOD/SCID mouse. Subcutaneously growing SW480 tumors were measured three times a week with a caliper in two perpendicular dimensions and tumor volumes calculated according to the formula: tumor volume=[(width²*length)/2].

6.4.2 Study Design

Six animals per group were intravenously treated with PBS control vehicle, non-relevant control BiTE or deimmunized anti-CD3×EA2(VHVL) for five consecutive days starting one hour after subcutaneous inoculation of CD3+ T-cell and SW480 tumor cells according to the table below.

Animals in groups I and J additionally received 2.5×10⁶ CD3+ effector cells via tail vein injection 5 minutes after SW480 tumor cell inoculation to simulate the presence of peripheral T-cells.

| | | Effector Cells | Target cells | E:T | | | |
|---|---|---|---|---|---|---|---|
| Group | N | (CD3+ T-cells) | (SW480) | Ratio | Treatment | Dose | |
| A | 6 | | 5 × 10⁶ | — | PBS i.v. | 100 | μl |
| | | | | | day 0-4 | | |
| B | 6 | | 5 × 10⁶ | — | deimmunized | 20 | μg |
| | | | | | anti-CD3xEA2 | | |
| | | | | | i.v. day 0-4 | | |
| C | 6 | 2.5 × 10⁶ | 5 × 10⁶ | 1:2 | PBS i.v. | 100 | μl |
| | | | | | day 0-4 | | |
| D | 6 | 2.5 × 10⁶ | 5 × 10⁶ | 1:2 | non-relevant | 100 | μg |
| | | | | | control BiTE i.v. | | |
| | | | | | day 0-4 | | |
| E | 6 | 2.5 × 10⁶ | 5 × 10⁶ | 1:2 | deimmunized | 100 | μg |
| | | | | | anti-CD3xEA2 | | |
| | | | | | i.v. day 0-4 | | |
| F | 6 | 2.5 × 10⁶ | 5 × 10⁶ | 1:2 | deimmunized | 20 | μg |
| | | | | | anti-CD3xEA2 | | |
| | | | | | i.v. day 0-4 | | |
| G | 6 | 2.5 × 10⁶ | 5 × 10⁶ | 1:2 | deimmunized | 5 | μg |
| | | | | | anti-CD3xEA2 | | |
| | | | | | i.v. day 0-4 | | |
| H | 6 | 2.5 × 10⁶ | 5 × 10⁶ | 1:2 | deimmunized | 1 | μg |
| | | | | | anti-CD3xEA2 | | |
| | | | | | i.v. day 0-4 | | |
| I | 6 | 2.5 × 10⁶ | 5 × 10⁶ | 1:2 | PBS i.v. | 100 | μl |
| | | +2.5 × 10⁶ i.v. | | | day 0-4 | | |
| J | 6 | 2.5 × 10⁶ | 5 × 10⁶ | 1:2 | deimmunized | 20 | μg |
| | | +2.5 × 10⁶ i.v. | | | anti-CD3xEA2 | | |
| | | | | | i.v. day 0-4 | | |

6.4.3 In Vivo Anti-Turn of Deimmunized Anti-CD3×EA2(VH/VL)

6.4.3.1. Specificity and Reproducibility of SW480 Model

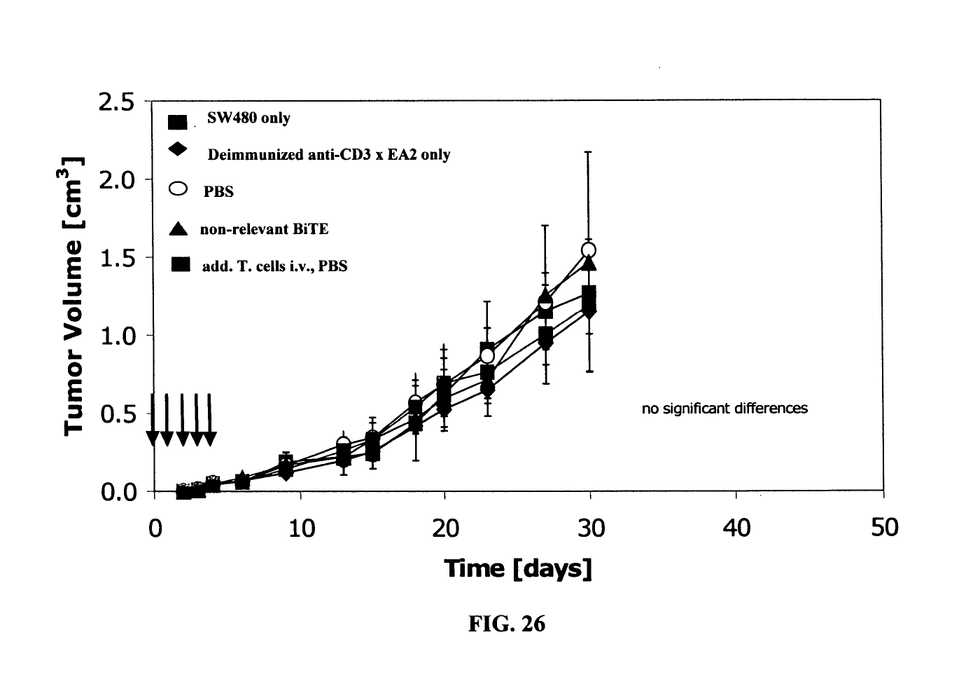

Inoculation of SW480 cells alone without effector cells followed by treatment with PBS (group A) or deimmunized anti-CD3×EA2 (VH/VL) (group B) lead to first palpable tumors on day 4 after inoculation and mice had to be sacrificed on day 30 due to large tumor masses (>1 cm³). There was no difference in the tumor growth kinetics between the PBS and deimmunized anti-CD3×EA2(VH/VL) treatment group indicating that EphA2 BiTE had no anti-tumor effect in the absence of effector cells. See FIG. 26.

Inoculation of mixtures of SW480 tumor and human T-cells followed by treatment with PBS control vehicle (group C), or non-relevant control BiTE (group D), which shares the CD3 binding arm with deimmunized anti-CD3×EA2(VH/VL) but has a different target arm did also not affect tumor growth demonstrating that the BiTE anti-CD3 arm per se nor T-cells alone had any anti-tumor activity. Thus, treatment with the non-relevant control BiTE showed the same effect as treatment with the vehicle PBS. See FIG. 26.

Lastly, intravenous injection of additional CD3+ T-cells 5 minutes prior to tumor cell inoculation mimicking the presence of peripheral T-cells did not influence tumor growth (group I). See FIG. 26. No significant differences in tumor growth were seen among all control conditions tested below. This also shows a high robustness and reproducibility of tumor growth in the SW480 NOD/SCID model with the selected tumor cell doses. See FIG. 26.

6.4.3.2. Dose Dependent Inhibition of Tumor Growth by Deimmunized Anti-CD3×EA2(VH/VL)

Deimmunized anti-CD3×EA2(VH/VL) treatment induced a dose-dependent inhibition of SW480 tumor outgrowth in the presence of CD3+ effector T-cells.

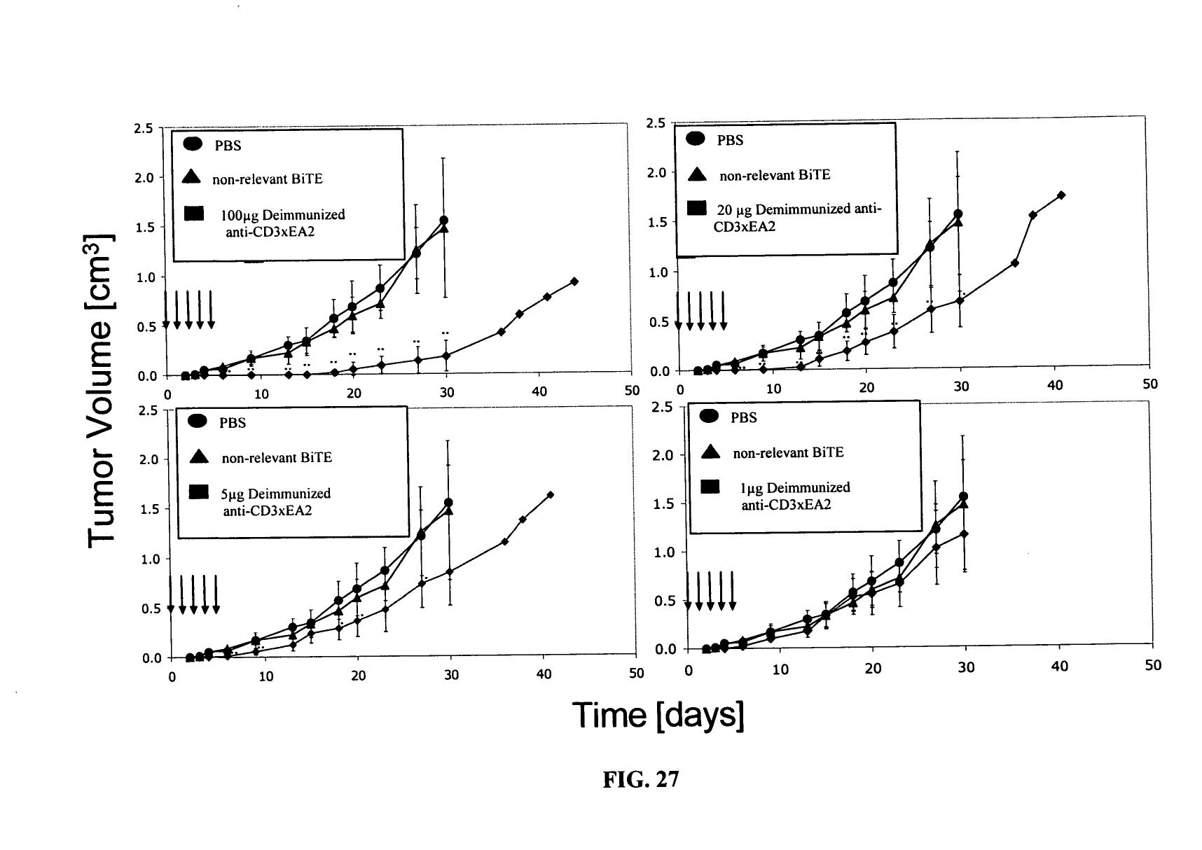

While treatment with 1 μg deimmunized anti-CD3×EA2 showed almost no effect on tumor growth, all other doses tested (5, 20 and 100 μg daily for five days) caused a significant inhibition of tumor outgrowth. With 5 daily 5-μg doses, tumor volume was significantly lower on days 3-9, and significantly lower on day 18, 20 and 27 compared to the non-relevant control BiTE group. Tumor volume of mice treated with 20 μg deimmunized anti-CD3×EA2(VH/VL) was significantly lower as compared to that of non-relevant control BiTE treated mice at all time points analyzed. Finally, treatment with 5×100 μg deimmunized anti-CD3×EA2(VH/VL) led to highly significant differences at all time points analyzed and showed no tumor growth at all for two weeks following BiTE treatment. Treatment with non-relevant control BiTE showed the same effect as treatment with the vehicle PBS. See FIG. 27.

6.4.3.3. No Effect of Peripheral T-Cells on Anti-Tumor Effect of Deimmunized Anti-CD3×EA2(VH/VL)

In contrast to the situation in the xenograft model where no human T-cells are present in the periphery, the efficacy of deimmunized anti-CD3×EA2 could be influenced by circulating T-cells that might trap the molecule in the periphery and therefore reduce the tumor-specific targeting of deimmunized anti-CD3×EA2(VH/VL). To mimic this scenario, two groups of mice were additionally injected intravenously with human T-cells to provide a peripheral human T-cell population.

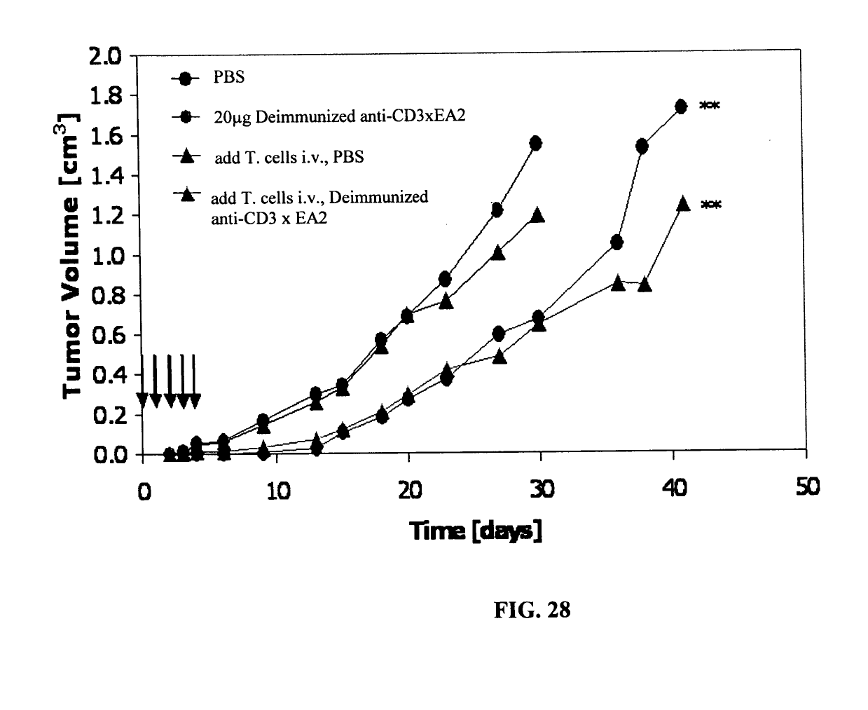

The i.v. administration of additional human T-cells had no influence on the efficacy of deimmunized anti-CD3×EA2(VH/VL). Growth of SW480 tumors in groups F (treatment with 20 μg deimmunized anti-CD3×EA2(VH/VL)/injection) and J (treatment with 20 μg deimmunized anti-CD3×EA2(VH/VL)/injection after i.v. injection of additional 2.5×10⁶ T-cells) was nearly identical. In both groups, deimmunized anti-CD3×EA2(VH/VL) treatment induced highly significant tumor growth inhibition as compared to PBS treated mice or PBS treated mice with additional circulating T-cells. See FIG. 28.

6.5 Humanized Anti-EphA2-BiTE Generation and Characterization

The following information details the generation and characterization of humanized anti-EphA2 antibodies used to construct anti-EphA2 BiTEs. Candidate BiTEs were constructed from antibodies that were humanized, able to bind human cynomolgus EphA2, and did not bind to normal human heart tissue.

Two murine anti-EphA2 monoclonal antibodies, B233 (see FIG. 29) and EA2 (FIG. 1), were humanized and produced 3F2 (derived from B233; FIG. 30) and 4H5 (derived from EA2). Affinity optimization was performed as described in detail below, and in Dall'Acqua et al., 2005, Methods 36:43-60, which is incorporated by reference herein in its entirety.

6.5.1 Affinity Optimization of EphA2 Antibodies

6.5.1.1. Affinity Optimization of 4H5

The following information details affinity optimization of the humanized anti-human EphA2 mAb 4H5 to produce the three affinity optimized variants 2A4, 2E7, and 12E2. For further details regarding the production of affinity optimized variants, and in particular, 2A4, 2E7, and 12E2, please see U.S. Provisional Appn. No. 60/751,964, filed Dec. 21, 2005, entitled “Affinity Optimized EphA2 Agonistic Antibodies and Methods of Use Thereof,” which is incorporated by reference herein in its entirety.

6.5.1.1.1 Reagents

All chemicals were of analytical grade. Restriction enzymes and DNA-modifying enzymes, and T4 ligase and T7 DNA polymerase were purchased from New England Biolabs, Inc. (Beverly, Mass.). Custom oligonucleotides were synthesized from Invitrogen (Carlsbad, Calif.). Human EphA2-Fc fusion protein (consisting of the human EphA2 ectodomain fused with the Fc portion of a human IgG1) was expressed in human embryonic kidney (HEK) 293 cells and purified by protein G affinity chromatography using standard protocols. Human EphA2-Fc biotinylation was carried out using an EZ-Link Sulfo-NHS-LC-Biotinylation Kit according to the manufacturer's instructions (Pierce, Rockford, Ill.).

6.5.1.1.2 Humanization of Murine Anti-Human EphA2 Antibody EA2 by Framework Shuffling Technology

The humanization of the parental murine mAb EA2 was accomplished using the framework shuffling technology as described in detail Dal'Acqua et al., 2005, Methods 36:43-60, and in U.S. Patent Pub. No. US-2005/0048617 A1, each of which is incorporated by reference herein in its entirety. Essentially, CDR regions of both EA2 VL and EA2 VH regions were grafted onto libraries of human framework germline sequences in a combinatorial fashion, creating mosaic, humanized variants retaining EphA2 binding. One such humanized clone, 4H5, exhibited approximately a 20-fold increase of affinity when compared with chimaeric Fab EA2. This clone was chosen as template for affinity maturation and was subsequently optimized as described below, resulting in the variants 2A4, 2E7 and 12E2.

6.5.1.2. Affinity Optimization of 4H5 scFv

6.5.1.2.1 scFv Template Construction

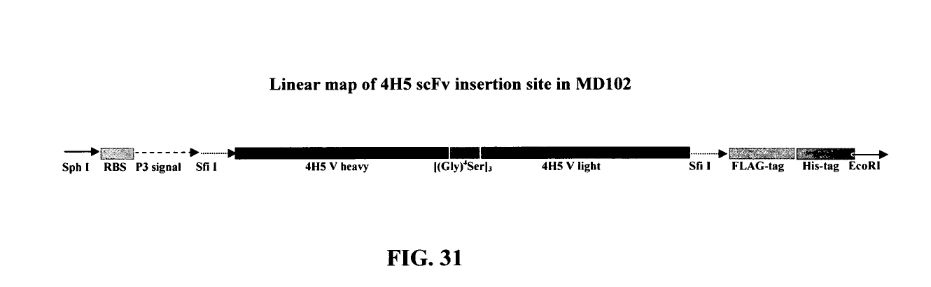

The variable regions of humanized mAb 4H5 were cloned as an scFv fragment into an M13 expression vector (Dal'Acqua et al., 2005, Methods 36:43-60, which is incorporated by reference herein in its entirety). The 4H5 variable light region was combined to the 3′ end of the 4H5 variable heavy chain by a [(Gly)4Ser]3 linker, and followed by a FLAG tag and a His tag on the C-terminal end (FIG. 31). Constructs were generated using PCR and the following primers to amplify the variable regions in separate reactions:

Medi-VH8: TTC TAT GCG GCC CAG CCG GCC CAG GTG CAG CTG TTG SAG TCT G (5′ primer to amplify VH, S═C/G) (SEQ ID NO:120)

Medi-JH1: GGA GCC GCC GCC GCC AGA ACC ACC ACC ACC TGA GGA GAC GGT GAC CAG GGT GCC (3′ primer to amplify VH) (SEQ ID NO:121)

Medi-VK1: GGC GGC GGC GGC TCC GGT GGT GGT GGT TCT GAC ATC CAG WTG ACC CAG TCT CC (5′ primer for VL, W=A/T) (SEQ ID NO: 122)

Medi-JK4: TGG AAT TCG GCC CCC GAG GCC ACG TTT GAT CTC CAC CTT GGT CCC (3′ primer for VL) (SEQ ID NO:123), where underlined sequences corresponds to the [(Gly)₄Ser]₃ linker, and bold italic letters denote the Sfi I restriction site. Overlapping PCR was used to construct the scFv fragment which was then restricted by Sfi I and cloned into the vector MD 102. The murine parental EA2 variable regions were cloned in the same manner to serve as an scFv control. The 4H5 scFv construct was then expressed in CJ236 to produce uridine+ssDNA as described in Wu and An, 2003, Methods Mol. Biol. 207:213-234, which is incorporated by reference herein in its entirety. This 4H5 scFv U+ssDNA was used as template for the mutagenic affinity optimization reactions that follow. The nucleotide and amino acid sequences of the 4H5 scFv are depicted in FIG. 32.

6.5.1.2.2 Affinity Optimization of scFv by Parsimonious Randomization of Each CDR Region

Each amino acid of all 6 Complementarity-Determining Regions (CDRs) was individually, randomly mutated using two separate libraries per amino acid (Wu and An, 2003, Methods Mol. Biol. 207:213-234, which is incorporated by reference herein in its entirety). Encoding either 8 amino acids (NSS) or 12 amino acids (NWS) at every CDR amino acid position, each individual degenerate primer was used in a single hybridization mutagenesis reaction (Wu, 2003, Methods Mol. Biol. 207:197-212, and Dal'Acqua et. al., 2005, Methods 36:43-60, each of which is incorporated by reference herein in its entirety), and then combined for generation of the corresponding CDR libraries. Briefly, each degenerate primer was phosphorylated, then used in a 10:1 ratio with uridinylated 4H5 scFv single-stranded U+DNA template (prepared as described in Wu and An, 2003, Methods Mol. Biol. 207:213-234) in an annealing reaction where the temperature was lowered from 95° C. to 55° C. over 1 hour. T4 ligase and T7 DNA polymerase was added to the annealed reaction and the reaction was incubated for 1.5 hours at 37° C. Synthesis products for every amino acid of each CDR were pooled, however NSS and NWS libraries were kept segregated and screened independently. Typically, 1 μl of the pooled CDR library synthesized DNA was then electroporated into XL1-Blue for plaque formation on XL1-Blue bacterial lawn or production of scFv fragments as described in Wu, 2003, Methods Mol. Biol. 207:197-212.

6.5.1.3. Screening of the Libraries

6.5.1.3.1 Primary Screen

The primary screen consisted of a single point ELISA (SPE) which was carried out using supernatants containing soluble, secreted scFv protein prepared from 1 ml-bacterial culture grown in 96 deep-well plates and infected with individual recombinant M13 clones essentially as described in Wu, 2003, Methods Mol. Biol. 207:197-212, and Dal'Acqua et. al., 2005, Methods 36:43-60. Briefly, this Capture ELISA involves coating individual wells of a 96-well Maxisorp Immunoplate with approximately 30 ng of a mouse anti-FLAG antibody (Sigma), blocking with 3% BSA/PBS for 2 h at 37° C. and incubating with samples (soluble, secreted scFv) for 2 h at room temperature. 150-600 ng/well of biotinylated human EphA2-Fc was then added for 2 h at room temperature. This was followed by incubation with neutravidin-horseradish peroxidase (HRP) conjugate (Pierce, Ill.) for 40 min at room temperature. HRP activity was detected with tetra methyl benzidine (TMB) substrate and the reaction quenched with 0.2 M H2SO4. Plates were read at 450 nm.

6.5.1.3.2 Results of the Primary Screen

Typically, clones exhibiting an OD 450 nm signal approximately two times greater than the parental 4H5 scFv were re-grown at a 15 ml scale, and re-assayed by the same ELISA in duplicate wells to confirm the positive result. Clones which repeated were then sequenced and assayed using an Activity ELISA (see below) to estimate the folds increase of binding to human EphA2.

6.5.1.3.3 Secondary Screen

In order to further characterize the previously identified single-change, affinity optimized variants, a secondary screen using secreted scFv fragments expressed from 15 ml-bacterial culture (see Wu, 2003, Methods Mol. Biol. 207:197-212) was carried out. More precisely, two ELISAs were used: (i) an activity ELISA in which individual wells of a 96-well Maxisorp Immunoplate were coated with ˜0.5 μg of human EphA2-Fc and blocked with 3% BSA/PBS for 2 h at 37° C. 2-fold serially diluted samples were then added and incubated for 1 h at room temperature. Incubation with a goat anti-human kappa horseradish peroxydase (HRP) conjugate then followed. HRP activity was detected with TMB substrate and the reaction quenched with 0.2 M H2SO4. Plates were read at 450 nm; (ii) an anti-scFv quantification ELISA, which was carried out essentially as, described in Wu, 2003, Methods Mol. Biol. 207:197-212. Briefly, individual wells of a 96-well Ni NTA plate (Qiagen) incubated with 2-fold serially diluted samples or standard (50-0.78 ng/ml). Incubation with a mouse anti-FLAG horseradish peroxydase (HRP) conjugate then followed. HRP activity was detected with TMB substrate and the reaction quenched with 0.2 M H2SO4. Plates were read at 450 nm.

6.5.1.3.4 Results of the Secondary Screen

The two-part secondary ELISA screen described above allowed comparison of the scFv 4H5 and the affinity optimized variants to each other in terms of binding to human EphA2 by normalizing their scFv concentrations. All single-change, affinity optimized variant scFv clones exhibited better binding to human EphA2 when compared with the parental scFv 4H5 (data not shown).

6.5.1.4. Construction and Characterization of Combinatorial Variants from CDR Affinity Optimized Clones

To engineer combinatorial variants with further improvement in binding, all single amino acid changes which improved binding when compared to parental 4H5 scFv by activity/quantitative ELISA were combined to create a small, focused combinatorial library. Briefly, degenerate primers encoding all identified amino acid changes as well as the parental amino acid at the same position were designed. In an annealing reaction where all primers were included and synthesis followed (supra), a combinatorial library was constructed and screened as previously described supra.

6.5.1.4.1 Results of Primary Screening on EphA2

Typically, clones exhibiting an OD 450 nm signal greater than the parental scFv 4H5 were re-grown at a 15 ml scale, and re-assayed by ELISA (described supra) in duplicate wells to confirm the positive result. Sixteen combinatorial variants were then selected and sequenced identifying 11 unique combinations of CDR amino acid changes thus making each variant different from one another by one to three amino acids at the primary sequence level.

6.5.1.4.2 Results of Secondary Screening on EphA2

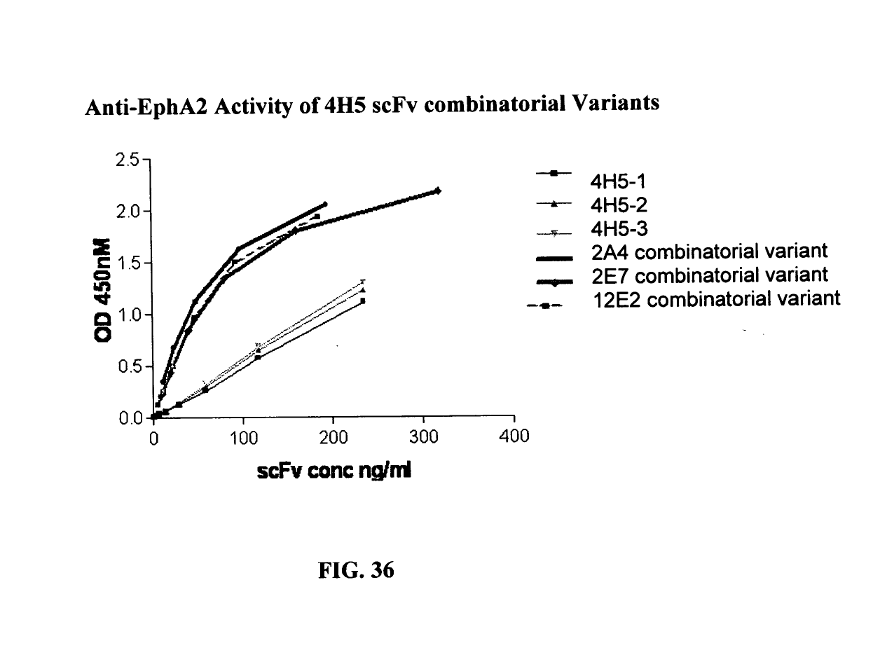

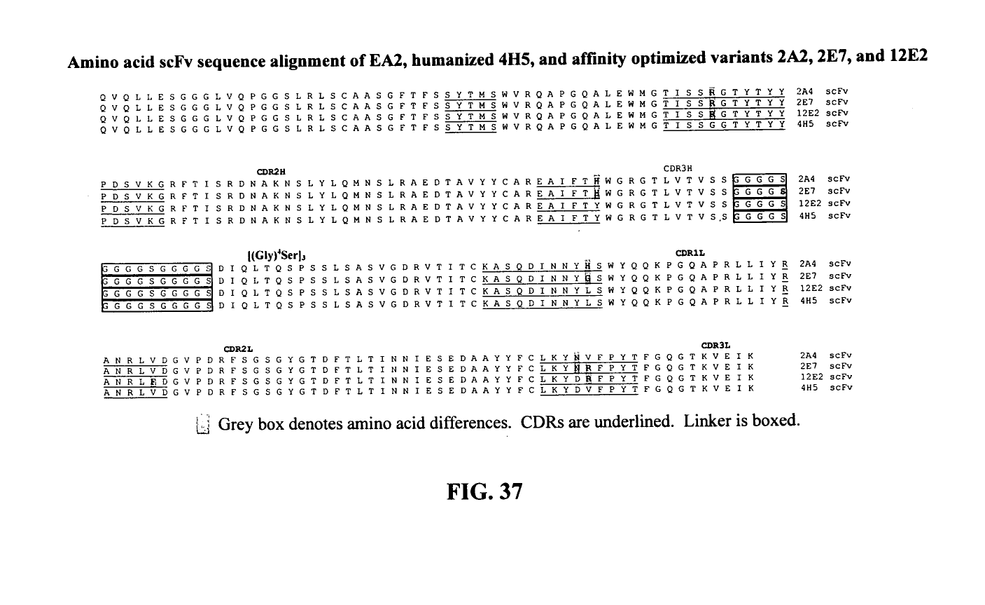

The 11 unique combinatorial variants described above were analyzed by a secondary screen as described above to estimate the improved binding affinities of the combinatorial variants. All variants had significantly improved affinities for human EphA2 when compared to 4H5 scFv. The nucleotide and amino acid sequences of the three affinity optimized combinatorial variants 2A4, 2E7 and 12E2 are shown in FIGS. 33, 34 and 35, respectively. Data for three affinity optimized combinatorial variants 2A4, 2E7, and 12E2 are shown in FIG. 36. FIG. 37 depicts the amino acid sequence alignment of the affinity optimized variants 2A4, 2E7 and 12E2 with the humanized 4H5 scFv.

6.5.1.4.3 Binding Analysis

2A4, 2E7 and 12E2 as well as parental EA2 scFv and humanized 4H5 scFv were induced for expression in E. coli in a 1 L culture volume. The supernatants containing soluble, secreted scFv fragments were spun to remove cellular debris then passed over an anti-FLAG column (Sigma) to purify and isolate the variant proteins. The purified affinity optimized variants were analyzed by surface plasmon resonance detection using a BIAcore 3000 instrument (Pharmacia Biosensor, Uppsala, Sweden). Humanized, affinity optimized variants of EA2 exhibited 110-150-fold affinity improvement when compared to the parental anti-EphA2 scFv EA2. For affinity measurement results, see the table in section 6.6, infra.

6.5.2 Affinity Optimization of Murine Anti-Human EphA2 Antibody B233

The humanization of the parental molecule mAb B233 was accomplished using the framework shuffling technology as described in detail by Dal'Acqua et al., 2005, Methods 36:43-60, which is incorporated by reference herein in its entirety. Essentially, CDR regions of both B233 VL and B233 VH regions were grafted onto libraries of human framework germline sequences in a combinatorial fashion, creating mosaic, humanized variants retaining EphA2 binding. One such humanized clone, 2G6, exhibited approximately a 10-fold loss of affinity when compared with chimaeric mAb B233. This clone was chosen as template for affinity maturation and was subsequently optimized as described below, resulting in the variant 3F2.

6.5.2.1. Construction and Characterization of Combinatorial Variants

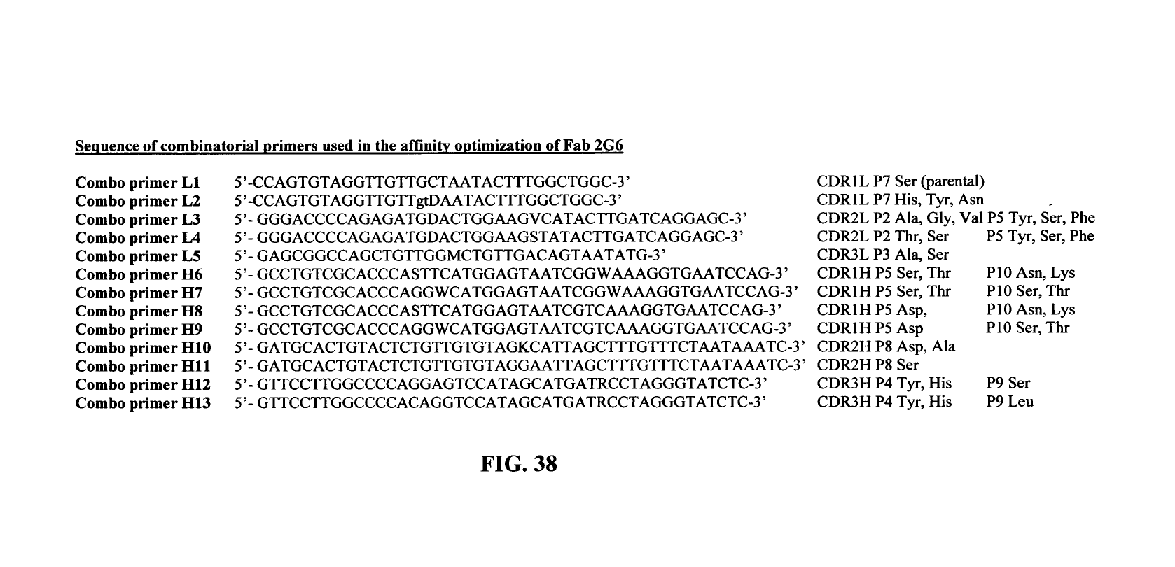

To engineer a combinatorial variant with improved binding to human EphA2, all single amino acid changes which improved binding when compared to parental B233 were combined to create a small, focused combinatorial library. Briefly, degenerate primers (see FIG. 38) encoding all identified amino acid changes as well as the parental amino acid at the same position were designed. An annealing reaction was carried out where all primers were included and synthesis followed, essentially as described in Dal'Acqua et al., 2005, Methods 36: 43-60 and Wu, 2003. Methods Mol. Biol. 207:197-212, which are both incorporated by reference herein in their entireties. The combinatorial library thus constructed was then screened.

6.5.2.1.1 Screening of the Libraries

6.5.2.1.1.1 Primary Screen

The primary screen consisted of a single point ELISA (SPE) which was carried out using periplasmic Fab extracts prepared from 1 ml-bacterial culture grown in 96 deep-well plates and infected with individual recombinant M13 clones essentially as described in Dal'Acqua et al., 2005, Methods 36: 43-60 and Wu, 2003. Methods Mol. Biol. 207:197-212. Briefly, this capture ELISA involves coating individual wells of a 96-well Maxisorp Immunoplate with approximately 20 ng of a goat anti-human Fab antibody, blocking with 3% BSA/PBS for 2 h at 37° C. and incubating with samples (periplasm-expressed Fabs) for 2 h at room temperature. 300 ng/well of biotinylated human EphA2-Fc was then added for 2 h at room temperature. This was followed by incubation with neutravidin-horseradish peroxydase (HRP) conjugate (Pierce, Ill.) for 40 min at room temperature. HRP activity was detected with tetra methyl benzidine (TMB) substrate and the reaction quenched with 0.2 M H2SO4. Plates were read at 450 nm.

6.5.2.1.1.2 Results of the Primary Screen

Typically, clones exhibiting an OD 450 nm signal at least two times greater than the parental 2G6 were re-grown at a 15 ml scale, and re-assayed by the same ELISA in duplicate wells to confirm the positive result. Clones that repeated were then sequenced and assayed by using an activity ELISA (see below) to estimate the folds increase of binding to human EphA2.

6.5.2.1.1.3 Secondary Screen

In order to further characterize the previously identified combinatorial affinity optimized variants (see above), a secondary screen using Fab fragments expressed in periplasmic extracts prepared from 15 ml-bacterial culture (Wu, 2003, Methods Mol. Biol. 207:197-212) was carried out. More precisely, two ELISAs were used: (i) an activity ELISA in which individual wells of a 96-well Maxisorp Immunoplate were coated with ˜1 μg of human EphA2-Fc and blocked with 3% BSA/PBS for 2 h at 37° C. 2-fold serially diluted samples were then added and incubated for 1 h at room temperature. Incubation with a goat anti-human kappa horseradish peroxydase (HRP) conjugate then followed. HRP activity was detected with TMB substrate and the reaction quenched with 0.2 M H2SO4. Plates were read at 450 nm; (ii) an anti-human Fab quantification ELISA that was carried out essentially as described (Wu, 2003, Methods Mol. Biol. 207:197-212). Briefly, individual wells of a 96-well Biocoat plate (BD Biosciences, CA) were incubated with 2-fold serially diluted samples or standard (human IgG Fab, 100-1.56 ng/ml). Incubation with a goat anti-human kappa horseradish peroxydase (HRP) conjugate then followed. HRP activity was detected with TMB substrate and the reaction quenched with 0.2 M H2SO4. Plates were read at 450 nm.

6.5.2.1.1.4 Results of the Secondary Screen

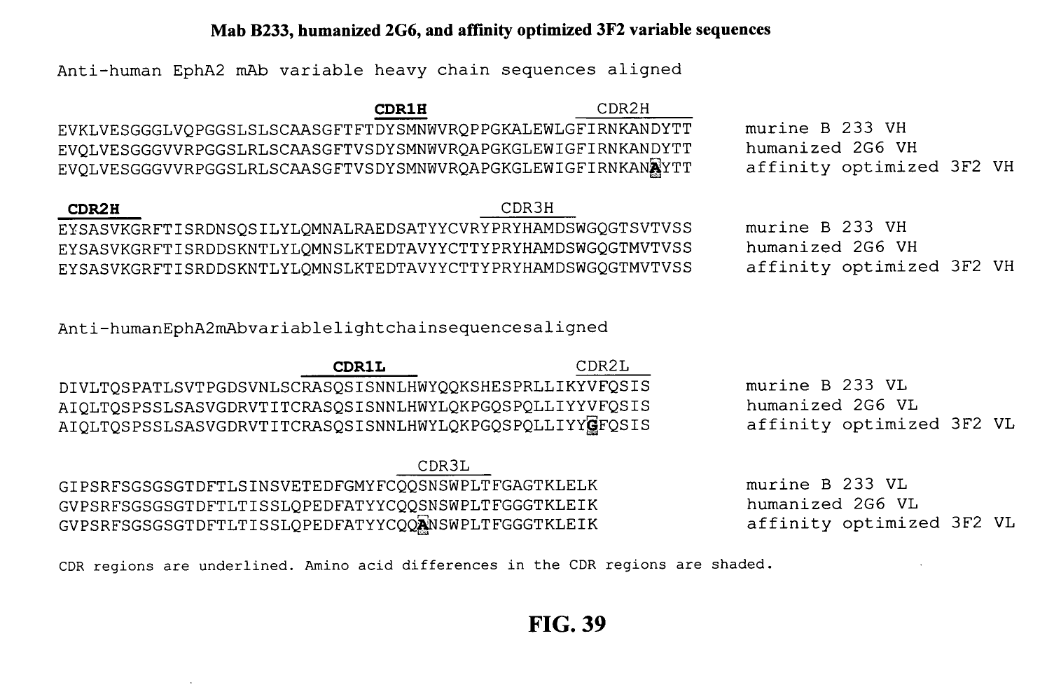

The two-part secondary ELISA screen described in the section above allowed comparison of Fab 2G6 and the affinity optimized combinatorial variants to each other in terms of binding to human EphA2. One of these affinity optimized combinatorial variant Fabs was chosen for more extensive analysis (variant named 3F2 thereafter). Amino acid sequence of the variable regions of murine B233, humanized 2G6 and affinity optimized 3F2 mAbs are aligned in FIG. 39.

6.5.2.2. Cloning, Expression and Purification of the Various Humanized, Affinity Optimized Versions of mAb B233 in a Human IgG1 Format

The variable regions of humanized, framework-shuffled clone 2G6 and the affinity optimized variant 3F2 were PCR-amplified from the corresponding V region-encoding M13 phage vectors using pfu DNA polymerase. They were then individually cloned into mammalian expression vectors encoding a human cytomegalovirus major immediate early (hCMVie) enhancer, promoter and 5′-untranslated region (Boshart et al., 1985, Cell 41:521-530, which is incorporated by reference herein in its entirety). In this system, a human γ1 chain is secreted along with a human κ chain (Johnson et al., 1997, Infect. Dis. 176:1215-1224, which is incorporated by reference herein in its entirety). The different constructs were expressed transiently in HEK 293 cells and harvested 72 and 144 hours post-transfection. The secreted, soluble human IgG1s were purified from the conditioned media directly on 1 ml HiTrap protein A or protein G columns according to the manufacturer's instructions (APBiotech, Inc., Piscataway, N.J.). Purified human IgG1s (typically >95% homogeneity, as judged by SDS-PAGE) were dialyzed against phosphate buffered saline (PBS), flash frozen and stored at −70° C.

6.5.2.2.1 Biacore Analysis

The interaction of soluble B233, 2G6, and 3F2 IgGs with immobilized EphA2-Fc was monitored by surface plasmon resonance detection using a Biacore® 3000 instrument (Pharmacia Biosensor, Uppsala, Sweden) essentially as described in W. F. Dal'Acqua et al., 2005, Methods 36 (2005) 4360. See also section 6.6.2, infra. Affinity measurements of chimeric B233, humanized 2G6, and affinity-optimized 3F2 are given in the table below.

| Kinetics to human | | | |
|---|---|---|---|
| EphA2 | kₒₙ M⁻¹s⁻¹ | K_{D}(pM) | |
| Chimaeric B 233 | 2.4 × 10⁵ | 8.0 × 10⁻⁵ | 300 |
| Humanized 2G6 | 6.4 × 10⁴ | 1.9 × 10⁻⁴ | 3000 |
| Affinity optimized | 1.89 × 10⁵ | 1.27 × 10⁻⁴ | 671 |
| 3F2 | | | |

6.6 Affinity Measurements OF EphA2-BiTEs

The following describes the affinity constants of the EphA2-BiTEs of the invention as measured by surface plasmon resonance.

6.6.1 Anti-Human CD3

Surface plasmon resonance measurement using immobilized soluble CD3εγ surface is provided. Biphasic association of BiTE molecules to CD3εγ prevents accurate calculations of binding rate and affinity constants.

Deimmunized anti-CD3×EA2 VH/VL 400-500 nM (est.)

6.6.2 Anti-Human EphA2

Surface plasmon resonance measurements using immobilized EphA2-Fc surface are provided:

| | | |
|---|---|---|
| Deimmunized anti-CD3xEA2 VH/VL | (MT) | 45 nM |
| Deimmunized anti-CD3xEA2 VH/VL | (MedI) | 113 nM |

6.6.3 Affinity Measurements (K_{D}) of scFVs

The table below provides a summary of the binding kinetics of the anti-human EphA2 scFvs. Monomeric anti-human EphA2 scFv association to EphA2 as determined by surface plasmon resonance binding. Affinity optimization produced three scFv's with 20-30 fold increase in affinity (K_{D}) to human EphA2 as compared to 4H5.

| | | | | | Fold |
|---|---|---|---|---|---|
| | kₒₙ | K_{off} | | | Difference |
| scFv | (1/Ms) | (1/s) | K_{D} | | (vs 4H5) |
| EA2 | 3.4 × 10⁴ | 3.0 × 10⁻² | 870 | nM | −4.8 |
| 4H5 | 1.0 × 10⁵ | 1.8 × 10⁻² | 180 | nM | 1 |
| 2A4 | 6.5 × 10⁵ | 3.7 × 10⁻³ | 5.7 | nM | 32 |
| 2E7 | 5.6 × 10⁵ | 4.1 × 10⁻³ | 7.3 | nM | 25 |
| 12E2 | 5.2 × 10⁵ | 4.1 × 10⁻³ | 7.8 | nM | 23 |

6.7 Characterization of Humanized Anti-Human EphA2-BiTEs

6.7.1 Flow Cytometric Binding Analysis of Humanized Anti-EphA2 Parental Antibodies to Human and Cynomolgus EphA2

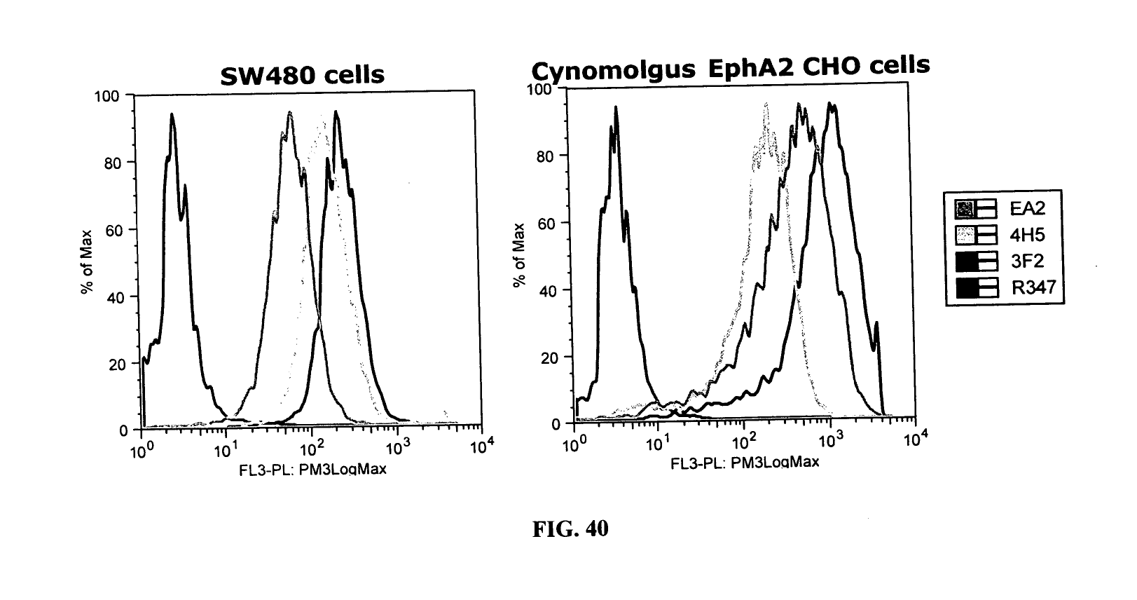

Subconfluent monolayers of SW480 colon carcinoma or cynomolgus EphA2 transfected CHO cells were quickly trypsinized, washed, counted, plated into 96-well round bottom plates, and stained with 10 μg/ml of a negative control antibody (R347), the anti-human EphA2 murine antibody, EA2 (Coffman 2003), or humanized antibodies, 3F2 and 4H5. Cells were resuspended in AlexaFluor 488 anti-mouse or anti-human IgG H+L (Invitrogen) and then analyzed by flow cytometry using a FACSCalibur (Becton Dickinson). As shown in FIG. 40, EA2, 3F2, and 4H5 each bound to human and cynomolgus EphA2.

6.7.2 Epitope Exclusion Properties of Humanized Anti-EphA2 Parental Antibodies

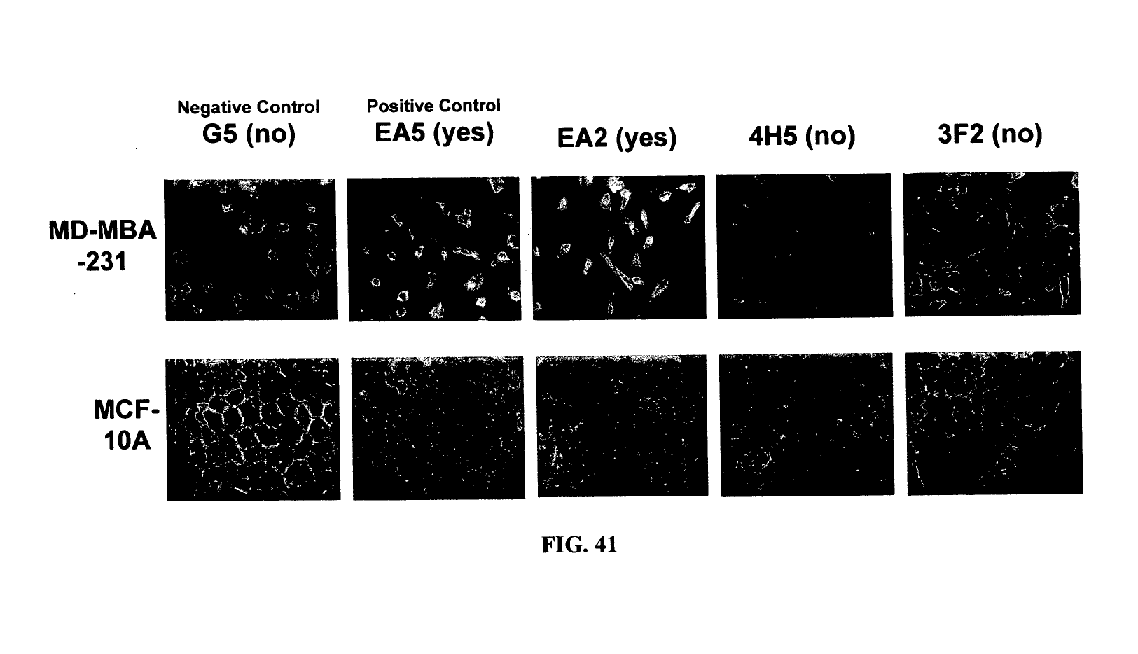

Monolayers of MCF-10A or MDA-MB-231 cells were cultured atop glass coverslips for at least 24 h at 37° C. before staining. Cell monolayers were fixed in 4% paraformaldehyde (2 min, 25° C.) before incubation with primary antibody (clone G5 (negative control), EA5, EA2, 3F2, or 4H5) for 30 min followed by subsequent staining with AlexaFluor 488-conjugated goat anti-mouse IgG or AlexaFluor 488-conjugated goat anti-human IgG (The Jackson Laboratory). Cells were fixed for analyses by immunofluorescence microscopy. As shown in FIG. 41, 3F2 and 4H5 bound to EphA2 on both nontransformed and transformed breast epithelial cells. Thus, 3F2 and 4H5 did not share EA2's unique ability to bind an EphA2 epitope accessible on malignant cells but selectively excluded by the normal architecture of nontransformed epithelial cells. The amino acid sequence of the VL and VH domains of the G5 antibody are depicted in FIG. 42.

6.7.3 Tissue Cross Reactivity Analysis

Tissue cross-reactivity assessment of the humanized anti-EphA2 antibodies to human heart tissue determined that 3F2 and 4H5 did not bind to normal human heart tissue at concentrations up to 10 μg/ml.

Tissue cross-reactivity was also assessed for the combinatorial variant scFvs 2A4, 12E2, and 2E7. None of the combinatorial variant scFvs (2A4, 12E2, and 2E7) stained human heart tissue (2 donors) at concentrations of up to 50 10 μg/ml (data not shown).

6.7.4 Characterization of 3F2 Anti-EphA2 BiTE Constructs

The 3F2-based BiTE constructs produced are depicted in the table below and were further characterized by cytoxicity assays.

| N-terminal Position | C-terminal Position |
|---|---|
| 3F2 (VH/VL) | deimmunized anti-CD3 |
| 3F2 (VL/VH) | (VH/VL) |
| deimmunized anti-CD3 | 3F2 (VH/VL) |
| (VH/VL) | 3F2 (VL/VH) |

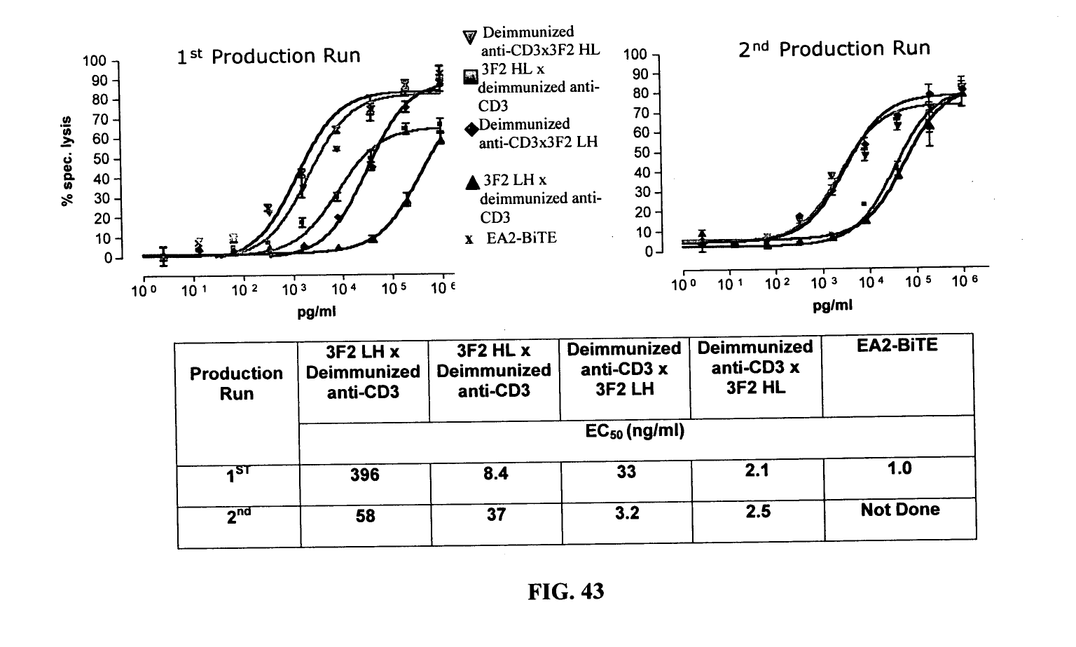

Cytotoxicity assays based on Chromium-51 release were performed to determine redirected target cell lysis by the various anti-EphA2 BiTE constructs in the presence of stimulated human CD8+ T-cells. EphA2-positive tumor cells line MDA-MB-231 was loaded with Chromium-51 and served as target cells. The various anti-EphA2 BiTE constructs were titrated over a broad range of concentrations. The assay duration was 18 hours, and the effector-to-target ratio 10:1. EphA2-BiTE concentrations required for half-maximal lysis (i.e., EC₅₀) with different production batches were estimated using a four-parameter non-linear fit model. See FIG. 43.

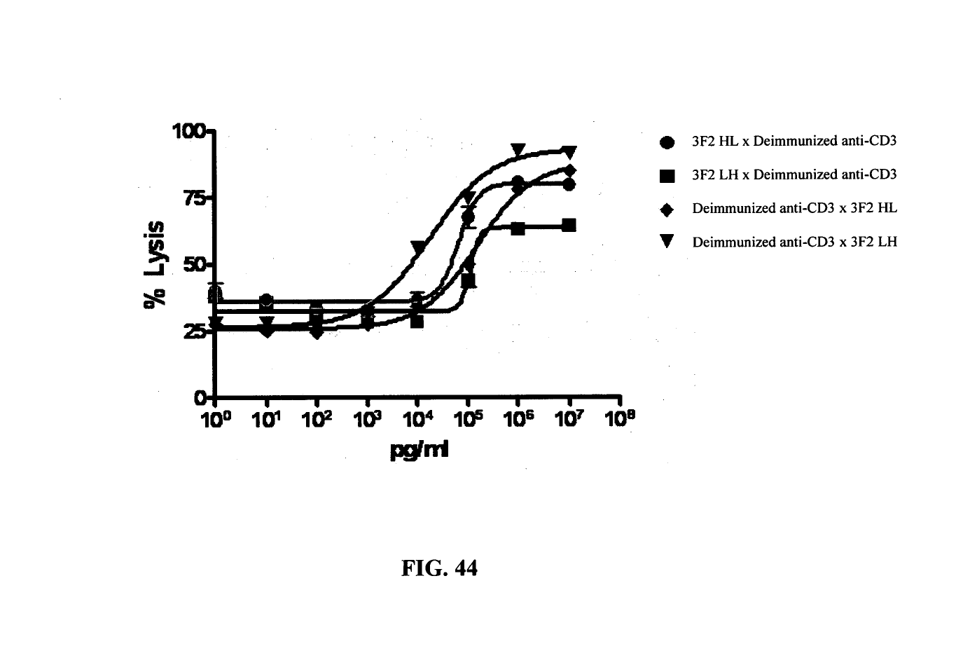

Flow cytometry-based redirected cellular cytotoxicity assays were conducted as listed above using CD3+ T-cell enriched human peripheral blood mononuclear cells (PBMC) as effector cells and EphA2+SW480 colon carcinoma cells. See FIG. 44.

In vitro cytotoxicity measurements (FIGS. 43 and 44) of the four single-chain 3F2-based anti-EphA2 BiTE constructs (table above) suggested that the potency (i.e., EC₅₀) of the 3F2 BiTEs was not superior to murine EA2 BiTE. These results established that humanized anti-EphA2 BiTEs redirected human T-cells to lyse EphA2+ tumor cells.

6.7.5 Characterization of 4H5 Anti-EphA2 BiTE Constructs

The 4H5-based anti-EphA2 BiTE constructs produced are depicted in the table below and were further characterized.

| N-terminal Position | C-terminal Position |
|---|---|
| 4H5 (VH/VL) | deimmunized anti-CD3 |
| 4H5 (VL/VH) | (VH/VL) |
| deimmunized anti-CD3 | 4H5 (VH/VL) |
| (VH/VL) | 4H5 (VL/VH) |

6.7.5.1. Specificity of Target Cell Binding of 4H5-Based Anti-EphA2 BiTES to EphA2+ and CD3+ Expressing Cells

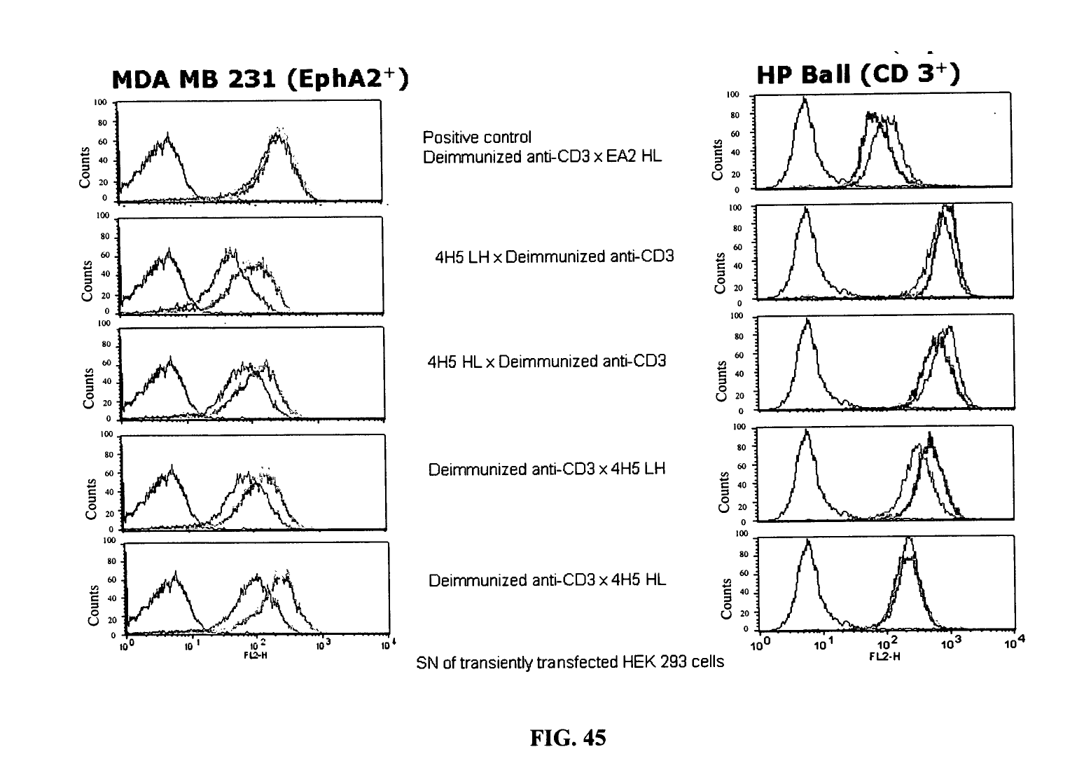

The target binding specificity of the various 4H5-based BiTE constructs was examined using the flow cytometry-based assay described above. EphA2+MDA MB 231 breast cancer cells and CD3+HP Ball human T-cells were used as target cells. The deimmunized anti-CD3×EA2 (VH/VL) BiTE was used as a positive control. As shown in FIG. 45, each of the 4H5-based EphA2-BiTE constructs bound to cells expressing both EphA2 and CD3.

6.7.5.2. Specificity of Target Cell Binding of the Humanized Affinity Matured 4H5-based BiTE Constructs 12E2, 2E7 and 2A4

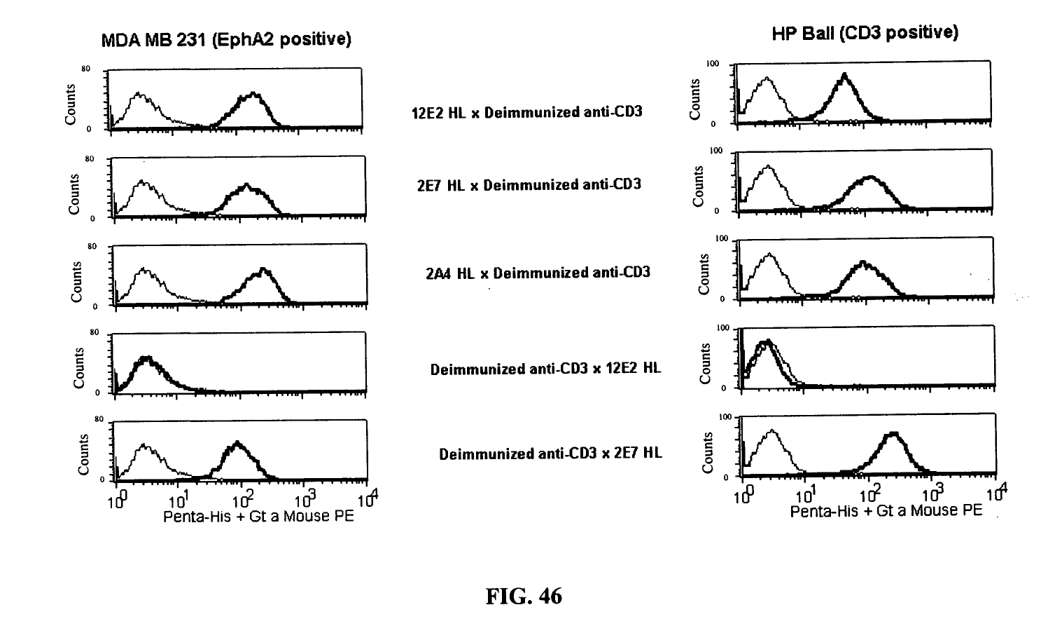

The target binding specificity of the various 4H5-based BiTE constructs was examined using the flow cytometry-based assay described above. EphA2+MDA MB 231 breast cancer cells and CD3+HP Ball human T-cells were used as target cells. As shown in FIG. 46, each of the 4H5-based EphA2-BiTE constructs bound to cells expressing both EphA2 and CD3.

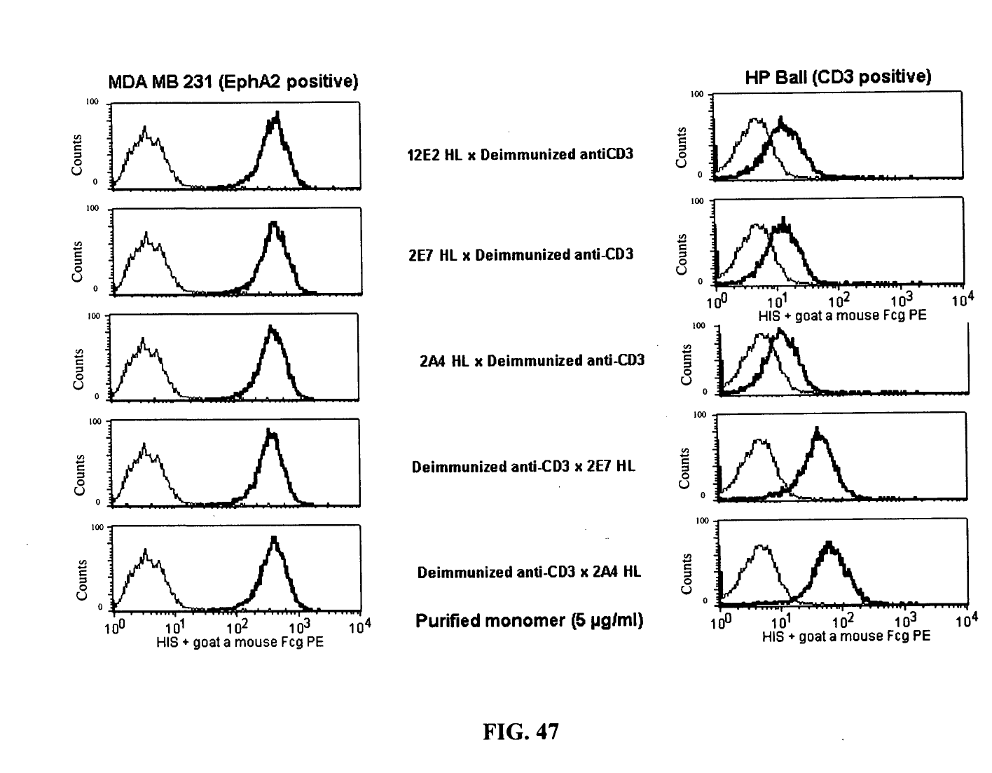

As shown in FIG. 47, the purified monomers of the affinity matured 4H5-based EphA2-BiTE constructs bound to EphA2+MDA MB 231 breast cancer cells and CD3+HP Ball human T-cells target cells at a concentration of 5 μg/ml using the flow cytometry-based assay as described above.

To determine the ability of the affinity matured 4H5-based scFvs, monolayers of MCF-10A or MDA-MB-231 cells were cultured atop glass coverslips for at least 24 h at 37° C. before staining. Cell monolayers were fixed in 4% paraformaldehyde (2 min, 25° C.) before incubation with primary scFv antibody (2A4, 2E7 or 12E2) for 30 min followed by subsequent staining with AlexaFluor 488-conjugated goat anti-mouse IgG or AlexaFluor 488-conjugated goat anti-human IgG (The Jackson Laboratory). Cells were fixed for analyses by immunofluorescence microscopy. The 2A4 and 12E2 scFvs bound to EphA2 on both nontransformed and transformed breast epithelial cells. The 2E7 scFv, however, shared EA2's unique ability to bind an EphA2 epitope accessible on maligant cells but not selectively excluded by the normal architecture of nontransformed epithelial cells (data not shown).

6.7.5.3. Cytotoxic Potency Comparison of Four EphA2-Specific BiTEs of the Humanized 4H5 Monoclonal Antibody

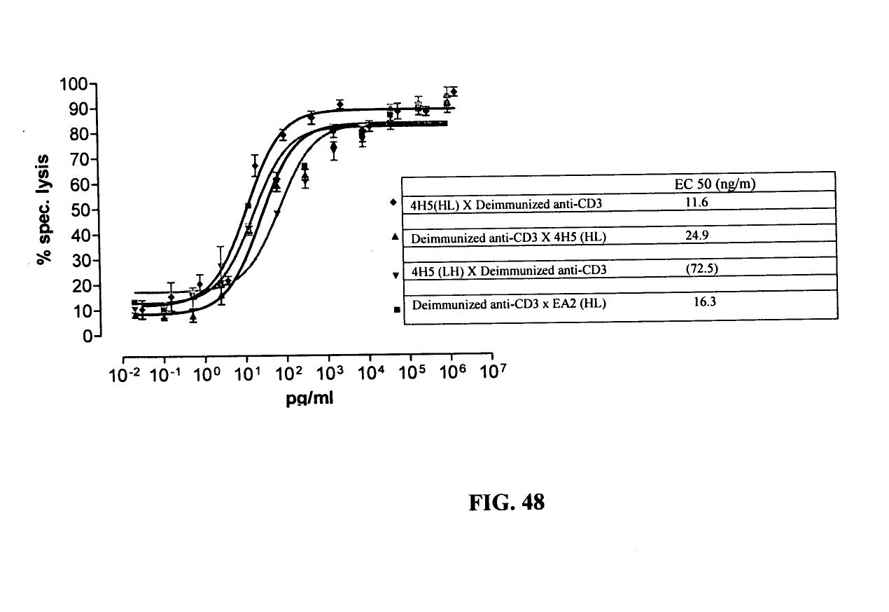

Cytotoxicity assays based on Chromium-51 release were performed to determine redirected target cell lysis by the various anti-EphA2 BiTE constructs in the presence of stimulated human CD8+ T-cells. See FIG. 48. The EphA2-positive tumor cell line MDA-MB-231 was loaded with Chromium-51 and served as target cells. The various anti-EphA2 BiTE constructs were titrated over a broad range of concentrations. The assay duration was 18 hours, and the effector-to-target ratio 10:1. EphA2-BiTE concentrations required for half-maximal lysis (i.e., EC50) with different production batches were estimated using a four-parameter non-linear fit model.

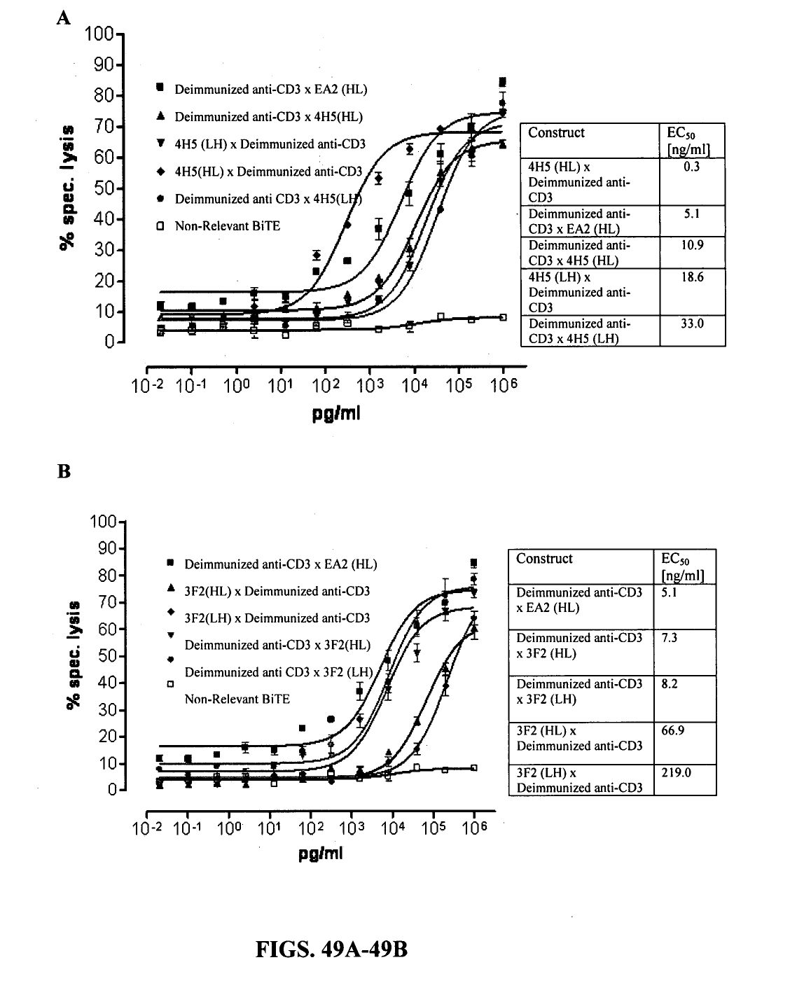

FIG. 49 provides a direct comparison of the potency of target cell lysis of the various 3F2- and 4H5-based EphA2 constructs. The Chromium-51-based cytotoxicity assays were performed as described above.

6.7.5.4. Cytotoxicity Activity Induced by Affinity Matured 4H5-Based EphA2-BiTEs 12E4, 2E7 and 2A4

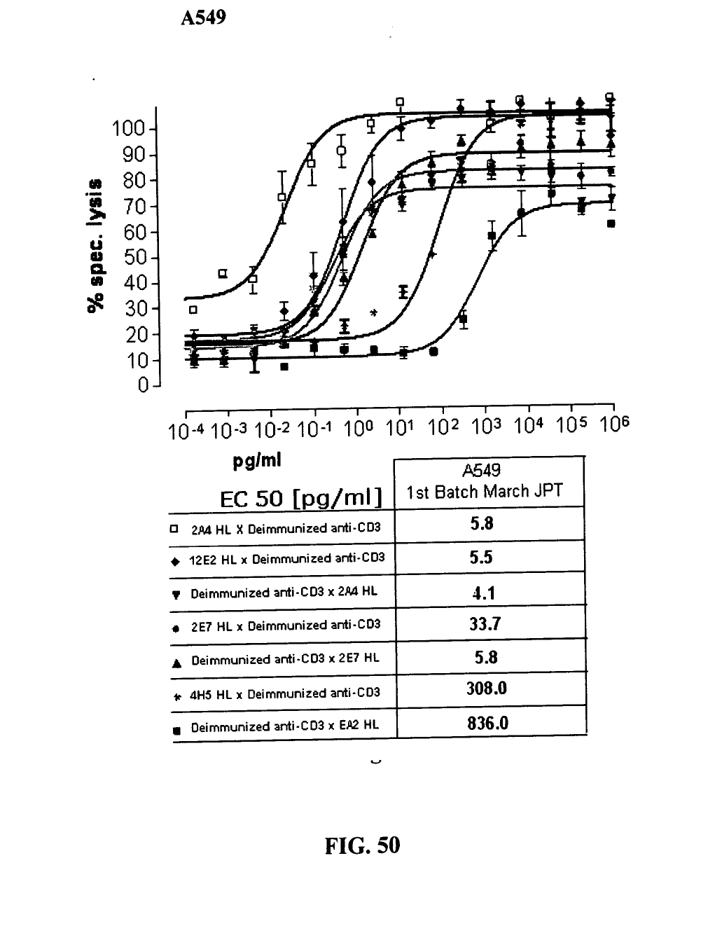

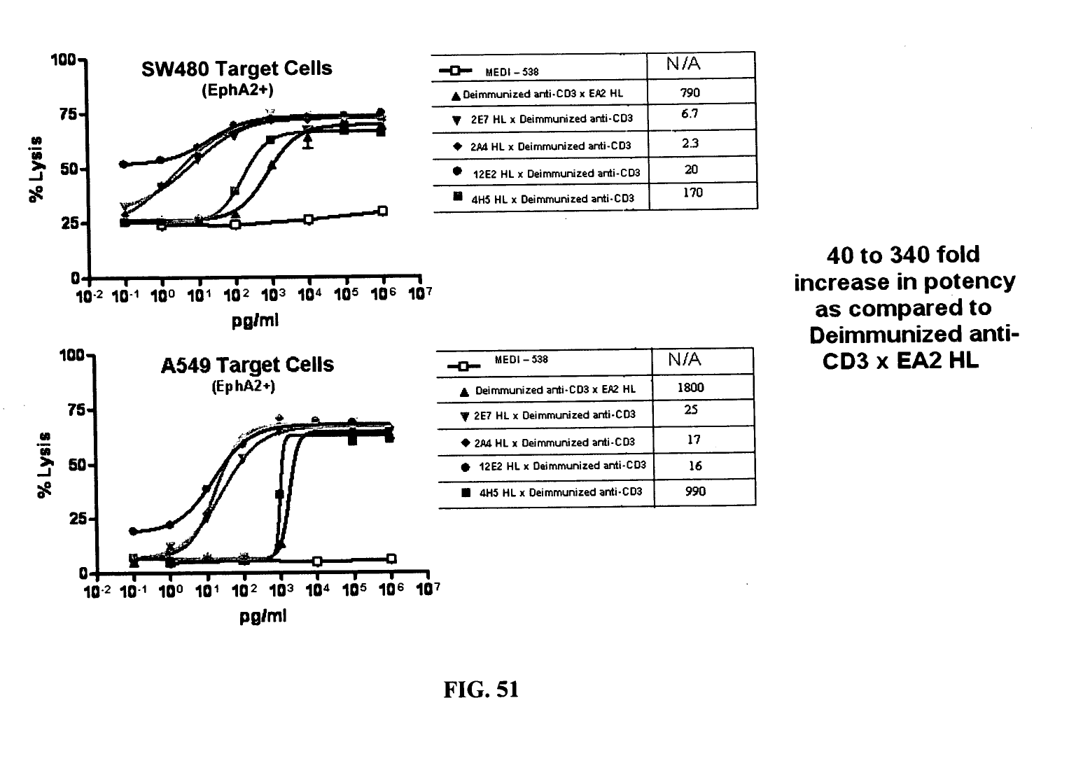

FIGS. 50 and 51 demonstrate the potency of target cell lysis of the various 4H5-based EphA2-BiTE constructs (12E4, 2E7 and 2A4). The EphA2-positive tumor cell lines A549 and SW480 were loaded with Chromium-51 and served as target cells. Stimulated human CD8+ (FIG. 50) and unstimulated human CD3+ (FIG. 51) T-cells served as effector cells. The various anti-EphA2 BiTE constructs were titrated over a broad range of concentrations. The assay duration was 18 hours (FIG. 50) or 42 hours (FIG. 51), and the effector-to-target ratio 10:1 (FIG. 50) or 5:1 (FIG. 51). EphA2-BiTE concentrations required for half-maximal lysis (i.e., EC50) with different production batches were estimated using a four-parameter non-linear fit model.

6.7.5.5. No Activation of EphA2 by 4H5-Based EphA2-BiTEs

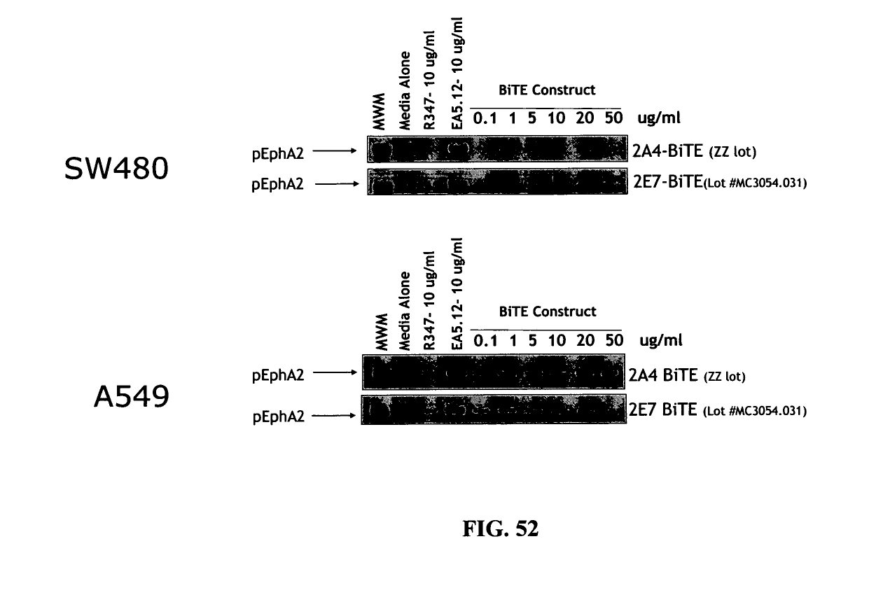

FIG. 52 demonstrates that neither the 2A4 nor the 2E7 BiTE construct induced EphA2 phosphorylation in EphA2-expressing cells. EphA2+SW480 and A549 cells were treated with either the 2A4-BiTE or the 2E7-BiTE, EA5 IgG (positive control), or R347 (negative control) for 15 min at the indicated concentrations. Cell extracts were then immunoprecipitated using the anti-EphA2 monoclonal antibody D7 (Upstate, Charlottesville, Va.) and probed using the anti-phosphotyrosine antibody 4G10 (Upstate). Sample extraction, immunoprecipitation and Western blot analyses were performed as detailed previously (Coffman K. et al., 2003, Cancer Res. 63:7907-12; and is incorporated by reference herein in its entirety). For all experiments, protein levels were measured using standard Coomassie assays (Pierce, Rockford, Ill.), and equal amounts of protein were resolved by SDS-PAGE (10%) and transferred to PVDF membranes (Invitrogen, Carlsbad, Calif.).

6.7.6 In Vivo Efficacy of Affinity Matured 4H5-Based EphA2-BiTE Constructs

The in vivo potency of the 2A4- and 2E7-BiTE constructs were evaluated in immunodeficient NOD/SCID mice with a human colon carcinoma xenograft model (described above in section 6.4 and below in section 6.8.7). The human colon carcinoma cell line SW480 was selected for the establishment of a human xenograft model since SW480 cells express EphA2.

6.7.6.1. Study Design

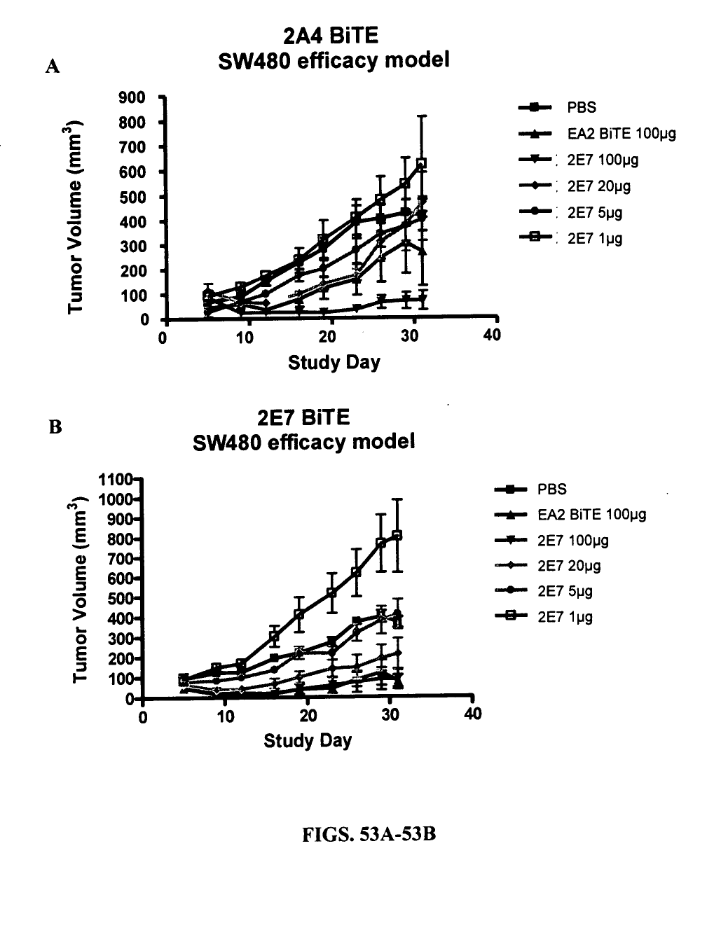

5×10⁶ SW480 cells were mixed with 2.5×10⁶ human CD3+ T-cells from the same donor in a final volume of 0.2 ml PBS resulting in an E:T an ratio of 1:2. The T-cell effector/SW480 cell mixtures were subcutaneously injected into the right flank of each NOD/SCID mouse. Subcutaneously growing SW480 tumors were measured three times a week with a caliper in two perpendicular dimensions and tumor volumes calculated according to the formula: tumor volume=[(width²*length)/2]. Animals were intravenously treated with the 2A4-BiTE or 2E7-BiTE at a concentration of 1, 5, 20 and 100 μg/dose, PBS control vehicle, or deimmunized anti-CD3×EA2(VHVL) at 100 μg/dose as a positive control for five consecutive days starting one hour after subcutaneous inoculation of CD3+ T-cell and SW480 tumor cells. The results of the study are depicted in FIG. 53.

6.8 Further Description of Assays Performed Herein

6.8.1 Cell Lines and Culture

CHO dhfr−, SW480, MCF-7, PC3, M14, A549, MDA-MB-231, MCF10A, MDA-MB-468, SK-MEL-28, ACHN cells were obtained from the ATCC and cultured according to their recommendations. HeyA8 was a kind gift from Dr. Anil Sood, M.D. Anderson Cancer Center.

6.8.2 Immunohistochemical and Immunofluorescence Microscopy

Frozen human tissue sections were stained with the human EphA2-reactive monoclonal antibody, EA2, to determine if the antibody specifically binds to normal tissue. Briefly, a complex of primary and secondary antibodies was produced, the unbound secondary binding sites were blocked with human gamma globulins. Frozen sections were adhered to slides in 10% formalin and rinsed with 1× Tris-buffered saline (TBS) with 0.01% Tween 20. Endogenous peroxidases were blocked with a solution containing glucose oxidase (Sigma), β-D(+)-glucose (Sigma), and sodium azide (Sigma). An avidin/biotin vector kit (Vector Labs) blocked avidin/biotin reactive sites. The slides were then incubated with a protein blocking solution consisting of bovine serum albumin, casein, and normal goat serum. Tissue sections were incubated with pre-complexed antibodies and then Vectastain ABC Elite Kit (Vector Labs), rinsed with 1×TBS, treated with DAB (Sigma), and counterstained with Mayer's hematoxylin. Tissue sections were dehydrated, coverslipped, and imaged.

6.8.3 Surface Plasmon Resonance Biosensor Analysis

All studies were performed using Sensor Chip CM5 (Biacore AB, Uppsala, Sweden) which contains a carboxymethyl (CM) dextran matrix and a Biacore® 3000 surface plasmon resonance (SPR) biosensor (Biacore AB, Uppsala, Sweden). CD3εγ was covalently attached to the CM dextran matrix using amine coupling chemistry. A reference surface was created by omission of the CD3εγ coupling step. EphA2-Fc was captured via a high-affinity interaction between the Fc portion of EphA2Fc and a goat anti-human IgG (Fc) (KPL, Inc., Gaithersburg, Md.). Goat anti-human IgG (Fc) was covalently attached to the CM dextran matrix using amine coupling chemistry. Two anti-human IgG (Fc)-specific surfaces were created. One of these surfaces was used as a reference surface while the other surface was used to create an EphA2-Fc-specific surface. Different concentrations of bscEphA2×CD3 were prepared by serial dilution in HBS-EP (0.01 M HEPES pH 7.4, 0.15 M NaCl, 3 mM EDTA, 0.005% surfactant P20). EphA2-BiTEs were injected in a serial-flow manner across the CD3εγ-specific or EphA2-specific surface and its corresponding reference surface. Dissociation of bound EphA2-BiTEs was monitored in the presence of HBS-EP. Remaining bound material was removed with 10 mM disodium tetraborate pH 8.5, 1 M NaCl (for CD3εγ-specific surface) or 10 mM glycine pH 1.7 (for EphA2-Fc-specific surface).

6.8.4 Cell Surface Antigen Density

The number of EphA2 surface binding sites on cells was estimated using Qifikit (DakoCytomation). Briefly, subconfluent monolayers of cells were quickly trypsinized, washed, counted, plated into 96-well round bottom plates, and stained with 100 μl of a two-fold serial dilution of the anti-human EphA2 antibody, B233 (Coffman 2003). Cells and mouse IgG-conjugated calibration beads were resuspended in AlexaFluor 647 anti-mouse IgG H+L (Invitrogen) and then analyzed by flow cytometry using a FACSCalibur (Becton Dickinson). The number of surface bindings sites was estimated by non-linear regression analysis from the bead calibration curve.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A bispecific single chain antibody comprising:
(a) a first heavy chain variable domain (VH domain) and a first light chain variable domain (VL domain) each from an antibody that immunospecifically binds to CD3, said first VH domain covalently linked to said first VL domain by a first linker of sufficient length such that said first VH domain and said first VL domain fold to form a first binding domain that binds to CD3; and
(b) a second VH domain and a second VL domain from an antibody that immunospecifically binds an epitope of EphA2 exposed on the cell surface, said second VH domain covalently linked to said second VL domain by a second linker of sufficient length such that said second VH domain and said second VL domain fold to form a second binding domain that binds said epitope of EphA2;
wherein said first binding domain and said second binding domain are covalently linked by a third linker of a sufficient length such that said first binding domain and said second binding domain fold independently of each other.

2. The bispecific single chain antibody of claim 1, wherein the first binding domain that immunospecifically binds to CD3 binds to the epsilon (ε) subunit of CD3.

3. The bispecific single chain antibody of claim 2, wherein the first binding domain specific for the ε subunit of CD3 is located N-terminally relative to the second binding domain.

4. The bispecific single chain antibody of claim 2, wherein the first binding domain specific for the ε subunit of CD3 is located C-terminally relative to the second binding domain.

5. The bispecific single chain antibody of claim 2, wherein the first binding domain and the second binding domain are arranged in the order VH_{CD3}-VL_{CD3}-VH_{EphA2}-VL_{EphA2}.

6. The bispecific single chain antibody of claim 2, wherein the first binding domain that immunospecifically binds to the ε subunit of CD3 is deimmunized.

7. The bispecific antibody of claim 2, wherein the VH and/or VL domains of the second binding domain are the VH and/or VL domains of EA2, EA3, EA4, EA5, 3F2, 4H5, 2A4, 2E7, 12E2, Eph099B-102.147, Eph099B-208.261, Eph099B-210.248, Eph099B-233.152, Eph1010.530.241, 233 or G5.

8. The bispecific single chain antibody of claim 2, wherein the length of the first, second and third linker sequences comprise at least 5 residues, at least 10 residues, at least 15 residues, at least 20 residues, at least 25 residues or at least 30 residues.

9. The bispecific single chain antibody of claim 2, wherein said first linker between the first heavy chain variable domain and the first light chain variable domain of said first binding domain that binds to the ε subunit of CD3 comprises the sequence SEQ ID NO:57.

10. The bispecific single chain antibody of claim 2, wherein said second linker between the second heavy chain variable domain and the second light chain variable domain of said second binding domain that binds to EphA2 comprises the sequence SEQ ID NO:59.

11. The bispecific single chain antibody of claim 2, wherein the third linker between said first binding domain and said second binding domain comprises the sequence SEQ ID NO:58.

12. The bispecific single chain antibody of claim 2, wherein the VH and/or VL domains of the first binding domain are from an anti-CD3 antibody that is humanized.

13. The bispecific single chain antibody of claim 2, wherein the VH and/or VL domains of the second binding domain are from an anti-EphA2 antibody that is humanized.

14. The bispecific single chain antibody of claim 2, wherein said first binding domain binds the ε subunit of CD3 with a lower affinity than said second binding domain binds EphA2.

15. The bispecific single chain antibody of claim 14, wherein the dissociation constant of the first binding domain that binds to ε subunit of CD3 is 4×10⁻⁷ M.

16. The bispecific single chain antibody of claim 14, wherein the dissociation constant of the second binding domain that binds to EphA2 is 1.13×10⁻⁷ M.

17. The bispecific single chain antibody of claim 2 which comprises the sequence of SEQ ID NO:65.

18. A composition comprising the bispecific single chain antibody of, claim 1, and a pharmaceutically acceptable carrier.

19. A method of treating, preventing or managing cancer in which the cancer cells express EphA2 in a subject in need thereof, said method comprising administering to said subject an effective amount of the bispecific single chain antibody of claim 1, and a pharmaceutically acceptable carrier.

20-23. (canceled)

24. A method of treating, preventing or managing an infection in a subject in need thereof, said method comprising administering to said subject an effective amount of the bispecific single chain antibody of claim 1, and a pharmaceutically acceptable carrier.

25. The method of claim 24, wherein said infection is an intracellular pathogen infection.

26-28. (canceled)

29. A method of treating, preventing or managing a non-cancer hyperproliferative cell disorder, said method comprising administering to said subject an effective amount of the bispecific single chain antibody of claim 1, and a pharmaceutically acceptable carrier.

30. The method of claim 29, wherein said non-cancer hyperproliferative cell disorder is asthma, COPD, lung fibrosis, asbestosis, IPF, DIP, UIP, kidney fibrosis, liver fibrosis, other fibroses, bronchial hyper-responsiveness, psoriasis, seborrheic dermatitis, cystic fibrosis, or a hyperproliferative endothelial cell disorder, such as restenosis, hyperproliferative vascular disease, Behcet's Syndrome, atherosclerosis, macular degeneration, or a hyperproliferative fibroblast disorder.

31-32. (canceled)

33. The method of claim 19, 24 or 29, wherein said subject is a human.


SEQUENCE LISTING (123 sequences)

SEQ ID NO: 1 - DNA, 321 bp, Artificial Sequence
  EA2 Variable Light Chain Nucleotide Sequence
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctgaaatatgatgagtttccgtacacgttcggaggggggaccaagctggaaataaaa

SEQ ID NO: 2 - protein, 107 aa, Artificial Sequence
  EA2 Variable Light Chain Amino Acid Sequence
DIKMTQSPSSMYASLGERVTITCKASQDINNYLSWFQQKPGKSPKTLIYRANRLVDGVPSRFSGSGSGQDYSLTISSLEYEDMGIYYCLKYDEFPYTFGGGTKLEIK

SEQ ID NO: 3 - DNA, 33 bp, Artificial Sequence
  EA2 Variable Light Chain CDR1 Nucleotide Sequence
aaggcgagtcaggacattaataactatttaagc

SEQ ID NO: 4 - protein, 11 aa, Artificial Sequence
  EA2 Variable Light Chain CDR1 Amino Acid Sequence
KASQDINNYLS

SEQ ID NO: 5 - DNA, 21 bp, Artificial Sequence
  EA2 Variable Light Chain CDR2 Nucleotide Sequence
cgtgcaaacagattggtagat

SEQ ID NO: 6 - protein, 7 aa, Artificial Sequence
  EA2 Variable Light Chain CDR2 Amino Acid Sequence
RANRLVD

SEQ ID NO: 7 - DNA, 27 bp, Artificial Sequence
  EA2 Variable Light Chain CDR3 Nucleotide Sequence
ctgaaatatgatgagtttccgtacacg

SEQ ID NO: 8 - protein, 9 aa, Artificial Sequence
  EA2 Variable Light Chain CDR3 Amino Acid Sequence
LKYDEFPYT

SEQ ID NO: 9 - DNA, 345 bp, Artificial Sequence
  EA2 Variable Heavy Chain Nucleotide Sequence
gacgtgaagctggtggagtctgggggaggcttagtgaagcctggagggtccctgaaactctcctgtgcagcctctggattcactttcagtagctataccatgtcttgggttcgccagactccggagaagaggctggagtgggtcgcaaccattagtagtggtggtacttacacctactatccagacagtgtgaagggccgattcaccatctccagagacaatgccaagaacaccctgtacctgcaaatgagcagtctgaagtctgaggacacagccatgtattactgtacaagagaagctatctttacttactggggccaagggactctggtcactgtctctgca

SEQ ID NO: 10 - protein, 115 aa, Artificial Sequence
  EA2 Variable Heavy Chain Amino Acid Sequence
DVKLVESGGGLVKPGGSLKLSCAASGFTFSSYTMSWVRQTPEKRLEWVATISSGGTYTYYPDSVKGRFTISRDNAKNTLYLQMSSLKSEDTAMYYCTREAIFTYWGQGTLVTVSA

SEQ ID NO: 11 - DNA, 30 bp, Artificial Sequence
  EA2 Variable Heavy Chain CDR1 Nucleotide Sequence
ggattcactttcagtagctataccatgtct

SEQ ID NO: 12 - protein, 10 aa, Artificial Sequence
  EA2 Variable Heavy Chain CDR1 Amino acid Sequence
GFTFSSYTMS

SEQ ID NO: 13 - DNA, 48 bp, Artificial Sequence
  EA2 Variable Heavy Chain CDR2 Nucleotide Sequence
accattagtagtggtggtacttacacctactatccagacagtaagggc

SEQ ID NO: 14 - protein, 17 aa, Artificial Sequence
  EA2 Variable Heavy Chain CDR2 Amino acid Sequence
TISSGGTYTYYPDSVKG

SEQ ID NO: 15 - DNA, 18 bp, Artificial Sequence
  EA2 Variable Heavy Chain CDR3 Nucleotide Sequence
gaagctatctttacttac

SEQ ID NO: 16 - protein, 6 aa, Artificial Sequence
  EA2 Variable Heavy Chain CDR3 Amino Acid Sequence
EAIFTY

SEQ ID NO: 17 - DNA, 336 bp, Artificial Sequence
  EA5.12 Light Chain Variable Nucleotide Sequence
gatgtkgtkatgacbcagactccactcactttgtcggttaccattggacaaccagcctctatctcttgcaagtcaagtcagagcctcttatatagtaatggaaaaacctatttgaattggttgttacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggttcacattttccgtggacgttcggtggaggcaccaagctggaaatcaaa

SEQ ID NO: 18 - protein, 112 aa, Artificial Sequence
  EA5.12 Light Chain Variable  Amino Acid Sequence
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGSHFPWTFGGGTKLEIK

SEQ ID NO: 19 - DNA, 27 bp, Artificial Sequence
  EA5.12 Light Chain Variable CDR1 Nucleotide Sequence
aagtcaagtcagagcctcttatatagt

SEQ ID NO: 20 - protein, 16 aa, Artificial Sequence
  EA5.12 Light Chain Variable CDR1 Amino Acid Sequence
KSSQSLLYSNGKTYLN

SEQ ID NO: 21 - DNA, 21 bp, Artificial Sequence
  EA5.12 Light Chain Variable CDR2 Nucleotide Sequence
ctggtgtctaaactggactct

SEQ ID NO: 22 - protein, 7 aa, Artificial Sequence
  EA5.12 Light Chain Variable CDR2 Amino Acid Sequence
LVSKLDS

SEQ ID NO: 23 - DNA, 27 bp, Artificial Sequence
  EA5.12 Light Chain Variable CDR3 Nucleotide Sequence
gtgcaaggttcacattttccgtggacg

SEQ ID NO: 24 - protein, 9 aa, Artificial Sequence
  EA5.12 Light Chain Variable CDR3 Amino Acid Sequence
VQGSHFPWT

SEQ ID NO: 25 - DNA, 345 bp, Artificial Sequence
  EA5.12 Heavy Chain Variable Nucleotide Sequence
gaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgttactagctacaaccagaagttcaagggcaaggccacatttactgtagacacatcctccagcacagcctacatgcagttcaacagcctgacatctgaagactctgcggtctattactgtgcaagatctcatgctatggactactggggtcaaggaacctcagtcaccgtctcctca

SEQ ID NO: 26 - protein, 115 aa, Artificial Sequence
  EA5.12 Heavy Chain Variable Region - Amino Acid Sequence
EVQLQQSGPELVKTGASVKISCKASGYSFTGYYMHWVKQSHGKSLEWIGYISCYNGVTSYNQKFKGKATFTVDTSSSTAYMQFNSLTSEDSAVYYCARSHAMDYWGQGTSVTVSS

SEQ ID NO: 27 - DNA, 15 bp, Artificial Sequence
  EA5.12 Heavy Chain Variable CDR1 Nucleotide Sequence
ggttactacatgcac

SEQ ID NO: 28 - protein, 5 aa, Artificial Sequence
  EA5.12 Heavy Chain Variable CDR1 Amino Acid Sequence
GYYMH

SEQ ID NO: 29 - DNA, 51 bp, Artificial Sequence
  EA5.12 Heavy Chain Variable CDR2 Nucleotide Sequence
tatattagttgttacaatggtgttactagctacaaccagaagttcaagggc

SEQ ID NO: 30 - protein, 17 aa, Artificial Sequence
  EA5.12 Heavy Chain Variable CDR2 Amino Acid Sequence
YISCYNGVTSYNQKFKG

SEQ ID NO: 31 - DNA, 18 bp, Artificial Sequence
  EA5.12 Heavy Chain Variable CDR3 Nucleotide Sequence
tctcatgctatggactac

SEQ ID NO: 32 - protein, 6 aa, Artificial Sequence
  EA5.12 Heavy Chain Variable CDR3 Amino Acid Sequence
SHAMDY

SEQ ID NO: 33 - protein, 123 aa, Artificial Sequence
  233 Variable Light Chain Amino Acid Sequence
DIVLTQSPATLSVTPGDIVLTQSPATLSVTPGDSVNLSCRASQSISNNLHWYQQKSHESPRLLIKYVFQSISGIPSRFSGSGSGTDFTLSINSVETEDFGMYFCQQSNSWPLTFGAGTKLELK

SEQ ID NO: 34 - protein, 11 aa, Artificial Sequence
  233 Variable Light Chain CDR1 Amino Acid Sequence
RASQSISNNLH

SEQ ID NO: 35 - protein, 7 aa, Artificial Sequence
  233 Variable Light Chain CDR2 Amino Acid Sequence
YVFQSIS

SEQ ID NO: 36 - protein, 9 aa, Artificial Sequence
  233 Variable Light Chain CDR3 Amino Acid Sequence
QQSNSWPLT

SEQ ID NO: 37 - protein, 120 aa, Artificial Sequence
  233 Variable Heavy Chain Amino Acid Sequence
EVKLVESGGGLVQPGGSLSLSCAASGFTFTDYSMNWVRQPPGKALEWLGFIRNKANDYTTEYSASVKGRFTISRDNSQSILYLQMNALRAEDSATYYCVRYPRYHAMDSWGQGTSVTVSS

SEQ ID NO: 38 - protein, 5 aa, Artificial Sequence
  233 Variable Heavy Chain CDR1 Amino Acid Sequence
DYSMN

SEQ ID NO: 39 - protein, 19 aa, Artificial Sequence
  233 Variable Heavy Chain CDR2 Amino Acid Sequence
FIRNKANDYTTEYSASVKG

SEQ ID NO: 40 - protein, 9 aa, Artificial Sequence
  233 Variable Heavy Chain CDR3 Amino Acid Sequence
YPRYHAMDS

SEQ ID NO: 41 - protein, 107 aa, Artificial Sequence
  3F2 Variable Light Chain Amino Acid Sequence
AIQLTQSPSSLSASVGDRVTITCRASQSISNNLHWYLQKPGQSPQLLIYYGFQSISGVPSRFSGSGSGTDFTLTISSLQPEDFATYYCQQANSWPLTFGGGTKLEIK

SEQ ID NO: 42 - protein, 11 aa, Artificial Sequence
  3F2 Variable Light Chain CDR1 Amino Acid Sequence
RASQSISNNLH

SEQ ID NO: 43 - protein, 7 aa, Artificial Sequence
  3F2 Variable Light Chain CDR2 Amino Acid Sequence
YGFQSIS

SEQ ID NO: 44 - protein, 9 aa, Artificial Sequence
  3F2 Variable Light Chain CDR3 Amino Acid Sequence
QQANSWPLT

SEQ ID NO: 45 - protein, 120 aa, Artificial Sequence
  3F2 Variable Heavy Chain Amino Acid Sequence
EVQLVESGGGVVRPGGSLRLSCAASGFTVSDYSMNWVRQAPGKGLEWIGFIRNKANAYTTEYSASVKGRFTISRDDSKNTLYLQMNSLKTEDTAVYYCTTYPRYHAMDSWGQGTMVTVSS

SEQ ID NO: 46 - protein, 5 aa, Artificial Sequence
  3F2 Variable Heavy Chain CDR1 Amino Acid Sequence
DYSMN

SEQ ID NO: 47 - protein, 19 aa, Artificial Sequence
  3F2 Variable Heavy Chain CDR2 Amino Acid Sequence
FIRNKANAYTTEYSASVKG

SEQ ID NO: 48 - protein, 9 aa, Artificial Sequence
  3F2 Variable Heavy Chain CDR3 Amino Acid Sequence
YPRYHAMDS

SEQ ID NO: 49 - protein, 107 aa, Artificial Sequence
  G5 Variable Light Chain Amino Acid Sequence
DIQMTQSPSSLSASVGDRVTITCRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 50 - protein, 11 aa, Artificial Sequence
  G5 Variable Light Chain CDR1 Amino Acid Sequence
RASQSISNNLH

SEQ ID NO: 51 - protein, 7 aa, Artificial Sequence
  G5 Variable Light Chain CDR2 Amino Acid Sequence
YVFQSIS

SEQ ID NO: 52 - protein, 9 aa, Artificial Sequence
  G5 Variable Light Chain CDR3 Amino Acid Sequence
QQSNSWPLT

SEQ ID NO: 53 - protein, 120 aa, Artificial Sequence
  G5 Variable Heavy Chain Amino Acid Sequence
QMQLVQSGPEVKKPGTSVKVSCKASGFTFTDYSMNWVRQARGQRLEWIGFIRNKANDYTTEYSASVKGRVTITRDMSTSTAYMELSSLRSEDTAVYYCARYPRYHAMDSWGQGTSVTVSS

SEQ ID NO: 54 - protein, 5 aa, Artificial Sequence
  G5 Variable Heavy Chain CDR1 Amino Acid Sequence
DYSMN

SEQ ID NO: 55 - protein, 19 aa, Artificial Sequence
  G5 Variable Heavy Chain CDR2 Amino Acid Sequence
FIRNKANDYTTEYSASVKG

SEQ ID NO: 56 - protein, 9 aa, Artificial Sequence
  G5 Variable Heavy Chain CDR3 Amino Acid Sequence
YPRYHAMDS

SEQ ID NO: 57 - protein, 18 aa, Artificial Sequence
  Amino Acid Linker Sequence of EphA2-BiTe construct
GEGTSTGSGGSGGSGGAD

SEQ ID NO: 58 - protein, 5 aa, Artificial Sequence
  Amino Acid Linker Sequence of EphA2-BiTe construct
GGGGS

SEQ ID NO: 59 - protein, 15 aa, Artificial Sequence
  Amino Acid Linker Sequence of EphA2-BiTe construct
GGGGSGGGGSGGGGS

SEQ ID NO: 60 - DNA, 1554 bp, Artificial Sequence
  Deimmunized anti-CD3 (VH-Vl) x EA2 (VH-VL) Nucleotide Sequence
gaattcaccatgggatggagctgtatcatcctcttcttggtagcaacagctacaggtgtacactccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatccggaggtggtggatccgacgtgaagctggtggagtctgggggaggcttagtgaagcctggagggtccctgaaactctcctgtgcagcctctggattcactttcagtagctataccatgtcttgggttcgccagactccagagaagaggctggagtgggtcgcaaccattagtagtggtggtacttacacctactatccagacagtgtgaagggccgattcaccatctccagagacaatgccaagaacaccctgtacctgcaaatgagcagtctgaagtctgaggacacagccatgtattactgtacaagagaagctatctttacttactggggccaagggactctggtcactgtctcttcctccggtggtggtggttctggcggcggcggctccggtggtggtggttctgacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctgaaatatgatgagtttccgtacacgttcggaggggggaccaagctggaaataaaacatcatcaccatcatcattaggtcgac

SEQ ID NO: 61 - DNA, 55 bp, Artificial Sequence
  Nucleotide Linker Sequence of EphA2-BiTE construct
ggcgaaggtactagtactggttctggtggaagtggaggttcaggtggagcagacg

SEQ ID NO: 62 - DNA, 19 bp, Artificial Sequence
  Nucleotide Linker Sequence of EphA2-BiTE construct
tccggaggtggtggatccg

SEQ ID NO: 63 - DNA, 48 bp, Artificial Sequence
  Nucleotide Linker Sequence of EphA2-BiTe construct
tccggtggtggtggttctggcggcggcggctccggtggtggtggttct

SEQ ID NO: 64 - DNA, 21 bp, Artificial Sequence
  Nucleotide Linker Sequence of EphA2-BiTE construct
catcatcaccatcatcattag

SEQ ID NO: 65 - protein, 492 aa, Artificial Sequence
  Amino Acid Sequence of Deimmunized anti-CD3 (VH-VL) x EA2 (VH-VL) EphA2-Bite
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSDVKLVESGGGLVKPGGSLKLSCAASGFTFSSYTMSWVRQTPEKRLEWVATISSGGTYTYYPDSVKGRFTISRDNAKNTLYLQMSSLKSEDTAMYYCTREAIFTYWGQGTLVTVSSGGGGSGGGGSGGGGSDIKMTQSPSSMYASLGERVTITCKASQDINNYLSWFQQKPGKSPKTLIYRANRLVDGVPSRFSGSGSGQDYSLTISSLEYEDMGIYYCLKYDEFPYTFGGGTKLEIKHHHHHH

SEQ ID NO: 66 - protein, 6 aa, Artificial Sequence
  C-terminal hexa-histidine sequence of EphA2-BiTe cDNA
HHHHHH

SEQ ID NO: 67 - DNA, 711 bp, Artificial Sequence
  4h5 VH VL scFV Nucleotide sequence
caggtgcagctgttggagtctgggggaggcttggtacagcctggggggtccctgagactctcctgtgcagcctctggattcacctttagcagctataccatgtcttgggtgcgacaggcccctggacaagcgcttgagtggatgggaaccattagtagtggtggtacttacacctactatccagacagtgtgaagggccgattcaccatctccagagacaacgccaagaactcactgtatctgcaaatgaacagcctgagagccgaggacacggctgtgtattactgtgcgagagaagctatctttacttactggggccgtggcaccctggtcaccgtctcctcaggtggtggtggttctggcggcggcggctccggtggtggtggttctgacatccagttgacccagtctccatcctccctgtctgcatctgtaggagacagagtcaccatcacttgcaaggcgagtcaggacattaataactatttaagctggtaccagcagaaacctggccaggctcccaggctcctcatctatcgtgcaaacagattggtagatggggtcccagacaggttcagtggcagcgggtatggaacagattttaccctcacaattaataacatagaatctgaggatgctgcatattacttctgtctgaaatatgatgtgtttccgtacacgttcggccaagggaccaaggtggagatcaaa

SEQ ID NO: 68 - protein, 237 aa, Artificial Sequence
  4H5 VH VL scFv Amino Acid Sequence
QVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTVSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 69 - protein, 5 aa, Artificial Sequence
  4H5 VH VL scFv CDR1 (VH) Amino Acid Sequence
SYTMS

SEQ ID NO: 70 - protein, 17 aa, Artificial Sequence
  4H5 VH VL scFv CDR2 (VH) Amino Acid Sequence
TISSGGTYTYYPDSVKG

SEQ ID NO: 71 - protein, 6 aa, Artificial Sequence
  4H5 VH VL scFv CDR3 (VH) Amino Acid Sequence
EAIFTY

SEQ ID NO: 72 - protein, 11 aa, Artificial Sequence
  4H5 VH VL scFv CDR1 (VL) Amino Acid Sequence
KASQDINNYLS

SEQ ID NO: 73 - protein, 7 aa, Artificial Sequence
  4H5 VH VL scFv CDR2 (VL) Amino Acid Sequence
RANRLVD

SEQ ID NO: 74 - protein, 9 aa, Artificial Sequence
  4H5 VH VL scFv CDR3 (VL) Amino Acid Sequence
LKYDVFPYT

SEQ ID NO: 75 - DNA, 711 bp, Artificial Sequence
  2A4 VH VL scFv Nucleotide Sequence
caggtgcagctgttggagtctgggggaggcttggtacagcctggggggtccctgagactctcctgtgcagcctctggattcacctttagcagctataccatgtcttgggtgcgacaggcccctggacaagcgcttgagtggatgggaaccattagtagtcgtggtacttacacctactatccagacagtgtgaagggccgattcaccatctccagagacaacgccaagaactcactgtatctgcaaatgaacagcctgagagccgaggacacggctgtgtattactgtgcgagagaagctatctttactcactggggccgtggcaccctggtcaccgtctcctcaggtggtggtggttctggcggcggcggctccggtggtggtggttctgacatccagttgacccagtctccatcctccctgtctgcatctgtaggagacagagtcaccatcacttgcaaggcgagtcaggacattaataactatcacagctggtaccagcagaaacctggccaggctcccaggctcctcatctatcgtgcaaacagattggtcgatggggtcccagacaggttcagtggcagcgggtatggaacagattttaccctcacaattaataacatagaatctgaggatgctgcatattacttctgtctgaaatataatgtgtttccgtacacgttcggccaagggaccaaggtggagatcaaa

SEQ ID NO: 76 - protein, 237 aa, Artificial Sequence
  2A4 VH VL scFv Amino Acid Sequence
QVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSSGGGGSGGGGSGGGGSDIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIK

SEQ ID NO: 77 - protein, 5 aa, Artificial Sequence
  2A4 VH VL scFv CDR1 (VH) Amino Acid Sequence
SYTMS

SEQ ID NO: 78 - protein, 17 aa, Artificial Sequence
  2A4 VH VL scFv CDR2 (VH) Amino Acid Sequence
TISSRGTYTYYPDSVKG

SEQ ID NO: 79 - protein, 6 aa, Artificial Sequence
  2A4 VH VL scFv CDR3 (VH) Amino Acid Sequence
EAIFTH

SEQ ID NO: 80 - protein, 11 aa, Artificial Sequence
  2A4 VH VL scFv CDR1 (VL) Amino Acid Sequence
KASQDINNYHS

SEQ ID NO: 81 - protein, 7 aa, Artificial Sequence
  2A4 VH VL scFv CDR2 (VL) Amino Acid Sequence
RANRLVD

SEQ ID NO: 82 - protein, 9 aa, Artificial Sequence
  2A4 VH VL scFv CDR3 (VL) Amino Acid Sequence
LKYNVFPYT

SEQ ID NO: 83 - DNA, 711 bp, Artificial Sequence
  2E7 VH VL scFv Nucleotide Sequence
caggtgcagctgttggagtctgggggaggcttggtacagcctggggggtccctgagactctcctgtgcagcctctggattcacctttagcagctataccatgtcttgggtgcgacaggcccctggacaagcgcttgagtggatgggaaccattagtagtcgtggtacttacacctactatccagacagtgtgaagggccgattcaccatctccagagacaacgccaagaactcactgtatctgcaaatgaacagcctgagagccgaggacacggctgtgtattactgtgcgagagaagctatctttactcactggggccgtggcaccctggtcaccgtctcctcaggtggtggtggttctggcggcggcggctccggtggtggtggttctgacatccagttgacccagtctccatcctccctgtctgcatctgtaggagacagagtcaccatcacttgcaaggcgagtcaggacattaataactatggcagctggtaccagcagaaacctggccaggctcccaggctcctcatctatcgtgcaaacagattggtcgatggggtcccagacaggttcagtggcagcgggtatggaacagattttaccctcacaattaataacatagaatctgaggatgctgcatattacttctgtctgaaatataatcggtttccgtacacgttcggccaagggaccaaggtggagatcaaa

SEQ ID NO: 84 - protein, 333 aa, Artificial Sequence [sequence omitted]

SEQ ID NO: 85 - protein, 5 aa, Artificial Sequence
  2E7 VH VL scFv CDR1 (VH) Amino Acid Sequence
SYTMS

SEQ ID NO: 86 - protein, 17 aa, Artificial Sequence
  2E7 VH VL scFv CDR2 (VH) Amino Acid Sequence
TISSRGTYTYYPDSVKG

SEQ ID NO: 87 - protein, 6 aa, Artificial Sequence
  2E7 VH VL scFv CDR3 (VH) Amino Acid Sequence
EAIFTH

SEQ ID NO: 88 - protein, 11 aa, Artificial Sequence
  2E7 VH VL scFv CDR1 (VL) Amino Acid Sequence
KASQDINNYGS

SEQ ID NO: 89 - protein, 7 aa, Artificial Sequence
  2E7 VH VL scFv CDR2 (VL) Amino Acid Sequence
RANRLVD

SEQ ID NO: 90 - protein, 9 aa, Artificial Sequence
  2E7 VH VL scFv CDR3 (VL) Amino Acid Sequence
LKYNRFPYT

SEQ ID NO: 91 - DNA, 711 bp, Artificial Sequence
  12E2 VH VL scFv Nucleotide Sequence
caggtgcagctgttggagtctgggggaggcttggtacagcctggggggtccctgagactctcctgtgcagcctctggattcacctttagcagctataccatgtcttgggtgcgacaggcccctggacaagcgcttgagtggatgggaaccattagtagtcgtggtacttacacctactatccagacagtgtgaagggccgattcaccatctccagagacaacgccaagaactcactgtatctgcaaatgaacagcctgagagccgaggacacggctgtgtattactgtgcgagagaagctatctttacttactggggccgtggcaccctggtcaccgtctcctcaggtggtggtggttctggcggcggcggctccggtggtggtggttctgacatccagttgacccagtctccatcctccctgtctgcatctgtaggagacagagtcaccatcacttgcaaggcgagtcaggacattaataactatttaagctggtaccagcagaaacctggccaggctcccaggctcctcatctatcgtgcaaacagattgttcgatggggtcccagacaggttcagtggcagcgggtatggaacagattttaccctcacaattaataacatagaatctgaggatgctgcatattacttctgtctgaaatatgatcggtttccgtacacgttcggccaagggaccaaggtggagatcaaa

SEQ ID NO: 92 - protein, 237 aa, Artificial Sequence
  12E2 VH VL scFv Amino Acid Sequence
QVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTYWGRGTLVTVSSGGGGSGGGGSGGGGSDIQLTQSPSSLSASVGDRVTITCKASQDINNYLSWYQQKPGQAPRLLIYRANRLFDGVPDRFSGSGYGTDFTLTINNIESEDAAYYFCLKYDRFPYTFGQGTKVEIK

SEQ ID NO: 93 - protein, 5 aa, Artificial Sequence
  12E2 VH VL scFv CDR1 (VH) Amino Acid Sequence
SYTMS

SEQ ID NO: 94 - protein, 17 aa, Artificial Sequence
  12E2 VH VL scFv CDR2 (VH) Amino Acid Sequence
TISSRGTYTYYPDSVKG

SEQ ID NO: 95 - protein, 6 aa, Artificial Sequence
  12E2 VH VL scFv CDR3 (VH) Amino Acid Sequence
EAIFTY

SEQ ID NO: 96 - protein, 11 aa, Artificial Sequence
  12E2 VH VL scFv CDR1 (VL) Amino Acid Sequence
KASQDINNYLS

SEQ ID NO: 97 - protein, 7 aa, Artificial Sequence
  12E2 VH VL scFv CDR2 (VL) Amino Acid Sequence
RANRLFD

SEQ ID NO: 98 - protein, 9 aa, Artificial Sequence
  12E2 VH VL scFv CDR3 (VL) Amino Acid Sequence
LKYDRFPYT

SEQ ID NO: 99 - DNA, 33 bp, Artificial Sequence
  Combinatorial Primer L1 used in affinity optimization of Fab 2G6
ccagtgtaggttgttgctaatactttggctggc

SEQ ID NO: 100 - DNA, 33 bp, Artificial Sequence
  Combinatorial Primer L2 used in affinity optimization of Fab 2G6
ccagtgtaggttgttgtdaatactttggctggc

SEQ ID NO: 101 - DNA, 42 bp, Artificial Sequence
  Combinatorial Primer L3 used in affinity optimization of Fab 2G6
gggaccccagagatgdactggaagvcatacttgatcaggagc

SEQ ID NO: 102 - DNA, 42 bp, Artificial Sequence
  Combinatorial Primer L4 used in affinity optimization of Fab 2G6
gggaccccagagatgdactggaagstatacttgatcaggagc

SEQ ID NO: 103 - DNA, 35 bp, Artificial Sequence
  Combinatorial Primer L5 used in affinity optimization of Fab 2G6
gagcggccagctgttggmctgttgacagtaatatg

SEQ ID NO: 104 - DNA, 46 bp, Artificial Sequence
  Combinatorial Primer H6 used in affinity optimization of Fab 2G6
gcctgtcgcacccasttcatggagtaatcggwaaaggtgaatccag

SEQ ID NO: 105 - protein, 46 aa, Artificial Sequence
  Combinatorial Primer H7 used in affinity optimization of Fab 2G6
GCCTGTCGCACCCAGGWCATGGAGTAATCGGWAAAGGTGAATCCAG

SEQ ID NO: 106 - DNA, 46 bp, Artificial Sequence
  Combinatorial Primer H8 used in affinity optimization of Fab 2G6
gcctgtcgcacccasttcatggagtaatcgtcaaaggtgaatccag

SEQ ID NO: 107 - DNA, 46 bp, Artificial Sequence
  Combinatorial Primer H9 used in affinity optimization of Fab 2G6
gcctgtcgcacccaggwcatggagtaatcgtcaaaggtgaatccag

SEQ ID NO: 108 - protein, 49 aa, Artificial Sequence
  Combinatorial Primer H10 used in affinity optimization of Fab 2G6
GATGCACTGTACTCTGTTGTGTAGKCATTAGCTTTGTTTCTAATAAATC

SEQ ID NO: 109 - DNA, 49 bp, Artificial Sequence
  Combinatorial Primer H11 used in affinity optimization of Fab 2G6
gatgcactgtactctgttgtgtaggaattagctttgtttctaataaatc

SEQ ID NO: 110 - DNA, 45 bp, Artificial Sequence
  Combinatorial Primer H12 used in affinity optimization of Fab 2G6
gttccttggccccaggagtccatagcatgatrcctagggtatctc

SEQ ID NO: 111 - DNA, 45 bp, Artificial Sequence
  Combinatorial Primer H13 used in affinity optimization of Fab 2G6
gttccttggccccacaggtccatagcatgatrcctagggtatctc

SEQ ID NO: 112 - protein, 120 aa, Artificial Sequence
  Humanized 2G6 Variable Heavy Chain Amino Acid Sequence
EVQLVESGGGVVRPGGSLRLSCAASGFTVSDYSMNWVRQAPGKGLEWIGFIRNKANDYTTEYSASVKGRFTISRDDSKNTLYLQMNSLKTEDTAVYYCTTYPRYHAMDSWGQGTMVTVSS

SEQ ID NO: 113 - protein, 5 aa, Artificial Sequence
  Humanized 2G6 Variable Heavy Chain CDR1 Amino Acid Sequence
DYSMN

SEQ ID NO: 114 - protein, 19 aa, Artificial Sequence
  Humanized 2G6 Variable Heavy Chain CDR2 Amino Acid Sequence
FIRNKANDYTTEYSASVKG

SEQ ID NO: 115 - protein, 9 aa, Artificial Sequence
  Humanized 2G6 Variable Heavy Chain CDR3 Amino Acid Sequence
YPRYHAMDS

SEQ ID NO: 116 - protein, 106 aa, Artificial Sequence
  Humanized 2G6 Variable Light Chain Amino Acid Sequence
IQLTQSPSSLSASVGDRVTITCRASQSISNNLHWYLQKPGQSPQLLIYYVFQSISGVPSRFSGSGSGTDFTLTISSLQPEDFATYYCQQSNSWPLTFGGGTKLEIK

SEQ ID NO: 117 - protein, 11 aa, Artificial Sequence
  Humanized 2G6 Variable Light Chain CDR1 Amino Acid Sequence
RASQSISNNLH

SEQ ID NO: 118 - protein, 7 aa, Artificial Sequence
  Humanized 2G6 Variable Light Chain CDR2 Amino Acid Sequence
YVFQSIS

SEQ ID NO: 119 - protein, 9 aa, Artificial Sequence
  Humanized 2G6 Variable Light Chain CDR3 Amino Acid Sequence
QQSNSWPLT

SEQ ID NO: 120 - DNA, 43 bp, Artificial Sequence
  Medi-VH8 5′ primer used to amplify VH
ttctatgcggcccagccggcccaggtgcagctgttgsagtctg

SEQ ID NO: 121 - DNA, 54 bp, Artificial Sequence
  Medi-JH1 3′ primer used to amplify VH
ggagccgccgccgccagaaccaccaccacctgaggagacggtgaccagggtgcc

SEQ ID NO: 122 - DNA, 53 bp, Artificial Sequence
  Medi-VK1 5′ primer used for VL
ggcggcggcggctccggtggtggtggttctgacatccagwtgacccagtctcc

SEQ ID NO: 123 - DNA, 45 bp, Artificial Sequence
  Medi-JK4 3′ primer used for VL
tggaattcggcccccgaggccacgtttgatctccaccttggtccc